(19) United States
(12) Patent Application Publication
(10) Pub. No.: US 2004/0010815 A1
(43) Pub. Date: Jan. 15, 2004

(54) Identification and characterization of plant genes

(76) Inventors: B. Markus Lange, San Diego, CA (US); Majid Ghassemian, Carlsbad, CA (US); Steven P. Briggs, Del Mar, CA (US); Bret Cooper, La Jolla, CA (US); Jane Glazebrook, San Diego, CA (US); Stephen Arthur Goff, Encinitas, CA (US); Fumiaki Katagiri, San Diego, CA (US); Joel Kreps, Carlsbad, CA (US); Todd Moughamer, San Diego, CA (US); Nicholas Provart, Toronto (CA); Darrell Ricke, San Diego, CA (US); Tong Zhu, San Diego, CA (US)

Correspondence Address: TORREY MESA RESEARCH INSTITUTE, INTELLECTUAL PROPERTY DEPARTMENT, 3115 MERRYFIELD ROW, SAN DIEGO, CA, 92121, US

(21) Appl. No.: 10/259,194

(22) Filed: Sep. 26, 2002

Related U.S. Application Data
(60) Provisional application No. 60/325,277, filed on Sep. 26, 2001.
(60) Provisional application No. 60/370,743, filed on Apr. 4, 2002.
(60) Provisional application No. 60/370,620, filed on Apr. 4, 2002.
(60) Provisional application No. 60/325,277, filed on Sep. 26, 2001.

(51) Int. Cl. A01H 1/00; C07H 21/04; C12N 9/10; C12N 15/82

(52) U.S. Cl. 800/278; 536/23.2; 435/193; 435/419

(57) ABSTRACT

Disclosed are polynucleotide and polypeptide sequences involved in or associated with isoprenoid biosynthesis in plants. Also disclosed are uses for such sequences.

### CROSS REFERENCE TO RELATED APPLICATIONS

This application claims the benefits of U.S. Provisional Application Serial No. 60/325,277 filed Sep. 26, 2001, U.S. Provisional Application Serial No. 60/370,743 filed Apr. 4, 2002, and U.S. Provisional Application Serial No. 60/370,620 filed Apr. 4, 2002, each of which is incorporated herein by reference in its entirety for all purposes

### REFERENCE TO MATERIAL SUBMITTED ON COMPACT DISC

The sequence listing accompanying this application is contained on compact disc. The material on the CD-ROM (filed in duplicate herewith), on CD volume labeled “Copy 1” and “Copy 2”, each containing a text file named “70029NP_SEQ_LST” created Sep. 26, 2002, having a size of 1.46 MB, is hereby incorporated by reference in its entirety pursuant to 37 C.F.R. §1.52(e)(5).

### BACKGROUND

The present invention is in the area of plant biotechnology. In particular, the invention relates to a set of genes the expression products of which are involved in isoprenoid metabolism.

The isoprenoid biosynthetic pathway provides intermediates for the synthesis of a multitude of natural products which serve numerous biochemical functions in plants: sterols (isoprenoids with a C30 backbone) are essential components of membranes; carotenoids (C40) and chlorophylls (which contain a C20 isoprenoid side chain) act as photosynthetic pigments; plastoquinone, phylloquinone and ubiquinone (all of which contain long-chain isoprenoid side chains) participate in electron transport chains; gibberellins (C20), brassinosteroids (C30) and abscisic acid (C15) are phytohormones derived from isoprenoid intermediates; prenylation of proteins (with C15 or C20 isoprenoid moieties) is involved in subcellular targeting and regulation of activity; and several monoterpenes (C 10), sesquiterpenes (C 15) and diterpenes (C20) have been demonstrated to be involved in plant defense.

Conceptually, isoprenoid biosynthesis can be divided into four stages. Stage 1 includes the formation of isopentenyl diphosphate (IPP) and dimethylallyl alcohol (DMAPP). In plants, two separate pathways are utilized for the synthesis of these universal C5 intermediates (Lange et al., Proc Natl. Acad. Sci. USA, 97:13172-13177, 2000): the mevalonate pathway, which is responsible for the synthesis of certain sesquiterpenes, sterols (Nes and Venkatrarnesh, Crit. Rev. Biochem. Mol. Biol., 34:81-93, 1999) and for the side chain of ubiquinone (Disch et al., Biochem. J, 15:615-621, 1998), operates in the cytosol; the mevalonate-independent pathway, which is used for the synthesis of isoprene, monoterpenes, certain sesquiterpenes, diterpenes, carotenoids, as well as the side chains of tocopherol, phylloquinone and chlorophyll, is localized to plastids (Eisenreich et al., Trends Plant Sci., 6:78-84, 2001). Stage 2 begins with the isomerization of IPP to DMAPP, the latter of which then serves as the reactive starter molecule for subsequent condensation reactions with IPP to form geranyl diphosphate (GPP, C₁₀), famesyl diphosphate (FPP, C15), geranylgeranyl diphosphate (GGPP, C20), squalene (C30), phytyl diphosphate (C40), phytoene (C40) and higher prenyl diphosphates (Ogura, In: Comprehensive Natural Products Chemistry, Vol 2: Isoprenoids Including Carotenoids and Steroids, D. E. Cane, ed., Pergamon Press, 1999). In stage 3, these prenyl diphosphates, or compounds directly derived from them, undergo a range of cyclization reactions to produce the parent skeletons of the different monoterpens from GPP, sesquiterpenes from FPP, diterpenes from GGPP, triterpenes (sterols) from 2,3-epoxysqualene, and tetraterpenes (carotenoids) from lycopene. The cyclic parent compounds are then transformed in stage 4 by redox, isomerization, substitution and conjugation reactions to yield the various isoprenoid end-products.

### SUMMARY

Within the scope of the present invention a set of genes is now provided which are involved in isoprenoid metabolism, but especially a set of genes comprising nucleic acid sequences derived from the rice genome including homologs and orthologs thereof. One of the main objectives of the present invention is thus to provide an isolated nucleic acid molecule comprising a nucleotide sequence encoding a polypeptide which is involved in isoprenoid metabolism of plants, but especially of cereal plants such as rice, wheat, barley, maize, etc.

The majority of the genes within this group encode protein products that are directly involved in or associated with the major pathways of isoprenoid metabolism: plastididal biosynthetic pathways; cytosolic and mitochondrial biosynthetic pathways; pathways leading to monoterpenes and sesquiterpenes; pathways leading to protein phenylation.

In one embodiment, the invention thus relates to an isolated nucleic acid molecule comprising a nucleotide sequence encoding a polypeptide that is involved in at least one of the major pathways of isoprenoid metabolism selected from the group consisting of SEQ ID NOs: 1-414 including homologs and orthologs thereof or the complement thereof.

Another subset of genes provided herein comprises a number of genes that encode polypeptides that are involved in or associated with plastididal biosynthetic pathways. Exemplary representatives of such genes and polypeptides are those identified in Table 1 and listed in the Sequence Listing.

Still another subset of genes that is provided as part of the invention comprises nucleic acid molecules that encode polypeptides that are involved in or associated with the cytosolic and mitochondrial biosynthetic pathways. Exemplary representatives of such genes and polypeptides are those identified in Table 1 and listed in the Sequence Listing.

A further subset of genes provided herein comprise nucleic acid molecules the expression products of which are involved in or associated with pathways leading to monoterpenes and sesquiterpenes. Exemplary representatives of such genes and polypeptides are those identified in Table 1 and listed in the Sequence Listing.

Still another subset of genes provides nucleic acid molecules the expression products of which are involved in or associated with pathways leading to protein phenylation. Exemplary representatives of such genes and polypeptides are those identified in Table 1 and listed in the Sequence Listing.

In a collective embodiment, applicable to all of the nucleic acid molecules disclosed herein, the invention relates to the use of the nucleic acid molecules according to the invention as hybridization probes, for chromosome and gene mapping, in PCR technologies, in the production of sense or antisense nucleic acids, in screening for new therapeutic molecules, in production of plants and seeds having desirable, inheritable, commercially useful phenotypes, or in discovery of inhibitory compounds.

The invention further relates to any polypeptides encoded by the nucleic acid molecules according to the invention, or any antigene sequences thereof, which have numerous applications using techniques that are known to those skilled in the art of molecular biology, biotechnology, biochemistry, genetics, physiology or pathology.

In a further collective embodiment, the present invention provides the ability to modulate isoprenoid metabolism, by over-expressing, under-expressing or knocking out one or more of the genes disclosed herein or their gene products, in a plant cell, in vitro or in planta. Expression vectors comprising at least one nucleic acid molecule according to the invention, or any antigenes thereof, operably linked to at least one suitable promoter and/or regulatory sequence can be used to study the role of polypeptides encoded by said sequences, for example by transforming a host cell with said expression vector and measuring the effects of overexpression and underexpression of said nucleic acid molecules. Suitable promoter and/or regulatory sequences include especially those that are preferentially or specifically active in specific plant tissues such as, for example, green tissues (leaf, stem), grain tissues (the grain endosperm or the grain embryo), flowers, fruits, etc.

A host cell transformed with at least one expression vector comprising at least one nucleic acid molecule of the invention, operably linked to suitable promoters and/or regulatory sequences, can be useful to produce a plant with improved nutritional or dietary pharmacological properties.

In a further collective embodiment, the present invention provides a transformed plant host cell, or one obtained through breeding, capable of over-expressing, under-expressing, or having a knock out of at least one of the genes according to the invention and/or their gene products.

Such a plant cell, transformed with at least one expression vector comprising a nucleic acid molecule of the invention, operably linked to suitable promoters and/or regulatory sequences, can be used to regenerate plant tissue or an entire plant, or seed there from, in which the effects of expression, including overexpression or underexpression, of the introduced sequence or sequences can be measured in vitro or in planta.

In a further embodiment the present invention provides nucleotide sequences including regions of nucleotide sequence encoding polypeptides having homology to at least one functional protein domain (FPD). Embodiments of the invention further provide polypeptides including regions of amino acid sequence having homology to an FPD. In cases where the polypeptide has homology to an FPD in the same or closely related species, the polypeptide may represent a paralogous sequence or paralog, or may represent a variant allele of a gene encoding the FPD. In cases where the polypeptide has homology to an FPD in another species, including other plant species and especially non-plant species, polypeptides may represent orthologous sequences, or orthologs, of the FPD.

In a further collective embodiment of the invention the nucleic acid molecules disclosed herein or respresentative parts thereof can be used in hybridization-based assays for detecting and identifying nucleic acid molecules that encode protein products that are involved in isoprenoid metabolism in plants other than rice, but especially in plants belonging to the cereal group.

Embodiments of the present invention provide a unique oligonucleotide having a sequence identical to or complementary to a region of a polynucleotide sequence encoding at least a portion of a homologue of a protein according to the invention representatives of which are identified by the SEQ ID NOs provided in the Sequence Listing and/or an FPD thereof, the oligonucleotide being identified by the methods disclosed herein. In one embodiment, the unique oligonucleotide has a length of between 12 and 250 nucleotide bases.

Embodiments of the present invention also provide a nucleotide microarray comprising the unique oligonucleotide having a sequence identical to or complementary to a region of polynucleotide sequence encoding at least a portion of a homologue of a protein according to the invention representatives of which are identified by the SEQ ID NOs provided in the Sequence Listing and/or an FPD thereof. Preferably, the microarray includes a plurality of different, unique oligonucleotides, the sequences corresponding to a plurality of homologues of a protein according to the invention representatives of which are identified by the SEQ ID NOs provided in the Sequence Listing and/or an FPD thereof. Equally preferably, the microarray contains at least about 96 different unique oligonucleotides, wherein each of the 96 different unique oligonucleotides has a sequence that is identical, complementary, or has substantial similarity to a segment of a nucleotide sequence as given in the SEQ ID NOs provided in the Sequence Listing.

Embodiments of the present invention also provide a kit for detecting the presence of a polynucleotide, the kit containing a first nucleotide probe which can hybridize with a region of a nucleotide sequence including the nucleotide sequences of the SEQ ID NOs provided in the Sequence Listing, a fragment or a variant thereof, and a complementary sequence thereto, the kit further containing at least one additional component such as, for example: a second nucleotide probe, a buffer, an enzyme, a label, a molecular weight standard, a reaction chamber, and a micropipette tip.

Embodiments of the present invention further provide a kit for detecting the presence of a polypeptide, the kit containing a first probe which can hybridize with a region of a polypeptide including the amino acid sequences of the SEQ ID NOs provided in the Sequence Listing, a fragment or a variant thereof, and optionally, the kit further containing at least one additional component such as, for example: a probe, a buffer, an enzyme, a label, a molecular weight standard, a reaction chamber, and a micropipette tip. Probes useful in kit embodiments include antibodies, affinity tags, protein A, protein G, or protein-binding substances including chromatographic media.

In a further embodiment of the invention a computer readable medium containing one or more of the nucleotide sequences of the invention is provided as well as methods of use for the computer readable medium. This medium allows a nucleotide sequence corresponding to at least one of the SEQ ID NOs provided in the Sequence Listing (open reading frames or fragments thereof), to be used as a reference sequence to search against a database. This medium also allows for computer-based manipulation of a nucleotide sequence corresponding to at least one of SEQ ID NOs provided in the Sequence Listing. Additional embodiments include An isolated polynucleotide comprising nucleotide sequence encoding a polypeptide involved in or associated with the biosynthesis of isoprenoids in a plant, wherein said nucleotide sequence comprises a) a nucleotide sequence selected from the group consisting of SEQ ID NO 1, 3, 5, 7, 9, 11, 13, 15, 17, 19, 21, 23, 25, 27, 28, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, and 413; b) a nucleotide sequence having substantial similarity to a nucleotide sequence of a); c) a nucleotide sequence of at least 15 nucleotides in length that hybridizes with a nucleotide sequence of a) or the complement thereof under stringent conditions d) a fragment of a nucleotide sequence of a) wherein said fragment encodes a polypeptide involved in or associated with the biosynthesis of isoprenoids in a plant; e) a nucleotide sequence complementary to a nucleotide sequence of a), b) or c); or f) a nucleotide sequence comprising a reverse complement to a nucleotide sequence of a), b) or c).

Another embodiment provides isolated polynucleotides comprising nucleotide sequence encoding a polypeptide involved in or associated with the biosynthesis of isoprenoids in a plant, wherein said polypeptide comprises an amino acid sequence selected from the group consisting of SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 314, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412 and 414.

Yet another embodiment provides an isolated polypeptide involved in or associated with the biosynthesis of isoprenoids in a plant, comprising an amino acid sequence selected from the group consisting of SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 314, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412 and 414. Also included are amino acid sequences having substantial similarity to the sequences disclosed above including those having conservative amino acid substitutions.

One embodiment provides an isolated polynucleotide comprising a nucleotide sequence or fragment thereof encoding a polypeptide involved in or associated with the biosynthesis of isopentenyl diphosphate (IPP) and dimethylallyl alcohol (DMAPP) in a plant plastid said nucleotide sequence selected from the group consisting of SEQ ID NO 99, 113, 153, 161, 267, and 413 as well as sequences having substantial similarity to these sequences.

Still another embodiment provides an isolated polynucleotide comprising a nucleotide sequence encoding a polypeptide involved in or associated with the biosynthesis of isopentenyl diphosphate (IPP) and dimethylallyl alcohol (DMAPP) in a plant plastid, said polypeptide comprising an amino acid sequence selected from the group consisting of SEQ ID NO 100, 114, 154, 162, 268 and 414.

Also provided are isolated polynucleotides comprising a nucleotide sequence encoding a polypeptide or fragment thereof with enzymatic activity involved in or associated with the biosynthesis of short-chain plastid prenyltransferases, said nucleotide sequence selected from the group consisting of SEQ ID NO 11, 47, 83, 125, 135, 229, 235, 243, 265, 299, 345, 371, 409 and 411 and sequences having substantial similarity to these sequences. Likewise is provided an isolated polynucleotide comprising a nucleotide sequence encoding a polypeptide with enzymatic activity involved in or associated with the biosynthesis of short-chain plastid prenyltransferases said polypeptide comprising an amino acid sequence selected from the group consisting of SEQ ID NO 12, 48, 84, 126, 136, 230, 236, 244, 266, 300, 346, 372, 410 and 412.

Yet another embodiment provides an isolated polynucleotide comprising a nucleotide sequence or fragment thereof encoding a polypeptide the activity of which is involved in or associated with the biosynthesis of gibberellins in plant, said nucleotide sequence selected from the group consisting of SEQ ID NO 25, 67, 75, 79, 97, 107, 141, 155, 173, 193, 203, 205, 223, 225, 253, 259, 261, 291, 297, 349, 359, 375, 377, and 399; isolated polynucleotides comprising a nucleotide sequence encoding a polypeptide the activity of which is involved in or associated with the biosynthesis of gibberellins in plants, said polypeptide comprising an amino acid sequence selected from the group consisting of SEQ ID NO 26, 68, 76, 80, 98, 108, 142, 156, 174, 194, 204, 206, 224, 226, 254, 260, 262, 292, 298, 350, 360, 376, 378, and 400; and sequences substantially similar to any of the preceding sequences and

A futher embodiment provides isolated polynucleotides comprising a nucleotide sequence or fragment thereof encoding a polypeptide which exhibits enzymatic activity involved in or associated with the biosynthesis of carotenoids and/or abscisic acids in plants, said nucleotide sequence selected from the group consisting of SEQ ID NO 27, 89, 103, 121, 205, 237, 245, 271, 275, 301, 313, 317, 383, 391, 395, 397 and 407; isolated polynucleotide comprising a nucleotide sequence encoding a polypeptide which exhibits enzymatic activity involved in or associated with the biosynthesis of carotenoids and/or abscisic acids in plants, said polypeptide comprising an amino acid sequence selected from the group consisting of SEQ ID NO 28, 90, 104, 122, 206, 238, 246, 272, 276, 302, 314, 318, 384, 392, 396, 398 and 408; and sequences substantially similar to any of the preceding sequences.

Still a further embodiment provides isolated polynucleotides comprising a nucleotide sequence or fragment thereof encoding a polypeptide which has enzymatic activity involved in or associated with the biosynthesis of tocopherols in plants, said nucleotide sequence selected from the group consisting of SEQ ID NO 89 and 275; isolated polynucleotides comprising a nucleotide sequence encoding a polypeptide which has enzymatic activity involved in or associated with the biosynthesis of tocopherols in plants, said polypeptide comprising an amino acid sequence selected from the group consisting of SEQ ID NO 90 and 276; and sequences substantially similar to the preceding sequences.

Another embodiment provides isolated polynucleotides comprising a nucleotide sequence or fragment thereof encoding a polypeptide with enzymatic activity involved in or associated with plastoquinone and/or phylloquinone biosynthesis in a plant, said nucleotide sequence selected from the group consisting of SEQ ID NO 17, 29, 85, 87, 89, 207, 213, 285, 393, 401; isolated polynucleotides comprising a nucleotide sequence encoding a polypeptide with enzymatic activity involved in or associated with plastoquinone and/or phylloquinone biosynthesis in a plant, said polypeptide comprising an amino acid sequence selected from the group consisting of SEQ ID NO 18, 30, 86, 88, 90, 208, 214, 286, 394, 402; and sequences substantially similar to the preceding sequences.

Also provides are isolated polynucleotides comprising a nucleotide sequence or fragment thereof encoding a polypeptide with enzymatic activity involved in or associated with the mevalonate pathway in plants, said nucleotide sequence selected from the group consisting of SEQ ID NO 111, 133, 165, 293, 315, 353, 365; isolated polynucleotide comprising a nucleotide sequence encoding a polypeptide with enzymatic activity involved in or associated with the mevalonate pathway in plants, said polypeptide comprising an amino acid sequence selected from the group consisting of SEQ ID NO 112, 134, 166, 294, 316, 354, 366; and sequences substantially similar to any of the preceding sequences.

Yet another aspect provides isolated polypeptides comprising a nucleotide sequence or a fragment thereof encoding a polypeptide with enzymatic activity involved in or associated with phytosterol and brassinosteroid metabolism in a plant, said nucleotide sequence selected from the group consisting of SEQ ID NO 3, 5, 13, 15, 21, 33, 35, 37, 45, 49, 59, 61, 65, 91, 95, 119, 123, 131, 147, 163, 169, 173, 183, 187, 195, 197, 219, 251, 269, 287, 303, 309, 311, 323, 329, 331, 333, 337, 341, 361, 367, 379, 389, and 405; isolated polypeptide comprising a nucleotide sequence encoding a polypeptide with enzymatic activity involved in or associated with phytosterol and brassinosteroid metabolism in a plant, said polypeptide comprising an amino acid selected from the group consisting of SEQ ID NO 4, 6, 14, 16, 22, 34, 36, 38, 46, 50, 60, 62, 66, 92, 96, 120, 124, 132, 148, 164, 170, 174, 184, 188, 196, 198, 220, 252, 270, 288, 304, 310, 312, 324, 330, 332, 334, 338, 342, 362, 368, 380, 390 and 406; and sequences substantially similar to any of the preceding sequences.

Still another aspect provides isolated polypeptides comprising a nucleotide sequence or fragment thereof encoding a polypeptide with activity involved in or associated with biosynthesis of ubiquinone in a plant said nucleotide sequence selected from the group consisting of SEQ ID NO 7, 239 and 381; isolated polypeptides comprising a nucleotide sequence encoding a polypeptide with activity involved in or associated with biosynthesis of ubiquinone in a plant said polypeptide comprising an amino acid sequence selected from the group consisting of SEQ ID NO 8, 240 and 382; and sequences having substantial similarity to any of the preceding sequences.

An additional embodiment provides an isolated polynucleotide comprising a nucleotide sequence or fragment thereof encoding a polypeptide with activity involved in or associated with the biosynthesis of monterpenes and sesquiterpenes in a plant, said polynucleotide comprising a nucleotide sequence selected from the group consisting of SEQ ID NO 1, 9, 19, 39, 41, 43, 51, 55, 57, 69, 71, 77, 81, 99, 101, 109, 115, 129, 137, 145, 151, 157, 159, 167, 171, 179, 181, 185, 201, 209, 211, 217, 227, 231, 233, 241, 247, 249, 257, 263, 273, 279, 281, 283, 305, 319, 321, 327, 347, 351, 357, 369, 375, 385, and 387; An isolated polynucleotide comprising a nucleotide sequence encoding a polypeptide with activity involved in or associated with the biosynthesis of monterpenes and sesquiterpenes in a plant, said polypeptide comprising an amino acid sequence selected from the group consisting of SEQ ID NO 2, 10, 20, 40, 42, 44, 52, 56, 58, 70, 72, 78, 82, 100, 102, 110, 116, 130, 138, 146, 152, 158, 160, 168, 172, 180, 182, 186, 202, 210, 212, 218, 228, 232, 234, 242, 248, 250, 258, 264, 274, 280, 282, 284, 306, 320, 322, 328, 348, 352, 358, 370, 376, 386, 388, and 404; and sequences substantially similar to any of the preceding sequences.

Another aspect provides isolated polynucleotide comprising a nucleotide sequence or fragment thereof encoding a polypeptide that is involved in or associated with protein prenylation, said nucleotide sequence selected from the group consisting of SEQ ID NO 23, 63, 189, 255, 289, 343, and 373; isolated polynucleotide comprising a nucleotide sequence encoding a polypeptide that is involved in or associated with protein prenylation, said polypeptide comprising an amino acid sequence selected from the group consisting of SEQ ID NO 24, 64, 190, 256, 290, 344, and 374; and sequences substantially similar to any of the preceding sequences.

Yet another embodiment provides an isolated polynucleotide comprising a nucleotide sequence or fragment thereof encoding a polypeptide involved in or associated with the biosynthesis of chlorophyll or heme, said nucleotide sequence selected from the group consisting of SEQ ID NO 31, 47, 53, 105, 117, 127, 139, 143, 149, 175, 177, 191, 199, 221, 277, 295, 307, 325, 335, 339, 355, and 363; isolated polynucleotides comprising a nucleotide sequence encoding a polypeptide involved in or associated with the biosynthesis of chlorophyll or heme, said polypeptide comprising an amino acid sequence selected from the group consisting of SEQ ID NO 32, 48, 54, 106, 118, 128, 140, 144, 150, 176, 178, 192, 200, 222, 278, 296, 308, 326, 336, 340, 356, and 364; and sequences substantially similar to any of the previous sequences.

Additional embodiments provides expression cassettes comprising any of the nucleotide sequences disclosed herein along with host cells and transgenic plants comprising said expressions cassettes.

Further aspects, features and advantages of this invention will become apparent from the detailed description of the preferred embodiments that follow.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, public databases, public database entries, and other references cited in this application are herein incorporated by reference in their entirety as if each individual publication, patent, patent application, public database, public database entry, or other reference was specifically and individually indicated to be incorporated by reference.

For clarity, certain terms used in the specification are defined and presented as follows:

Definitions

The term “gene” is used broadly to refer to any segment of nucleic acid associated with a biological function. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “native” or “wild type” gene refers to a gene that is present in the genome of an untransformed cell, i.e., a cell not having a known mutation.

A “marker gene” encodes a selectable or screenable trait.

The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

An “oligonucleotide” corresponding to a nucleotide sequence of the invention, e.g., for use in probing or amplification reactions, may be about 30 or fewer nucleotides in length (e.g., 9, 12, 15, 18, 20, 21 or 24, or any number between 9 and 30). Generally specific primers are upwards of 14 nucleotides in length. For optimum specificity and cost effectiveness, primers of 16 to 24 nucleotides in length may be preferred. Those skilled in the art are well versed in the design of primers for use processes such as PCR. If required, probing can be done with entire restriction fragments of the gene disclosed herein which may be 100's or even 1000's of nucleotides in length.

The terms “protein,” “peptide” and “polypeptide” are used interchangeably herein.

The nucleotide sequences of the invention can be introduced into any plant. The genes to be introduced can be conveniently used in expression cassettes for introduction and expression in any plant of interest. Such expression cassettes will comprise the transcriptional initiation region of the invention linked to a nucleotide sequence of interest. Preferred promoters include constitutive, tissue-specific, developmental-specific, inducible and/or viral promoters. Such an expression cassette is provided with a plurality of restriction sites for insertion of the gene of interest to be under the transcriptional regulation of the regulatory regions. The expression cassette may additionally contain selectable marker genes. The cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions. See also, Guerineau et al., Mol. Gen. Genet., 262:141, 1991; Proudfoot, Cell, 64:671, 1991; Sanfacon et al., Genes Devel., 5:141, 1991; Mogen et al., Plant Cell, 2:1261, 1990; Munroe et al., Gene, 91:151, 1990; Ballas et al., Nuc. Acids Res., 17:7891 1989; Joshi et al., Nuc. Acids, Res. 1987.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225, 1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell, 1:671, 1989.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

“Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription.

“Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Constitutive promoter” refers to a promoter that is able to express the open reading frame (ORF) that it controls in all or nearly all of the plant tissues during all or nearly all developmental stages of the plant. Each of the transcription-activating elements do not exhibit an absolute tissue-specificity, but mediate transcriptional activation in most plant parts at a level of ≧1% of the level reached in the part of the plant in which transcription is most active.

“Regulated promoter” refers to promoters that direct gene expression not constitutively, but in a temporally- and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (Biochem. Plants, 15:1, 1989). Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysome-inducible systems.

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

“Inducible promoter” refers to those regulated promoters that can be turned on in one or more cell types by an external stimulus, such as a chemical, light, hormone, stress, or a pathogen.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, ORF or portion thereof, or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

“Specific expression” is the expression of gene products which is limited to one or a few plant tissues (spatial limitation) and/or to one or a few plant developmental stages (temporal limitation). It is acknowledged that hardly a true specificity exists: promoters seem to be preferably switch on in some tissues, while in other tissues there can be no or only little activity. This phenomenon is known as leaky expression. However, with specific expression in this invention is meant preferable expression in one or a few plant tissues.

The “expression pattern” of a promoter (with or without enhancer) is the pattern of expression levels which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore, the steady state level is the product of synthesis rates and degradation rates.

The rate of degradation can however be considered to proceed at a fixed rate when the transcribed sequences are identical, and thus this value can serve as a measure of synthesis rates. When promoters are compared in this way techniques available to those skilled in the art are hybridization S1-RNAse analysis, northern blots and competitive RT-PCR. This list of techniques in no way represents all available techniques, but rather describes commonly used procedures used to analyze transcription activity and expression levels of mRNA.

The analysis of transcription start points in practically all promoters has revealed that there is usually no single base at which transcription starts, but rather a more or less clustered set of initiation sites, each of which accounts for some start points of the mRNA. Since this distribution varies from promoter to promoter the sequences of the reporter mRNA in each of the populations would differ from each other. Since each mRNA species is more or less prone to degradation, no single degradation rate can be expected for different reporter mRNAs. It has been shown for various eukaryotic promoter sequences that the sequence surrounding the initiation site (‘initiator’) plays an important role in determining the level of RNA expression directed by that specific promoter. This includes also part of the transcribed sequences. The direct fusion of promoter to reporter sequences would therefore lead to suboptimal levels of transcription.

A commonly used procedure to analyze expression patterns and levels is through determination of the ‘steady state’ level of protein accumulation in a cell. Commonly used candidates for the reporter gene, known to those skilled in the art are β-glucuronidase (GUS), chloramphenicol acetyl transferase (CAT) and proteins with fluorescent properties, such as green fluorescent protein (GFP) from Aequora victoria. In principle, however, many more proteins are suitable for this purpose, provided the protein does not interfere with essential plant functions. For quantification and determination of localization a number of tools are suited. Detection systems can readily be created or are available which are based on, e.g., immunochemical, enzymatic, fluorescent detection and quantification. Protein levels can be determined in plant tissue extracts or in intact tissue using in situ analysis of protein expression.

Generally, individual transformed lines with one chimeric promoter reporter construct will vary in their levels of expression of the reporter gene. Also frequently observed is the phenomenon that such transformants do not express any detectable product (RNA or protein). The variability in expression is commonly ascribed to ‘position effects’, although the molecular mechanisms underlying this inactivity are usually not clear.

“Overexpression” refers to the level of expression in transgenic cells or organisms that exceeds levels of expression in normal or untransformed (nontransgenic) cells or organisms.

“Antisense inhibition” refers to the production of antisense RNA transcripts capable of suppressing the expression of protein from an endogenous gene or a transgene.

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes (English et al., Plant Cell, 8:179, 1996). Gene silencing includes virus-induced gene silencing (Ruiz et al., Plant Cell, 10:937, 1998).

The terms “heterologous DNA sequence,” “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Homologous to” in the context of nucleotide sequence identity refers to the similarity between the nucleotide sequence of two nucleic acid molecules or between the amino acid sequences of two protein molecules. As used herein, “homology” and “homologous” refer to an evaluation of the similarity between two sequences based on measurements of sequence identity adjusted for variables including gaps, insertions, frame shifts, conservative substitutions, and sequencing errors, as described below. Two nucleotide sequences or polypeptides are the to be “identical” if the sequence of nucleotides or amino acid residues, respectively, in the two sequences is the same when aligned for maximum correspondence as described below. The term “complementary to” is used herein to mean that the sequence can form a Watson-Crick base pair with a reference polynucleotide sequence. Complementary sequences can include nucleotides, such as inosine, that neither disrupt Watson-Crick base pairing nor contribute to the pairing. A “reverse complement” of a sequence corresponds to the complementary sequence, but in the opposite orientation of bases from 5′ to 3′, or to the complement of the primary sequence, if the primary sequence is in a reverse orientation of bases from 5′ to 3′.

Homology is evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, Proc Natl Acad Sci (USA) 85:2444 (1988); Altschul et al., J Mol Biol 215:403 (1990)). In a particularly preferred embodiment, protein and nucleic acid sequence homologies are evaluated using the Basic Local Alignment Search Tool (“BLAST”) which is well known in the art (Karlin and Altschul, Proc Natl Acad Sci USA 87:2264 (1990); Altschul et al. (1990) supra, Altschul et al., Nucleic Acids Res 25:3389 (1997)). In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (aligned) by means of a scoring matrix selected from the many scoring matrices known in the art. Preferably, the scoring matrix used is the BLOSUM62 matrix (Gonnet et al., Science 256:1443 (1992); Henikoff and Henikoff, Proteins 17:49 (1993)). Likewise, the PAM or PAM250 matrices may also be used (Schwartz and Dayhoff, In Atlas of Protein Sequence and Structure, Dayhoff, ed., Natl. Biomed. Res. Found., pp. 353-358 (1978)). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (Karlin and Altschul (1990) supra).

Percentage of sequence identity can be determined from alignments performed using algorithms known in the art. Alignment of nucleotide or polypeptide sequences for comparison may be conducted by the local homology algorithm of Smith and Waterman (Add APL Math 2:482 (1981)), by the homology alignment algorithm of Needleman and Wunsch (J Mol Biol 48:443 (1970)), by the search for similarity method of Pearson and Lipman (Proc Natl Acad Sci USA 85:2444 (1988)), by computerized implementations of these algorithms (GAP, BESTFIT, BLAST, PASTA, and TFASTA in the Wisconsin Genetics Software Package, Genetics Computer Group), or by inspection. When two sequences have been identified for comparison, GAP and BESTFIT are preferably employed to determine their optimal alignment. Typically, the default values of 5.00 for gap weight and 0.30 for gap weight length are used. In a preferred embodiment, percenty identity is determined using the GAP program for global alignment using default parameters, using the version of GAP found in the GCG package (Wisconsin Package Version 10.1, Genetics Computer Group, 575 Science Dr., Madison, Wis.).

Percentage of sequence identity is determined by comparing two optimally aligned sequences over a comparison window, wherein the portion of the sequence in the comparison window may include additions or deletions, including for example gaps or overhangs, as compared to the reference sequence (which does not include additions or deletions) for optimal alignment of the two sequences. The percentage is calculated by determining the number of positions at which the identical nucleotide base or amino acid residue occurs in both sequences to yield the number of matched positions, dividing the number of matched positions by the total number of positions in the window of comparison and multiplying the result by 100 to yield the percentage of sequence identity.

In a broad sense, the term “substantially similar”, when used herein with respect to a nucleotide sequence, means a nucleotide sequence corresponding to a reference nucleotide sequence, wherein the corresponding sequence encodes a polypeptide having substantially the same structure as the polypeptide encoded by the reference nucleotide sequence. Desirably, the substantially similar nucleotide sequence encodes the polypeptide encoded by the reference nucleotide sequence. Preferably, “substantially similar” refers to nucleotide sequences having at least 50% sequence identity, preferably at least 60%, 70%, 80% or 85%, more preferably at least 90% or 95%, and even more preferably, at least 96%, 97% or 99% sequence identity compared to a reference sequence containing nucleotide sequences of Table I, that encode a protein having at least 50% identity, more preferably at least 85% identity, yet still more preferably at least 90% identity to a region of sequence of a BIOPATH protein and/or an FPD, wherein the protein sequence comparisons are conducted using GAP analysis as described below. Also, “substantially similar” preferably also refers to nucleotide sequences having at least 50% identity, more preferably at least 80% identity, still more preferably 95% identity, yet still more preferably at least 99% identity, to a region of nucleotide sequence encoding a BIOPATH protein and/or an FPD, wherein the nucleotide sequence comparisons are conducted using GAP analysis as described herein. The term “substantially similar” is specifically intended to include nucleotide sequences wherein the sequence has been modified to optimize expression in particular cells.

A polynucleotide including a nucleotide sequence “substantially similar” to the reference nucleotide sequence preferably hybridizes to a polynucleotide including the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

The term “substantially similar”, when used herein with respect to a protein or polypeptide, means a protein or polypeptide corresponding to a reference protein, wherein the protein has substantially the same structure and function as the reference protein, where only changes in amino acids sequence that do not materially affect the polypeptide function occur. When used for a protein or an amino acid sequence the percentage of identity between the substantially similar and the reference protein or amino acid sequence desirably is preferably at least 30%, more preferably at least 40%, 50%, 60%, 70%, 80%, 85%, or 90%, still more preferably at least 95%, still more preferably at least 99% with every individual number falling within this range of at least 30% to at least 99% also being part of the invention, using default GAP analysis parameters with the University of Wisconsin GCG (version 10), SEQWEB application of GAP, based on the algorithm of Needleman and Wunsch (1970), supra. As used herein the term “polypeptide of the present invention,” or any similar term refers to an amino acid sequence encoded by a DNA molecule including a nucleotide sequence substantially similar to an AC sequence. Homologs of BIOPATH protein and/or FPDs include amino acid sequences that are at least 30% identical to BIOPATH protein and/or FPD sequences found in searchable databases, as measured using the parameters described above.

“Target gene” refers to a gene on the replicon that expresses the desired target coding sequence, functional RNA, or protein. The target gene is not essential for replicon replication. Additionally, target genes may comprise native non-viral genes inserted into a non-native organism, or chimeric genes, and will be under the control of suitable regulatory sequences. Thus, the regulatory sequences in the target gene may come from any source, including the virus. Target genes may include coding sequences that are either heterologous or homologous to the genes of a particular plant to be transformed. However, target genes do not include native viral genes. Typical target genes include, but are not limited to genes encoding a structural protein, a seed storage protein, a protein that conveys herbicide resistance, and a protein that conveys insect resistance. Proteins encoded by target genes are known as “foreign proteins”. The expression of a target gene in a plant will typically produce an altered plant trait.

The term “altered plant trait” means any phenotypic or genotypic change in a transgenic plant relative to the wild-type or non-transgenic plant host.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The term “transformation” refers to the transfer of a nucleic acid fragment into the genome, including the plastid or mitochondrial genome, of a host cell, resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere et al., Meth. Enzymol., 143:277, 1987) and particle bombardment technology (Klein et al., Nature, 327:70, 1987; U.S. Pat. No. 4,945,050). Whole plants may be regenerated from transgenic cells by methods well known to the skilled artisan (see, for example, Fromm et al., Bio/Technology, 8:833, 1990).

“Transformed,” “transgenic,” and “recombinant” refer to a host organism such as a bacterium or a plant into which a heterologous nucleic acid molecule has been introduced. The nucleic acid molecule can be stably integrated into the genome generally known in the art and are disclosed in Sambrook et al., Molecular Cloning, 2^{nd} ed., Cold Spring Harbor Laboratory Press, 1989. See also Innis et al., PCR Protocols, Academic Press, 1995 and Gelfand, PCR Strategies, Academic Press, 1995; and Innis and Gelfand, PCR Methods Manual, Academic Press, 1999. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” plants or calli have been through the transformation process and contain a foreign gene integrated into their chromosome. The term “untransformed” refers to normal plants that have not been through the transformation process.

“Transiently transformed” refers to cells in which transgenes and foreign DNA have been introduced (for example, by such methods as Agrobacterium-mediated transformation or biolistic bombardment), but not selected for stable maintenance.

“Stably transformed” refers to cells that have been selected and regenerated on a selection media following transformation.

“Transient expression” refers to expression in cells in which a virus or a transgene is introduced by viral infection or by such methods as Agrobacterium-mediated transformation, electroporation, or biolistic bombardment, but not selected for its stable maintenance.

“Genetically stable” and “heritable” refer to chromosomally-integrated genetic elements that are stably maintained in the plant and stably inherited by progeny through successive generations.

“Primary transformant” and “T0 generation” refer to transgenic plants that are of the same genetic generation as the tissue which is initially transformed (i.e., not having gone through meiosis and fertilization since transformation).

“Secondary transformants” and the “T1, T2, T3, etc. generations” refer to transgenic plants derived from primary transformants through one or more mciotic and fertilization cycles. They may be derived by self-fertilization of primary or secondary transformants or crosses of primary or secondary transformants with other transformed or untransformed plants.

“Wild-type” refers to a virus or organism found in nature without any known mutation.

“Genome” refers to the complete genetic material of an organism. The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nuc. Acids Res., 19:5081, 1991; Ohtsuka et al., J. Biol. Chem., 260:2605, 1985; Rossolini et al. Mol. Cell. Probes, 8:91, 1994). A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals.

The nucleotide sequences of the invention include both the naturally occurring sequences as well as mutant (variant) forms. Such variants will continue to possess the desired activity, i.e., either promoter activity or the activity of the product encoded by the open reading frame of the non-variant nucleotide sequence.

Thus, by “variants” is intended substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

The nucleic acid molecules of the invention can be “optimized” for enhanced expression in plants of interest. See, for example, EPA 035472; WO 91/16432; Perlak et al., Proc. Natl. Acad. Sci. USA, 88:3324: 1991; and Murray et al., Nuc. Acids. Res., 17:477, 1989. In this manner, the open reading frames in genes or gene fragments can be synthesized utilizing plant-preferred codons. See, for example, Campbell and Gowri, Plant Physiol., 92:1, 1990 for a discussion of host-preferred codon usage. Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, Nature, 370:389, 1994; Stemmer, Proc. Natl. Acad. Sci. USA, 91:10747, 1994; Crameri et al., Nature Biotech., 15:436, 1997; Moore et al., J. Mol. Biol., 272:336, 1997; Zhang et al., Proc. Natl. Acad. Sci. USA, 94:4504, 1997; Crameri et al., Nature, 391:288, 1998; and U.S. Pat. Nos. 5,605,793 and 5,837,458.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488, 1985; Kunkel et al., Methods Enzymol., 154:367, 1987; U.S. Pat. No. 4,873,192; Walker and Gaastra, Techniques in Molecular Biology, MacMillan Publishing, 1983 and the references cited therein. Guidance as to appropriate amino acid substitutions that do not affect biological activity of the protein of interest may be found in the model of Dayhoff et al. (Atlas of Protein Sequence and Structure, Natl. Biomed. Res. Found., 1978). Conservative substitutions, such as exchanging one amino acid with another having similar properties, are preferred.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (O). See also, Creighton, 1984. In addition, individual substitutions, deletions or additions which alter, add or delete a single amino acid or a small percentage of amino acids in an encoded sequence are also “conservatively modified variations.”

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

A “transgenic plant” is a plant having one or more plant cells that contain an expression vector.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, CABIOS, 4:11, 1988; the local homology algorithm of Smith et al. Adv. Appl. Math., 2:482, 1981; the homology alignment algorithm of Needleman and Wunsch J. Mol. Biol., 48:443, 1970; the search-for-similarity-method of Pearson and Lipman, Proc. Natl. Acad. Sci. USA, 85:2444, 1988; the algorithm of Karlin and Altschul, Proc. Natl. Acad. Sci. USA, 87:2264, 1990, modified as in Karlin and Altschul, Proc. Natl. Acad. Sci. USA, 90:5873, 1993.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. Gene, 73:237 1988; Higgins et al. CABIOS, 5:151, 1989; Corpet et al. Nuc. Acids Res., 16:10881, 1988; Huang et al., CABIOS, 8:155, 1992; and Pearson et al. Meth. Mol. Biol., 24:307, 1994. The ALIGN program is based on the algorithm of Myers and Miller, supra. The BLAST programs of Altschul et al., J. Mol. Biol., 215:403, 1990, are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., J. Mol. Biol., 215:403, 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences (see, e.g., Karlin & Altschul (Proc. Natl. Acad. Sci. USA, 90:5873, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., Nuc. Acids Res., 25:3389, 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA, 89:10915, 1989). See http://www.ncbi.nlm.nih.gov. Alignment may also be performed manually by inspection.

For purposes of the present invention, comparison of nucleotide sequences for determination of percent sequence identity to the sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch (J. Mol. Biol., 48:443, 1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridization are sequence dependent, and are different under different environmental parameters. The Tm is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267, 1984; Tₘ 81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tm, hybridization, and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point for the specific sequence and its complement at a defined ionic strength and pH. However, severely stringent conditions can utilize a hybridization and/or wash at 1, 2, 3, or 4° C. lower than the thermal melting point; moderately stringent conditions can utilize a hybridization and/or wash at 6, 7, 8, 9, or 10° C. lower than the thermal melting point; low stringency conditions can utilize a hybridization and/or wash at 11, 12, 13, 14, 15, or 20° C. lower than the thermal melting point. Using the equation, hybridization and wash compositions, and desired T, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology-Hybridization with Nucleic Acid Probes, part I, Chap 2, Elsevier, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5×to 1×SSC at 55 to 60° C.

The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in O. 1×SSC, 0.1% SDS at 65° C.

“DNA shuffling” is a method to introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA preferably encodes a variant polypeptide modified with respect to the polypeptide encoded by the template DNA, and may have an altered biological activity with respect to the polypeptide encoded by the template DNA.

“Recombinant DNA molecule’ is a combination of DNA sequences that are joined together using recombinant DNA technology and procedures used to join together DNA sequences as described, for example, in Sambrook et al., Molecular Cloning, 2^{nd} ed., Cold Spring Harbor Laboratory Press, 1989.

The word “plant” refers to any plant, particularly to seed plant, and “plant cell” is a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ.

“Significant increase” is an increase that is larger than the margin of error inherent in the measurement technique, preferably an increase by about 2-fold or greater.

“Significantly less” means that the decrease is larger than the margin of error inherent in the measurement technique, preferably a decrease by about 2-fold or greater.

Within the scope of the present invention a set of genes is provided which comprises nucleic acid molecules that are involved in or asscociated with isoprenoid metabolism in plants, but especially in cereal plants such as rice, wheat, banana, maize, barley, etc.

According to one embodiment, the present invention is directed to a nucleic acid molecule comprising a nucleotide sequence isolated or obtained from any plant which encodes a polypeptide that has at least 70% amino acid sequence identity to a polypeptide encoded by a gene comprising any one of SEQ ID NOs provided in the Sequence Listing.

The genes within this subgroup are useful tools for generating plants, but especially cereal plants, which exhibit modified compositional characteristics leading to improved nutritional and pharmacological properties.

Exemplary representatives of such genes and polypeptides are those selected from the group consisting of SEQ ID NO. 1-414 including homologs and orthologs thereof, for example SEQ ID NO. 415-661, or the complements thereof.

In one embodiment, the present invention thus relates to an isolated nucleic acid molecule comprising a nucleotide sequence encoding a polypeptide, which is involved in or associated with isoprenoid metabolism and the use of said molecule for modifying the nutritional and pharmacological composition and thus the quality of the plant.

In particular, the subsets of nucleic acid molecules provided herein, which comprise genes that are involved in isoprenoid biosynthesis is a valuable tool box from which an appropriate nucleic acid molecule can be chosen to modify the quantity and/or quality of the nutritional and pharmacological constituents of the plant, respectively.

This can be achieved by introducing and overexpressing at least one nucleic acid molecule from the various subsets of genes provided herein in the plant, but preferentially in the approproate tissues of the plant such as, for example, the plant seed, leaf, stem, flower, fruit, etc, and, within the cells of said tissue within the appropriate cellular compartment such as, for example, plastids, mitochondria, ER, the cytosol, etc. In the alternative, the expression level of the corresponding endogenous gene can be reduced by methods known in the art including anti-sense and dsRNAi techniques.

A representative group of sequences which can be used for that purpose is provided in the Sequence Listing as SEQ ID NOs: 1-414 The majority of the genes within this group encode protein products that are directly involved in or associated with the major pathways of isoprenoid metabolism: plastididal biosynthetic pathways; cytosolic and mitochondrial biosynthetic pathways; pathways leading to monoterpenes and sesquiterpenes; pathways leading to protein phenylation.

In one embodiment, the present invention identifies and provides a subset of genes that is involved in plastididal biosynthetic pathways. By modifying the expression level of at least one nucleic acid molecule from this subgroup in a plant, but preferably in the approproate tissues of the plant such as, for example, the green tissue parts of the plant, and even more preferably in the plant leaf or stem, it is possible to modify the nutritional or pharmacological composition of the plant accordingly.

Thus, in one aspect, the present invention relates to an isolated nucleic acid molecule comprising a nucleotide sequence encoding a polypeptide the activity of which is involved in or associated with the biosynthesis of isoprenoids in the plant plastids, which nucleotide sequence is substantially similar to a sequence encoding a polypeptide as given in any one of the SEQ ID NOs of Table 1.

In particular, the invention relates to an isolated nucleic acid molecule comprising a nucleotide sequence encoding a polypeptide the activity of which is involved in or associated with the biosynthesis of isoprenoids in plant plastids, but especially the plastids of a cereal plant such as rice, wheat, banana, barley, maize, etc, and which is substantially similar, and preferably has at least between 70%, and 99% amino acid sequence identity to at least one polypeptide as given in any one of the SEQ ID NOs of Table 1, with any individual number within this range of between 70% and 99% also being part of the invention.

The invention further relates to an isolated nucleic acid molecule comprising a nucleotide sequence encoding a polypeptide the activity of which is involved in or associated with the biosynthesis of isoprenoids in the plant plastids, but especially the plastids of a cereal plant such as rice, wheat, banana, barley, maize, etc, and which is immunologically reactive with antibodies raised against a polypeptide as given in any one of the SEQ ID NOs of Table 1.

More particularly, the invention relates to an isolated nucleic acid molecule comprising a nucleotide sequence

a) as given in in any one of the SEQ ID NOs of Table 1;

b) having substantial similarity to (a);

c) having at least 15 nucleotides capable of hybridizing to (a) or the complement thereof, perferably under stringent, highly stringent or very highly stringent conditions;

d) having at least 15 nucleotides and capable of hybridizing to a nucleic acid comprising 50 to 200 or more consecutive nucleotides of a nucleotide sequence as given in any one of the SEQ ID NOs of Table 1. or the complement thereof, perferably under stringent, highly stringent or very highly stringent conditions;

e) complementary to (a), (b) or (c); and

f) which is a reverse complement of (a), (b) or (c).

By providing these genes it is now possible to regulate the expression levels of the encoded protein products in the plant, but especially a cereal plant such as rice, wheat, banana, barley, maize, etc, by applying methods known in the art including overexpressing or down-regulating the nucleic acid molecule in a plant, thereby modifying the isoprenoid content of the plant.

In one specific embodiment, the present invention relates to a subset of genes that encode a protein that exhibits an enzymatic activity that is involved in or associated with the mevalonate-independent biosynsethis of plastidial isoprenoids.

The mevalonate-independent pathway is responsible for the biosynthesis of plastidial isoprenoids. Evidence has emerged over the last few years that isopentenyl diphosphate, the central intermediate of isoprenoid biosynthesis, originates from pyruvate and D-glyceraldehyde-3-phosphate via a new mevalonate-independent pathway in several eubacteria (Rohmer, M., et al., Biochem. J. 295, 517-524, 1993; Broers, S. T. J. (1994) Ph.D. Thesis, Eidgenossische Technische Hochschule, Zuirich, Switzerland; Rohmer, M., et al., J. Am. Chem. Soc. 118, 2564-2566, 1996), algae (Schwender, J., et al., Biochem. J. 316, 73-80, 1996), and plant plastids (Schwarz, M. K. (1994) Ph.D. Thesis, Eidgenossische Technische Hochschule, Zurich, Switzerland; Lichtenthaler, H. K., et al., FFBS Lett. 400, 271-274, 1997).

The enzymes responsible for the biosynthesis of IPP (isopentenyl diphosphate) and DMAPP (dimethylallyl alcohol) via the mevalonate-independent pathway in plants are localized to plastids. For the first enzyme, 1-deoxy-D-xylulose 5-phosphate synthase (DXPS), the A. thaliana genome contains three copies, for one of which functional data have been obtained (Estevez et al., J. Biol. Chem., 22901, 2001). The subsequent steps are catalyzed by 1-deoxy-D-xylulose 5-phosphate reductoisomerase (DXR), 2-C-methyl-D-erythritol 4-phosphate cytidyltransferase (MCT), 4-(cytidine 5′-diphospho)-2-C-methyl-D-erythritol kinase (CMK) and 2-C-methyl-D-erythritol 2,4-cyclodiphosphate synthase (MECPS). Functional data are available for A. thaliana orthologues of DXR (Schwender et al., FEBS Lett., 455:140, 1999) and MCT (Rohdich et al., Proc. Natl. Acad. Sci. USA, 97:6451, 2000), whereas functional data for plant CMKs are obtained only for the tomato enzyme (Rohdich et al., Proc. Natl. Acad. Sci. USA, 97:8251, 2000), and MECPS has been functionally characerized from a non-plant source. Two additional open reading frames from E. coli, annotated as lytB and gepE, have been characterized recently to encode enzyme products capable of generating IPP and DMAPP from 2-C-methyl-D-erythritol 2,4-cyclodiphosphate (Hintz et al., FEBS Lett., 509:317, 2001; Hecht et al., Proc. Natl. Acad. Sci. USA, 98:14837, 2001; Rohdich et al., Proc. Natl. Acad. Sci. USA, 99:20925, 2002). Genes homologous to lytB and gcpE occur as single copies in A. thaliana.

In one specific embodiment, the invention relates to an isolated nucleic acid molecule comprising a nucleotide sequence encoding a polypeptide the activity of which is involved in or associated with the biosynthesis of isopentenyl diphosphate (IPP) and dimethylallyl alcohol (DMAPP) in plant plastids, which nucleotide sequence is substantially similar to a sequence encoding a polypeptide as given in the SEQ ID NOs identified in Table 1 such as SEQ ID NOs: 100, 114, 154, 162, 268 and 414.

In particular, the invention relates to an isolated nucleic acid molecule comprising a nucleotide sequence encoding a polypeptide the activity of which is involved in or associated with the biosynthesis of IPP and DMAPP in plant plastids, and which is substantially similar, and preferably has at least between 70%, and 99% amino acid sequence identity to at least one polypeptide of SEQ ID NOs given in table 1 such as SEQ ID NOs: 100, 114, 154, 162, 268 and 414, with any individual number within this range of between 70% and 99% also being part of the invention.

The invention further relates to an isolated nucleic acid molecule comprising a nucleotide sequence encoding a polypeptide the activity of which is involved in or associated with the biosynthesis of IPP and DMAPP in plant plastids, and which is immunologically reactive with antibodies raised against a polypeptide as given in the SEQ ID NOs of table 1 such as SEQ ID NOs: 100, 114, 154, 162, 268 and 414.

More particularly, the invention relates to an isolated nucleic acid molecule comprising a nucleotide sequence

a) as given in any one of SEQ ID NOs of table 1 such as SEQ ID NOs:

99, 113, 153, 161, 267 and 413;

b) having substantial similarity to (a);

c) having at least 15 nucleotides and capable of hybridizing to (a) or the complement thereof under stringent, highly stringent or very highly stringent conditions;

d) having at least 15 nucleotides and capable of hybridizing to a nucleic acid comprising 50 to 200 or more consecutive nucleotides of a nucleotide sequence given in (a) or the complement thereof under stringent, highly stringent or very highly stringent conditions;

e) complementary to (a), (b) or (c); and

f) which is a reverse complement of (a), (b) or (c).

By providing these genes it is now possible to regulate the expression levels of the encoded protein products in the plant by applying methods known in the art including overexpressing or down-regulating the nucleic acid molecule in a plant, thereby modifying the isoprenoid content of the plant.

An up-regulation of genes and encoded enzymes of the mevalonate-independent pathway is expected to increase the accumulation of the end-products.

In addition, the over-expression of DXR resulted in an increased production of isoprenoid essential oil components in peppermint (Mahmoud and Croteau, Proc. Nat. Acad. Sci. USA 98: 8915, 2001).

Arabidopsis T-DNA knock-out lines for DXPS, DXR and MCT show an albino phenotype (Budziszewski et al., Genetics, 159: 1765, 2001), thus indicating their potential as targets for herbicides.

In marigold, the homolog to the DXP synthase is shown to produce a deep red when in the presence of lycopene.

A regulatory role of DXPS is also reported for carotenoid biosynthesis during tomato fruit development (Lois et al., Plant J., 22: 503, 2000).

A gene (or other nucleic acid molecule) encoding any one of the proteins given in SEQ ID NOs: 100, 114, 154, 162, 268 and 414, but especially a gene encoding 1-deoxy-D-xylulose-5-phosphate reductoisomerase such as that given in SEQ ID NO: 268 may be incorporated into any organism (intact plant, animal, microbe, etc.), or cell culture derived therefrom, but especially into a plant, for example, a cereal plant including rice, wheat, barley, banana, maize, etc. The enzyme 1-deoxy-D-xylulose-5-phosphate reductoisomerase catalyzes the first committed step in the conversion of 1-deoxy-D-xylulose-5-phosphate to isopentenyl diphosphate which, in turn, is converted to a variety of molecules including, for example, carotenoids, and the prenyl side chains of chlorophyll, plastoquinone and tocopherols. Thus, a 1-deoxy-D-xylulose-5-phosphate reductoisomerase gene (or other nucleic acid molecule) may be introduced into any organism for a variety of purposes including, but not limited to: production of 1-deoxy-D-xylulose-5-phosphate reductoisomerase, or its product 2-C-methyl-D-erythritol-4-phosphate; enhancement of chlorophyll production by increasing the synthesis of the phytol side-chain; enhancement of production of terpenoids, phytoalexins, toxins, and deterrent compounds to improve defense against pathogens, insects and other herbivores; enhance the production of monoterpene flavor and aroma compounds in essential oil plants, fruits and vegetables to improve the flavor and aroma profiles, or improve the yield of flavor and aroma compounds extracted from plants, to prepare synthetic intermediates in plants and microbes for industrial uses, such as the synthesis of adhesives, inks and polymers; to enhance the production of natural pigments, such as carotenoids, in plants, and to improve the yield of natural pigments extracted from plants for medicinal or culinary uses, to enhance the yield in plants of compounds having anti-cancer or other nutraceutical properties, such as vitamin A and vitamin E; and to produce 2C-methyl-D-erythritol phosphate as an enzymatic or chemical intermediate. While the nucleic acid molecules of the present invention can be introduced into any organism, the nucleic acid molecules of the present invention will preferably be introduced into a plant species, but especially into a cereal plant including rice, wheat, barley, banana, maize, etc.

A further subset of genes provided herein comprises genes that encode polypeptides with an activity that is involved in or associated with the biosynthesis of short-chain prenyltransferases of the plastid.

The invention thus also relates to an isolated nucleic acid molecule comprising a nucleotide sequence encoding a polypeptide the enzymatic activity of which is involved in or associated with the biosynthesis of short-chain prenyltransferases of the plastid and, which nucleic acid is substantially similar to a nucleic acid encoding a polypeptide as given in any one of the SEQ ID NOs of Table 1 such as SEQ ID NOs: 12, 48, 84, 126, 136, 130, 236, 244, 266, 300, 346, 372, 410 and 412.

More specifically, the invention relates to an isolated nucleic acid molecule comprising a nucleotide sequence encoding a polypeptide the enzymatic activity of which involved in or associated with the biosynthesis of short-chain prenyltransferases of the plastid and has at least between 70%, and 99% amino acid sequence identity to at least one polypeptide as given in any one of the SEQ ID NOs of Table 1 such as SEQ ID NOs: 12, 48, 84, 126, 136, 130, 236, 244, 266, 300, 346, 372 410 and 412, with any individual number within this range of between 70% and 99% also being part of the invention.

The invention further relates to an isolated nucleic acid molecule comprising a nucleotide sequence encoding a polypeptide the enzymatic activity of which is involved in or associated with the biosynthesis of short-chain prenyltransferases of the plastid and which is immunologically reactive with antibodies raised against a polypeptide as given in any one of the SEQ ID NOs of Table 1 such as SEQ ID NOs: 12, 48, 84, 126, 136, 130, 236, 244, 266, 300, 346, 372, 410 and 412.

More particularly, the invention relates to an isolated nucleic acid molecule comprising a nucleotide sequence

a) as given in any one of the SEQ ID NOs of Table 1 such as SEQ ID NOs: 11, 47, 83, 125, 135, 229, 235, 243, 265, 299, 345, 371, 409 and 411;

b) having substantial similarity to (a);

c) having at least 15 nucletoides and capable of hybridizing to (a) or the complement thereof under stringent, highly stringent or very highly stringent conditions;

d) having at least 15 nucleotides and capable of hybridizing to a nucleic acid comprising 50 to 200 or more consecutive nucleotides of a nucleotide sequence (a) or the complement thereof under stringent, highly stringent, or very highly stringent conditions;

e) complementary to (a), (b) or (c); and

f) which is a reverse complement of (a), (b) or (c).

As part of the plastidial stage 2 reactions, A. thaliana genes encoding two isoenzymes of isopentenyl diphosphate isomerase and a geranyl diphosphate synthase (GPPS; single copy gene in A. thaliana genome), have been characterized (Campbell et al., Plant Mol. Biol., 36:323, 1997; Bouvier et al., Plant J., 24:241, 2000). Geranylgeranyl diphosphate synthase (GGPPS) is encoded by a multigene family with 12 members. In A. thaliana; for five members of this gene family functional data have been obtained (Okada et al., Plant Physiol., 122:1045, 2000), and two isoenzymes of these have been shown to occur in plastids. Based on the sequence characteristics of the remaining 7 members of the GGPPS gene family, all encoded proteins contain a plastidial targeting sequence and should thus be localized to plastids.

A further subset of genes that is provided as part of the invention comprises genes that are involved in the biosynthesis of gibberellins.

Gibberellins are classified on the basis of structure as well as function. All gibberellins are derived from the ent-gibberellane skeleton. All gibberellins are acidic compounds and are therefore also called gibberellic acids (GA) with a different subscript to distinguish between them. GA₃ has historically been called gibberellic acid but the term is also often used in describing all gibberellins. GA's are widespread and so far ubiquitous in both flowering (angiosperms) and non-flowering (gymnosperms) plants as well as ferns. They have also been isolated from lower plants such as mosses and algae, at least two fungal species and most recently from two bacterial species.

Active gibberellins show many physiological effects, each depending on the type of gibberellin present as well as the species of plant. Some of the physiological processes stimulated by gibberellins are outlined below (Davies, 1995; Mauseth, 1991; Raven, 1992; Salisburv and Ross, 1992).

1. Stimulate stem elongation by stimulating cell division and elongation.

2. Stimulates bolting/flowering in response to long days.

3. Breaks seed dormancy in some plants which require stratification or light to induce germination.

4. Stimulates enzyme production (a-amylase) in germinating cereal grains for mobilization of seed reserves.

5. Induces maleness in dioecious flowers (sex expression).

6. Can cause parthenocarpic (seedless) fruit development.

7. Can delay senescence in leaves and citrus fruits.

Gibberellins are tetracyclic diterpenes with a role as hormones controling stem elongation and affecting reproductive processes. Two diterpene synthases are encoded in the A. thaliana genome which code for the two plastidial entry enzymes into the gibberellin biosynthetic pathway, copalyl diphosphate synthase (Sun and Kamiya, Plant Cell, 6:1509, 1994) and ent-kaurene synthase (Yamaguchi et al., Plant Physiol., 116:1271, 1998). The gibberellin end-products are generated by a series of extraplastidial oxidative modifications, starting with the multifunctional cytochrome P450 monooxygenases ent-kaurene oxidase (Helliwell et al., Plant Physiol., 119:507, 1999; single gene in A. thaliana) and ent-kaurenoic acid oxidase (Helliwell et al., Proc. Natl. Acad. Sci. USA, 98:2065, 2001; 2 isogenes), which are localized to the endoplasmatic reticulum. Further modifications are catalyzed by multifunctional oxoglutarate-dependent enzymes, and include gibberellin 20-oxidase (Xu et al., Proc. Natl. Acad. Sci. USA, 92:6640, 1995; Phillips et al., Plant Physiol., 108:1049, 1995; 5 isogenes in A. thaliana) and gibberellin 3β-oxidase (Williams et al., Plant Physiol., 117:559, 1998), which are localized in the cytosol. Gibberellin deactivation reactions are initiated by gibberellin 2-oxidase (Thomas et al., Proc. Natl. Acad. Sci. USA, 96:4698, 1999; 3 isogenes in A. thaliana), another multifunctional oxoglutarate-dependent dioxygenase with cytosolic localization. Further catabolic reactions are undefined at this time.

By providing these genes it is now possible to regulate the expression levels of the encoded protein products in the plant by applying methods known in the art including overexpressing or down-regulating the nucleic acid molecule in a plant, thereby modifying the giberrelin content of the plant.

Over-expression of gibberellin biosynthetic genes may result in an increased seed production in nice in deepwater areas.

Deepwater rice is a subsistence crop for ˜100 Mio people in areas of Southeast Asia. An important factor for yield is the submergence-induced rapid internodal elongation of deepwater rice stems, which is regulated, at least in part, by gibberellins (Kende et al., Plant Physiol., 118: 1105, 1998). By overexpressing one or more of the genes that are involved or associated with gibberllin biosynthesis such as those given in Table 1 and in particular SEQ ID NOs: 25, 67, 75, 79, 97, 107, 141, 155, 173, 193, 203, 205, 223, 225, 253, 259, 261, 291, 297, 349, 359, 375, 377, and 399, gibberllin levels can be manipulated such as to increase the speed of internodal elongation and thus yield in rice.

Thus, in one aspect, the present invention relates to an isolated nucleic acid molecule comprising a nucleotide sequence encoding a polypeptide the activity of which is involved in or associated with the biosynthesis of gibberellins in the plant plastids, which nucleotide sequence is substantially similar to a sequence encoding a polypeptide as given in any one of the SEQ ID NOs of Table 1 such as SEQ ID NOs: 26, 68, 76, 80, 98, 108, 142, 156, 174, 194, 204, 206, 224, 226, 254, 260, 262, 292, 298, 350, 360, 376, 378, and 400.

In particular, the invention relates to an isolated nucleic acid molecule comprising a nucleotide sequence encoding a polypeptide the activity of which is involved in or associated with the biosynthesis of gibberellins in the plant plastids, and which is substantially similar, and preferably has at least between 70%, and 99% amino acid sequence identity to at least one polypeptide as given in any one of the SEQ ID NOs of Table 1 such as SEQ ID NOs: 26, 68, 76, 80, 98, 108, 142, 156, 174, 194, 204, 206, 224, 226, 254, 260, 262, 292, 298, 350, 360, 376, 378, and 400, with any individual number within this range of between 70% and 99% also being part of the invention.

The invention further relates to an isolated nucleic acid molecule comprising a nucleotide sequence encoding a polypeptide the activity of which is involved in or associated with the biosynthesis of gibberellins in the plant plastids, and which is immunologically reactive with antibodies raised against a polypeptide as given in any one of the SEQ ID NOs of Table 1 such as SEQ ID NOs: 26, 68, 76, 80, 98, 108, 142, 156, 174, 194, 204, 206, 224, 226, 254, 260, 262, 292, 298, 350, 360, 376, 378, and 400.

More particularly, the invention relates to an isolated nucleic acid molecule comprising a nucleotide sequence

g) as given in in any one of the SEQ ID NOs of Table 1 such as SEQ ID NOs: 25, 67, 75, 79, 97, 107, 141, 155, 173, 193, 203, 205, 223, 225, 253, 259, 261, 291, 297, 349, 359, 375, 377, and 399;

h) having substantial similarity to (a);

i) having at least 15 nucleotides and capable of hybridizing to (a) or the complement thereof under stringent, highly stringent or very highly stringent conditions;

j) having at least 15 nucleotides and capable of hybridizing to a nucleic acid comprising 50 to 200 or more consecutive nucleotides of a nucleotide sequence of (a) or the complement thereof under stringent, highly stringent, or very highly stringent conditions;

k) complementary to (a), (b) or (c); and

l) which is a reverse complement of (a), (b) or (c).

In still another embodiment the invention discloses a subset of genes comprising genes that encode polypeptides, which are involved in or associated with the biosynthesis of carotenoids and/or abscisic acids in the plant plastid and cytosol, respectively.

Carotenoids are essential components of photosynthetic membranes and are involved in the dissipation of excess light energy during photosynthesis; thus, the enzymes involved in carotenoid biosynthesis are attractive targets for herbicides; conversely, the over-expression, in tobacco, of a phytoene desaturase gene conferred tolerance to the herbicide norflurazon (Misawa et al., Plant J., 4:833, 1993).

Carotenoids also play an important role in human nutrition (Parker, FASEB J., 10: 542, 1996; Mayne, FASEB J., 10: 690, 1996). Lycopene, beta-carotene and zeaxanthin are used as poultry and fish farming additives, as well as in foods as colorants (Klaui and Bauemfeind, Carotenoids as Colorants and Vitamin A Precursors, Academic Press, 1981).

The biosynthesis of carotenoids, which protect the photosynthetic apparatus from photooxidative damage and play a structural role in the assemby of light-harvesting complexes, is entirely localized to plastids. Phytoene synthase, which catalyzes the first commited step in this pathway, was first cloned from tomato (Bartley et al., J. Biol. Chem., 267:5036, 1992), and is encoded by a single gene in A. thaliana. Further metabolic conversions are catalyzed by phytoene desaturase (Bartley et al., Proc. Natl. Acad. Sci. USA, 88:6532, 1991) and ζ-carotene desaturase (Albrecht et al., FEBS Lett., 372:199, 1995) to yield lycopene (genes for both enzymes occur as single copies in the A. thaliana genome). In addition, phytoene desaturation also requires plastoquinone (Norris et al., Plant Cell, 7:2139, 1995) and a plastidial terminal oxidase activity (Wu et al., Plant Cell, 11:43, 1999). Lycopene is cyclized on both ends to generate either β-carotene (catalyzed by lycopene β-cyclase, Pecker et al., Plant Mol. Biol., 30:807, 1996) or α-carotene (combination of lycopene β-cyclase and lycopene ε-cyclase (Cunningham at al., Plant Cell, 8:1613, 1996)). Hydroxylation of β-carotene by β-carotene hydroxylase (Sun et al., J. Biol. Chem., 271:24349, 1996; 2 isogenes detectable in the A. thaliana genome) results in the formation of zeaxanthin. Hydroxylation of a-carotene, possibly by the action of the same β-hydroxylase and an as yet unidentified ε-hydroxylase, produces lutein, the most abundant xanthophyll in plant plastids. Zeaxanthin epoxidase, which was first cloned from tobacco (Marin et al., EMBO J, 15:2331, 1996), catalyzes the conversion of zeaxanthin to violaxanthin. A homologous sequence, represented by a single copy gene, is detectable in the A. thaliana genome. Under high light intensity, violaxanthin is converted back to zeanxanthin (violaxanthin de-epoxidase; Bugos et al., J. Biol. Chem., 273:15321, 1998), which participates in the thermal dissipation of excess absorbed light energy (xanthophyll cycle). Both violaxanthin and its allene derivative neoxanthin can be precursors for the carotenoid-derived plant hormone abscisic acid (ABA). In potato and tobacco, violaxanthin can be converted to neoxanthin by neoxanthin synthase, an enzyme with high homology to carotenoid cyclases and capsanthin/capsorubin synthase from pepper (Bouvier et al., Eur. J. Biochem, 267:6346, 2000; Al-Babili et al., FEBS Lett., 485:168, 2000). However, in A. thaliana, no additional candidate genes, besides the already characterized carotenoid cyclases, are detectable with reasonable sequence homology, indicating that a neoxanthin synthase activity may either not exist in this organism, or that it has evolved independently from a divergent enzyme, or that it uses violaxanthin as the precursor for ABA biosynthesis. Thus far, two further enzymes involved in the breakdown of xanthophylls to ABA have been cloned: genes for epoxycarotenoid cleavage enzyme (Schwartz et al., 2001) occur as a family of 7 members in the A. thaliana genome, whereas abscisic aldehyde oxidase (Seo et al., Proc. Natl. Acad. Sci. USA, 97:12908, 2000) is encoded by a single copy gene. ABA 8′-hydroxylase, a membrane-bound cytochrome P450 monooxygenase has been characterized biochemically (Krochko et al., Plant Physiol., 118:849, 1998).

In a specific embodiment, the present invention provides nucleic acid molecules such as those represented in Table 1 such as SEQ ID NOs: 27, 89, 103, 121, 205, 237, 245, 271, 275, 301, 313, 317, 383, 391, 395, 397 and 407 that encode polypeptides which exhibit an enzymatic activity that is involved in or associated with the biosynthesis of carotenoids and/or abscisic acids in the plant plastid and cytosol, respectively.

The invention thus relates to an isolated nucleic acid molecule comprising a nucleotide sequence encoding a polypeptide which exhibits an enzymatic activity that is involved in or associated with the biosynthesis of carotenoids and/or abscisic acids in the plant plastid and cytosol, respectively, which nucleotide sequence is substantially similar to a sequence encoding a polypeptide as given in any one of the SEQ ID NOs of Table 1 such as SEQ ID Nos: 28, 90, 104, 122, 206, 238, 246, 272, 276, 302, 314, 318, 384, 392, 396, 398 and 408.

More specifically, the invention relates to an isolated nucleic acid molecule comprising a nucleotide sequence encoding a polypeptide which exhibits an enzymatic activity that is involved in or associated with the biosynthesis of carotenoids and/or abscisic acids in the plant plastid and cytosol, respectively and has at least between 70%, and 99% amino acid sequence identity to at least one polypeptide as given in any one of the SEQ ID NOs of Table 1 such as SEQ ID Nos: 28, 90, 104, 122, 206, 238, 246, 272, 276, 302, 314, 318, 384, 392, 396, 398 and 408, with any individual number within this range of between 70% and 99% also being part of the invention.

The invention further relates to an isolated nucleic acid molecule comprising a nucleotide sequence encoding a polypeptide which exhibits an enzymatic activity that is involved in or associated with the biosynthesis of carotenoids and/or abscisic acids in the plant plastid and cytosol, respectively and immunologically reactive with antibodies raised against a polypeptide as given in any one of the SEQ ID NOs of Table 1 such as SEQ ID Nos: 28, 90, 104, 122, 206, 238, 246, 272, 276, 302, 314, 318, 384, 392, 396, 398 and 408.

More particularly, the invention relates to an isolated nucleic acid molecule comprising a nucleotide sequence

a) as given in any one of the SEQ ID NOs of Table 1 such as SEQ ID Nos: 27, 89, 103, 121, 205, 237, 245, 271, 275, 301, 313, 317, 383, 391, 395, 397 and 407;

b) having substantial similarity to (a);

c) having at least 15 nucelotides and capable of hybridizing to (a) or the complement thereof under stringent, highly stringenty or very highly stringent conditions;

d) having at least 15 nucleotides and capable of hybridizing to a nucleic acid comprising 50 to 200 or more consecutive nucleotides of a nucleotide sequence of (a) or the complement thereof under stringent, highly stringenty or very highly stringent conditions;

e) complementary to (a), (b) or (c); and

f) which is a reverse complement of (a), (b) or (c).

By providing these genes it is now possible to regulate the expression levels of the encoded protein products in the plant by applying methods known in the art including overexpressing or down-regulating the nucleic acid molecule in a plant, thereby modifying the carotenoid and abscisic acid content of the plant, respectively.

Over-expression of caroteinoid biosynthetic genes may result in plants producing enhanced levels of caroteinoids such as lycopene, beta-carotene and zeaxanthin and thus to plant products with enhanced nutritional value.

Abscisic acid is a key factor regulating transpiration, stress responses, germination of seeds and embryogenesis. Most effects of ABA seem to be related to water availability—it apparently acts as a signal of reduced water availability. ABA conserves water by reducing water loss, slowing growth and mediating adaptive responses. However, ABA influences most aspects of plant growth and development to some extent—partly due to interactions with other phytohormones.

By over-expressing or knocking-out one or more of the genes that are involved in or associated with abscisic acid biosynthesis it is possible to modify the level of abscisic acid in plants and hence regulation of transpiration, stress responses, germination of seeds and embryogenesis. For example, it has been shown that an over-expression of 9-cis-epoxycarotenoid dioxygenase gene, which encodes an enzyme catalyzing the first step in the breakdown of carotenoids to abscisic acid, enhances drought tolerance in tobacco (Qin and Zeevart, Plant Physiol., 2002).

In still another embodiment the invention discloses a subset of genes comprising genes that encode polypeptides, which are involved in or associated with the biosynthesis of tocopherols in the plant plastid and cytosol, respectively.

Tocopherols are essential for the prevention of photo-oxidative deterioration of biomembranes (Fryer, Plant Cell Environ., 15:381, 1992). Tocopherols, and in particular alpha-tocopherol, exhibit vitamin E activity.

A daily intake of 100 to 1000 International Units of vitamin E is acssociated with a decreased risk of cardiovascular disease and some cancers, improved immune function, and slowing of the progression of a number of degenerative human conditions (Traber and Sies, Annu. Rev. Nutr., 16:321, 1996).

Tocopherols, collectively known as vitamin E, are lipid-soluble antioxidants synthesized exclusively by photosynthetic organisms. The first committed step in tocopherol biosynthesis involves the condensation of homogentisate, which is derived from tyrosine (tyrosine aminotransferase; Lopukhina et al., Plant Physiol., 126:1678, 2001; family of 7 genes in A. thaliana genome) viap-hydroxyphenylpyruvate (HPPD synthase, Norris et al., Plant Physiol., 117:1317, 1998; single copy gene in A. thaliana), and phytyl diphosphate, which is generated from GGPP by geranylgeranyl reductase (GGR; Keller et al., Eur. J. Biochem., 251:413, 1998; single copy gene in A. thaliana). This condensation reaction is catalyzed by a membrane-bound plastid-localized homogentisate phytyltransferase (Collakova and DellaPenna, Plant Physiol., 127:1113, 2001; single copy gene in A. thaliana). Further conversion involve a C-methyltransferase and tocopherol-specific cyclase to yield γ-tocopherol. An alternative pathway from 2-methyl-6-phytylquinol via δ-tocopherol or β-tocopherol to γ-tocopherol has been proposed, but no enzymes involved in these steps have as yet been identified (Schultz et al., Physiol. Plant., 64:123, 1985). As the last step of the biosynthetic sequence, γ-tocopherol is converted to α-tocopherol by γ-tocopherol O-methyltransferase (Shintant and DellaPenna, Science, 282:2098, 1998), for which a single gene is detectable in the A. thaliana genome.

In one specific embodiment the present invention thus relates to an isolated nucleic acid molecule comprising a nucleotide sequence encoding a polypeptide which has an enzymatic activity that is involved in or associated with the biosynthesis of tocopherols in the plant plastid and cytosol, respectively, which nucleotide sequence is substantially similar to a sequence encoding a polypeptide as given in any one of the SEQ ID NOs of Table 1 such as SEQ ID Nos: 90 and 276.

In particular, the invention relates to an isolated nucleic acid molecule comprising a nucleotide sequence encoding a polypeptide which has an enzymatic activity that is involved in or associated with the biosynthesis of tocopherols in the plant plastid and cytosol, respectively, and is substantially similar, and preferably has at least between 70%, and 99% amino acid sequence identity to at least one polypeptide as given in any one of the SEQ ID NOs of Table 1 such as SEQ ID NOs: 90 and 276, with any individual number within this range of between 70% and 99% also being part of the invention.

The invention further relates to an isolated nucleic acid molecule comprising a nucleotide sequence encoding a polypeptide which has an enzymatic activity that is involved in or associated with the biosynthesis of tocopherols in the plant plastid and cytosol, respectively, and is immunologically reactive with antibodies raised against a polypeptide as given in any one of the SEQ ID NOs of Table 1 such as SEQ ID Nos: 90 and 276.

More particularly, the invention relates to an isolated nucleic acid molecule comprising a nucleotide sequence

a) as given in any one of the SEQ ID NOs of Table 1 such as SEQ ID Nos: 89 and 275;

b) having substantial similarity to (a);

c) having at least 15 nucleotides and capable of hybridizing to (a) or the complement thereof under stringent, highly stringent or very highly stringent conditions;

d) having at least 15 nucleotides and capable of hybridizing to a nucleic acid comprising 50 to 200 or more consecutive nucleotides of a nucleotide sequence of (a) or the complement thereof under stringent, highly stingent or very highly stringent conditions;

e) complementary to (a), (b) or (c); and

f) which is a reverse complement of (a), (b) or (c).

By providing these genes it is now possible to regulate the expression levels of the encoded protein products in the plant by applying methods known in the art including overexpressing or down-regulating the nucleic acid molecule in a plant, thereby modifying the tocopherol content of the plant, respectively.

As it is nearly impossible to reach the levels of vitamin E which are believed to be associated with a decreased risk of cardiovascular disease and some cancers, improved immune function, and slowing of the progression of a number of degenerative human conditions with an average Western diet, it has been proposed to use transgenic approaches (e.g. by over-expression of genes involved in tocopherol biosynthesis) to adjust the vitamin E levels in foods.

Over-expression of one or more genes that are involved in or associated with tocopherol biosynthesis such as those provided in SEQ ID NOs: 89 and 275 may result in plants producing enhanced levels of Vitamin E and thus produce plants with enhanced nutritional value. For example, gamma-Tocopherol O-methyltransferase can be used to convert oils rich in gamma-tocopherol into oils consisting mainly or exclusively of alpha-tocopherol (which has a much higher vitamin E activity).

In still another embodiment the present invention provides a further subset of nucleic acid molecules comprising genes that are involved in or associated with plastoquinone and/or phylloquinone biosynthesis in the plant plastid and cytosol, respectively.

These quinones are integral parts of the photosynthetic machinery of plants. Substituted quinones are employed as cofactors in electron transport chains of the two photosystems of plants and cyanobacteria. In photosystem II, plastoquinone-9 functions as a one-electron cofactor in the QA site and as a two-electron/two proton cofactor in the QB site. The biosynthesis involves, as in the case of tocopherols, a condensation of homogentisate with a prenyl diphosphate. The localization of enzymes responsible for the biosynthesis of the polyprenyl side chain of plastoquinone is still a matter of debate. Swiezewska et al, (J. Biol. Chem., 268:1494, 1993) have presented biochemical evidence that microsomal preparation from spinach contain polyprenyl diphosphate (solanesyl diphosphate) synthase and prenyltransferase activity for plastoquinone biosynthesis, and they have hypothesized that a specific transport and targeting system is required for the transfer of intermediates of plastoquinone biosynthesis into plastids. In contrast, using feeding experiments with isotopically labeled precursors, Lichtenthaler et al. (FEBS Lett., 400:271, 1997) and Disch et al., (Biochem. J., 15:615, 1998) have shown that the polyprenyl side chain of plastoquinone is synthesized in plastids via the mevalonate-independent pathway. The A. thaliana genome contains two predicted proteins with homology to solanesyl diphosphate synthase from Rhodobacter capsulatus (Okada et al., J. Bacteriol., 179:5992, 1997). One of these SDS-like isoenzymes apears to be, according to a TargetP analysis, localized to mitochondria (Atlg78510). For the second isogene (Atlg17050) a cytosolic localization is indicated by TargetP analysis; however, an alternative gene model for the same sequence (AAD50025) results in a gene product that is predicted to be localized to mitochondria. A plastidial SDS-like isoform has not been predicted yet. The A. thaliana genome contains a small gene family of prenyltransferases one of which has been characterized as being the tocopherol biosynthetic enzyme homogentisate phytyltransferase. A second prenyltransferase encodes chlorophyll synthetase (Gaubier et al., Mol. Gen. Genet., 249:58, 1995), whereas a third homologue is predicted, by TargetP analysis, to be localized to plastids (Table I), which is consitent with a homogentisate polyprenyltransferase for plastoquinone biosynthesis based on the isotope labeling data (Lichtenthaler et al., FEBS Lett., 400:271, 1997; Disch et al., Biochem J., 15:615, 1998). Phylloquinone, a second plastidial quinone of plants, functions as a one-electron cofactor in the Al site of photosystem I. Although the biosynthetic route to phylloquinone in plants has not been described in plants yet, the pathway is likely to be similar to the pathway of menaquinone biosynthesis in eubacteria. Menaquinone differs from phylloquinone by the presense of a partly unsaturated C-40 side chain rather than a mostly saturated, C-20 phytyl side chain. The genome database for the cyanobacterium Synechocystis, which also produces phylloquinone and not menaquinone, contains homologues for several genes that encode enzymes for menaquinone biosynthesis in E. coli, two of which, 1,4-dihydroxy-2-naphthoate (DHNA) synthase (menB) and DHNA phytyltransferase (mena) have been characterized using a mutant approach (Johnson et al., J. Biol. Chem. 275:8523, 2000). Based on a homology search, the putative A. thaliana orthologues for these genes are identified (Table I). In addition, homologues of the E. coli menaquinone biosynthetic genes O-succinylbenzoate (OSB) synthase (menC), 2-succinl-6-hydroxy-2,4-cycohexadiene-1-carboxylate (SHCHC) synthase (menD), and OSB-CoA ligase (menE) are detectable in the A. thaliana genome (Table I).

Two genes for isochorismate synthase are present in the A. thaliana genome, one of which is involved in salicylic acid biosynthesis (Wildermuth et al., Nature, 414:562, 2001), whereas the second isoenzyme could be involved in the phylloquinone biosynthetic pathway (Table I). In E. coli, the menaquinone and the ubiquinone biosynthetic pathways use the same C-methyltransferase (ubiE); however, in plants the pathway to the menaquinone analogue phylloquinone is localized to plastids whereas ubiquinone biosynthesis is restricted to mitochondria. Interestingly, the A. thaliana genome contains five genes with homology to ubiE. One ubiE homologue appears to specify an enzyme with a mitochondrial targeting sequence (candidate gene for ubiquinone biosynthesis) and a second gene encodes, based on a TargetP analysis, a plastidial isoenzyme which may be involved phylloquinone biosynthesis (Table I). The three additional copies are, based on current gene prediction, localized to the cytosolic compartment.

In one embodiment, the invention relates to an isolated nucleic acid molecule comprising a nucleotide sequence encoding a polypeptide with an enzymatic activity that is involved in or associated with plastoquinone and/or phylloquinone biosynthesis in the plant plastid and cytosol, respectively, which nucleotide sequence is substantially similar to a sequence encoding a polypeptide as given in SEQ ID NOs: 18, 30, 86, 88, 90, 208, 214, 286, 394, and 402.

In particular, the invention relates to an isolated nucleic acid molecule comprising a nucleotide sequence encoding a polypeptide with an enzymatic activity that is involved in or associated with plastoquinone and/or phylloquinone biosynthesis in the plant plastid and cytosol, respectively, and preferably has at least between 70%, and 99% amino acid sequence identity to at least one polypeptide as given in the SEQ ID NOs identified in Table 1 such as SEQ ID NOs: 18, 30, 86, 88, 90, 208, 214, 286, 394, and 402, with any individual number within this range of between 70% and 99% also being part of the invention.

The invention further relates to an isolated nucleic acid molecule comprising a nucleotide sequence encoding a polypeptide polypeptide with an enzymatic activity that is involved in or associated with plastoquinone and/or phylloquinone biosynthesis in the plant plastid and cytosol, respectively, and is immunologically reactive with antibodies raised against a polypeptide as given in the SEQ ID NOs identified in Table 1 such as SEQ ID NOs: 18, 30, 86, 88, 90, 208, 214, 286, 394, and 402.

More particularly, the invention relates to an isolated nucleic acid molecule comprising a nucleotide sequence

a) as given in the SEQ ID NOs identified in Table 1 such as SEQ ID NOs: 17, 29, 85, 87, 89, 207, 213, 285, 393, and 401;

b) having substantial similarity to (a);

c) having at least 15 nucleotides and capable of hybridizing to (a) or the complement thereof under stringent, highly stringent or very highly stringent conditions;

d) having at least 15 nucleotides and capable of hybridizing to a nucleic acid comprising 50 to 200 or more consecutive nucleotides of a nucleotide sequence of (a), or the complement thereof under stringent, highly stringent or very highly stringent conditions;

e) complementary to (a), (b) or (c); and

f) which is a reverse complement of (a), (b) or (c).

By providing these genes it is now possible to regulate the expression levels of the encoded protein products in the plant by applying methods known in the art including overexpressing or down-regulating the nucleic acid molecule in a plant, thereby modifying the plastoquinone and/or phylloquinone content of the plant, respectively.

Plastoquinones and tocopherols are essential structures for plants. Inhibitors of the biosynthesis of plastoquinones and tocopherols are therefore potential herbicides.

An over-expression of one or more genes involved in or associated with plastoquinone and phylloquinone biosynthesis such as those provided in SEQ ID NOs: 63256; 7568; 5714; 151018; and 26784 may result in an increased plant fittness under high light conditions.

One of most important and valuable activities plants are engaged in is photosynthesis, the light harvesting system of plants. Chlorophylls are light harvesting pigments present in all green photosynthetic organisms and are essential components of this light harvesting system. Its biosynthesis depends on the complex interaction of multiple enzymes.

Cyanobacteria and chloroplasts of algae and plants typically synthesize chlorophyll a and chlorophyll b for light harvesting and energy-generating charge separation. The Mg-tetrapyrrols of chlorophylls are derived from L-glutamate. Their biosynthesis starts with the conversion of L-glutamate to glutamate 1-semialdehyde, catalyzed by glutamyl-tRNA synthetase (Day et al., Biochim. Biophys. Acta, 1399:219, 1998; single copy gene in A. thaliana) and glutamyl-tRNA reductase (Kumar and Soll, Proc. Natl. Acad. Sci. USA, 89:10842, 1992; Kumar et al., Plant Mol. Biol., 30:419, 1996; 3 isogenes detectable in A. thaliana genome). For both of the subsequent enzymes, glutamate 1-semialdehyde aminotransferase, that was first cloned from barley (Grimm, Proc. Natl. Acad. Sci. USA, 87:4169, 1990), and aminolevulinate dehydratase, that was first cloned from pea (Boese et al., J. Biol. Chem., 266:17060, 1991), the A. thaliana genome contains two gene copies. Porphobilinogen deaminase catalyzes the condensation of four molecules of porphobilinogen to form 1-hydroxymethylbilane (Lim et al., Plant Mol. Biol., 26:863, 1994; single copy gene in A. thaliana). For uropophyrinogen decarboxylase, which was first cloned from tobacco and barley (Mock et al., Plant Mol. Biol., 28:245, 1995), two copies can be detected in the A. thaliana genome. Catalysis by coproporphyrinogen III oxidase (Ishikawa et al., Plant J., 27:89, 2001; single copy gene in A. thaliana) and protoporphyrinogen IX oxidase (Narita et al., Gene, 182:169, 1996; 2 isogenes inA. thaliana genome) lead to the formation of protoporphyrin IX. At this stage, the pathway branches to yield phytochromobilin, the chromophore of the plant photoreceptor phytochrome, via an iron-dependent pathway, or the chlorophylls via a Mg-dependent pathway. The synthesis of phytochromobilin requires three enzymes, ferrochelatase (Chow et al., Plant J., 15:531, 1998; two isogenes in A. thaliana genome), heme oxygenase (Muramoto et al., Plant Cell, 11:335, 1999; Davis et al., Proc. Natl. Acad. Sci. USA, 96:6541 1999; Davis et al., Plant Physiol., 126:656, 2001; 4 isogenes in A. thaliana genome), and phytochromobilin synthase (Kohchi et al., Plant Cell, 13:425, 2001; single copy gene in A. thaliana). The Mg-dependent pathway to chlorophylls starts with Mg-protoporphyrinogen IX chelatase (Papenbrock et al., Plant J., 12:981, 1997; 3 isogenes in A. thaliana genome). Subsequent reactions involve Mg-protoporphyrinogen IX methyltransferase, that was cloned from Synechocystis (Smith et al., Plant Mol. Biol., 30:1307, 1996) and has homologues in plants (single copy gene in A. thaliana), and two enzymes, Mg-protoporphyrinogen IX monomethylester cyclase and 8-vinyl reductase. The conversion to chlorophyllide a is catalyzed by protochlorophyllide oxidoreductase (Benli et al., Plant Mol. Biol., 16:615, 1991; Armstrong et al., Plant Physiol., 108:1505, 1995; Su et al., Plant Mol. Biol., 47:805, 2001), for which three copies are encoded in the A. thaliana genome. The synthesis of chlorophyll a involves the attachment of the phytyl side chain by chlorophyll synthetase (Gaubier et al., Mol. Gen. Genet., 249:58 1995; single copy gene in A. thaliana). Chlorophyll b differs from chlorophyll a in the conversion of a methyl group to a formyl side group, a reaction catalyzed by chlorophyll a oxygenase (Espineda et al., Proc. Natl. Acad. Sci. USA, 96:10507, 1999; single copy gene in A. thaliana). Senescence and subsequent death are terminal phases in the development of all organs of a plant, including leaves, stems, roots, and flowers. During senescence, one of the clearest symptoms is the loss of green color due to chlorophyll breakdown. The first step in the catabolic cascade is the conversion of chlorophyll to chlorophyllide in a reaction catalyzed by chlorophyllase (Benedetti et al., Plant Physiol., 116:1037, 1998; Tsuchiya et al., Proc. Natl. Acad. Sci. USA, 96:15362, 1999; 2 isogenes in A. thaliana). The other product of chlorophyllase action is phytol, which usually accumulates in the lipid globules of gerontoplasts in the form of esters. Mg-de-chelatase is required to remove Mg from chlorophyllide, a reaction which is followed by a ring closure catalyzed by pheophorbide a oxygenase (both genes not cloned yet). The resulting catabolite (red chlorophyll catabolite; RCC) is further metbolized by RCC reductase (Mach et al., Proc. Natl. Acad. Sci. USA, 98:771, 2001; single copy gene in A. thaliana), which catalyzes the ferredoxin-dependent reduction of a double bond in the pyrrol system of RCC to produce fluorescent chlorophyll catabolite (FCC). FCC is then exported and further metabolized in the cytosolic and vacuolar compartments to yield the nonfluorescent chlorophyll catabolites (Hortensteiner, Phytochemistry, 49:953, 1998). The regulation of chlorophyll biosynthesis is complex; thus far, the characterized mediators include light (activation of protochlorophyllide oxidoreductase; Armstrong et al., Plant Physiol, 108:1505, 1995; Su et al., Plant Mol. Biol., 47:805, 2001), heme (negative regulator of glutamyl-tRNA reductase; Pontoppidan and Kannangara, Eur. J. Biochem., 225:529, 1995) and FLU (negative regulator of several enzymes of the Mg-dependent branch of the pathway; Meskauskiene et al., Proc. Natl. Acad. Sci. USA, 98:12826, 2001).

Apart from conventional breeding and mutagenesis techniques, recombinant DNA techniques are now increasingly used in order to specifically interfere with the photosynthetic activity of plants. A prerequisite for this is that DNA sequences be provided which encode enzymes involved in the photosynthetic processes, such as those involved in cholorphyll metabolism.

The present invention now provides a subset of nucleic acid molecules that are involved in or associated with the chlorophyll biosynthesis pathway. Representative examples of those subset genes are provided in SEQ ID NOs: 31, 47, 53, 105, 117, 127, 139, 143, 149, 175, 177, 191, 199, 221, 277, 295, 307, 325, 335, 339, 355, and 363 of the Sequence Listing.

In a particular embodiment, the present invention relates to an isolated nucleic acid molecule comprising a nucleotide sequence encoding a polypeptide the enzymatic activity of which is involved in associated with chlorophyll metabolism in the plant plastid and cytosol, respectively, which nucleic acid molecule is substantially similar to a nucleic acid encoding a polypeptide as given in any one of the SEQ ID NOs of Table 1 such as SEQ ID NOs: 32, 48, 54, 106, 118, 128, 140, 144, 150, 176, 178, 192, 200, 222, 278, 296, 308, 326, 336, 340, 356, and 364.

More specifically, the invention relates to an isolated nucleic acid molecule comprising a nucleotide sequence encoding a polypeptide the enzymatic activity of which is involved in associated with chlorophyll metabolism in the plant plastid and cytosol, respectively, and which is substantially similar, and preferably has at least between 70%, and 99% amino acid sequence identity to at least one polypeptide as given in any one of the SEQ ID NOs of Table 1 such as SEQ ID NOs: 32, 48, 54, 106, 118, 128, 140, 144, 150, 176, 178, 192, 200, 222, 278, 296, 308, 326, 336, 340, 356, and 364, with any individual number within this range of between 70% and 99% also being part of the invention.

The invention further relates to an isolated nucleic acid molecule comprising a nucleotide sequence encoding a polypeptide the enzymatic activity of which is involved in associated with chlorophyll metabolism in the plant plastid and cytosol, respectively, and which is immunologically reactive with antibodies raised against a polypeptide as given in any one of the SEQ ID NOs of Table 1 such as SEQ ID NOs: 32, 48, 54, 106, 118, 128, 140, 144, 150, 176, 178, 192, 200, 222, 278, 296, 308, 326, 336, 340, 356, and 364.

More particularly, the invention relates to an isolated nucleic acid molecule comprising a nucleotide sequence

a) as given in any one of the SEQ ID NOs of Table 1 such as SEQ ID NOs: 31, 47, 53, 105, 117, 127, 139, 143, 149, 175, 177, 191, 199, 221, 277, 295, 307, 325, 335, 339, 355, and 363;

b) having substantial similarity to (a);

c) having at least 15 nucleotides and capable of hybridizing to (a) or the complement thereof under stringent, highly stringent or very highly stringent conditions;

d) having at least 15 nucleotides and capable of hybridizing to a nucleic acid comprising 50 to 200 or more consecutive nucleotides of a nucleotide sequence of (a), or the complement thereof under stringent, highly stringent or very highly stringent conditions;

e) complementary to (a), (b) or (c); and

f) which is a reverse complement of (a), (b) or (c).

By providing a subset of genes encoding polypeptides that are involved in chlorophyll metabolism it is now possible to interfere with chlorophyll metabolism to produce chlorophyll with modified physico/chemical characteristics and increased photosynthetic efficiency.

An increased production of chlorophylls, e.g. by over-expression of genes involved in the biosynthetic pathway may result in improved plant fitness.

Enzymes of the biosynthetic pathway have been used successfully as targets for hebicides. For example, protoprophyrinogen oxidase is the target enzyme for phthalimide and diphenylether herbicides (Duke et al., Am. Chem. Soc. Symp., Ser. 559:191, 1994).

In a further embodiment, the present invention provides a subset of nucleic acid molecules comprising genes encoding polypeptides the enzymatic activity of which is involved in or associated with isoprenoid biosynthesis in the cytosol and/or mitochondria.

Cytosolic and mitochondrial isoprenoids are derived from acetyl-CoA, which itself is obtained from carbon fixation of CO₂ through a sequence of glycolytic enzymes. The early steps of the biosynthetic pathway include acetoacetyl-CoA thiolase, which was cloned from radish (Vollack and Bach, Plant Physiol., 111:1097, 1996; two homologous genes are detectable in A. thaliana genome), 3-hydroxy-3-mthylglutaryl (HMG) CoA synthase (Montamat et al., Gene, 167:197, 1995; single copy gene in A. thaliana genome), and HMG-CoA reductase (Caelles et al., Plant Mol. Biol., 13:627, 1989; Enjuto et al., Proc. Natl. Acad. Sci. USA, 91:927, 1994; two gene copies exist in the A. thaliana genome) to yield mevalonate. Two successive phosphorylations, catalyzed by mevalonate kinase (Riou et al., 1994; single copy gene in A. thaliana) and phosphomevalonate kinase (a distant homologue of the yeast (Tsay and Robinson, Mol. Cell. Biol., 11:620, 1991) gene is detectable in the A. thaliana genome), and a decarboxylation (mevalonate diphosphate decarboxylase; Cordier et al., Plant Mol. Biol., 39:953, 1999; 2 isogenes in A. thaliana genome) lead to the formation of IPP. Interestingly, the A. thaliana genome does not contain obvious additional homologues to the two plastidial IPPI isoenzymes; however, a phylogenetic analysis has indicated that an alternative translation start site may allow the generation of cytosolic isoenzymes from the same genes (Cunningham and Gantt, Plant Cell Physiol., 41:119, 2000). A. thaliana contains two farnesyl diphosphate synthase (FPPS) genes, coding for one cytosolic isoenzyme (Delourme et al., Plant Mol. Biol., 26:1867, 1994; involved in phytosterol and sesquiterpene biosynthesis) and one mitochondrial isoenzyme (Cunillera et al., J. Biol. Chem., 272:15381, 1997; involved in ubiquinone biosynthesis).

In a specific embodiment the invention relates to an isolated nucleic acid molecule comprising a nucleotide sequence encoding a polypeptide the enzymatic activity of which is involved in or associated with the mevalonate pathway in the cytosol and/or mitochondria which nucleic acid molecule is substantially similar to a nucleic acid encoding a polypeptide as given in the SEQ ID NOs identified in Table 1 such as SEQ ID NOs: 112, 134, 166, 294, 316, 354, and 366.

More specifically, the invention relates to an isolated nucleic acid molecule comprising a nucleotide sequence encoding a polypeptide the enzymatic activity of which is involved in or associated with the mevalonate pathway in the cytosol and/or mitochondria, respectively, and has at least between 70%, and 99% amino acid sequence identity to at least one polypeptide as given in the SEQ ID NOs identified in Table 1 such as SEQ ID NOs: 112, 134, 166, 294, 316, 354, and 366, with any individual number within this range of between 70% and 99% also being part of the invention.

The invention further relates to an isolated nucleic acid molecule comprising a nucleotide sequence encoding a polypeptide the enzymatic activity of which is involved in or associated with the mevalonate pathway in the cytosol and/or mitochondria, respectively, and is immunologically reactive with antibodies raised against a polypeptide as given in the SEQ ID NOs identified in Table 1 such as SEQ ID NOs: 112, 134, 166, 294, 316, 354, and 366.

More particularly, the invention relates to an isolated nucleic acid molecule comprising a nucleotide sequence

a) as given given in the SEQ ID NOs identified in Table 1 such as SEQ ID NOs: 111, 133, 165, 293, 315, 353, and 365;

b) having substantial similarity to (a);

c) having at least 15 nucleotides and capable of hybridizing to (a) or the complement thereof under stringent, highly stringent or very highly stringent conditions;

d) having at least 15 nucleotides and capable of hybridizing to a nucleic acid comprising 50 to 200 or more consecutive nucleotides of nucleotide sequences given in (a) or the complement thereof under stringent, highly stringent or very highly stringent conditions;

e) complementary to (a), (b) or (c); and

f) which is a reverse complement of (a), (b) or (c).

A further subset of genes provided herein comprises genes that encode polypeptides with an activity that is involved in or associated with the biosynthesis of long-chain polyprenyl diphoshates in the cytosol.

The invention thus also relates to an isolated nucleic acid molecule comprising a nucleotide sequence encoding a polypeptide the enzymatic activity of which is involved in or associated with the biosynthesis of long-chain polyprenyl diphoshates in the cytosol and which nucleic acid is substantially similar to a nucleic acid encoding a polypeptide as given in any one of the SEQ ID NOs of table 1.

The biosynthetic sequence to dolichols, which are long-chain polyprenols required for the synthesis of glycoproteins, starts with dehydrodolichyl diphosphate synthase (Cunillera et al., FEBS Lett., 477:170, 2000). In the A. thaliana genome, three copies of this cis-prenyltransferase are detectable, two of which appear to encode cytosolic isoenzymes (ER-associated), whereas one isoenzymes, based on a TargetP analysis, appears to be localized to mitochondria. Further steps towards dolichyl-sugars are completely undefined in plants. In addition, Oh et al. (2000) have reported on a cis-prenyltransferase, homologous to undecaprenyl diphosphate synthase from eubacteria, which catalyzes the formation of polyprenyl diphosphates ranging from C100 to C130. The A. thaliana genome contains three copies for genes putatively encoding cis-prenyltransferases, thus producing cytosolic polyprenyl diphosphates the metabolic fate of which is currently unknown.

A further subset of genes provided herein comprises genes that encode polypeptides with an activity that is involved in or associated with phytosterol and brassinosteroid metabolism in the plant cytosol.

The biosynthesis of sterols, which are important in controlling membrane fluidity and are involved in plant embryogenesis, starts with the condensation of two molecules of famesyl diphosphate (squalene synthase; Nakashima et al., Proc. Natl. Acad. Sci. USA, 92:2328, 1995; 2 gene copies detectable in A. thaliana genome) and a subsequent oxidation (squalene monooxygenase; Schafer et al., Plant Mol. Biol., 39:721, 1999; 6 isogenes in A. thaliana genome). The resulting 2,3-epoxysqualene serves as a substrate for triterpene synthases which catalyze cyclization reactions that ultimately lead to, besides the typical membrane sterols, a vast array of triterpenoid compounds. A sequence relationship tree of the A. thaliana triterpenoid synthases indicates the presence of three distinct subfamilies: one family codes for cycloartenol synthase (Corey et al., Proc. Natl. Acad. Sci. USA, 90:11628, 1993; two copies in A. thaliana), a second family includes lupeol synthase-like sequences (Herrera et al., Phytochemistry, 49:1905, 1998; 4 members in A. thaliana), and the funtion of a third family, which comprises six members, has yet to be characterized (Husselstein-Muller et al., Plant Mol. Biol., 45:75, 2001). The further conversions of the lupeol and amyrin intermediates synthesized by the members of the second triterpene synthase family are unknown. The membrane sterol pathway involves methyltransferases acting on cycloartenol (sterol C24-methyltransferase 1; first cloned from soybean (Shi et al., J. Biol. Chem., 271:9384, 1996); one homologue in A. thaliana genome) and 24-methylenelophenol (sterol C24-methyltransferase 2; Husselstein et al., FEBS Lett., 381:87, 1996; Bouvier-Nave et al., Eur. J. Biochem., 246:518, 1997; 2 isogenes in A. thaliana). The substrates for demethylases include obtusifoliol (obtusifoliol C14-methyl oxidase (CYP51); Kushiro et al., Biochim. Biophys. Res. Commun., 285:98, 2001; 2 isogenes in A. thaliana), 24-methylenelophenol/citrostadienol (C4-methyl oxidase; Damet et al., FEBS Lett., 508:39, 2001; isogenes in A. thaliana) and 24-methylenecycloartenol (24-methylenecycloartenol C4-methyl oxidase; not cloned yet; two candidate genes based on sequence relatedness to CYP51 in A. thaliana genome; Table I). Isomerases are involved in the conversion of cycloeucalenol to obtusifoliol (Lovato et al., J. Biol. Chem., 275:13394, 2000) and of 4α-methylfecosterol to 24-methylenelophenol (Grebenok et al., Plant Mol. Biol., 38:807, 1998). Three sterol reductases have been cloned, comprising obtusifoliol C14-reductase (Schrick et al., Genes Dev., 14:1471 2000), sterol C7-reductase (Lecain et al., J. Biol. Chem., 271:10866, 1996) and a sterol C24-reductase (Klahre et al., Plant Cell, 10:1677, 1998). Thus far, one desaturase has been cloned (sterol C5-desaturase; Gachotte et al., Plant J., 9:391, 1996; 2 closely related genes in A. thaliana genome), with a sterol C22-desaturase, leading from β-sitosterol to stigmasterol, still undiscovered. Besides free sterols, steryl glycosides and acylated steryl glycosides are abundant in plant cell membranes. The first two enzymes involved in these conversions have been characterized as UCP-glucose:sterol glucosyltransferases (Wameke et al., Plant Mol. Biol., 35:597, 1997; two gene copies detectable in the A. thaliana genome). The biosynthetic steps leading from campesterol to the plant hormone brassinolide have been proposed, but few enzymes have been characterized. Thus far, a sterol 5α-reductase (DET2; Li et al., Science, 272:398, 1996), a C22α-hydroxylase (Choe et al., Plant Cell, 10:231, 1998) and a C23α-hydroxylase (Szekeres et al., Cell, 85:171, 1996) have been cloned. The biosynthetic sequence from campesterol to brassinolide involves 4 steps for which a catalysis by a 3β-hydroxy dehydrogenase or a 3-keto-reductase is required. Interestingly, the A. thaliana genome contains three copies of a gene with homology to 3β-hydroxysteroid dehydrogenases of animals, in which this bifunctional enzyme catalyzes multiple oxidative conversions of Δ⁵-ene-3 β-hydroxysteroids and is also able to act as a 3-keto reductase (Rogerson et al., J. Steroid Biochem. Mol. Biol., 55:481, 1995). In addition, the A. thaliana genome contains a copy for a putative 17β-hydroxysteroid dehydrogenase and 8 copies of a putative 11β-hydroxysteroid dehydrogenase, the function of which in plants has yet to be demonstrated. The closest plant homologue of the A. thaliana 11β-hydroxysteroid dehydrogenase family is a gene from Sesamum indicum that is annotated as a steroleosin (AF302806), a reductase of plant oil bodies. Steroid sulfotransferases play an important role in the modulation of the biological activity of a number of metabolites, including steroid hormones and neurotransmitters. Thus far, only one plant gene, from Brassica napus, has been cloned, and its encoded enzyme has been demonstrated to catalyze the sulfonation of a brassinosteroid, which results in the loss of biological activity (Rouleau et al., J. Biol. Chem., 274:20925 1999). The A. thaliana genome contains a family of 15 genes with homology to mamalian steroid sulfotransferases.

In one further embodiment, the invention thus relates to an isolated nucleic acid molecule comprising a nucleotide sequence encoding a polypeptide the enzymatic activity of which is involved in or associated with phytosterol and brassinosteroid metabolism in the plant cytosol, which nucleotide sequence is substantially similar to a nucleic acid sequence encoding a polypeptide as given in in the SEQ ID NOs identified in Table 1 such as SEQ ID: 4, 6, 14, 16, 22, 34, 36, 38, 46, 50, 60, 62, 66, 92, 96, 120, 124, 132, 148, 164, 170, 174, 184, 188, 196, 198, 220, 252, 270, 288, 304, 310, 312, 324, 330, 332, 334, 338, 342, 362, 368, 380, 390, and 406.

More specifically, the invention relates to an isolated nucleic acid molecule comprising a nucleotide sequence encoding a polypeptide the enzymatic activity of which is involved in or associated with phytosterol and brassinosteroid metabolism in the plant cytosol, and which has at least between 70%, and 99% amino acid sequence identity to at least one polypeptide as given in the SEQ ID NOs identified in Table 1 such as SEQ ID: 4, 6, 14, 16, 22, 34, 36, 38, 46, 50, 60, 62, 66, 92, 96, 120, 124, 132, 148, 164, 170, 174, 184, 188, 196, 198, 220, 252, 270, 288, 304, 310, 312, 324, 330, 332, 334, 338, 342, 362, 368, 380, 390, and 406, with any individual number within this range of between 70% and 99% also being part of the invention.

The invention further relates to an isolated nucleic acid molecule comprising a nucleotide sequence encoding a polypeptide the enzymatic activity of which is involved in or associated with phytosterol and brassinosteroid metabolism in the plant cytosol, and which is immunologically reactive with antibodies raised against a polypeptide as given in the SEQ ID NOs identified in Table 1 such as SEQ ID: 4, 6, 14, 16, 22, 34, 36, 38, 46, 50, 60, 62, 66, 92, 96, 120, 124, 132, 148, 164, 170, 174, 184, 188, 196, 198, 220, 252, 270, 288, 304, 310, 312, 324, 330, 332, 334, 338, 342, 362, 368, 380, 390, and 406.

More particularly, the invention relates to an isolated nucleic acid molecule comprising a nucleotide sequence

a) as given in the SEQ ID NOs identified in Table 1 such as SEQ ID: 3, 5, 13, 15, 21, 33, 35, 37, 45, 49, 59, 61, 65, 91, 95, 119, 123, 131, 147, 163, 169, 173, 183, 187, 195, 197, 219, 251, 269, 287, 303, 309, 311, 323, 329, 331, 333, 337, 341, 361, 367, 379, 389, and 405;

b) having substantial similarity to (a);

c) having at least 15 nucleotides and capable of hybridizing to (a) or the complement thereof;

d) having at least 15 nucleotides and capable of hybridizing to a nucleic acid comprising 50 to 200 or more consecutive nucleotides of a nucleotide sequence of (a), or the complement thereof under stringent, highly stringent, or very highly stringent conditions;

e) complementary to (a), (b) or (c); and

f) which is a reverse complement of (a), (b) or (c).

By providing these genes it is now possible to regulate the expression levels of the encoded protein products in the plant by applying methods known in the art including overexpressing or down-regulating the nucleic acid molecule in a plant, thereby modifying the phytosterol and brassinosteroid content of the plant, respectively.

Because of their well-established cholesterol-lowering activities, plant sterols have been implicated with providing an improved protection against coronary heart disease; in addition, certain reports indicate that phytosterols may have an impact on reducing the risk of certain cancers (Jones and Raeini-Sarjaz, Nutr. Rev. 59:21, 2001). The over-expression of genes involved in the biosynthesis of sterols may thus be desireable to obtain high sterol plants to be used in margerines and nutritional supplements.

In a further embodment the invention further relates to an isolated nucleic acid molecule comprising a nucleotide sequence encoding a polypeptide the activity of which is involved in or associated with the biosynthesis of ubiquinone in mitochondria which nucleotide sequence is substantially similar to a sequence encoding a polypeptide as given in any one of the SEQ ID NOs of Table 1 such as SEQ ID NOs: 8, 240, and 382.

Ubiquinone is an integral part of the respiratory chain in mitochondria. The first committed step of the biosynthetic pathway to ubiquinone, which is an integral part of the mitochondrial electron transfer chain, is catalyzed by p-hydroxybenzoate solanesyltransferase. A homologue of the eubacterial (ubiA) and the yeast (CoQ2) genes for this enyzme (Meganathan, FEMS Microbiol. Lett., 203:131, 2001) is detectable in the A. thaliana genome and, according to a TargetP analysis, specifies a protein with the required N-terminal mitochondrial targeting sequence. Additional homologues to eubacterial/yeast genes involved in this pathway include a monooxygenase (ubiF/CoQ7-like), a bifuctional O-methyltransferase (CoQ3-like; Avelange-Macharel and Joyard, Plant J., 14:203 1998), two C-methyltransferases (ubiH/CoQ6-like and ubiE/CoQ5-like), and a CoQ4-like sequence (function unknown). The C-methyltransferases are part of a larger gene family and their involvement in ubiquinone biosynthesis is suggested only by the presence of an encoded mitochondrial targeting sequence.

In particular, the invention relates to an isolated nucleic acid molecule comprising a nucleotide sequence encoding a polypeptide the activity of which is involved in or associated with the biosynthesis of ubiquinone in mitochondria, and which is substantially similar, and preferably has at least between 70%, and 99% amino acid sequence identity to at least one polypeptide as given in any one of the SEQ ID NOs of Table 1 such as SEQ ID NOs: 8, 240, and 382, with any individual number within this range of between 70% and 99% also being part of the invention.

The invention further relates to an isolated nucleic acid molecule comprising a nucleotide sequence encoding a polypeptide the activity of which is involved in or associated with the biosynthesis of ubiquinone in mitochondria, and which is immunologically reactive with antibodies raised against a polypeptide as given in any one of the SEQ ID NOs of Table 1 such as SEQ ID NOs: 8, 240, and 382

More particularly, the invention relates to an isolated nucleic acid molecule comprising a nucleotide sequence

m) as given in in any one of the SEQ ID NOs of Table 1 such as SEQ ID NOs: 7, 239, and 381;

n) having substantial similarity to (a);

o) having at least 15 nucleotides and capable of hybridizing to (a) or the complement thereof under stringent, highly stringent or very highly stringent conditions;

p) having at least 15 nucleotides and capable of hybridizing to a nucleic acid comprising 50 to 200 or more consecutive nucleotides of a nucleotide sequence of (a) or the complement thereof under stringent, highly stingent or very highly stringent conditions;

q) complementary to (a), (b) or (c); and

r) which is a reverse complement of (a), (b) or (c).

By providing these genes it is now possible to regulate the expression levels of the encoded protein products in the plant by applying methods known in the art including overexpressing or down-regulating the nucleic acid molecule in a plant, thereby modifying the ubiquinone content of the plant, respectively.

Coenzyme Q10 (CoQ10) has a pathophysiologic role in many disease states, including heart failure, angina, and hypertension (Tran et al., Pharmacotherapy, 21:797, 2001). Thus, an over-expression of genes involved in ubiqionone biosynthesis in transgenic plants may be of use in the nutraceutical marketplace.

The invention provides a further subset of genes comprising a nucleotide sequence that encodes polypeptides which exhibit an enzymatic activity that is involved in or associated with the biosynthesis of monoterpenes and sesquiterpenes.

In a particular embodiment, the invention thus relates to an isolated nucleic acid molecule comprising a nucleotide sequence encoding a polypeptide the activity of which is involved in or associated with the biosynthesis of monoterpenes and sesquiterpenes, which nucleotide sequence is substantially similar to a nucleic acid sequence encoding a polypeptide as given in the SEQ ID NOs identified in Table 1 such as SEQ ID NOs: 2, 10, 20, 40, 42, 44, 52, 56, 58, 70, 72, 78, 82, 100, 102, 110, 116, 130, 138, 146, 152, 158, 160, 168, 172, 180, 182, 186, 202, 210, 212, 218, 228, 232, 234, 242, 248, 250, 258, 264, 274, 280, 282, 284, 306, 320, 322, 328, 348, 352, 358, 370, 376, 386, 388, and 404.

More specifically, the invention relates to an isolated nucleic acid molecule comprising a nucleotide sequence encoding a polypeptide the activity of which is involved in or associated with the biosynthesis of monoterpenes and sesquiterpenes, and which has at least between 70%, and 99% amino acid sequence identity to at least one polypeptide as given in the SEQ ID NOs identified in Table 1 such as SEQ ID NOs: 2, 10, 20, 40, 42, 44, 52, 56, 58, 70, 72, 78, 82, 100, 102, 110, 116, 130, 138, 146, 152, 158, 160, 168, 172, 180, 182, 186, 202, 210, 212, 218, 228, 232, 234, 242, 248, 250, 258, 264, 274, 280, 282, 284, 306, 320, 322, 328, 348, 352, 358, 370, 376, 386, 388 and 404, with any individual number within this range of between 70% and 99% also being part of the invention.

The invention further relates to an isolated nucleic acid molecule comprising a nucleotide sequence encoding a polypeptide the activity of which is involved in or associated with the biosynthesis of monoterpenes and sesquiterpenes, and which is immunologically reactive with antibodies raised against a polypeptide as given in the SEQ ID NOs identified in Table 1 such as SEQ ID NOs: 2, 10, 20, 40, 42, 44, 52, 56, 58, 70, 72, 78, 82, 100, 102, 110, 116, 130, 138, 146, 152, 158, 160, 168, 172, 180, 182, 186, 202, 210, 212, 218, 228, 232, 234, 242, 248, 250, 258, 264, 274, 280, 282, 284, 306, 320, 322, 328, 348, 352, 358, 370, 376, 386, 388 and 404.

More particularly, the invention relates to an isolated nucleic acid molecule comprising a nucleotide sequence

g) as given in any one of the SEQ ID NOs identified in Table 1 such as SEQ ID NOs:1, 9, 19, 39, 41, 43, 51, 55, 57, 69, 71, 77, 81, 99, 101, 109, 115, 129, 137, 145, 151, 157, 159, 167, 171, 179, 181, 185, 201, 209, 211, 217, 227, 231, 233, 241, 247, 249, 257, 263, 273, 279, 281, 283, 305, 319, 321, 327, 347, 351, 357, 369, 375, 385, 387, and 403;

h) having substantial similarity to (a);

i) having at least 15 nucleotides and capable of hybridizing to (a) or the complement thereof under stringent, highly stringent or very highly stringent conditions;

j) having at least 15 nucleotides and capable of hybridizing to a nucleic acid comprising 50 to 200 or more consecutive nucleotides of a nucleotide sequence of (a), or the complement thereof under stringent, highly stringent or very highly stringent conditions;

k) complementary to (a), (b) or (c); and

l) which is a reverse complement of (a), (b) or (c).

By providing these genes it is now possible to regulate the expression levels of the encoded protein products in the plant by applying methods known in the art including overexpressing or down-regulating the nucleic acid molecule in a plant, thereby modifying the monoterpene and sesquiterpene content of the plant, respectively.

For example, A. thaliana has been reported to emit a complex mixture of volatiles as a response to herbivory, which also includes monoterpenoids (Van Poeke et al., J. Chem. Ecol., 27:1911, 2001). The A. thaliana genome encodes a large family of enzymes with homology to monoterpene and sesquiterpene synthases, which catalyze the first committed step to terpenoids. Thus far, only one gene product, a myrcine/β-ocimene synthase, has been characterized from A. thaliana (Bohlmann et al., Arch. Biochem. Biophys., 375:261, 2000).

In a further embodiment the invention provides a subset of genes which encode polypeoptides that are involved in or associated with protein prenylation.

Protein prenylation, a post-translational modification which involves the covalent attachment of famesyl (C15) or geranylgeranyl (C20) isoprenoid moieties to target proteins in the cytosol, plays important roles in signal transduction and intracellular trafficking pathways (Crowell, Prog. Lipid Res., 39:393, 2000). The transfer of the prenyl group is catalyzed by famesyltransferase (FTase) and geranylgeranyltransferase (GGTase). FTase and GGTase I act as heterodimers with a common α-subunit (Qian et al, Plant Cell, 8:2381, 1996; Yalovski et al., Mol. Cell. Biol., 17:1986, 1997) and a distict β-subunit (Caldelari et al., Plant Physiol., 126:1416, 2001). All of these subunits occur as single copy genes in A. thaliana genome. Protein geranylgeranyltransferase II was first cloned and characterized from rat (Armstrong et al., J. Biol. Chem., 268:12221, 1993), and homologues for both the α-subunit and the α-subunit are detectable in the A. thaliana genome (two copies each). Geranylgeranyl diphosphate, the precursor for geranylgeranylations, stems most likely from a cytosolic pool provided by two cytosolic GGPPS isoenzymes (Okada et al., Plant Physiol., 122:1045, 2000). A second protein prenylation pathway, that involves a modification with phytol (C20), has been described for plant plastids.

In one specific embodiment, the invention relates to an isolated nucleic acid molecule encoding a polypeptide that is involved in or associated with protein prenylation, which nucleotide sequence is substantially similar to a nucleic acid sequence encoding a polypeptide as given in any one of the SEQ ID NOs of Table 1 such as SEQ ID NOs: 24, 64, 190, 256, 290, 344, and 374.

More specifically, the invention relates to an isolated nucleic acid molecule the expression product of which is involved in or associated with protein prenylation comprising a nucleotide sequence encoding a polypeptide that is involved in or associated with protein prenylation and has at least between 70%, and 99% amino acid sequence identity to at least one polypeptide as given in any one of the SEQ ID NOs of Table 1 such as SEQ ID NOs: 24, 64, 190, 256, 290, 344, and 374, with any individual number within this range of between 70% and 99% also being part of the invention.

The invention further relates to an isolated nucleic acid molecule the expression product of which is involved in or associated with protein prenylation comprising a nucleotide sequence encoding a polypeptide that is involved in or associated with protein prenylation and immunologically reactive with antibodies raised against a polypeptide as given in any one of the SEQ ID NOs of Table I such as SEQ ID NOs: 24, 64, 190, 256, 290, 344, and 374.

More particularly, the invention relates to an isolated nucleic acid molecule comprising a nucleotide sequence

a) as given in any one of the SEQ ID NOs of Table 1 such as SEQ ID NOs: 23, 63, 189, 255, 289, 343, and 373;

b) having substantial similarity to (a);

c) having at least 15 nucleotides and capable of hybridizing to (a) or the complement thereof under stringent, highly stringent or very highly stringent conditions;

d) having at least 15 nucleotides and capable of hybridizing to a nucleic acid comprising 50 to 200 or more consecutive nucleotides of a nucleotide sequences of (a) or the complement thereof under stringent, highly stringent or very highly stringent conditions;

e) complementary to (a), (b) or (c); and

f) which is a reverse complement of (a), (b) or (c).

By providing these genes it is now possible to regulate the expression levels of the encoded protein products in the plant by applying methods known in the art including overexpressing or down-regulating the nucleic acid molecule in a plant, thereby modifying the protein prenylation pattern of the plant.

The demonstration that the human Ras protein requires prenylation for its cancer-causing activity has led to an intense search for famesyltransferase and geranylgeranyl-transferase inhibitors as potential anticancer drugs (Sebti and Hamilton, Pharmacol. Ther., 74:103, 1997).

The plant enzymes could be used in small molecule screens to disrupt protein prenylation. The assembly of the core oligosaccharide region of asparagine-linked glycoproteins proceeds by means of the dolichol pathway (Lerouge et al., Curr. Pharm. Biotechnol., 1:347, 2000).

The number of therapeutic proteins successfully produced in plants is steadily increasing and is expected to grow even more rapidly in the future. Most therapeutic proteins are glycoproteins and N-glycosylation is often essential for their stability, folding and biological activity.

Recombinant glycoproteins of mammalian origin expressed in transgenic plants largely retain their biological activity. However, plants are not ideal for production of pharmaceutical proteins because they produce molecules with glycans that are not compatible with therapeutic applications in humans.

As a consequence, strategies to humanise plant N-glycans are now developed, and expression changes in the prenyltransferases necessary to produce the dolichol chains of glycans may be of use. A knock-out mutant of the beta-subunit of farnesyltransferase shows a reduction in transpirational water loss in dry growing conditions (Pei et al., Science 282:287, 1998), The present invention provides a set of genes, which are shown to be involved in isoprenoid metabolism as specified hereinbefore. The genes within this subgroup are useful tools for generating plants with modified compositional characteristics leading to improved nutritional and/or pharmaceutical properties.

Based on the Oryza nucleic acid sequences of the present invention as given in the SEQ ID NOs of the Sequence Listing, orthologs may be identified or isolated from the genome of any desired organism, preferably from another plant, but especially from a cereal plant such as wheat, banana, barley, maize, etc, according to well known techniques based on their sequence similarity to the Oryza nucleic acid sequences, e.g., hybridization, PCR or computer generated sequence comparisons. For example, all or a portion of a particular Oryza nucleic acid sequence is used as a probe that selectively hybridizes to other gene sequences present in a population of cloned genomic DNA fragments or cDNA fragments (i.e., genomic or cDNA libraries) from a chosen source organism. Further, suitable genomic and cDNA libraries may be prepared from any cell or tissue of an organism. Such techniques include hybridization screening of plated DNA libraries (either plaques or colonies; see, e.g., Sambrook et al., Molecular Cloning, 2^{nd} ed. Cold Spring Harbor Laboratory Press, 1989) and amplification by PCR using oligonucleotide primers preferably corresponding to sequence domains conserved among related polypeptide or subsequences of the nucleotide sequences provided herein (see, e.g., Innis et al., supra). These methods are particularly well suited to the isolation of gene sequences from organisms closely related to the organism from which the probe sequence is derived. The application of these methods using the Oryza sequences as probes is well suited for the isolation of gene sequences from any source organism, preferably other plant species. In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the sequence of the invention. Methods for preparation of probes for hybridization and for construction of cDNA and genomic libraries are generally known in the art and are disclosed in Sambrook et al., supra. In general, sequences that hybridize to the sequences disclosed herein will have at least 40% to 50%, about 60% to 70% and even about 80% 85%, 90%, 95% to 98% or more identity with the disclosed sequences. That is, the sequence similarity of sequences may range, sharing at least about 40% to 50%, about 60% to 70%, and even about 80%, 85%, 90%, 95% to 98% sequence similarity, with each individual number within the ranges given above also being part of the invention.

The nucleic acid molecules of the invention can also be identified by, for example, a search of known databases for genes encoding polypeptides having a specified amino acid sequence identity or DNA having a specified nucleotide sequence identity. Methods of alignment of sequences for comparison are well known in the art and are described hereinabove.

The present invention further provides a composition, an expression cassette or a recombinant vector containing the nucleic acid molecule of the invention as disclosed herinbefore, and host cells comprising the expression cassette or vector, e.g., comprising a plasmid.

In particular, the present invention provides an expression cassette or a recombinant vector comprising a suitable promoter linked to a nucleic acid segment of the invention, representative examples of which are provided in the SEQ ID NOs of the Sequence Listing, which, when present in a plant, plant cell or plant tissue, results in transcription of the linked nucleic acid segment.

Promoters which are useful for plant transgene expression include those that are inducible, viral, synthetic, constitutive (Odell et al., Nature, 313:810, 1985), temporally regulated, spatially regulated, tissue-specific, and spatio-temporally regulated.

Where expression in specific tissues or organs is desired, tissue-specific promoters may be used. In contrast, where gene expression in response to a stimulus is desired, inducible promoters are the regulatory elements of choice. Where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

Suitable promoter and/or regulatory sequences further include those that are preferentially or specifically active in selected plant tissues such as, for example, the gree tissue (leaf, stem), grain (grain endosperm or the grain embryo), fruit, flower, etc.

Further, the invention provides isolated polypeptides encoded by any one of the open reading frames of the invention, representative examples of which are provided in the SEQ ID NOs of the Sequence Listing, or a fragment thereof, which encodes a polypeptide which has substantially the same activity as the corresponding polypeptide encoded by an ORF given in the SEQ ID NOs of the Sequence Listing, or the orthologs thereof.

Virtually any DNA composition may be used for delivery to recipient plant cells, e.g., monocotyledonous cells, to ultimately produce fertile transgenic plants in accordance with the present invention. For example, DNA segments or fragments in the form of vectors and plasmids, or linear DNA segments or fragments, in some instances containing only the DNA element to be expressed in the plant, and the like, may be employed. The construction of vectors which may be employed in conjunction with the present invention will be known to those of skill of the art in light of the present disclosure (see, e.g., Sambrook et al., 1989; Gelvin et al., Plant Molecular Biology Manual, Kluwer Academic Publishers, 1990).

It is one of the objects of the present invention to provide recombinant DNA molecules comprising a nucleotide sequence which directs transcription according to the invention operably linked to a nucleic acid segment or sequence of interest.

The nucleic acid segment of interest can, for example, code for a ribosomal RNA, an antisense RNA or any other type of RNA that is not translated into protein. In another preferred embodiment of the invention, the nucleic acid segment of interest is translated into a protein product. The nucleotide sequence which directs transcription and/or the nucleic acid segment may be of homologous or heterologous origin with respect to the plant to be transformed. A recombinant DNA molecule useful for introduction into plant cells includes that which has been derived or isolated from any source, that may be subsequently characterized as to structure, size and/or function, chemically altered, and later introduced into plants. An example of a nucleotide sequence or segment of interest “derived” from a source, would be a nucleotide sequence or segment that is identified as a useful fragment within a given organism, and which is then chemically synthesized in essentially pure form. An example of such a nucleotide sequence or segment of interest “isolated” from a source, would be nucleotide sequence or segment that is excised or removed from said source by chemical means, e.g., by the use of restriction endonucleases, so that it can be further manipulated, e.g., amplified, for use in the invention, by the methodology of genetic engineering. Such a nucleotide sequence or segment is commonly referred to as “recombinant.”

Therefore a useful nucleotide sequence, segment or fragment of interest includes completely synthetic DNA, semi-synthetic DNA, DNA isolated from biological sources, and DNA derived from introduced RNA. Generally, the introduced DNA is not originally resident in the plant genotype which is the recipient of the DNA, but it is within the scope of the invention to isolate a gene from a given plant genotype, and to subsequently introduce multiple copies of the gene into the same genotype, e.g., to enhance production of a given gene product such as a storage protein or a protein that is involved in carbohydrate metabolism or any other gene of interest as provided in the SEQ ID NOs of the sequence listing.

The introduced recombinant DNA molecule includes but is not limited to, DNA from plant genes, and non-plant genes such as those from bacteria, yeasts, animals or viruses. The introduced DNA can include modified genes, portions of genes, or chimeric genes, including genes from the same or different genotype. The term “chimeric gene” or “chimeric DNA” is defined as a gene or DNA sequence or segment comprising at least two DNA sequences or segments from species which do not combine DNA under natural conditions, or which DNA sequences or segments are positioned or linked in a manner which does not normally occur in the native genome of untransformed plant.

The introduced recombinant DNA molecule used for transformation herein may be circular or linear, double-stranded or single-stranded. Generally, the DNA is in the form of chimeric DNA, such as plasmid DNA, that can also contain coding regions flanked by regulatory sequences which promote the expression of the recombinant DNA present in the resultant plant.

Generally, the introduced recombinant DNA molecule will be relatively small, i.e., less than about 30 kb to minimize any susceptibility to physical, chemical, or enzymatic degradation which is known to increase as the size of the nucleotide molecule increases. As noted above, the number of proteins, RNA transcripts or mixtures thereof which is introduced into the plant genome is preferably preselected and defined, e.g., from one to about 5-10 such products of the introduced DNA may be formed.

This expression cassette or vector may be contained in a host cell. The expression cassette or vector may augment the genome of a transformed plant or may be maintained extrachromosomally. The expression cassette may be operatively linked to a structural gene, the open reading frame thereof, or a portion thereof. The expression cassette may further comprise a Ti plasmid and be contained in an Agrobacterium tumefaciens cell; it may be carried on a microparticle, wherein the microparticle is suitable for ballistic transformation of a plant cell; or it may be contained in a plant cell or protoplast. Further, the expression cassette or vector can be contained in a transformed plant or cells thereof, and the plant may be a dicot or a monocot. In particular, the plant may be a cereal plant.

Obtaining sufficient levels of transgene expression in the appropriate plant tissues is an important aspect in the production of genetically engineered crops. Expression of heterologous DNA sequences in a plant host is dependent upon the presence of an operably linked promoter that is functional within the plant host. Choice of the promoter sequence will determine when and where within the organism the heterologous DNA sequence is expressed.

For example, for overexpression, a plant promoter fragment may be employed which will direct expression of the gene in all tissue; of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill. Such genes include for example, the AP2 gene, ACT11 from Arabidopsis (Huang et al., Plant Mol. Biol., 33:125, 1996), Cat3 from Arabidopsis (GenBank No. U43147, Zhong et al., Mol. Gen. Genet., 251:196, 1996), the gene encoding stearoyl-acyl carrier protein desaturase from Brassica napus (Genbank No. X74782, Solocombe et al., Plant Physiol., 104:1167, 1994), GPcl from maize (GenBank No. X15596, Martinez et al., J. Mol. Biol., 208:551, 1989), and Gpc2 from maize (GenBank No. U45855, Manjunath et al., Plant Mol. Biol., 33:97, 1997).

Alternatively, the plant promoter may direct expression of the nucleic acid molecules of the invention in a specific tissue or may be otherwise under more precise environmental or developmental control. Examples of environmental conditions that may effect transcription by inducible promoters include anaerobic conditions, elevated temperature, or the presence of light. Such promoters are referred to here as “inducible” or “tissue-specific” promoters. One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

Examples of promoters under developmental control include promoters that initiate transcription only (or primarily only) in certain tissues, such as fruit, seeds, or flowers. Promoters that direct expression of nucleic acids in ovules, flowers or seeds are particularly useful in the present invention. As used herein a seed-specific or preferential promoter is one which directs expression specifically or preferentially in seed tissues, such promoters may be, for example, ovule-specific, embryo-specific, endosperm-specific, integument-specific, seed coat-specific, or some combination thereof. Examples include a promoter from the ovule-specific BEL1 gene described in Reiser et al., Cell 83:735, 1995; (GenBank No. U39944). Other suitable seed specific promoters are derived from the following genes: MACI from maize (Sheridan et al., Genetics 142:1009, 1996), Cat3 from maize (GenBank No. L05934, Abler et al. Plant Mol. Biol., 22:10131, 1993), the gene encoding oleosin 18 kD from maize (GenBank No, J05212, Lee et al. Plant Mol. Biol., 26:1981, 1994), vivparous-1 from Arabidopsis (Genbank No. U93215), the gene encoding oleosin from Arabidopsis (Genbank No. Z17657), Atmycl from Arabidopsis (Urao et al., Plant Mol. Biol., 32:571, 1996), the 2s seed storage protein gene family from Arabidopsis (Conceicao et al., Plant 5:493, 1994) the gene encoding oleosin 20 kD from Brassica napus (GenBank No. M63985), napA from Brassica napus (GenBank No. J02798, Josefsson et al. J. Biol. Chem., 262:12196, 1987), the napin gene family from Brassica napus (Sjodahl et al., Planta, 197:264, 1995), the gene encoding the 2S storage protein from Brassica napus (Dasgupta et al., Gene 133:301, 1993), the genes encoding oleosin A (Genbank No. U09118) and oleosin B (Genbank No. U09119) from soybean and the gene encoding low molecular weight sulphur rich protein from soybean (Choi et al., Mol Gen, Genet., 246:266, 1995).

It is specifically contemplated that one could use one of the promoters that are disclosed in co-pending provisional U.S. application serial no 60/325,448, filed Sep. 26, 2001 in unaltered or altered form. Especially preferred are promoters that direct transcription of an associated nucleic acid molecule specifically or preferentially in tissues of the plant grain such as those provided in SEQ ID NOs: 2275-2672.

Mutagenization of a promoter such as those mentioned hereinbefore or those provided in provisional U.S. application serial no 60/325,448 may potentially improve the utility of the elements for the expression of transgenes in plants. The mutagenesis of these elements can be carried out at random and the mutagenized promoter sequences screened for activity in a trial-by-error procedure.

Alternatively, particular sequences which provide the promoter with desirable expression characteristics, or the promoter with expression enhancement activity, could be identified and these or similar sequences introduced into the sequences via mutation. It is further contemplated that one could mutagenize these sequences in order to enhance their expression of transgenes in a particular species.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters.

A variety of 5′ and 3′ transcriptional regulatory sequences are available for use in the present invention. Transcriptional terminators are responsible for the termination of transcription and correct mRNA polyadenylation. The 3′ nontranslated regulatory DNA sequence preferably includes from about 50 to about 1,000, more preferably about 100 to about 1,000, nucleotide base pairs and contains plant transcriptional and translational termination sequences. Appropriate transcriptional terminators and those which are known to function in plants include the CaMV ³⁵S terminator, the tml terminator, the nopaline synthase terminator, the pea rbcS E9 terminator, the terminator for the T7 transcript from the octopine synthase gene of Agrobacterium tumefaciens, and the 3′ end of the protease inhibitor I or II genes from potato or tomato, although other 3′ elements known to those of skill in the art can also be employed. Alternatively, one also could use a gamma coixin, oleosin 3 or other terminator from the genus Coix.

Preferred 3′ elements include those from the nopaline synthase gene of Agrobacterium tumefaciens (Bevan et al., Nature, 304:184, 1983), the terminator for the T7 transcript from the octopine synthase gene of Agrobacterium tumefaciens, and the 3′ end of the protease inhibitor I or II genes from potato or tomato.

As the DNA sequence between the transcription initiation site and the start of the coding sequence, i.e., the untranslated leader sequence, can influence gene expression, one may also wish to employ a particular leader sequence. Preferred leader sequences are contemplated to include those which include sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will be most preferred.

Other sequences that have been found to enhance gene expression in transgenic plants include intron sequences (e.g., from Adhl, bronzel, actin 1, actin 2 (WO 00/760067), or the sucrose synthase intron) and viral leader sequences (e.g., from TMV, MCMV and AMV). For example, a number of non-translated leader sequences derived from viruses are known to enhance expression. Specifically, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie et al., Nuc. Acids Res., 15:3257, 1987; Skuzeski et al., Plant Mol. Biol, 15;65, 1990). Other leaders known in the art include but are not limited to: Picornavirus leaders, for example, EMCV leader (Encephalomyocarditis 5 noncoding region) (Elroy-Stein et al., Proc. Natl. Acad. Sci. USA., 86:6126, 1989); Polyvirus leaders, for example, TEV leader (Tobacco Etch Virus); MDMV leader (Maize Dwarf Mosaic Virus); Human immunoglobulin heavy-chain binding protein (BiP) leader, (Macejak et al., Nature, 353:90, 1991); Untranslated leader from the coat protein mRNA of alfalfa mosaic virus (AMV RNA 4), (Jobling et al., Nature, 325:622, 1987; Tobacco mosaic virus leader (TMV), (Gallie et al., Plant Cell, 1:301, 1989; and Maize Chlorotic Mottle Virus leader (MCMV) (Lommel et al., Virology, 181:382, 1991. See also, Della-Cioppa et al., Plant Physiol., 84:965, 1987.

Regulatory elements such as Adh intron 1 (Callis et al., Genes Dev., 1:1183, 1987), sucrose synthase intron (Vasil et al., Mol. Microbiol., 3:371, 1989) or TMV omega element (Gallie, et al., Plant Cell, 1:301, 1989), may further be included where desired.

Examples of enhancers include elements from the CaMV ³⁵S promoter, octopine synthase genes (Ellis el al., EMBO J, 6:3203, 1987), the rice actin I gene, the maize alcohol dehydrogenase gene (Callis et al., Genes Dev., 1:1183, 1987), the maize shrunken I gene (Vasil et al., Mol. Microbiol., 3:371, 1989), TMV Omega element (Gallie et al., Plant Cell, 1:301, 1989) and promoters from non-plant eukaryotes (e.g. yeast; Ma et al., Nature, 334:631, 1988).

Two principal methods for the control of expression are known, viz.: overexpression and underexpression. Overexpression can be achieved by insertion of one or more than one extra copy of the selected gene. It is, however, not unknown for plants or their progeny, originally transformed with one or more than one extra copy of a nucleotide sequence, to exhibit the effects of underexpression as well as overexpression. For underexpression there are two principle methods which are commonly referred to in the art as “antisense downregulation” and “sense downregulation” (sense downregulation is also referred to as “cosuppression”). Generically these processes are referred to as “gene silencing”. Both of these methods lead to an inhibition of expression of the target gene.

Within the scope of the present invention, the alteration in expression of the nucleic acid molecule of the present invention may be achieved in one of the following ways:

(1) “Sense” Suppression

Alteration of the expression of a nucleotide sequence of the present invention, preferably reduction of its expression, is obtained by “sense” suppression (referenced in e.g. Jorgensen et al., Plant Mol. Biol., 31:957, 1996). In this case, the entirety or a portion of a nucleotide sequence of the present invention is comprised in a DNA molecule. The DNA molecule is preferably operatively linked to a promoter functional in a cell comprising the target gene, preferably a plant cell, and introduced into the cell, in which the nucleotide sequence is expressible. The nucleotide sequence is inserted in the DNA molecule in the “sense orientation”, meaning that the coding strand of the nucleotide sequence can be transcribed. In a preferred embodiment, the nucleotide sequence is fully translatable and all the genetic information comprised in the nucleotide sequence, or portion thereof, is translated into a polypeptide. In another preferred embodiment, the nucleotide sequence is partially translatable and a short peptide is translated. In a preferred embodiment, this is achieved by inserting at least one premature stop codon in the nucleotide sequence, which bring translation to a halt. In another more preferred embodiment, the nucleotide sequence is transcribed but no translation product is being made. This is usually achieved by removing the start codon, e.g. the “ATG”, of the polypeptide encoded by the nucleotide sequence. In a further preferred embodiment, the DNA molecule comprising the nucleotide sequence, or a portion thereof, is stably integrated in the genome of the plant cell. In another preferred embodiment, the DNA molecule comprising the nucleotide sequence, or a portion thereof, is comprised in an extrachromosomally replicating molecule.

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

(2) “Anti-Sense” Suppression

In another preferred embodiment, the alteration of the expression of a nucleotide sequence of the present invention, preferably the reduction of its expression is obtained by “anti-sense” suppression. The entirety or a portion of a nucleotide sequence of the present invention is comprised in a DNA molecule. The DNA molecule is preferably operatively linked to a promoter functional in a plant cell, and introduced in a plant cell, in which the nucleotide sequence is expressible. The nucleotide sequence is inserted in the DNA molecule in the “anti-sense orientation”, meaning that the reverse complement (also called sometimes non-coding strand) of the nucleotide sequence can be transcribed. In a preferred embodiment, the DNA molecule comprising the nucleotide sequence, or a portion thereof, is stably integrated in the genome of the plant cell. In another preferred embodiment the DNA molecule comprising the nucleotide sequence, or a portion thereof, is comprised in an extrachromosomally replicating molecule. Several publications describing this approach are cited for further illustration (Green, P. J. et al., Ann. Rev. Biochem., 55:569, 1986); Mol and van der Krol, A. R., eds., Antisense Nuc. Acids & Proteins, Marcel Dekker, pp. 125-141, 1991; Abel, P. P. et al., Proc. Natl. Acad. Sci. USA, 86:6949, 1989; Ecker, J. R. et al., Proc. Natl. Acad. Sci. USA, 83:5372, 1986).

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

(3) Homologous Recombination

In another preferred embodiment, at least one genomic copy corresponding to a nucleotide sequence of the present invention is modified in the genome of the plant by homologous recombination as further illustrated in Paszkowski et al., EMBO J, 7:4021, 1988. This technique uses the property of homologous sequences to recognize each other and to exchange nucleotide sequences between each by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one embodiment, the regulatory elements of the nucleotide sequence of the present invention are modified. Such regulatory elements are easily obtainable by screening a genomic library using the nucleotide sequence of the present invention, or a portion thereof, as a probe. The existing regulatory elements are replaced by different regulatory elements, thus altering expression of the nucleotide sequence, or they are mutated or deleted, thus abolishing the expression of the nucleotide sequence. In another embodiment, the nucleotide sequence is modified by deletion of a part of the nucleotide sequence or the entire nucleotide sequence, or by mutation. Expression of a mutated polypeptide in a plant cell is also contemplated in the present invention. More recent refinements of this technique to disrupt endogenous plant genes have been described (Kempin et al., Nature, 389:802, 1997 and Miao and Lam, Plant J., 7:359, 1995.

In another preferred embodiment, a mutation in the chromosomal copy of a nucleotide sequence is introduced by transforming a cell with a chimeric oligonucleotide composed of a contiguous stretch of RNA and DNA residues in a duplex conformation with double hairpin caps on the ends. An additional feature of the oligonucleotide is for example the presence of 2′-O-methylation at the RNA residues. The RNA/DNA sequence is designed to align with the sequence of a chromosomal copy of a nucleotide sequence of the present invention and to contain the desired nucleotide change. For example, this technique is further illustrated in U.S. Pat. No. 5,501,967 and Zhu et al., Proc. Natl. Acad. Sci. USA, 96:8768, 1999.

(4) Ribozymes

In a further embodiment, the RNA coding for a polypeptide of the present invention is cleaved by a catalytic RNA, or ribozyme, specific for such RNA. The ribozyme is expressed in transgenic plants and results in reduced amounts of RNA coding for the polypeptide of the present invention in plant cells, thus leading to reduced amounts of polypeptide accumulated in the cells. This method is further illustrated in U.S. Pat. No. 4,987,071.

(5) Dominant-Negative Mutants

In another preferred embodiment, the activity of the polypeptide encoded by the nucleotide sequences of this invention is changed. This is achieved by expression of dominant negative mutants of the proteins in transgenic plants, leading to the loss of activity of the endogenous protein.

(6) Aptamers

In a further embodiment, the activity of polypeptide of the present invention is inhibited by expressing in transgenic plants nucleic acid ligands, so-called aptamers, which specifically bind to the protein. Aptamers are preferentially obtained by the SELEX (Systematic Evolution of Ligands by EXponential Enrichment) method. In the SELEX method, a candidate mixture of single stranded nucleic acids having regions of randomized sequence is contacted with the protein and those nucleic acids having an increased affinity to the target are partitioned from the remainder of the candidate mixture. The partitioned nucleic acids are amplified to yield a ligand enriched mixture. After several iterations a nucleic acid with optimal affinity to the polypeptide is obtained and is used for expression in transgenic plants. This method is further illustrated in U.S. Pat. No. 5,270,163.

(7) Zinc Finger Proteins

A zinc finger protein that binds a nucleotide sequence of the present invention or to its regulatory region is also used to alter expression of the nucleotide sequence. Preferably, transcription of the nucleotide sequence is reduced or increased. Zinc finger proteins are for example described in Beerli et al., Proc. Natl. Acad. Sci. USA, 95:14628, 1998 or in WO 95/19431, WO 98/54311, or WO 96/06166.

(8) dsRNA

Alteration of the expression of a nucleotide sequence of the present invention is also obtained by dsRNA interference as described for example in WO 99/32619, WO 99/53050 or WO 99/61631.

(9) Insertion of a DNA Molecule (Insertional Mutagenesis)

In another preferred embodiment, a DNA molecule is inserted into a chromosomal copy of a nucleotide sequence of the present invention, or into a regulatory region thereof. Preferably, such DNA molecule comprises a transposable element capable of transposition in a plant cell, such as e.g. Ac/Ds, Em/Spm, mutator. Alternatively, the DNA molecule comprises a T-DNA border of an Agrobacterium T-DNA. The DNA molecule may also comprise a recombinase or integrase recognition site which can be used to remove part of the DNA molecule from the chromosome of the plant cell. An example of this method is set forth in the examples. Methods of insertional mutagenesis using T-DNA, transposons, oligonucleotides or other methods known to those skilled in the art are also encompassed. Methods of using T-DNA and transposon for insertional mutagenesis are described in Winkler et al., Methods Mol. Biol. 82:129, 1989 and Martienssen, Proc Natl. Acad. Sci. USA, 95:2021, 1998.

(10) Deletion mutagenesis

In yet another embodiment, a mutation of a nucleic acid molecule of the present invention is created in the genomic copy of the sequence in the cell or plant by deletion of a portion of the nucleotide sequence or regulator sequence. Methods of deletion mutagenesis are known to those skilled in the art. See, for example, Miao et al.,

Plant J., 7:359, 1995.

In yet another embodiment, this deletion is created at random in a large population of plants by chemical mutagenesis or irradiation and a plant with a deletion in a gene of the present invention is isolated by forward or reverse genetics. Irradiation with fast neutrons or gamma rays is known to cause deletion mutations in plants (Silverstone et al., Plant Cell, 10:155, 1998; Bruggemann et al., Plant J., 10:755, 1996; Redei and Koncz in Methods in Arabidopsis Research, World Scientific Press, pp. 16-82, 1992). Deletion mutations in a gene of the present invention can be recovered in a reverse genetics strategy using PCR with pooled sets of genomic DNAs as has been shown in C. elegans (Liu et al., Genome Res., 9:859, 1999.). A forward genetics strategy would involve mutagenesis of a line displaying PTGS followed by screening the M2 progeny for the absence of PTGS. Among these mutants would be expected to be some that disrupt a gene of the present invention. This could be assessed by Southern blot or PCR for a gene of the present invention with genomic DNA from these mutants.

(11) Overexpression in a Plant Cell

In yet another preferred embodiment, a nucleotide sequence of the present invention encoding a polypeptide comprising a 3′-5′ exonuclease domain and/or activity in a plant cell is overexpressed. Examples of nucleic acid molecules and expression cassettes for overexpression of a nucleic acid molecule of the present invention are described above. Methods known to those skilled in the art of over-expression of nucleic acid molecules are also encompassed by the present invention.

In still another embodiment, the expression of the nucleotide sequence of the present invention is altered in every cell of a plant. This is, for example, obtained though homologous recombination or by insertion in the chromosome. This is also, for example, obtained by expressing a sense or antisense RNA, zinc finger protein or ribozyme under the control of a promoter capable of expressing the sense or antisense RNA, zinc finger protein or ribozyme in every cell of a plant. Constitutive expression, inducible, tissue-specific or developmentally-regulated expression are also within the scope of the present invention and result in a constitutive, inducible, tissue-specific or developmentally-regulated alteration of the expression of a nucleotide sequence of the present invention in the plant cell. Constructs for expression of the sense or antisense RNA, zinc finger protein or ribozyme, or for overexpression of a nucleotide sequence of the present invention, are prepared and transformed into a plant cell according to the teachings of the present invention, e.g. as described infra.

The invention hence also provides sense and anti-sense nucleic acid molecules corresponding to the open reading frames identified in the SEQ ID NOs of the Sequence Lisitng as well as their orthologs.

The genes and open reading frames according to the present invention which are substantially similar to a nucleotide sequence encoding a polypeptide as given in any one of the SEQ ID NOs of the Sequence Lisiting including any corresponding anti-sense constructs can be operably linked to any promoter that is functional within the plant host including the promoter sequences according to the invention or mutants thereof.

The present invention further provides a method of augmenting a plant genome by contacting plant cells with a nucleic acid molecule of the invention, e.g., one having a nucleotide sequence that directs tissue-specific, tissue-preferential and/or mycorrhizal fungi- or phosphate-induced transcription of a linked nucleic acid segment isolatable or obtained from a plant gene encoding a polypeptide that is substantially similar to a polypeptide encoded by the an Oryza gene having a sequence according to any one of SEQ ID NOs provided in the Sequence Listing so as to yield transformed plant cells; and regenerating the transformed plant cells to provide a differentiated transformed plant, wherein the differentiated transformed plant expresses the nucleic acid molecule in the cells of the plant, preferably in the appropriate tissues of the plant grain. The nucleic acid molecule may be present in the nucleus, chloroplast, mitochondria and/or plastid of the cells of the plant.

Plant species may be transformed with the DNA construct of the present invention by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a vector of the present invention. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or TI) transformed plants may be selfed to give homozygous second generation (or T2) transformed plants, and the T2 plants further propagated through classical breeding techniques. A dominant selectable marker (such as npt II) can be associated with the expression cassette to assist in breeding.

Thus, the present invention provides a transformed (transgenic) plant cell, in planta or explanta, including a transformed plastid or other organelle, e.g., nucleus, mitochondria or chloroplast. The present invention may be used for transformation of any plant species, including, but not limited to, cells from corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, vegetables, ornamentals, and conifers.

Duckweed (Lemna, see WO 00/07210) includes members of the family Lemnaceae. There are known four genera and 34 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza, S. punctata); genus Woffia (Wa. Angusta, Wa. Arrhiza, Wa. Australina, Wa. Borealis, Wa. Brasiliensis, Wa. Columbiana, Wa. Elongata, Wa. Globosa, Wa. Microscopica, Wa. Neglecta) and genus Wofiella (Wl. ultila, Wl. ultilanen, Wl. gladiata, Wl. ultila, Wl. lingulata, Wl. repunda, Wl. rotunda, and Wl. neotropica). Any other genera or species of Lemnaceae, if they exist, are also aspects of the present invention. Lemna gibba, Lemnaminor, and Lemna miniscula are preferred, with Lemnaminor and Lemna miniscula being most preferred. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich (1986)).

Vegetables within the scope of the invention include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop.

Other plants within the scope of the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, and zucchini.

Ornamental plants within the scope of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Other plants within the scope of the invention are shown in Table 1 (above).

Preferably, transgenic plants of the present invention are crop plants and in particular cereals (for example, corn, alfalfa, sunflower, rice, Brassica, canola, soybean, barley, soybean, sugarbeet, cotton, safflower, peanut, sorghum, wheat, millet, tobacco, etc.), and even more preferably corn, rice and soybean.

The present invention also provides a transgenic plant prepared by this method, a seed from such a plant and progeny plants from such a plant including hybrids and inbreds. Preferred transgenic plants are transgenic maize, soybean, barley, alfalfa, sunflower, canola, soybean, cotton, peanut, sorghum, tobacco, sugarbeet, rice, wheat, rye, turfgrass, millet, sugarcane, tomato, or potato.

A transformed (transgenic) plant of the invention includes plants, the genome of which is augmented by a nucleic acid molecule of the invention, or in which the corresponding gene has been disrupted, e.g., to result in a loss, a decrease or an alteration, in the function of the product encoded by the gene, which plant may also have increased yields and/or produce a better-quality product than the corresponding wild-type plant. The nucleic acid molecules of the invention are thus useful for targeted gene disruption, as well as markers and probes.

The invention also provides a method of plant breeding, e.g., to prepare a crossed fertile transgenic plant. The method comprises crossing a fertile transgenic plant comprising a particular nucleic acid molecule of the invention with itself or with a second plant, e.g., one lacking the particular nucleic acid molecule, to prepare the seed of a crossed fertile transgenic plant comprising the particular nucleic acid molecule. The seed is then planted to obtain a crossed fertile transgenic plant. The plant may be a monocot or a dicot. In a particular embodiment, the plant is a cereal plant.

The crossed fertile transgenic plant may have the particular nucleic acid molecule inherited through a female parent or through a male parent. The second plant may be an inbred plant. The crossed fertile transgenic may be a hybrid. Also included within the present invention are seeds of any of these crossed fertile transgenic plants.

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

A variety of techniques are available and known to those skilled in the art for introduction of constructs into a plant cell host. These techniques generally include transformation with DNA employing A. tumefaciens or A. rhizogenes as the transforming agent, liposomes, PEG precipitation, electroporation, DNA injection, direct DNA uptake, microprojectile bombardment, particle acceleration, and the like (See, for example, EP 295959 and EP 138341) (see below). However, cells other than plant cells may be transformed with the expression cassettes of the invention. The general descriptions of plant expression vectors and reporter genes, and Agrobacterium and Agrobacterium-mediated gene transfer, can be found in Gruber et al., Vectorsfor Plant Transformation, in Methods in Plant Molecular Biology, Glich et al., eds, pp. 89-119, CRC Press, 1993.

Expression vectors containing genomic or synthetic fragments can be introduced into protoplasts or into intact tissues or isolated cells. Preferably expression vectors are introduced into intact tissue. General methods of culturing plant tissues are provided for example by Maki et al., Methods in Plant Molecular Biology, Glich et al., eds, pp. 67-88, CRC Press, 1993; and by Phillips et al. in Corn and Corn Improvement, 3d ed, Sprague et al., eds., Amer. Soc of Agronomy, 1988. Preferably, expression vectors are introduced into maize or other plant tissues using a direct gene transfer method such as microprojectile-mediated delivery, DNA injection, electroporation and the like. More preferably expression vectors are introduced into plant tissues using the microprojectile media delivery with the biolistic device. See, for example, Tomes et al., Plant Cell, Tissue and Organ Culture: Fundamental Methods, Springer-Verlag, 1995. The vectors of the invention can not only be used for expression of structural genes but may also be used in exon-trap cloning, or promoter trap procedures to detect differential gene expression in varieties of tissues, (Lindsey et al., Transgen. Res., 2:3347, 1993; Auch & Reth et al., Nuc. Acids Res., 18:6743, 1990).

It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti et al., Bio/Technology, 3:241, 1985: Byrne et al., Plant Cell Tissue Org Culture, 8:3, 1987; Sukhapinda et al., Plant Mol. Biol., 8:209, 1987; Lorz et al., Mol. Gen. Genet., 199:178, 1985; Potrykus, Trends Biotech., 7:269 1985; Park et al., J. Plant Biol., 38:365, 1985: Hiei et al., Plant J., 6:271, 1994). The use of T-DNA to transform plant cells has received extensive study and is amply described (EP 120516; Hoekema, in The Binary Plant Vector System, Offset-drukkcrij Kanters B. V., 1985; Knauf, et al., Analysis ofHost Range Expression by Agrobacterium, in Molecular Genetics of the Bacteria-Plant Interaction, Puhler, ed., Springer-Verlag, 1983; and An et al., EMBO J, 4:277, 1985). For introduction into plants, the chimeric genes of the invention can be inserted into binary vectors as described in the examples.

Other transformation methods are available to those skilled in the art, such as direct uptake of foreign DNA constructs (see EP 295959), techniques of electroporation (Fromm et al., Nature, 319:791 1986) or high velocity ballistic bombardment with metal particles coated with the nucleic acid constructs (Kline et al., Nature, 327:70, 1987, and U.S. Pat. No. 4,945,050). Once transformed, the cells can be regenerated by those skilled in the art. Of particular relevance are the recently described methods to transform foreign genes into commercially important crops, such as rapeseed (De Block et al., Plant Physiol., 91:694, 1989), sunflower (Everett et al., Bio/Technology, 5:1201, 1987), soybean (McCabe et al., Bio/Technology, 6:923, 1988; Hinchee et al., Bio/Technology, 6:915, 1988; Chee et al., Plant Physiol., 91:1212, 1989; Christou et al., Proc. Natl. Acad. Sci. USA, 86:7500, 1989; EP 301749), rice (Hiei et al., Plant J., 6:271, 1994), and corn (Gordon Kamm et al., Plant Cell, 2:603, 1990; Fromm et al., Bio/Technology, 8:833, 1990).

Those skilled in the art will appreciate that the choice of method might depend on the type of plant, i.e., monocotyledonous or dicotyledonous, targeted for transformation. Suitable methods of transforming plant cells include, but are not limited to, microinjection (Crossway et al., Bio/Techniques, 4:320, 1986), electroporation (Riggs et al., Proc. Natl. Acad. Sci. USA, 83:5602, 1986), Agrobacterium-mediated transformation (Hinchee et al., Bio/Technology, 6:915, 1988), direct gene transfer (Paszkowski et al., EMBO J, 3:2717, 1984), and ballistic particle acceleration using devices available from Agracetus, Inc., Madison, Wis. And BioRad, Hercules, Calif. (see, for example, Sanford et al., U.S. Pat. No. 4,945,050; and McCabe et al., Bio/Technology, 6:923, 1988). Also see, Weissinger et al., Ann. Rev. Genet., 22:421, 1988; Sanford et al., Particulate Sci. Tech., 5:27, 1987 (onion); Christou et al., Plant Physiol, 87:671, 1988 (soybean); McCabe et al., Bio/Technology, 6:923, 1988 (soybean); Datta et al., Bio/Technology, 8:736, 1990 (rice); Klein et al., Bio/Technology, 6:559, 1988 (maize); Klein et al., 1988 (maize); Klein et al., 1988 (maize); Fromm et al., Bio/Technology, 8:833, 1990 (maize); and Gordon-Kamm et al., Plant Cell, 2:603, 1990 (maize); Svab et al., Proc. Natl. Acad. Sci. USA, 87:8526, 1990 (tobacco chloroplast); Koziel et al., Biotechnology, 11:194, 1993 (maize); Shimamoto et al., Nature, 338:274, 1989 (rice); Christou et al., Biotechnology, 9:957, 1991 (rice); European Patent Application EP 0 332 581 (orchardgrass and other Pooideae); Vasil et al., Biotechnology, 11: 1553, 1993 (wheat); Weeks et al., Plant Physiol., 102:1077, 1993 (wheat). In one embodiment, the protoplast transformation method for maize is employed (European Patent Application EP 0 292 435, U.S. Pat. No. 5,350,689).

In another embodiment, a nucleotide sequence of the present invention is directly transformed into the plastid genome. Plastid transformation technology is extensively described in U.S. Pat. Nos. 5,451,513, 5,545,817, and 5,545,818, in PCT application no. WO 95/16783, and in McBride et al., Proc. Natl. Acad. Sci. USA, 91:7301, 1994. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate orthologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., Proc. Natl. Acad. Sci. USA, 87:8526, 1990; Staub et al., Plant Cell, 4:39, 1992). This resulted in stable homoplasmic transformants at a frequency of approximately one per 100 bombardments of target leaves. The presence of cloning sites between these markers allowed creation of a plastid targeting vector for introduction of foreign genes (Staub et al., EMBO J., 12:601, 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3N-adenyltransferase (Svab et al., EMBO J., 12:601, 1993). Other selectable markers useful for plastid transformation are known in the art and encompassed within the scope of the invention. Typically, approximately 15-20 cell division cycles following transformation are required to reach a homoplastidic state. Plastid expression, in which genes are inserted by orthologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid targeting vector and transformed into the plastid genome of a desired plant host. Plants homoplastic for plastid genomes containing a nucleotide sequence of the present invention are obtained, and are preferentially capable of high expression of the nucleotide sequence.

Agrobacterium tumefaciens cells containing a vector comprising an expression cassette of the present invention, wherein the vector comprises a Ti plasmid, are useful in methods of making transformed plants. Plant cells are infected with an Agrobacterium tumefaciens as described above to produce a transformed plant cell, and then a plant is regenerated from the transformed plant cell. Numerous Agrobacterium vector systems useful in carrying out the present invention are known.

For example, vectors are available for transformation using Agrobacterium tumefaciens. These typically carry at least one T-DNA border sequence and include vectors such as pBIN19 (Bevan, Nuc. Acids Res., 12:8711, 1984). In one preferred embodiment, the expression cassettes of the present invention may be inserted into either of the binary vectors pCIB200 and pCIB2001 for use with Agrobacterium. These vector cassettes for Agrobacterium-mediated transformation were constructed in the following manner. PTJS75kan was created by NarI digestion of pTJS75 (Schmidhauser & Helinski, J. Bacteriol., 164:446, 1985) allowing excision of the tetracycline-resistance gene, followed by insertion of an AccI fragment from pUC4K carrying an NPTII (Messing & Vierra, Gene, 19:259, 1982; Bevan et al., Nature, 304:184, 1983; McBride et al., Plant Mol. Biol., 14:266, 1990). XhoI linkers are ligated to the EcoRV fragment of pCIB7 which contains the left and right T-DNA borders, a plant selectable nos/nptll chimeric gene and the pUC polylinker (Rothstein et al., Gene, 53:153, 1987), and the XhoI-digested fragment was cloned into SalI-digested pTJS75kan to create pCIB200 (see also EP 0 332 104, example 19). PCIB200 contains the following unique polylinker restriction sites: EcoRI, SstI, KpnI, BglII, XbaI, and SalI. The plasmid pCIB2001 is a derivative of pCIB200 which was created by the insertion into the polylinker of additional restriction sites. Unique restriction sites in the polylinker of pCIB2001 are EcoRI, SstI, KpnI, BglII, XbaI, SalI, MluI, BclI, AvrII, ApaI, HpaI, and Stul. PCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

An additional vector useful for Agrobacterium-mediated transformation is the binary vector pCIB 10, which contains a gene encoding kanamycin resistance for selection in plants, T-DNA right and left border sequences and incorporates sequences from the wide host-range plasmid pRK252 allowing it to replicate in both E. coli and Agrobacterium. Its construction is described by Rothstein et al., Gene, 53:153, 1987. Various derivatives of pCIB10 have been constructed which incorporate the gene for hygromycin B phosphotransferase described by Gritz et al., Gene, 25:179, 1983. These derivatives enable selection of transgenic plant cells on hygromycin only (pCIB743), or hygromycin and kanamycin (pCIB715, pCIB717).

Methods using either a form of direct gene transfer or Agrobacterium-mediated transfer usually, but not necessarily, are undertaken with a selectable marker which may provide resistance to an antibiotic (e.g., kanamycin, hygromycin or methotrexate) or a herbicide (e.g., phosphinothricin). The choice of selectable marker for plant transformation is not, however, critical to the invention.

For certain plant species, different antibiotic or herbicide selection markers may be preferred. Selection markers used routinely in transformation include the nptll gene which confers resistance to kanamycin and related antibiotics (Messing & Vierra, Gene, 19:259, 1982; Bevan et al., Nature, 304:184, 1983), the bar gene which confers resistance to the herbicide phosphinothricin (White et al., Nuc. Acids Res., 18:1062, 1990, Spencer et al., Theor. Appl. Genet., 79:625, 1990), the hph gene which confers resistance to the antibiotic hygromycin (Blochinger & Diggelmann Mol. Cell. Biol., 4:2929, 1984), and the dhfr gene, which confers resistance to methotrexate (Bourouis et al., EMBO J. 2:1099 1983).

Selection markers resulting in positive selection, such as a phosphomannose isomerase gene, as described in patent application WO 93/05163, are also used. Other genes to be used for positive selection are described in WO 94/20627 and encode xyloisomerases and phosphomanno-isomerases such as mannose-6-phosphate isomerase and mannose-1-phosphate isomerase; phosphomanno mutase; mannose epimerases such as those which convert carbohydrates to mannose or mannose to carbohydrates such as glucose or galactose; phosphatases such as mannose or xylose phosphatase, mannose-6-phosphatase and mannose-1-phosphatase, and permeases which are involved in the transport of mannose, or a derivative, or a precursor thereof into the cell. The agent which reduces the toxicity of the compound to the cells is typically a glucose derivative such as methyl-3-O-glucose or phloridzin. Transformed cells are identified without damaging or killing the non-transformed cells in the population and without co-introduction of antibiotic or herbicide resistance genes. As described in WO 93/05163, in addition to the fact that the need for antibiotic or herbicide resistance genes is eliminated, it has been shown that the positive selection method is often far more efficient than traditional negative selection.

One vector useful for direct gene transfer techniques in combination with selection by the herbicide Basta (or phosphinothricin) is pCIB3064. This vector is based on the plasmid pCIB246, which comprises the CaMV ³⁵S promoter in operational fusion to the E. coli GUS gene and the CaMV ³⁵S transcriptional terminator and is described in the PCT published application WO 93/07278. One gene useful for conferring resistance to phosphinothricin is the bar gene from Streptomyces viridochromogenes (Thompson et al., EMBO J., 6:2519, 1987). This vector is suitable for the cloning of plant expression cassettes containing their own regulatory signals.

An additional transformation vector is pSOG35 which utilizes the E. coli gene dihydrofolate reductase (DHFR) as a selectable marker conferring resistance to methotrexate. PCR was used to amplify the ³⁵S promoter (about 800 bp), intron 6 from the maize Adh1 gene (about 550 bp) and 18 bp of the GUS untranslated leader sequence from pSOG10. A 250 bp fragment encoding the E. coli dihydrofolate reductase type II gene was also amplified by PCR and these two PCR fragments are assembled with a SacI-PstI fragment from pBI221 (Clontech) which comprised the pUC19 vector backbone and the nopaline synthase terminator. Assembly of these fragments generated pSOG19 which contains the ³⁵S promoter in fusion with the intron 6 sequence, the GUS leader, the DHFR gene and the nopaline synthase terminator. Replacement of the GUS leader in pSOG19 with the leader sequence from Maize Chlorotic Mottle Virus check (MCMV) generated the vector pSOG35. pSOG19 and pSOG35 carry the pUC-derived gene for ampicillin resistance and have HindIII, SphI, PstI and EcoRI sites available for the cloning of foreign sequences.

Binary backbone vector pNOV2117 contains the T-DNA portion flanked by the right and left border sequences, and including the Positech™ (Syngenta) plant selectable marker and the “candidate gene” gene expression cassette. The Positech™ plant selectable marker confers resistance to mannose and in this instance consists of the maize ubiquitin promoter driving expression of the PMI (phosphomannose isomerase) gene, followed by the cauliflower mosaic virus transcriptional terminator.

Transgenic plant cells are then placed in an appropriate selective medium for selection of transgenic cells which are then grown to callus. Shoots are grown from callus and plantlets generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA which has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region which is not native to the gene from which the transcription-initiation-region is derived.

To confirm the presence of the transgenes in transgenic cells and plants, a variety of assays may be performed. Such assays include, for example, “molecular biological” assays well known to those of skill in the art, such as Southern and Northern blotting, in situ hybridization and nucleic acid-based amplification methods such as PCR or RT-PCR; “biochemical” assays, such as detecting the presence of a protein product, e.g., by immunological means (ELISAs and Western blots) or by enzymatic function; plant part assays, such as seed assays; and also, by analyzing the phenotype of the whole regenerated plant, e.g., for disease or pest resistance.

DNA may be isolated from cell lines or any plant parts to determine the presence of the preselected nucleic acid segment through the use of techniques well known to those skilled in the art. Note that intact sequences will not always be present, presumably due to rearrangement or deletion of sequences in the cell.

The presence of nucleic acid elements introduced through the methods of this invention may be determined by polymerase chain reaction (PCR). Using this technique discreet fragments of nucleic acid are amplified and detected by gel electrophoresis. This type of analysis permits one to determine whether a preselected nucleic acid segment is present in a stable transformant, but does not prove integration of the introduced preselected nucleic acid segment into the host cell genome. In addition, it is not possible using PCR techniques to determine whether transformants have exogenous genes introduced into different sites in the genome, i.e., whether transformants are of independent origin. It is contemplated that using PCR techniques it would be possible to clone fragments of the host genomic DNA adjacent to an introduced preselected DNA segment.

Positive proof of DNA integration into the host genome and the independent identities of transformants may be determined using the technique of Southern hybridization. Using this technique specific DNA sequences that are introduced into the host genome and flanking host DNA sequences can be identified. Hence the Southern hybridization pattern of a given transformant serves as an identifying characteristic of that transformant. In addition it is possible through Southern hybridization to demonstrate the presence of introduced preselected DNA segments in high molecular weight DNA, i.e., confirm that the introduced preselected DNA segment has been integrated into the host cell genome. The technique of Southern hybridization provides information that is obtained using PCR, e.g., the presence of a preselected DNA segment, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of a preselected DNA segment.

Both PCR and Southern hybridization techniques can be used to demonstrate transmission of a preselected DNA segment to progeny. In most instances the characteristic Southern hybridization pattern for a given transformant will segregate in progeny as one or more Mendelian genes (Spencer et al., Theor. Appl. Genet., 79:625, 1992); Laursen et al., Plant Mol. Biol., 24:51, 1994) indicating stable inheritance of the gene. The nonchimeric nature of the callus and the parental transformants (Ro) was suggested by germline transmission and the identical Southern blot hybridization patterns and intensities of the transforming DNA in callus, R₀ plants and R₁ progeny that segregated for the transformed gene.

Whereas DNA analysis techniques may be conducted using DNA isolated from any part of a plant, RNA may only be expressed in particular cells or tissue types and hence it will be necessary to prepare RNA for analysis from these tissues. PCR techniques may also be used for detection and quantitation of RNA produced from introduced preselected DNA segments. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and will only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the protein products of the introduced preselected DNA segments or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed.

Very frequently the expression of a gene product is determined by evaluating the phenotypic results of its expression. These assays also may take many forms including but not limited to analyzing changes in the chemical composition, morphology, or physiological properties of the plant. Morphological changes may include greater stature or thicker stalks. Most often changes in response of plants or plant parts to imposed treatments are evaluated under carefully controlled conditions termed bioassays.

The compositions of the invention include plant nucleic acid molecules, and the amino acid sequences for the polypeptides or partial-length polypeptides encoded by the nucleic acid molecule which comprises an open reading frame. These sequences can be employed to alter expression of a particular gene corresponding to the open reading frame by decreasing or eliminating expression of that plant gene or by overexpressing a particular gene product. Methods of this embodiment of the invention include stably transforming a plant with the nucleic acid molecule of the invention which includes an open reading frame operably linked to a promoter capable of driving expression of that open reading frame (sense or antisense) in a plant cell. By “portion” or “fragment”, as it relates to a nucleic acid molecule which comprises an open reading frame or a fragment thereof encoding a partial-length polypeptide having the activity of the full length polypeptide, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention. Thus, to express a particular gene product, the method comprises introducing to a plant, plant cell, or plant tissue an expression cassette comprising a promoter linked to an open reading frame so as to yield a transformed differentiated plant, transformed cell or transformed tissue. Transformed cells or tissue can be regenerated to provide a transformed differentiated plant. The transformed differentiated plant or cells thereof preferably expresses the open reading frame in an amount that alters the amount of the gene product in the plant or cells thereof, which product is encoded by the open reading frame. The present invention also provides a transformed plant prepared by the method, progeny and seed thereof.

The invention further includes a nucleotide sequence which is complementary to one (hereinafter “test” sequence) which hybridizes under stringent conditions with a nucleic acid molecule of the invention as well as RNA which is transcribed from the nucleic acid molecule. When the hybridization is performed under stringent conditions, either the test or nucleic acid molecule of invention is preferably supported, e.g., on a membrane or DNA chip. Thus, either a denatured test or nucleic acid molecule of the invention is preferably first bound to a support and hybridization is effected for a specified period of time at a temperature of, e.g., between 55 and 70° C., in double strength citrate buffered saline (SC) containing 0.1% SDS followed by rinsing of the support at the same temperature but with a buffer having a reduced SC concentration. Depending upon the degree of stringency required such reduced concentration buffers are typically single strength SC containing 0.1% SDS, half strength SC containing 0.1% SDS and one-tenth strength SC containing 0.1% SDS.

In a further embodiment, the present invention provides a transformed plant host cell, or one obtained through breeding, capable of over-expressing, under-expressing, or having a knock out of amino acid genes and/or their gene products. The plant cell is transformed with at least one such expression vector wherein the plant host cell can be used to regenerate plant tissue or an entire plant, or seed there from, in which the effects of expression, including overexpression or underexpression, of the introduced sequence or sequences can be measured in vitro or in planta.

The invention will be further described by reference to the following detailed examples. These examples are provided for purposes of illustration only, and are not intended to be limiting unless otherwise specified.

### EXAMPLES

### Example 1

### Isolation and Sequencing of DNA Fragments

1.1 Isolation and Sequencing of Genomic DNA Fragments

Genomic DNA is isolated from nuclei of Oryza sativa L. sspjaponica cv Nipponbare and then sheared to produce fragments of approximately 500 bp. Using a method derived from the method of Mao et al. (Genome Res 10:982 (2000)), seeds are germinated on cheese cloth immersed in water and grown for 4-6 weeks under greenhouse conditions. After plants reach a height of approximately 5-8 inches, the upper parts of the green leaves are harvested and wrapped in aluminum foil at 4° C. overnight. Leaf material is then stored at −80° C. or directly used for extraction of nuclei. Intact nuclei are isolated by homogenization (in a blender for fresh material or by grinding with mortar and pestle for frozen material) in a buffer containing 10 mM Trizma base, 80 mM KCl, 10 mM EDTA, 1 mM spermidine, 1 mM spermine, 0.5 M sucrose, 0.5% Triton-X-100, 0.15% β-mercaptoethanol pH 9.5. The homogenate is filtered and nuclei recovered by gentle centrifugation using a fixed-angle rotor at 1,800 g at 4° C. for 20 minutes. The pellet recovered after centrifugation is gently resuspended with the assistance of a small paint brush soaked in ice cold wash buffer and wash buffer added. Particulate matter remaining in the suspension is removed by filtering the resuspended nuclei into a 50 ml centrifuge tube through two layers of miracloth by gravity and centrifuging the filtrate at 57 g (500 rpm), 4° C. for 2 minutes to remove intact cells and tissue residues. The supernatant fluid is transferred into a fresh centrifuge tube and nuclei are pelleted by centrifugation at 1,800 g, 4° C. for 15 minutes in a swinging bucket centrifuge.

DNA is isolated from the nuclear preparation by phenol/chloroform extraction, as in Sambrook et al (supra). Isolated total genomic DNA is physically sheared (Hydroshear) to generate for generating random DNA fragments, and fragments of approximately 500 bp are recovered. DNA is eluted and the ends filled in using T₄ DNA polymerase, Klenow fragments, and dNTPs. Double-stranded DNA is linkered and cloned into a Novartis proprietary medium-copy vector derived from pSC101.

Vector inserts are amplified by PCR and sequenced using the MegaBACE sequencing system (Molecular Dynamics, Amersham). The amplification reaction is diluted before use and is not purified using an exonuclease/alkaline phosphatase procedure. Sequencing reactions are performed using DYEnamic ET Terminator Kit. The reactions contained approximately 50 ng of amplicon, DYEnamic ET Terminator premix, and 5 pmol of −40 M13 forward primer. The sequencing reaction is amplified for 30 cycles, and reaction products are concentrated and purified using ethanol precipitation. The sample is electrokinetically injected into the capillary at 3 kV for 45 sec and separated via electrophoresis at 9 kV for 120 min.

1.2 Isolation and Sequencing of cDNA Fragments

Construction of rice cDNA library. Total RNA is purified from rice plant tissue using standard total RNA purification methods. PolyA+ RNA is isolated from the total RNA using the Qiagen Oligotex mRNA purification system (Qiagen, Valencia, Calif.), and cDNA is generated using cDNA synthesis reagents from Life Technologies (Rockville, Md.). First strand cDNA synthesis is catalyzed by reverse transcriptase using oligo dT primers with a NotI restriction site. Second strand synthesis is catalyzed by DNA polymerase. An oligonucleotide linker with a SalI restriction endonuclease site is attached to the 5′ end of the cDNAs using DNA ligase. The cDNAs are digested with NotI and SalI restriction endonucleases and inserted into an E. coli-replicating plasmid harboring a selectable marker. E. coli is transfected with the recombinant plasmids and grown on selectable media. E. coli colonies are individually picked off the selectable media and placed into storage plates.

Sequencing the rice cDNA library, The DNA sequence of the cDNA cloned into the plasmid purified from an E. coli colony is determined using standard dideoxy sequencing methods. Oligonucleotide primers respectively corresponding to plasmid DNA regions upstream of the 5′ end of the cDNA insert (Forward reaction) and downstream of the 3′ end of the cDNA insert (Reverse reaction) are used in the dideoxy sequencing reactions. If the DNA sequence determined as a result of the Forward and Reverse reactions from the cDNA overlapped, the two sequences could be merged into a contig using computerized analysis software (Consed, University of Washington, Seattle), to assemble a full-length sequence of the cDNA. In cases case where DNA sequence from the Forward and Reverse reactions from a single clone did not overlap sufficiently to be assembled into a contig, such that there is a region of unsequenced DNA to bridge the DNA from the Forward and Reverse reaction in order to form a contig, the DNA sequence of the separating region is determined using one of two dideoxy sequencing methods. In a “primer walking” approach, a primer specifically corresponding to the 3′ end of the DNA sequence determined from the Forward reaction is used in a second dedeoxy sequencing reaction. The primer walking procedure is repeated until the DNA sequence that separated the original Forward and Reverse is resolved and a contig could be assembled. Alternatively, the clone harboring the cDNA is subjected to transposon in vitro insertion dideoxysequencing (Epicentre, Madison, Wis.). In this procedure, the insertion process is random and the result is multiple DNA sequence coverage over the targeted cDNA, where the sequences thus obtained are assembled into a contig.

### Example 2

### Cloning and Sequence of Nucleic Acid Molecules from Rice

Primers designed based on the genomic sequence can be used to PCR amplify the full-length cDNA (start to stop codon) from first strand cDNA prepared from rice cultivar Nipponbare leaf tissue.

The PCR fragment is then cloned into pCR2.1-TOPO per the manufacturer's instructions (Invitrogen, Carlsbad, Calif.), and several individual clones are subjected to sequencing analysis.

DNA preps for 2-4 independent clones are miniprepped following the manufacturer's instructions (Qiagen, Valencia, Calif.). DNA is subjected to sequencing analysis using the BigDye™ Terminator Kit according to manufacturer's instructions (Applied Bioscience Inc., Foster City, Calif.). Sequencing makes use of primers designed to both strands of the predicted gene of interest. All sequencing data are analyzed and assembled using the Phred/Phrap/Consed software package (University of Washington) to an error ratio equal to or less than 10⁻⁴ at the consensus sequence level.

The consensus sequence from the sequencing analysis is then to be validated as being intact and the correct gene in several ways. The coding region is checked for being full length (predicted start and stop codons present) and uninterrupted (no internal stop codons). Alignment with the gene prediction and BLAST analysis is used to ascertain that this is in fact the right gene.

### Example 3

### Vector Construction for Overexpression and Gene “Knockout” Experiments

Overexpression

Vectors used for expression of full-length “candidate genes” of interest in plants (overexpression) are designed to overexpress the protein of interest and are of two general types, biolistic and binary, depending on the plant transformation method to be used.

For biolistic transformation (biolistic vectors), the requirements are as follows:

1. a backbone with a bacterial selectable marker (typically, an antibiotic resistance gene) and origin of replication functional in Escherichia coli (E. coli; eg. ColE1), and

2. a plant-specific portion comprising:

a. a gene expression cassette containing a promoter (eg. ZmUBlint MOD), the gene of interest (typically, a full-length cDNA) and a transcriptional terminator (eg. Agrobacterium tumefaciens nos terminator);

b. a plant selectable marker cassette, containing a promoter (eg. rice Act1D-BV MOD), selectable marker gene (eg. phosphomannose isomerase, PMI) and transcriptional terminator (eg. CaMV terminator).

Vectors designed for transformation by Agrobacterium tumefaciens (A. tumefaciens; binary vectors) comprise:

1. a backbone with a bacterial selectable marker functional in both E. coli and A. tumefaciens (eg. spectinomycin resistance mediated by the aadA gene) and two origins of replication, functional in each of aforementioned bacterial hosts, plus the A. tumefaciens virG gene;

2. a plant-specific portion as described for biolistic vectors above, except in this instance this portion is flanked by A. tumefaciens right and left border sequences which mediate transfer of the DNA flanked by these two sequences to the plant.

Knock Out Vectors

Vectors designed for reducing or abolishing expression of a single gene or of a family or related genes (knockout vectors) are also of two general types corresponding to the methodology used to downregulate gene expression: antisense or double-stranded RNA interference (dsRNAi).

Anti-Sense

For antisense vectors, a full-length or partial gene fragment (typically, a portion of the cDNA) can be used in the same vectors described for full-length expression, as part of the gene expression cassette. For antisense-mediated down-regulation of gene expression, the coding region of the gene or gene fragment will be in the opposite orientation relative to the promoter; thus, mRNA will be made from the non-coding (antisense) strand in planta.

dsRNAi

For dsRNAi vectors, a partial gene fragment (typically, 300 to 500 basepairs long) is used in the gene expression cassette, and is expressed in both the sense and antisense orientations, separated by a spacer region (typically, a plant intron, eg. the OsSH1 intron 1, or a selectable marker, eg. conferring kanamycin resistance). Vectors of this type are designed to form a double-stranded mRNA stem, resulting from the basepairing of the two complementary gene fragments in planta.

Biolistic or binary vectors designed for overexpression or knockout can vary in a number of different ways, including eg. the selectable markers used in plant and bacteria, the transcriptional terminators used in the gene expression and plant selectable marker cassettes, and the methodologies used for cloning in gene or gene fragments of interest (typically, conventional restriction enzyme-mediated or Gateway™ recombinase-based cloning). An important variant is the nature of the gene expression cassette promoter driving expression of the gene or gene fragment of interest in most tissues of the plants (constitutive, eg. ZmUBIint MOD), in specific plant tissues (eg. maize ADP-gpp for endosperm-specific expression), or in an inducible fashion (eg. GAL4bsBzl for estradiol-inducible expression in lines constitutively expressing the cognate transcriptional activator for this promoter).

### Example 4

### Insertion of a “Candidate Gene” Involved in Isoprenoid Biosynthesis into Expression Vector

A validated rice cDNA clone in pCR2.1-TOPO is subcloned using conventional restriction enzyme-based cloning into a vector, downstream of the maize ubiquitin promoter and intron, and upstream of the Agrobacterium tumefaciens nos 3′ end transcriptional terminator. The resultant gene expression cassette promoter, “candidate gene” and terminator) is further subcloned, using conventional restriction enzyme-based cloning, into the pNOV2117 binary vector, generating pNOVCANDiso.

The pNOVCANDiso binary vector is designed for transformation and over-expression of the “candidate gene” in monocots. It consists of a binary backbone containing the sequences necessary for selection and growth in Escherichia coli DH-5alpha (Invitrogen) and Agrobacterium tumefaciens LBA4404, including the bacterial spectinomycin antibiotic resistance aadA gene from E. coli transposon Tn7, origins of replication for E. coli (ColE1) and A. tumefaciens (VS 1), and the A. tumefaciens virG gene. In addition to the binary backbone, pNOV2117 contains the T-DNA portion flanked by the right and left border sequences, and including the Positech™ (Syngenta) plant selectable marker and the “candidate gene” gene expression cassette. The Positech™ plant selectable marker confers resistance to mannose and in this instance consists of the maize ubiquitin promoter driving expression of the PMI (phosphomannose isomerase) gene, followed by the cauliflower mosaic virus transcriptional terminator.

### Example 5

### Plant Transformation

5.1 Rice Transformation

pNOVCANDiso is transformed into a rice cultivar (Kaybonnet) using Agrobacterium-mediated transformation, and mannose-resistant calli are selected and regenerated.

Agrobacterium is grown on YPC solid plates for 2-3 days prior to experiment initiation. Agrobacterial colonies are suspended in liquid MS media to an OD of 0.2 at λ600 nm. Acetosyringone is added to the agrobacterial suspension to a concentration of 200 μM and agro is induced for 30 min.

Three-week-old calli which are induced from the scutellum of mature seeds in the N6 medium (Chu et al., Sci. Sin., 18:659, 1975) are incubated in the agrobacterium solution in a 100×25 petri plate for 30 minutes with occasional shaking. The solution is then removed with a pipet and the callus transfered to a MSAs medium which is overlayed with sterile filter paper.

Co-Cultivation is continued for 2 days in the dark at 22° C.

Calli are then placed on MS-Timetin plates for 1 week. After that they are tranfered to PAA+mannose selection media for 3 weeks.

Growing calli (putative events) are picked and transfered to PAA+mannose media and cultivated for 2 weeks in light.

Colonies are tranfered to MS20SorbKinTim regeneration media in plates for 2 weeks in light. Small plantlets are transferred to MS20SorbKinTim regeneration media in GA7 containers. When they reach the lid, they are transfered to soil in the greenhouse.

Expression of the “candidate gene” in transgenic To plants is analyzed. Additional rice cultivars, such as but not limited to, Nipponbare, Taipei 309 and Fuzisaka 2 are also transformed and assayed for expression of the “candidate gene” product and enhanced protein expression.

5.2: Maize Transformation

Transformation of immature mazie embryos is performed essentially as described in Negrotto et al. (Plant Cell Rpts., 19:798, 2000). For this example, all media constituents are as described in Negrotto et al., supra. However, various media constituents described in the literature may be substituted.

The genes used for transformation are cloned into a vector suitable for maize transformation as described in Example 17. Vectors used contain the phosphomannose isomerase (PMI) gene (Negrotto et al., 2000).

Agrobacterium strain LBA4404 (pSB1) containing the plant transformation plasmid is grown on YEP (yeast extract (5 g/L), peptone (10 g/L), NaCl (5 g/L),15 g/l agar, pH 6.8) solid medium for 2 to 4 days at 28° C. Approximately 0.8×10⁹ Agrobacteria are suspended in LS-inf media supplemented with 100 μM acetosyringone (As) (Negrotto et al., 2000). Bacteria are pre-induced in this medium for 30-60 minutes.

Immature embryos from A188 or other suitable maize genotypes are excised from 8-12 day old ears into liquid LS-inf +100 μM As. Embryos are rinsed once with fresh infection medium. Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l) and silver nitrate (1.6 mg/l) and cultured in the dark for 28° C. for 10 days.

Immature embryos producing embryogenic callus are transferred to LSD1M0.5S medium. The cultures are selected on this medium for 6 weeks with a subculture step at 3 weeks. Surviving calli are transferred either to LSD1M0.5S medium to be bulked-up or to Reg1 medium. Following culturing in the light (16 hour light/8 hour dark regiment), green tissues are then transferred to Reg2 medium without growth regulators and incubated for 1-2 weeks. Plantlets are transferred to Magenta GA-7 boxes (Magenta Corp, Chicago Ill.) containing Reg3 medium and grown in the light. Plants that are PCR positive for the promoter-reporter cassette are transferred to soil and grown in the greenhouse.

### Example 6

### GeneChip® Standard Protocol

A rice gene array and probes derived from rice RNA extracted from different tissues and developmental stages can be used to identify the expression profile of genes on the chip.

The standard protocol for using the GeneChip® to quantitatively measure plant gene expression is carried out as outlined below:

Quantitation of Total RNA

1. Total RNA from plant tissue is extracted and quantified using GeneQuant (Amersham Biosciences, Piscataway, N.J.) IOD₂₆₀=40 mg RNA/ml; A₂₆₀/A₂₈₀=1.9 to about 2.1

2. Run gel to check the integrity and purity of the extracted RNA

Synthesis of Double-Stranded cDNA

Gibco/BRL SuperScript Choice System for cDNA Synthesis (Cat#1B090-019) is employed to prepare cDNAs. T7-(dT)₂₄ oligonucleotides are prepared and purified by HPLC. (5′-GGCCAGTGAATTGTAATACGACTCACTATAGGGAGGCGG-(dT)₂₄-3′; SEQ ID NO: 662).

Step 1. Primer Hybridization:

Incubate at 70° C. for 10 minutes

Spin quickly and put on ice briefly

Step 2. Temperature Adjustment:

Incubate at 42° C. for 2 minutes

Step 3. First Strand Synthesis Carried out Using:

DEPC-water-1:1

RNA (10:g final)-10:1

T7=(dT)₂₄ Primer (100 pmol final)-1:1 pmol

5×1^{st} strand cDNA buffer-4:1

0.1M DTT (10 mM final)-2:1

10 mM dNTP mix (500:M final)-1:1

Superscript II RT 200 U/:1-1:1

Total of 20:1

Mix well

Incubate at 42° C. for 1 hour

Step 4. Second Strand Synthesis:

Place reactions on ice, quick spin

DEPC-water-91:1

5×2^{nd} strand cDNA buffer-30:1

10 mM dNTP mix (250 mM final)-3:1

E. coli DNA ligase (10 U/:1)-1:1

E. coli DNA polymerase 1-10 U/:1-4:1

RnaseH 2U/:1-1:1

T4 DNA polymerase 5 U/:1-2:1

0.5 M EDTA (0.5 M final)-10:1

Total 162:1

Mix/spin down/incubate 16° C. for 2 hours

Step 5. Completing the Reaction:

Incubate at 16° C. for 5 minutes

Purification of Double Stranded cDNA

1. Centrifuge PLG (Phase Lock Gel, Eppendorf 5 Prime Inc., pI-188233) at 14,000×, transfered 162:1 of cDNA to PLG

2. Add 162:1 of Phenol:Chloroform:Isoamyl alcohol (pH 8.0), centrifuge 2 minutes

3. Transfer the supernatant to a fresh 1.5 ml tube, add

Glycogen (5 mg/ml) 2

0.5 M NH₄OAC (0.75×Vol) 120

ETOH (2.5×Vol, −20° C.) 400

4. Mix well and centrifuge at 14,000×, for 20 minutes

5. Remove supernatant, added 0.5 ml 80% EtOH (−20° C.)

6. Centrifuge for 5 minutes, air dry or by speed vac for 5-10 minutes

7. Added 44:1 DEPC H₂O

Analyze quantity and size distribution of cDNA

Run a gel using 1:1 ratio of the double-stranded synthesis product to loading buffer

Synthesis of Biotinylated cRNA

(Enzo BioArray High Yield RNA Transcript Labeling Kit Cat#900182)

| | |
|---|---|
| Purified cDNA | 22:1 |
| 10X Hy buffer | 4:1 |
| 10X biotin ribonucleotides | 4:1 |
| 10X DTT | 4:1 |
| 10X Rnase inhibitor mix | 4:1 |
| 20X T7 RNA polymerase | 2:1 |
| Total | 40:1 |

Centrifuge 5 seconds, and incubate for 4 hours at 37° C.

Gently mix every 30-45 minutes

Purification and Quantification of CRNA

(Qiagen Rneasy Mini kit Cat# 74103)

| | | |
|---|---|---|
| cRNA | 40:1 | |
| DEPC H₂O | 60:1 | |
| RLT buffer | 350:1 | mix by vortexing |
| EtOH | 250:1 | mix by pipetting |
| Total | 700:1 | |

Wait 1 minute or more for the RNA to stick

Centrifuge at 2000 rpm for 5 minutes

RPE buffer 500:1

Centrifuge at 10,000 rpm for 1 minute

RPE buffer 500:1

Centrifuge at 10,000 rpm for 1 minute

Centrifuge at 10,000 rpm for 1 minute to dry the column

DEPC H₂O 30:1

Wait for 1 minute, then elute CRNA from by centrifugation, 1 OK 1 minute

DEPC H₂O 30:1

Repeat previous step

Determine concentration and dilute to 1:g/:l concentration

Fragmentation of CRNA

| | |
|---|---|
| cRNA (1:g/:1) | 15:1 |
| 5X Fragmentation Buffer* | 6:1 |
| DEPC H₂O | 9:1 |
| | 30:1 |

*5x Fragmentation Buffer

| | | |
|---|---|---|
| 1M Tris (pH 8.1) | 4.0 | ml |
| MgOAc | 0.64 | g |
| KOAC | 0.98 | g |
| DEPC H₂O | | |
| Total | 20 | ml |
| Filter Sterilize | | |

Array Washed and Stained In:

Stringent Wash Buffer** **Stringent Buffer: 12×MES 83.3 ml, 5 M NaCl 5.2 ml, 10% Tween 1.0 ml, H₂O 910 ml, Filter Sterilize

Non-Stringent Wash Buffer*** ***Non-Stringent Buffer: 20×SSPE 300 ml, 10% Tween 1.0 ml, H₂O 698 ml, Filter Sterilize, Antifoam 1.0.

SAPE Stain**** ****SAPE stain: 2×Stain Buffer 600:1, BSA 48:1, SAPE 12:1, H₂O 540

Antibody Stain***** *****Antibody Stain: 2×Stain Buffer 300:1, H₂O 266.4:1, BSA 24:1, Goat IgG 6:1, Biotinylated Ab 3.6:1

Washed on Fluidics Station Using the Appropriate Antibody Amplification Protocol

### Example 6.2

### Characterization of Gene Expression Profiles

A rice gene array and probes derived from rice RNA extracted from different tissues and developmental stages are used to identify the expression profile of genes on the chip. The rice array contains over 23,000 genes (approximately 18,000 unique genes) or roughly 50% of the rice genome and is similar to the Arabidopsis GeneChip® (Affymetrix) with the exception that the 16 oligonucleotide probe sets do not contain mismatch probe sets. The level of expression is therefore determined by internal software that analyzes the intensity level of the 16 probe sets for each gene. The highest and lowest probes are removed if they do not fit into a set of predefined statistical criteria and the remaining sets are averaged to give an expression value. The final expression values are normalized by software, as described below. The advantages of a gene chip in such an analysis include a global gene expression analysis, quantitative results, a highly reproducible system, and a higher sensitivity than Northern blot analyses.

### Example 6.3

### Plant Growth Conditions and Sampling

Nipponbare rice is grown in the greenhouse with 12 hr light cycle and temperature of 29° C. during the day and 21° C. during the night. Humidity is maintained at 30%. Plants are grown in pots containing 50% sunshine mix and 50% nitrohumus. Individual tissues are collected from a minimum of five plants and pooled. Total RNA is extracted from one gram of tissue using the Qiagen RNA Easy Maxikit (Qiagen, Valencia, Calif.).

The experiments are carried out as described in Zhu et al., Plant Physiol. Biochem., 39:221, 2001.

### Example 6.4

### Data Analysis

Data analysis is done using GeneSpring (Silicon Genetics, Redwood, Calif.) and AlignAce. The genechip sequence is blasted to the AC rice contig sequences. The contig with the best alignment is extracted and five gene prediction programs are run on each contig. The programs used are Genscan trained on arabidopsis and maize, Gmhmm trained on rice and Arabidopsis, and Fgenesh and Glimmer trained on rice. All of the predicted CDSs are blasted against the genechip sequence again to extract the top hit predicted CDS. A Perl script is utilized to extract up to 2 kb of the putative promoter sequence. In some of the genechip sequences there is more than one perfect alignment to a predicted CDS; in such cases, both of the perfect alignments are accepted as the putative genes.

### Example 7

### Chromosomal Markers to Identify the Location of a Nucleic Acid Sequence

The sequences of the present invention can also be used for SSR mapping. SSR mapping in rice has been described by Miyao et al. (DNA Res 3:233 (1996)) and Yang et al. (Mol Gen Genet 245:187 (1994)), and in maize by Ahn et al. (Mol Gen Genet 241:483 (1993)). SSR mapping can be achieved using various methods. In one instance, polymorphisms are identified when sequence specific probes flanking an SSR contained within a sequence are made and used in polymerase chain reaction (PCR) assays with template DNA from two or more individuals or, in plants, near isogenic lines. A change in the number of tandem repeats between the SSR-flanking sequence produces differently sized fragments (U.S. Pat. No. 5,766,847). Alternatively, polymorphisms can be identified by using the PCR fragment produced from the SSR-flanking sequence specific primer reaction as a probe against Southern blots representing different individuals (Refseth et al., Electrophoresis 18:1519 (1997)). Rice SSRs can be used to map a molecular marker closely linked to functional gene, as described by Akagi et al. (Genome 39:205 (1996)).

The sequences of the present invention can be used to identify and develop a variety of microsatellite markers, including the SSRs described above, as genetic markers for comparative analysis and mapping of genomes.

Many of the polynucleotides described herein contain at least 3 consecutive di-, tri- or tetranucleotide repeat units in their coding region that can potentially be developed into SSR markers. Table 2. Trinucleotide motifs that can be commonly found in the coding regions of said polynucleotides and easily identified by screening the polynucleotides sequences for said motifs are, for example: CGG; GCC, CGC, GGC, etc. Once such a repeat unit has been found, primers can be designed which are complementary to the region flanking the repeat unit and used in any of the methods described below.

Sequences of the present invention can also be used in a variation of the SSR technique known as inter-SSR (ISSR), which uses microsatellite oligonucleotides as primers to amplify genomic segments different from the repeat region itself (Zietkiewicz et al., Genomics 20:176, 1994). ISSR employs oligonucleotides based on a simple sequence repeat anchored or not at their 5′- or 3′-end by two to four arbitrarily chosen nucleotides, which triggers site-specific annealing and initiates PCR amplification of genomic segments which are flanked by inversely orientated and closely spaced repeat sequences. In one embodiment of the present invention, microsatellite markers as disclosed herein, or substantially similar sequences or allelic variants thereof, may be used to detect the appearance or disappearance of markers indicating genomic instability as described by Leroy et al. (Electron. J Biotechnol, 3(2), at http://www.ejb.org (2000)), where alteration of a fingerprinting pattern indicated loss of a marker corresponding to a part of a gene involved in the regulation of cell proliferation. Microsatellite markers are useful for detecting genomic alterations such as the change observed by Leroy et al. (Electron. J Biotechnol, 3(2), supra (2000)) which appeared to be the consequence of microsatellite instability at the primer binding site or modification of the region between the microsatellites, and illustrated somaclonal variation leading to genomic instability. Consequently, sequences of the present invention are useful for detecting genomic alterations involved in somaclonal variation, which is an important source of new phenotypes.

In addition, because the genomes of closely related species are largely syntenic (that is, they display the same ordering of genes within the genome), these maps can be used to isolate novel alleles from wild relatives of crop species by positional cloning strategies. This shared synteny is very powerful for using genetic maps from one species to map genes in another. For example, a gene mapped in rice provides information for the gene location in maize and wheat.

### Example 8

### Ouantitative Trait Linked Breeding

Various types of maps can be used with the sequences of the invention to identify Quantitative Trait Loci (QTLs) for a variety of uses, including marker-assisted breeding. Many important crop traits are quantitative traits and result from the combined interactions of several genes. These genes reside at different loci in the genome, often on different chromosomes, and generally exhibit multiple alleles at each locus. Developing markers, tools, and methods to identify and isolate the QTLs involved in a trait, enables marker-assisted breeding to enhance desirable traits or suppress undesirable traits. The sequences disclosed herein can be used as markers for QTLs to assist marker-assisted breeding. The sequences of the invention can be used to identify QTLs and isolate alleles as described by Li et al. in a study of QTLs involved in resistance to a pathogen of rice. (Li et al., Mol Gen Genet 261:58, 1999). In addition to isolating QTL alleles in rice, other cereals, and other monocot and dicot crop species, the sequences of the invention can also be used to isolate alleles from the corresponding QTL(s) of wild relatives. Transgenic plants having various combinations of QTL alleles can then be created and the effects of the combinations measured. Once an ideal allele combination has been identified, crop improvement can be accomplished either through biotechnological means or by directed conventional breeding programs. (Flowers et al., J. Exp. Bot. 51:99, 2000; Tanksley and McCouch, Science 277:1063, 1997).

### Example 9

### Marker-Assisted Breeding

Markers or genes associated with specific desirable or undesirable traits are known and used in marker assisted breeding programs. It is particularly beneficial to be able to screen large numbers of markers and large numbers of candidate parental plants or progeny plants.

Marker-assisted selection uses information about marker associated QTLs in selection programs to select individuals with desirable combinations of QTLs. Markers of the present invention can also be used to introduce desired genes and/or QTLs into a population. For example, markers identified by the method of the present invention can be used to assist in the introduction of a QTL or gene into a population by introgression (Dekkers and Hospital, Nat. Rev. Genet. 3:22, 2002)

Marker assisted selection is a well-established technique in genetics and involves the process where organisms are selected based on the presence or absence of a nucleic acid sequence associated with a particular trait or phenotype. In addition, the markers identified by the methods disclosed herein can be used to produce hybridization probes that can be used in selection. Markers produced by the methods of the present invention can be used to identify and follow quantitative trait loci or QTLs. Marker assisted selection has the advantage of allowing organisms to be selected for the phenotype, trait or QTL prior to when it would be phenotypically apparent. Because it allows for early selection, marker-assisted selection decreases the amount of time need for selection and thus allows more rapid genetic progress.

Sequences disclosed herein that can be used for markers of OTLs are given in Tables 3, 4, 5 and 6. The sequences were further characterized by their change in expression in response to various conditions related to the traits listed. For example, if the trait of interest was abiotic stress, sequences associated with a QTL and that showed either at least a 2-fold or 4-fold change in expression in response to an abiotic stress were identified. A similar procedure was used for the additional traits listed in Tables 3, 4, 5, and 6. Thus Table 3A lists sequences associated with the listed rice QTLs and that show at least a 4-fold change in expression in relation to the listed trait. Table 4A lists sequences associated with the listed rice QTLs and that show at least a 2-fold change in expression in relation to the listed trait. Table 5A lists sequences associated with the listed maize QTLs and that show at least a 4-fold change in expression in relation to the listed trait. Table 6A lists sequences associated with the listed maize QTLs and that show at least a 2-fold change in expression in relation to the listed trait.

The sequences listed in Tables 3, 4, 5, and 6 can be used are markers for the associated QTLs in marker assisted breeding. In addition, the expression of the sequences listed in Tables 3, 4, 5, and 6 can be altered (e.g. either increased or decreased) in order to improved performance of plants, and in particular cereals, for the traits listed using standard techniques, including, but not limited to, those described herein.

TABLE 1
SEQ ID NOs and corresponding descriptions of rice sequences associated with
isoprenoid metabolism; SEQ ID NOs for corresponding homologous sequences
found in wheat, banana and maize; and the AGI number for
corresponding Arabidopsis sequences.

| | | | | | |
|---|---|---|---|---|---|
| Rice | Wheat | Banana | Maize | | |
| SEQ ID | SEQ ID | SEQ ID | SEQ ID | | AGI |
| NO | NO | NO | NO | Gene Product | number |

mevalonate-independent pathway (plus IPPI)

| | | | | | |
|---|---|---|---|---|---|
| | | | | 1-deoxy-D-xylulose 5-phosphate | |
| | | | | synthase (3 sequences) | |
| 113 | 500 | 430 | 635 | DXPS1 | At3g21500 |
| 113 | 500 | 430 | 635 | DXPS2 (CLA1) | At4g15560 |
| 99 | 508 | | | DXPS3 | At5g11380 |
| 267 | 555 | 418 | | 1-deoxy-D-xylulose 5-phosphate | At5g62790 |
| | | | | reductoisomerase (EC 1.1.1.-; | |
| | | | | DXR) | |
| 153 | | | | 2-C-methyl-D-erythritol 4- | At2g02500 |
| | | | | phosphate cytidyltransferase | |
| | | | | (MCT) | |
| | | | | 4-(cytidine 5″-diphospho)-2-C- | At2g26930 |
| | | | | methyl-D-erythritol kinase (CMK) | |
| 161 | 570 | 463 | 612 | 2-C-methyl-D-erythritol 2,4- | At1g63970 |
| | | | | cyclodiphosphate synthase | |
| | | | | (MECPS) | |
| | | | | lytB homologue | At4g34350 |
| | | | | gcpE homologue | At5g60600 |
| | | | | isopentenyl diphosphate: | |
| | | | | dimethylallyl diphosphate | |
| | | | | isomerase (EC 5.3.3.2) | |
| | | | | IPPI1 | At3g02780 |
| | | | | IPPI2 | At5g16440 |

prenyltransferases

| | | | | | |
|---|---|---|---|---|---|
| | | | | geranyl diphosphate synthase (EC) | At2g34630 |
| | | | | farnesyl diphosphate synthase | |
| | | | | (EC) | |
| 265 | 474 | 448 | 607 | FPPS1 | At4g17190 |
| 265 | 474 | 448 | 607 | FPPS2 | At5g47770 |
| | | | | geranylgeranyl diphosphate | |
| | | | | synthase (EC) | |
| | | | | GGPPS1 | At1g49530 |
| 371 | | | 587 | GGPPS2 | At2g18620 |
| 229 | | | | GGPPS3 | At2g18640 |
| | | | | GGPPS4 | At2g23800 |
| | | | | GGPPS5 | At3g14510 |
| | | | | GGPPS6 | At3g14530 |
| | | | | GGPPS7 | At3g14550 |
| | | | | GGPPS8 | At3g20160 |
| 47 | | | | GGPPS9 | At3g29430 |
| | | | | GGPPS10 | At3g32040 |
| | | | | GGPPS11 | At4g36810 |
| 83 | 574 | 468 | 604 | GGPPS12 | At4g38460 |
| 135 | 531 | 454 | 622 | geranylgeranyl reductase | At1g74470 |
| | | | | solanesyl diphosphate synthase- | |
| | | | | like | |
| | | | | SDS-like 1 (prephytoene PP | At1g17050 |
| | | | | dehydrogenase) | |
| | | | | SDS-like2 (prenyltransferase) | At1g78510 |
| | | | | undecaprenyldiphosphate | |
| | | | | synthase-like (3 sequences) | |
| 235 | | | | UPPS-like1 (hypothetical) | At2g17570 |
| 345 | | | | UPPS-like2 | At5g60500 |
| 243 | | | | UPPS-like3 | At5g60510 |
| 299 | | | | | |
| | | | | dehydrodolichol diphosphate | |
| | | | | synthase (3 sequences) | |
| 125 | | | | DPPS-like1 (hypothetical) | At2g23410 |
| 11 | | | | DPPS2 | At5g58770 |
| | | | | DPPS3 | At5g58780 |

protein prenyltransferases

| | | | | | |
|---|---|---|---|---|---|
| 189 | 488 | | 599 | farnesyltransferase/geranylgeranyl | At3g59380 |
| | | | | transferase I, a-subunit | |
| | | | | farnesyltransferase, b-subunit | At5g40280 |
| | | | | (ERA1) | |
| 255 | 489 | | 624 | geranylgeranyl transferase I, b- | At2g39550 |
| 63 | | | | subunit | |
| 373 | | | | | |
| | | | | (Rab) geranylgeranyl transferase | |
| | | | | II, a-subunit | |
| 23 | 580 | 419 | 591 | GGTase II a-1 | At4g24490 |
| 289 | | | | GGTase II a-2 | At5g41820 |
| | | | | (Rab) geranylgeranyl transferase | |
| | | | | II, b-subunit | |
| 343 | 478 | | | GGTase II b-1 | At3g12070 |
| 343 | 478 | | | GGTase II b-2 | At5g12210 |

terpene synthases (cyclases)

| | | | | | |
|---|---|---|---|---|---|
| 357 | | | | monoterpene synthases, | At1g31950 |
| | | | | sesquiterpene synthases and | |
| | | | | diterpene synthases | |
| 157 | | | | (not copalyl diphosphate synthase | At1g33750 |
| 137 | | | | and ent-kaurene synthase) (all by | |
| 43 | 505 | 464 | 623 | homology) | |
| 39 | 504 | | 661 | | |
| 233 | | | | | |
| | | | | monoterpene synthases & | |
| | | | | sesguiterpene synthases | |
| | | | | TPS3 | At1g48800 |
| 145 | | | | TPS4 | At1g48820 |
| 185 | | | | | |
| 385 | | | | | |
| 387, | | | | TPS5 | At1g61120 |
| 327 | 516 | | | | |
| 77 | 559 | | | TPS6 | At1g61680 |
| | | | | TPS7 | At1g66020 |
| 281 | | | | TPS8 | At1g70080 |
| | | | | TPS9 | At2g23230 |
| 101 | | | | TPS10 | At2g24210 |
| 55 | 507 | | 592 | TPS11 (short) | At2g37140 |
| 249 | 513 | | | TPS12 | At3g14490 |
| 81 | | | | TPS13 | At3g14520 |
| 57 | 526 | | | TPS14 | At3g14540 |
| 99 | 508 | | | TPS15 | At3g25810 |
| 167 | | | | | |
| 369 | 553 | 423 | 659 | | |
| 1 | | | | | |
| 305 | 560 | | | | |
| 247 | 527 | 420 | 627 | | |
| 71 | 487 | | | TPS15 | At3g25810 |
| 159 | | 438 | | | |
| 171 | | | | | |
| | | | | TPS16 | At3g25823 |
| 171 | | 438 | | TPS17 | At3g25830 |
| 351 | | | 598 | TPS18 | At3g29110 |
| 319 | 577 | 459 | | TPS19 | At3g29190 |
| 227 | | | | TPS20 | At3g29410 |
| | | | | TPS21 (short) | At3g31415 |
| 109 | 569 | 444 | 637 | TPS22 | At3g32030 |
| 151 | | | | TPS23 | At4g13280 |
| 375 | | | | | |
| 151 | 514 | | 585 | TPS24 | At4g13300 |
| 181 | | | | | |
| 151 | 514 | | 585 | TPS25 | At4g15870 |
| | | | | TPS26 | At4g16730 |
| | | | | TPS27 | At4g16740 |
| 201 | | | | TPS28 | At4g20200 |
| 347 | 509 | | 609 | | |
| 209 | | | | | |
| 211 | | | | | |
| 231 | 483 | 450 | | | |
| 241 | 573 | 456 | 657 | | |
| 69 | 573 | 456 | 657 | | |
| 319 | 577 | 459 | | TPS29 | At4g20210 |
| 263 | 568 | 416 | | TPS30 | At4g20230 |
| 321 | 494 | | 626 | TPS31 | At5g23960 |
| 257 | 554 | 435 | 601 | TPS32 | At5g44630 |
| 179 | 543 | | 611 | | |
| 279 | 515 | | 654 | TPS33 | At5g48110 |
| | | | | triterpene synthases (not | |
| | | | | membranesterol biosynthesis) | |
| | | | | lupeol synthase-like | |
| | | | | LS1 | At1g66960 |
| 115 | 548 | 458 | 640 | LS2 | At1g78950 |
| | | | | LS3(multifunctional) | At1g78960 |
| 115 | 548 | 458 | 640 | LS4(LUP1) | At1g78970 |
| | | | | 2,3-oxidosqualene synthase-like (6 | |
| | | | | sequences) | |
| | | | | TTPS1 | At1g78500 |
| | | | | TTPS2 | At4g15340 |
| 217 | | | | TTPS3 | At4g15370 |
| 283 | 490 | | | | |
| 51 | | | | | |
| 273 | 537 | 447 | 588 | TTPS4 | At5g36150 |
| 9 | | | | | |
| 19 | 584 | 460 | | | |
| 129 | | | | | |
| 41 | 540 | | 652 | | |
| | | | | TTPS5 | At5g42600 |
| | | | | TTPS6 | At5g48010 |

gibberellins

| | | | | | |
|---|---|---|---|---|---|
| | | | | copalyl diphosphate synthase | At4g02780 |
| | | | | (GA1) | |
| 107 | 491 | | 593 | ent-kaurene synthase (GA2) | At1g79460 |
| | | | | ent-kaurene oxidase (GA3) | At5g25900 |
| | | | | ent-kaurenoic acid oxidase | |
| 67 | | | | KAO1 | At1g05160 |
| 155 | 572 | | 621 | | |
| | | | | KAO2 | At2g32440 |
| 399 | 501 | 445 | 647 | gibberellin 7 oxidase | At5g05600 |
| | | | | gibberellin 13-hydroxylase | |
| | | | | gibberellin 20-oxidase | |
| 79 | | | | GA5-1 | At1g44090 |
| 25 | | | | GA5-2 | At1g60980 |
| 75 | | | | GA5-3 | At4g25420 |
| 79 | | | | GA5-4 | At5g07200 |
| | | | | GA5-5 | At5g51810 |
| | | | | gibberellin 3-hydroxylase | |
| 297 | 581 | | 646 | GA4-1 | At1g15550 |
| 359 | 533 | | | GA4-2 | At1g80330 |
| 225 | 575 | | 653 | | |
| 291 | 541 | 436 | 596 | GA4-3 | At1g80340 |
| 253 | 544 | | 630 | | |
| 377 | 552 | 466 | 650 | | |
| 75 | 539 | | 636 | GA4-4 | At4g25420 |
| | | | | gibberellin 2-oxidase | |
| 141 | 511 | 453 | 614 | 2OX-1 | At1g30040 |
| 141 | 511 | 453 | 614 | 2OX-2 | At1g78440 |
| 173 | 545 | | 629 | 2OX-3 | At2g34550 |
| 193 | 472 | | | | |
| 203 | 520 | | | | |
| 205 | | | | | |
| 259 | 499 | | 656 | | |
| 261 | 486 | | 638 | | |
| 349 | 542 | 467 | 590 | | |
| 375 | 512 | 465 | | | |
| 97 | | | | | |
| 223 | | | | | |

carotenoids and abscisic acid

| | | | | | |
|---|---|---|---|---|---|
| 317 | 582 | 469 | 628 | phytoene synthase | At5g17230 |
| | | | | phytoene desaturase (PDS) | At4g14210 |
| 383 | 506 | | | zeta-carotene desaturase (ZDS) | At3g04870 |
| 205 | 532 | 427 | 597 | lyeopene b-cyclase (LCYB) | At3g10230 |
| 237 | | | | | |
| 121 | | | | | |
| 27 | | | | | |
| | | | | lycopene e-cyelase (LCYE) | At5g57030 |
| | | | | beta carotene hydroxylase | |
| | | | | zeaxanthin epoxidase (ZEP) | At5g67030 |
| | | | | violaxanthin de-epoxidase (VDE1) | At1g08550 |
| | | | | epoxycarotenoid (neoxanthin) | |
| | | | | cleavage enzyme | |
| 301 | 517 | | 645 | NC1 | At1g30100 |
| | | | | NC2 | At1g78390 |
| | | | | NC3 | At3g14440 |
| 103 | | | | NC4 | At3g24220 |
| 245 | 561 | 442 | 613 | NC5 | At3g63520 |
| | | | | NC6 | At4g18350 |
| 271 | 524 | | 645 | NC7 | At4g19170 |
| 313 | 473 | | | abscisic aldehyde oxidase | At2g27150 |
| 391 | | 473 | | carotenoid isomerase 2 | At1g57770 |
| | | | | (CRTISO2) | |
| 397 | 567 | 446 | 615 | beta-carotene hydroxylase (CHY1) | At4g25700 |
| 395 | 481 | 431 | | plastid terminal oxidase (PTO) | At4g22260 |
| | | | | (immutans) | |

Tocopherols

| | | | | | |
|---|---|---|---|---|---|
| 275 | | | 658 | p-hydroxyphenylpyruvate | At1g06570 |
| | | | | dioxygenase (HPPD) (1 sequence) | |
| 89 | 535 | 439 | | C-methyltransferase | |
| | | | | tocopherol cyclase | |
| | | | | gama tocopherol C- | At1g64970 |
| | | | | methyltransferase (TMT) | |

Plastoquinone and phylloquinone

| | | | | | |
|---|---|---|---|---|---|
| 213 | 576 | | | homogentisate (hexaprenyl) | At3g11950 |
| | | | | solanesyltransferase (1 sequence) | |
| | | | | C-methyltransferase | |
| | | | | isochorismate synthase (EC | |
| | | | | 5.4.99.6; entC) | |
| | | | | | At1g18870 |
| 29 | | | | | At1g74710 |
| 17 | | | | | |
| | | | | SHCHC synthase (menD) | At1g68890 |
| | | | | (menaquinone biosynthesis) | |
| | | | | OSB synthase (menC) | At1g68900 |
| | | | | (hypothetical) | |
| 87 | 557 | 432 | 618 | OSB-CoA ligase (menE) | At3g48990 |
| | | | | (p-coumaroyl-CoA ligase) | |
| | | | | DHNA synthase | At1g60550 |
| 85 | 485 | | | DHNA phytyltransferase (menA) | At1g60600 |
| | | | | (hypothetical) | |
| | | | | C-methyltransferase (ubiE) | At1g23360 |
| | | | | (spore germination protein c2) | |
| 393 | 536 | 425 | | C-methyltransferase (ubiE) | At3g02770 |
| 207 | 536 | 425 | | | |
| 401 | 536 | 425 | 616 | C-methyltransferase (ubiE) | At5g16450 |
| 285 | 536 | 425 | 616 | | |
| 89 | 535 | 439 | | C-methyltransferase (ubiE) | At5g56260 |
| 213 | 576 | | | homogentisate | At3g11950 |
| | | | | polyprenyltransferase | |

Chlorophyll and heme

| | | | | | |
|---|---|---|---|---|---|
| | | | | glutamyl tRNA synthetase | At5g26707 |
| | | | | glutamyl tRNA reductase | |
| 149 | 547 | 417 | 644 | HEMA1 | At1g09940 |
| 149 | 547 | 417 | 644 | HEMA2 | At1g58290 |
| 149 | 547 | 417 | 644 | HEMA3 | At2g31250 |
| | | | | glutamate 1-semialdehyde | |
| | | | | aminotransferase | |
| | | | | GSA1 | At3g48730 |
| | | | | GSA2 | At5g63570 |
| | | | | aminolevulinate dehydratase | |
| | | | | ALAD1 | At1g44318 |
| | | | | ALAD2 | At1g69740 |
| | | | | porphobilinogen deaminase | At5g08280 |
| | | | | (HEM3) | |
| | | | | uroporphyrinogen decarboxylase | |
| 335 | 522 | 455 | 639 | UPD1 | At2g40490 |
| 191 | | | 634 | UPD2 | At3g14930 |
| | | | | coproporphyrinogen III oxidase | At1g03475 |
| | | | | (CPOX) | |
| | | | | protoporphyrinogen IX oxidase | |
| | | | | PPO1 | At4g01690 |
| | | | | PPO2 | At5g14220 |
| | | | | Mg-protoporphyrinogen IX | |
| | | | | chelatase | |
| | | | | CLD1 | At1g08520 |
| 295 | | | | CLD2 | At4g18480 |
| 175 | 546 | 438 | | | |
| 325 | | | | | |
| 139 | 525 | | | | |
| 53 | | | | CLD3 | At5g49530 |
| 31 | 484 | 461 | | Mg-protoporphyrinogen IX | At4g25080 |
| | | | | methyltransferase (PPMT) | |
| | | | | Mg-protoporphyrinogen IX | |
| | | | | monomethylester cyclase | |
| | | | | divinyl-protochlorophyllide a | |
| | | | | reductase | |
| | | | | protochlorophyllide reductase (EC | |
| | | | | 1.3.1.33) | |
| 339 | | | | POR1 (PORC; light-dependent) | At1g03630 |
| | | | | POR2 (PORB) | At4g27440 |
| | | | | POR3 (PORA) | At5g54190 |
| 105 | 571 | 433 | | chlorophyll synthetase | At3g51820 |
| | | | | chlorophyll a oxygenase | At1g44446 |
| | | | | (chlorophyll b synthase) | |
| | | | | chlorophyllase | |
| | | | | CLH1 | At1g19670 |
| 177 | | | | CLH2 | At5g43860 |
| 117 | 578 | | | red chlorophyll catabolite | At4g37000 |
| 363 | | | | reductase | |
| 355 | | | | | |
| 143 | 528 | | | | |
| | | | | ferrochelatase | |
| | | | | FC1 | At2g30390 |
| | | | | FC2 | At5g26030 |
| | | | | heme oxygenase | |
| | | | | HO1 | At1g58300 |
| 277 | 495 | | | HO2 | At1g69720 |
| 127 | | | | | |
| 47 | | | | HO3 | At2g26550 |
| 221 | 518 | | | | |
| | | | | HO4 | At2g26670 |
| 199 | | 428 | | phytochromobilin synthase (HY2) | At3g09150 |
| 307 | 510 | 421 | 642 | | |

mevalonate pathway

| | | | | | |
|---|---|---|---|---|---|
| | | | | acetoacetyl-CoA thiolase | |
| 165 | 558 | 452 | | AACT1 | At5g47720 |
| 165 | 558 | 452 | | AACT2 | At5g48230 |
| 315 | 564 | 470 | 606 | 3-hydroxy-3-methylglutaryl-CoA | At4g11820 |
| | | | | synthase | |
| | | | | 3-hydroxy-3-methylglutaryl-CoA | |
| | | | | reductase | |
| 133 | 476 | 426 | 641 | HMGR1 | At1g76490 |
| 353 | | 457 | 641 | HMGR2 | At2g17370 |
| 111 | 479 | | | mevalonate kinase | At5g27450 |
| | | | | phosphomevalonate kinase | At1g31910 |
| | | | | mevalonate diphosphate | |
| | | | | decarboxylase | |
| 293 | 551 | 443 | | MPDC1 | At2g38700 |
| 365 | 551 | 443 | | | |
| | | | | MPDC2 | At3g54250 |

sterols and brassinosteroids

| | | | | | |
|---|---|---|---|---|---|
| | | | | squalene synthase | |
| 169 | 566 | | 649 | SQSl | At4g34640 |
| 169 | 566 | | 649 | SQS2 | At4g34650 |
| | | | | squalene monooxygenase (EC | |
| | | | | 1.14.99.7) | |
| | | | | SE1 | At1g58440 |
| | | | | SE1 | At2g22830 |
| | | | | SE1 | At4g37760 |
| 309 | | | | SE1 | At5g24140 |
| 173 | 545 | | 629 | | |
| 147 | 523 | 424 | | SE1 | At5g24150 |
| 303 | | | | SE1 | At5g24160 |
| | | | | cycloartenol synthase (EC | |
| | | | | 5.4.99.8) | |
| | | | | 1 | At2g07050 |
| 361 | | 458 | | 2 | At3g45130 |
| 13 | 556 | 449 | 610 | cycloartenol C24 | At5g13710 |
| | | | | methyltransferase | |
| | | | | 24-methylenecycloartenol C-4 | At4g12110 |
| | | | | demethylase | |
| 195 | 475 | | | (additional candidate) | At4g22750 |
| | | | | cycloeucalenol cycloisomerase | At5g50375 |
| 123 | | | | sterol C14 reductase (fackel) | At3g50430 |
| 311 | 521 | 415 | | | |
| 59 | 503 | | | | |
| | | | | obtusifoliol 14-demethylase | |
| | | | | (CYP51) | |
| | | | | CYP51-1 | At1g11680 |
| | | | | CYPS1-2 | At2g17330 |
| 33 | 477 | | | C-8,7 sterol isomerase | At1g20050 |
| | | | | sterol C-methyltransferase 2 | |
| 251 | 529 | 471 | 625 | SMT2-1 | At1g20330 |
| 251 | 529 | 471 | 625 | SMT2-2 | At1g76090 |
| | | | | sterol C-4 methyl oxidase | |
| | | | | (demethylase) | |
| 219 | 482 | | | S4MO-1 | At1g07420 |
| | | | | S4MO-2 | At2g29390 |
| | | | | sterol C5-desaturase (2 sequences) | |
| 287 | 530 | 422 | 608 | DWF7-1 | At3g02580 |
| 287 | 530 | 422 | 608 | DWF7-2 | At3g02590 |
| | | | | sterol delta 7 reductase | At1g50430 |
| 333 | 562 | 440 | 620 | sterol C24-reductase (DWF1, | At3g19820 |
| | | | | diminuto) | |
| 337 | | | | sterol reductase (DET2) | At2g38050 |
| | | | | sterol C 22-desaturase | not |
| | | | | sterol glucosyltransferase (EC | At1g43620 |
| | | | | 2.4.1.173) | |
| | | | | | At3g07020 |
| 323 | | | 631 | steroid C22-hydroxylase | At3g50660 |
| 49 | | | 660 | (CYP90B1; DWF1) | |
| | | | | steroid C23-hydroxylase | At5g05690 |
| | | | | (CYP90A1) | |
| 45 | 565 | 462 | 589 | 3-oxo-5-a-sterol reductase | At5g16010 |
| 119 | | | 632 | | |
| | | | | steroid sulfotransferase-like (SST) | |
| 379 | 493 | | | SST1 | At1g13420 |
| 37 | | | | SST2 | At1g13430 |
| 183 | | | | SST3 | At1g28170 |
| 269 | | | | | |
| 65 | 538 | | 602 | SST4 | At2g03750 |
| 389 | | | | SST5 | At2g03760 |
| 15 | 492 | | 643 | SST6 | At2g27570 |
| 367 | | | | SST7 | At2g43420 |
| 131 | 497 | | 633 | SST8 | At3g45070 |
| 197 | 497 | | | | |
| 5 | | | | SST9 | At4g26280 |
| 329 | 492 | | 605 | | |
| 61 | | | | SST10 | At5g07000 |
| 3 | 492 | | 643 | SST11 | At5g07010 |
| 91 | 549 | | 595 | SST12 | At5g43690 |
| 331 | 583 | | 619 | | |
| 95 | 496 | | | 3-beta-hydroxysteroid | At2g33630 |
| | | | | dehydrogenase-like | |
| | | | | 11-beta-hydroxysteroid | |
| | | | | dehydrogenase-like (STDH) | |
| 187 | | | | STDH1 | At4g10020 |
| 341 | 480 | | 651 | STDH2 | At5g50600 |
| 35 | | | | STDH3 | At5g50770 |
| 163 | | | | | |
| 21 | 550 | 441 | | 17-beta-hydroxysteroid | At1g76150 |
| | | | | dehydrogenase-like | |

ubiquinone biosynthesis

| | | | | | |
|---|---|---|---|---|---|
| | | | | p-hydroxybenzoate (hexaprenyl) | At4g23660 |
| | | | | solanesyltransferase (CoQ2) | |
| | | | | monooxygenase (ubiF/ubiH-like) | At3g24200 |
| | | | | (monooxygenase) | |
| | | | | O-methyltransferase | At2g30920 |
| | | | | (multifunctional) (CoQ3) | |
| | | | | decarboxylase | |
| 239 | 498 | 451 | | C-methyltransferase (CoQ5/ubiE) | At5g57300 |
| 7 | 579 | 434 | 594 | CoQ 4 homologue (function | At2g03690 |
| 381 | 579 | 434 | 594 | unknown) | |

TABLE 2

This table provides the start and end points for
simple sequence repeats (SSRs) contained in the
sequences listed.

| | | | |
|---|---|---|---|
| Seq ID | Start | End | Sequence |

| | | | |
|---|---|---|---|
| 15 | 362 | 376 | CCG |
| 31 | 216 | 230 | CCG |
| 39 | 230 | 247 | AGG |
| | 371 | 385 | AGG |
| 41 | 101 | 115 | CGG |
| 45 | 7 | 27 | ATC |
| 51 | 1093 | 1107 | TCA |
| 59 | 71 | 85 | GCC |
| 63 | 664 | 678 | AAG |
| 73 | 616 | 633 | GAT |
| 77 | 83 | 103 | CTT |
| 83 | 53 | 70 | CGC |
| 107 | 28 | 45 | AGC |
| 109 | 1609 | 1623 | AGC |
| 117 | 81 | 95 | TGG |
| 119 | 146 | 160 | CTG |
| | 182 | 196 | GTG |
| | 244 | 258 | GGT |
| | 258 | 275 | TGA |
| 125 | 11 | 28 | CGG |
| 133 | 1727 1742 | GAAA | |
| 155 | 477 | 494 | TCC |
| 157 | 60 | 74 | ACC |
| 191 | 148 | 162 | GAG |
| 211 | 996 | 1013 | GA |
| 215 | 55 | 72 | CTG |
| 225 | 75 | 89 | AGG |
| 229 | 130 | 144 | CCG |
| 245 | 2331 | 2348 | AAG |
| 247 | 641 | 655 | CCG |
| 257 | 77 | 91 | ACC |
| 261 | 349 | 363 | GAT |
| 271 | 49 | 63 | CCG |
| 275 | 56 | 79 | AG |
| | 94 | 109 | GTGC |
| | 164 | 190 | CGG |
| 291 | 153 | 173 | CGA |
| | 338 | 352 | CCG |
| 299 | 28 | 42 | CTT |
| 313 | 8 | 22 | CGC |
| 321 | 324 | 338 | GAG |
| 323 | 289 | 303 | GAG |
| | 342 | 359 | CGG |
| 349 | 132 | 146 | GTC |
| 363 | 157 | 177 | AAG |
| | 465 | 482 | CGA |
| 371 | 95 | 115 | CGG |
| 389 | 236 | 250 | CGT |
| | 382 | 397 | CGCT |
| | 486 | 501 | AGCC |
| 399 | 194 | 208 | CGG |
| | 1059 1073 | CGC | |

TABLE 3A
Sequences associated with the listed rice quantitative trait loci
(QTLs) and showing at least a 4-fold alteration in expression associated
with the listed trait.

| QTL | TRAIT | SEQ ID |
|---|---|---|
| ABIOTIC_STRESS_OS-LFDRY-10-1 | ABIOTIC STRESS | 259 |
| ABIOTIC_STRESS_OS-LFDRY-11-1 | ABIOTIC STRESS | 283 |
| ABIOTIC_STRESS_OS-LFDRY-4-2 | ABIOTIC STRESS | 87 |
| ABIOTIC_STRESS_OS-RGR-12-1 | ABIOTIC STRESS | 233 |
| ABIOTIC_STRESS_OS-RGR-12-1 | ABIOTIC STRESS | 233 |
| ABIOTIC_STRESS_OS-RGR-12-1 | ABIOTIC STRESS | 233 |
| ABIOTIC_STRESS_OS-RRL-11-1 | ABIOTIC STRESS | 283 |
| ABIOTIC_STRESS_OS-SSL-10-1 | ABIOTIC STRESS | 259 |
| ABIOTIC_STRESS_OS-ST-1-2 | ABIOTIC STRESS | 285 |
| MATURITY_OS-DTF10-8-2 | MATURITY | 273 |
| MATURITY_OS-HT-2-2 | MATURITY | 273 |
| MATURITY_OS-HT-8-1 | MATURITY | 273 |
| PATHOGEN_RESISTANCE_OS-BP-3-1 | PATHOGEN RESISTANCE | 319 |
| PATHOGEN_RESISTANCE_OS-LS-2-1 | PATHOGEN RESISTANCE | 25 |
| PATHOGEN_RESISTANCE_OS-LS-3-1 | PATHOGEN RESISTANCE | 33 |
| PATHOGEN_RESISTANCE_OS-LS-3-3 | PATHOGEN RESISTANCE | 319 |
| PATHOGEN_RESISTANCE_OS-SB-11-1 | PATHOGEN RESISTANCE | 27 |
| PATHOGEN_RESISTANCE_OS-SB-2-1 | PATHOGEN RESISTANCE | 99 |
| QUALITY_OS-BDV-12-1 | GRAIN_QUALITY | 131 |
| QUALITY_OS-BDV-12-1 | GRAIN_QUALITY | 131 |
| QUALITY_OS-BDV-12-1 | GRAIN_QUALITY | 131 |
| QUALITY_OS-BDV-12-1 | GRAIN_QUALITY | 131 |
| QUALITY_OS-PKV-12-1 | GRAIN_QUALITY | 131 |
| QUALITY_OS-PKV-12-1 | GRAIN_QUALITY | 131 |
| QUALITY_OS-PKV-2-1 | GRAIN_QUALITY | 291 |
| QUALITY_OS-PKV-2-1 | GRAIN_QUALITY | 291 |
| QUALITY_OS-PKV-2-1 | GRAIN_QUALITY | 291 |

TABLE 3B
References for the listed quantitative trait loci (QTLs)
listed in Table 3A

| QTL | REFERENCE |
|---|---|
| OS-BDV-1-1 | THEOR APPL GENET (2000) 100:280-284 |
| OS-BDV-12-1 | THEOR APPL GENET (2000) 100:280-284 |
| OS-CSV-1-1 | THEOR APPL GENET (2000) 100:280-284 |
| OS-CPV-1-1 | THEOR APPL GENET (2000) 100:280-284 |
| OS-DTF10-8-2 | MOLECULAR BREEDING (2000) 6:145-155 |
| OS-CHALK-1-1 | THEOR APPL GENET (2000) 101:823-829 |
| OS-LFDRY-4-2 | MOLECULAR BREEDING (2000) 6:55-66 |
| OS-LFDRY-10-1 | MOLECULAR BREEDING (2000) 6:55-66 |
| OS-LFDRY-11-1 | MOLECULAR BREEDING (2000) 6:55-66 |
| OS-PKV-2-1 | THEOR APPL GENET (2000) 100:280-284 |
| OS-PKV-12-1 | THEOR APPL GENET (2000) 100:280-284 |
| OS-RGR-12-1 | MOLECULAR BREEDING (2000) 6:55-66 |
| OS-SBV-1-1 | THEOR APPL GENET (2000) 100:280-284 |
| OS-ST-1-2 | THEOR APPL GENET (2000) 101:1074-1081 |
| OS-LS-2-1 | THEOR APPL GENET (2000) 101:286-291 |
| OS-LS-3-1 | THEOR APPL GENET (2000) 101:286-291 |
| OS-LS-3-3 | THEOR APPL GENET (2000) 101:286-291 |
| OS-SB-2-1 | THEOR APPL GENET (2000) 101:569-573 |
| OS-SB-11-1 | THEOR APPL GENET (2000) 101:569-573 |
| OS-BP-3-1 | THEOR APPL GENET (2001) 102:929-934 |
| OS-RRL-11-1 | THEOR APPL GENET (2001) 102:1002-1010 |
| OS-SSL-10-1 | THEOR APPL GENET (2001) 102:1002-1010 |
| OS-HT-2-2 | THEOR APPL GENET (2001) 102:1236-1242 |
| OS-HT-8-1 | THEOR APPL GENET (2001) 102:1236-1242 |

TABLE 4A
Sequences associated with the listed rice quantitative trait loci
(QTLs) and showing at least a 2-fold alteration in expression associated
with the listed trait.

| QTL | TRAIT | SEQ ID |
|---|---|---|
| ABIOTIC_STRESS_OS-ALTOL-12-1 | ABIOTIC STRESS | 281 |
| ABIOTIC_STRESS_OS-ALTOL-12-1 | ABIOTIC STRESS | 281 |
| ABIOTIC_STRESS_OS-LFDRY-10-1 | ABIOTIC STRESS | 259 |
| ABIOTIC_STRESS_OS-LFDRY-10-1 | ABIOTIC STRESS | 259 |
| ABIOTIC_STRESS_OS-LFDRY-11-1 | ABIOTIC STRESS | 283 |
| ABIOTIC_STRESS_OS-LFDRY-11-1 | ABIOTIC STRESS | 283 |
| ABIOTIC_STRESS_OS-LFDRY-11-1 | ABIOTIC STRESS | 27 |
| ABIOTIC_STRESS_OS-LFDRY-11-1 | ABIOTIC STRESS | 283 |
| ABIOTIC_STRESS_OS-LFDRY-4-2 | ABIOTIC STRESS | 87 |
| ABIOTIC_STRESS_OS-LFDRY-4-2 | ABIOTIC STRESS | 87 |
| ABIOTIC_STRESS_OS-LFDRY-4-2 | ABIOTIC STRESS | 217 |
| ABIOTIC_STRESS_OS-LFDRY-4-2 | ABIOTIC STRESS | 87 |
| ABIOTIC_STRESS_OS-LFDRY-4-2 | ABIOTIC STRESS | 217 |
| ABIOTIC_STRESS_OS-LFDRY-5-1 | ABIOTIC STRESS | 71 |
| ABIOTIC_STRESS_OS-LFROL-4-1 | ABIOTIC STRESS | 217 |
| ABIOTIC_STRESS_OS-LFROL-4-1 | ABIOTIC STRESS | 217 |
| ABIOTIC_STRESS_OS-LFROL-5-1 | ABIOTIC STRESS | 71 |
| ABIOTIC_STRESS_OS-RGR-12-1 | ABIOTIC STRESS | 233 |
| ABIOTIC_STRESS_OS-RGR-12-1 | ABIOTIC STRESS | 233 |
| ABIOTIC_STRESS_OS-RGR-12-1 | ABIOTIC STRESS | 233 |
| ABIOTIC_STRESS_OS-RGR-12-1 | ABIOTIC STRESS | 27 |
| ABIOTIC_STRESS_OS-RGR-12-1 | ABIOTIC STRESS | 233 |
| ABIOTIC_STRESS_OS-RGR-12-1 | ABIOTIC STRESS | 281 |
| ABIOTIC_STRESS_OS-RGR-12-1 | ABIOTIC STRESS | 281 |
| ABIOTIC_STRESS_OS-RGR-12-1 | ABIOTIC STRESS | 233 |
| ABIOTIC_STRESS_OS-RGR-2-1 | ABIOTIC STRESS | 321 |
| ABIOTIC_STRESS_OS-RGR-2-1 | ABIOTIC STRESS | 305 |
| ABIOTIC_STRESS_OS-RGR-2-1 | ABIOTIC STRESS | 321 |
| ABIOTIC_STRESS_OS-RGR-5-1 | ABIOTIC STRESS | 3 |
| ABIOTIC_STRESS_OS-RGR-5-1 | ABIOTIC STRESS | 3 |
| ABIOTIC_STRESS_OS-RRL-11-1 | ABIOTIC STRESS | 283 |
| ABIOTIC_STRESS_OS-RRL-11-1 | ABIOTIC STRESS | 283 |
| ABIOTIC_STRESS_OS-RRL-11-1 | ABIOTIC STRESS | 283 |
| ABIOTIC_STRESS_OS-RWC-12-1 | ABIOTIC STRESS | 131 |
| ABIOTIC_STRESS_OS-RWC-12-1 | ABIOTIC STRESS | 197 |
| ABIOTIC_STRESS_OS-RWC-12-1 | ABIOTIC STRESS | 197 |
| ABIOTIC_STRESS_OS-RWC-12-1 | ABIOTIC STRESS | 131 |
| ABIOTIC_STRESS_OS-RWC-12-1 | ABIOTIC STRESS | 131 |
| ABIOTIC_STRESS_OS-RWC-6-1 | ABIOTIC STRESS | 191 |
| ABIOTIC_STRESS_OS-SSL-10-1 | ABIOTIC STRESS | 259 |
| ABIOTIC_STRESS_OS-SSL-10-1 | ABIOTIC STRESS | 259 |
| ABIOTIC_STRESS_OS-ST-1-2 | ABIOTIC STRESS | 285 |
| ABIOTIC_STRESS_OS-ST-1-2 | ABIOTIC STRESS | 285 |
| ABIOTIC_STRESS_OS-ST-1-2 | ABIOTIC STRESS | 285 |
| ABIOTIC_STRESS_OS-ST-11-1 | ABIOTIC STRESS | 131 |
| ABIOTIC_STRESS_OS-ST-11-1 | ABIOTIC STRESS | 197 |
| ABIOTIC_STRESS_OS-ST-11-1 | ABIOTIC STRESS | 197 |
| ABIOTIC_STRESS_OS-ST-11-1 | ABIOTIC STRESS | 131 |
| ABIOTIC_STRESS_OS-ST-11-1 | ABIOTIC STRESS | 131 |
| ABIOTIC_STRESS_OS-ST-12-1 | ABIOTIC STRESS | 131 |
| ABIOTIC_STRESS_OS-ST-12-1 | ABIOTIC STRESS | 197 |
| ABIOTIC_STRESS_OS-ST-12-1 | ABIOTIC STRESS | 197 |
| ABIOTIC_STRESS_OS-ST-12-1 | ABIOTIC STRESS | 131 |
| ABIOTIC_STRESS_OS-ST-12-1 | ABIOTIC STRESS | 131 |
| ABIOTIC_STRESS_OS-ST-4-1 | ABIOTIC STRESS | 237 |
| ABIOTIC_STRESS_OS-ST-4-2 | ABIOTIC STRESS | 237 |
| MATURITY_OS-DTF10-2-1 | MATURITY | 321 |
| MATURITY_OS-DTF10-8-2 | MATURITY | 273 |
| MATURITY_OS-DTF10-8-2 | MATURITY | 273 |
| MATURITY_OS-HT-2-2 | MATURITY | 273 |
| MATURITY_OS-HT-2-2 | MATURITY | 273 |
| MATURITY_OS-HT-8-1 | MATURITY | 273 |
| MATURITY_OS-HT-8-1 | MATURITY | 273 |
| PATHOGEN_RESISTANCE_OS-BP-3-1 | PATHOGEN RESISTANCE | 319 |
| PATHOGEN_RESISTANCE_OS-LS-1-1 | PATHOGEN RESISTANCE | 285 |
| PATHOGEN_RESISTANCE_OS-LS-1-1 | PATHOGEN RESISTANCE | 285 |
| PATHOGEN_RESISTANCE_OS-LS-2-1 | PATHOGEN RESISTANCE | 25 |
| PATHOGEN_RESISTANCE_OS-LS-2-1 | PATHOGEN RESISTANCE | 25 |
| PATHOGEN_RESISTANCE_OS-LS-3-1 | PATHOGEN RESISTANCE | 33 |
| PATHOGEN_RESISTANCE_OS-LS-3-3 | PATHOGEN RESISTANCE | 319 |
| PATHOGEN_RESISTANCE_OS-SB-11-1 | PATHOGEN RESISTANCE | 27 |
| PATHOGEN_RESISTANCE_OS-SB-2-1 | PATHOGEN RESISTANCE | 99 |
| QUALITY_OS-BDV-12-1 | GRAIN_QUALITY | 131 |
| QUALITY_OS-BDV-12-1 | GRAIN_QUALITY | 197 |
| QUALITY_OS-BDV-12-1 | GRAIN_QUALITY | 131 |
| QUALITY_OS-BDV-6-1 | GRAIN_QUALITY | 349 |
| QUALITY_OS-BDV-6-1 | GRAIN_QUALITY | 349 |
| QUALITY_OS-CHALK-6-1 | GRAIN_QUALITY | 349 |
| QUALITY_OS-CHALK-6-1 | GRAIN_QUALITY | 349 |
| QUALITY_OS-CPV-6-1 | GRAIN_QUALITY | 349 |
| QUALITY_OS-CPV-6-1 | GRAIN_QUALITY | 349 |
| QUALITY_OS-CPV-6-2 | GRAIN_QUALITY | 349 |
| QUALITY_OS-CPV-6-2 | GRAIN_QUALITY | 349 |
| QUALITY_OS-CSV-6-1 | GRAIN_QUALITY | 349 |
| QUALITY_OS-CSV-6-1 | GRAIN_QUALITY | 349 |
| QUALITY_OS-CSV-6-2 | GRAIN_QUALITY | 349 |
| QUALITY_OS-CSV-6-2 | GRAIN_QUALITY | 349 |
| QUALITY_OS-HPV-6-1 | GRAIN_QUALITY | 349 |
| QUALITY_OS-HPV-6-1 | GRAIN_QUALITY | 349 |
| QUALITY_OS-HPV-6-2 | GRAIN_QUALITY | 349 |
| QUALITY_OS-HPV-6-2 | GRAIN_QUALITY | 349 |
| QUALITY_OS-PKV-12-1 | GRAIN_QUALITY | 131 |
| QUALITY_OS-PKV-12-1 | GRAIN_QUALITY | 197 |
| QUALITY_OS-PKV-12-1 | GRAIN_QUALITY | 131 |
| QUALITY_OS-PKV-2-1 | GRAIN_QUALITY | 291 |
| QUALITY_OS-PKV-2-1 | GRAIN_QUALITY | 291 |
| QUALITY_OS-PKV-2-1 | GRAIN_QUALITY | 291 |
| QUALITY_OS-PKV-2-1 | GRAIN_QUALITY | 291 |
| QUALITY_OS-PKV-2-1 | GRAIN_QUALITY | 291 |
| QUALITY_OS-SBV-6-1 | GRAIN_QUALITY | 349 |
| QUALITY_OS-SBV-6-1 | GRAIN_QUALITY | 349 |
| QUALITY_OS-WC-6-1 | GRAIN_QUALITY | 349 |
| QUALITY_OS-WC-6-1 | GRAIN_QUALITY | 349 |
| YIELD_OS-DM-6-1 | YIELD | 349 |
| YIELD_OS-GP-6-1 | YIELD | 349 |
| YIELD_OS-GW-11-2 | YIELD | 131 |
| YIELD_OS-Y-6-1 | YIELD | 349 |
| YIELD_OS-YLD-11-1 | YIELD | 131 |

TABLE 4B
References for the listed quantitative trait loci (QTLs)
listed in Table 4A

| OTL | REFERENCE |
|---|---|
| OS-ALTOL-1-1 | THEOR APPL GENET (2000) 100:1295-1303 |
| OS-ALTOL-12-1 | THEOR APPL GENET (2000) 100:1295-1303 |
| OS-BDV-1-1 | THEOR APPL GENET (2000) 100:280-284 |
| OS-BDV-6-1 | THEOR APPL GENET (2000) 100:280-284 |
| OS-BDV-12-1 | THEOR APPL GENET (2000) 100:280-284 |
| OS-CSV-1-1 | THEOR APPL GENET (2000) 100:280-284 |
| OS-CSV-6-1 | THEOR APPL GENET (2000) 100:280-284 |
| OS-CSV-6-2 | THEOR APPL GENET (2000) 100:280-284 |
| OS-CPV-1-1 | THEOR APPL GENET (2000) 100:280-284 |
| OS-CPV-6-1 | THEOR APPL GENET (2000) 100:280-284 |
| OS-CPV-6-2 | THEOR APPL GENET (2000) 100:280-284 |
| OS-DIF10-2-1 | MOLECULAR BREEDING (2000) 6:145-155 |
| OS-DTF10-8-2 | MOLECULAR BREEDING (2000) 6:145-155 |
| OS-CHALK-1-1 | THEOR APPL GENET (2000) 101:823-829 |
| OS-CHALK-6-1 | THEOR APPL GENET (2000) 101:823-829 |
| OS-WC-6-1 | THEOR APPL GENET (2000) 101:823-829 |
| OS-GP-1-1 | THEOR APPL GENET (2000) 101:248-254 |
| OS-GP-6-1 | THEOR APPL GENET (2000) 101:248-254 |
| OS-HPV-6-1 | THEOR APPL GENET (2000) 100:280-284 |
| OS-HPV-6-2 | THEOR APPL GENET (2000) 100:280-284 |
| OS-LFDRY-4-2 | MOLECULAR BREEDING (2000) 6:55-66 |
| OS-LFDRY-5-1 | MOLECULAR BREEDING (2000) 6:55-66 |
| OS-LFDRY-10-1 | MOLECULAR BREEDING (2000) 6:55-66 |
| OS-LFDRY-11-1 | MOLECULAR BREEDING (2000) 6:55-66 |
| OS-LFROL-1-1 | MOLECULAR BREEDING (2000) 6:55-66 |
| OS-LFROL-4-1 | MOLECULAR BREEDING (2000) 6:55-66 |
| OS-LFROL-5-1 | MOLECULAR BREEDING (2000) 6:55-66 |
| OS-PKV-2-1 | THEOR APPL GENET (2000) 100:280-284 |
| OS-PKV-12-1 | THEOR APPL GENET (2000) 100:280-284 |
| OS-RGR-2-1 | MOLECULAR BREEDING (2000) 6:55-66 |
| OS-RGR-5-1 | MOLECULAR BREEDING (2000) 6:55-66 |
| OS-RGR-12-1 | MOLECULAR BREEDING (2000) 6:55-66 |
| OS-RWC-1-2 | MOLECULAR BREEDING (2000) 6:55-66 |
| OS-RWC-6-1 | MOLECULAR BREEDING (2000) 6:55-66 |
| OS-RWC-12-1 | MOLECULAR BREEDING (2000) 6:55-66 |
| OS-SBV-1-1 | THEOR APPL GENET (2000) 100:280-284 |
| OS-SBV-6-1 | THEOR APPL GENET (2000) 100:280-284 |
| OS-ST-1-1 | THEOR APPL GENET (2000) 101:1074-1081 |
| OS-ST-1-2 | THEOR APPL GENET (2000) 101:1074-1081 |
| OS-ST-4-1 | THEOR APPL GENET (2000) 101:1074-1081 |
| OS-ST-4-2 | THEOR APPL GENET (2000) 101:1074-1081 |
| OS-ST-11-1 | THEOR APPL GENET (2000) 101:1074-1081 |
| OS-ST-12-1 | THEOR APPL GENET (2000) 101:1074-1081 |
| OS-LS-1-1 | THEOR APPL GENET (2000) 101:286-291 |
| OS-LS-2-1 | THEOR APPL GENET (2000) 101:286-291 |
| OS-LS-3-1 | THEOR APPL GENET (2000) 101:286-291 |
| OS-LS-3-3 | THEOR APPL GENET (2000) 101:286-291 |
| OS-SB-2-1 | THEOR APPL GENET (2000) 101:569-573 |
| OS-SB-11-1 | THEOR APPL GENET (2000) 101:569-573 |
| OS-Y-6-1 | THEOR APPL GENET (2000) 101:248-254 |
| OS-BP-3-1 | THEOR APPL GENET (2001) 102:929-934 |
| OS-RRL-1-1 | THEOR APPL GENET (2001) 102:1002-1010 |
| OS-RRL-11-1 | THEOR APPL GENET (2001) 102:1002-1010 |
| OS-SRL-1-2 | THEOR APPL GENET (2001) 102:1002-1010 |
| OS-SRL-1-1 | THEOR APPL GENET (2001) 102:1002-1010 |
| OS-SSL-1-2 | THEOR APPL GENET (2001) 102:1002-1010 |
| OS-SSL-10-1 | THEOR APPL GENET (2001) 102:1002-1010 |
| OS-HT-2-2 | THEOR APPL GENET (2001) 102:1236-1242 |
| OS-HT-8-1 | THEOR APPL GENET (2001) 102:1236-1242 |
| OS-GW-1-2 | THEOR APPL GENET (2001) 102:41-52 |
| OS-GW-11-2 | THEOR APPL GENET (2001) 102:41-52 |
| OS-YLD-1-1 | THEOR APPL GENET (2001) 102:41-52 |
| OS-YLD-11-1 | THEOR APPL GENET (2001) 102:41-52 |
| OS-DM-6-1 | PLANT PHYSIOLOGY (2001) 125:406-422 |

TABLE 5A
Sequences associated with the listed maize quantitative trait loci
(QTLs) and showing at least a 4-fold alteration in expression associated
with the listed trait.

| QTL | TRAIT | SEQ ID |
|---|---|---|
| ABIOTIC_STRESS_ZM-DTOL-4-1 | ABIOTIC STRESS | 25 |
| ABIOTIC_STRESS_ZM-DTOL-4-3 | ABIOTIC STRESS | 25 |
| ABIOTIC_STRESS_ZM-DTOL-9-1 | ABIOTIC STRESS | 99 |
| ABIOTIC_STRESS_ZM-DTOL-9-1 | ABIOTIC STRESS | 99 |
| ABIOTIC_STRESS_ZM-DTOL-9-1 | ABIOTIC STRESS | 331 |
| ABIOTIC_STRESS_ZM-LABA-1-1 | ABIOTIC STRESS | 319 |
| ABIOTIC_STRESS_ZM-LABA-1-1 | ABIOTIC STRESS | 319 |
| ABIOTIC_STRESS_ZM-LABA-1-2 | ABIOTIC STRESS | 285 |
| ABIOTIC_STRESS_ZM-LABA-1-2 | ABIOTIC STRESS | 87 |
| ABIOTIC_STRESS_ZM-LABA-10-2 | ABIOTIC STRESS | 87 |
| ABIOTIC_STRESS_ZM-LABA-2-1 | ABIOTIC STRESS | 87 |
| ABIOTIC_STRESS_ZM-LABA-2-3 | ABIOTIC STRESS | 233 |
| ABIOTIC_STRESS_ZM-LABA-2-3 | ABIOTIC STRESS | 285 |
| ABIOTIC_STRESS_ZM-LABA-2-3 | ABIOTIC STRESS | 233 |
| ABIOTIC_STRESS_ZM-LABA-2-3 | ABIOTIC STRESS | 359 |
| ABIOTIC_STRESS_ZM-LABA-2-3 | ABIOTIC STRESS | 233 |
| ABIOTIC_STRESS_ZM-LABA-2-3 | ABIOTIC STRESS | 359 |
| ABIOTIC_STRESS_ZM-LABA-2-3 | ABIOTIC STRESS | 33 |
| ABIOTIC_STRESS_ZM-LABA-2-4 | ABIOTIC STRESS | 359 |
| ABIOTIC_STRESS_ZM-LABA-2-4 | ABIOTIC STRESS | 359 |
| ABIOTIC_STRESS_ZM-LABA-2-4 | ABIOTIC STRESS | 33 |
| ABIOTIC_STRESS_ZM-LABA-3-1 | ABIOTIC STRESS | 285 |
| ABIOTIC_STRESS_ZM-LABA-3-1 | ABIOTIC STRESS | 257 |
| ABIOTIC_STRESS_ZM-LABA-3-1 | ABIOTIC STRESS | 257 |
| ABIOTIC_STRESS_ZM-LABA-4-1 | ABIOTIC STRESS | 25 |
| ABIOTIC_STRESS_ZM-LABA-4-1 | ABIOTIC STRESS | 273 |
| ABIOTIC_STRESS_ZM-LABA-4-1 | ABIOTIC STRESS | 273 |
| ABIOTIC_STRESS_ZM-LABA-4-1 | ABIOTIC STRESS | 57 |
| ABIOTIC_STRESS_ZM-LABA-4-1 | ABIOTIC STRESS | 273 |
| ABIOTIC_STRESS_ZM-LABA-4-1 | ABIOTIC STRESS | 273 |
| ABIOTIC_STRESS_ZM-LABA-4-1 | ABIOTIC STRESS | 33 |
| ABIOTIC_STRESS_ZM-LABA-4-1 | ABIOTIC STRESS | 57 |
| ABIOTIC_STRESS_ZM-LABA-4-2 | ABIOTIC STRESS | 25 |
| ABIOTIC_STRESS_ZM-LABA-4-2 | ABIOTIC STRESS | 273 |
| ABIOTIC_STRESS_ZM-LABA-4-2 | ABIOTIC STRESS | 273 |
| ABIOTIC_STRESS_ZM-LABA-4-2 | ABIOTIC STRESS | 57 |
| ABIOTIC_STRESS_ZM-LABA-4-2 | ABIOTIC STRESS | 273 |
| ABIOTIC_STRESS_ZM-LABA-4-2 | ABIOTIC STRESS | 273 |
| ABIOTIC_STRESS_ZM-LABA-4-2 | ABIOTIC STRESS | 33 |
| ABIOTIC_STRESS_ZM-LABA-4-2 | ABIOTIC STRESS | 57 |
| ABIOTIC_STRESS_ZM-LABA-7-1 | ABIOTIC STRESS | 87 |
| ABIOTIC_STRESS_ZM-LABA-7-2 | ABIOTIC STRESS | 87 |
| ABIOTIC_STRESS_ZM-LABA-9-1 | ABIOTIC STRESS | 257 |
| ABIOTIC_STRESS_ZM-LABA-9-1 | ABIOTIC STRESS | 319 |
| ABIOTIC_STRESS_ZM-LABA-9-1 | ABIOTIC STRESS | 257 |
| ABIOTIC_STRESS_ZM-LABA-9-1 | ABIOTIC STRESS | 319 |
| GRAIN_PERFORMANCE_MAS12-22 | GRAIN_QUALITY | 25 |
| GRAIN_PERFORMANCE_MAS12-22 | GRAIN_QUALITY | 25 |
| GRAIN_PERFORMANCE_MAS12-23 | GRAIN_QUALITY | 131 |
| GRAIN_PERFORMANCE_MAS12-23 | GRAIN_QUALITY | 263 |
| GRAIN_PERFORMANCE_MAS12-23 | GRAIN_QUALITY | 131 |
| GRAIN_PERFORMANCE_MAS12-23 | GRAIN_QUALITY | 263 |
| GRAIN_PERFORMANCE_MAS12-23 | GRAIN_QUALITY | 263 |
| GRAIN_PERFORMANCE_MAS12-23 | GRAIN_QUALITY | 237 |
| GRAIN_PERFORMANCE_MAS12-24 | GRAIN_QUALITY | 263 |
| GRAIN_PERFORMANCE_MAS12-24 | GRAIN_QUALITY | 257 |
| GRAIN_PERFORMANCE_MAS12-24 | GRAIN_QUALITY | 263 |
| GRAIN_PERFORMANCE_MAS12-24 | GRAIN_QUALITY | 263 |
| GRAIN_PERFORMANCE_MAS12-29 | GRAIN_QUALITY | 281 |
| GRAIN_PERFORMANCE_MAS12-32 | GRAIN_QUALITY | 359 |
| GRAIN_PERFORMANCE_MAS12-32 | GRAIN_QUALITY | 233 |
| GRAIN_PERFORMANCE_MAS12-32 | GRAIN_QUALITY | 281 |
| GRAIN_PERFORMANCE_MAS12-32 | GRAIN_QUALITY | 233 |
| GRAIN_PERFORMANCE_MAS12-32 | GRAIN_QUALITY | 33 |
| GRAIN_PERFORMANCE_MAS12-33 | GRAIN_QUALITY | 87 |
| GRAIN_PERFORMANCE_MAS12-34 | GRAIN_QUALITY | 291 |
| GRAIN_PERFORMANCE_MAS12-34 | GRAIN_QUALITY | 273 |
| GRAIN_PERFORMANCE_MAS12-34 | GRAIN_QUALITY | 273 |
| GRAIN_PERFORMANCE_MAS12-34 | GRAIN_QUALITY | 291 |
| GRAIN_PERFORMANCE_MAS12-34 | GRAIN_QUALITY | 273 |
| GRAIN_PERFORMANCE_MAS12-34 | GRAIN_QUALITY | 57 |
| GRAIN_PERFORMANCE_MAS12-34 | GRAIN_QUALITY | 291 |
| GRAIN_PERFORMANCE_MAS12-34 | GRAIN_QUALITY | 273 |
| GRAIN_PERFORMANCE_MAS12-35 | GRAIN_QUALITY | 25 |
| GRAIN_PERFORMANCE_MAS12-35 | GRAIN_QUALITY | 25 |
| GRAIN_PERFORMANCE_MAS12-35 | GRAIN_QUALITY | 321 |
| GRAIN_PERFORMANCE_MAS12-37 | GRAIN_QUALITY | 25 |
| GRAIN_PERFORMANCE_MAS12-37 | GRAIN_QUALITY | 25 |
| GRAIN_PERFORMANCE_MAS12-38 | GRAIN_QUALITY | 281 |
| GRAIN_PERFORMANCE_MAS12-40 | GRAIN_QUALITY | 87 |
| GRAIN_PERFORMANCE_MAS12-40 | GRAIN_QUALITY | 217 |
| GRAIN_PERFORMANCE_MAS12-40 | GRAIN_QUALITY | 33 |
| GRAIN_PERFORMANCE_MAS12-41 | GRAIN_QUALITY | 359 |
| GRAIN_PERFORMANCE_MAS12-41 | GRAIN_QUALITY | 233 |
| GRAIN_PERFORMANCE_MAS12-41 | GRAIN_QUALITY | 281 |
| GRAIN_PERFORMANCE_MAS12-41 | GRAIN_QUALITY | 233 |
| GRAIN_PERFORMANCE_MAS12-41 | GRAIN_QUALITY | 33 |
| GRAIN_PERFORMANCE_MAS12-42 | GRAIN_QUALITY | 57 |
| GRAIN_PERFORMANCE_MAS12-43 | GRAIN_QUALITY | 131 |
| GRAIN_PERFORMANCE_MAS12-43 | GRAIN_QUALITY | 131 |
| GRAIN_PERFORMANCE_MAS12-44 | GRAIN_QUALITY | 321 |
| GRAIN_PERFORMANCE_MAS12-47 | GRAIN_QUALITY | 131 |
| GRAIN_PERFORMANCE_MAS12-47 | GRAIN_QUALITY | 263 |
| GRAIN_PERFORMANCE_MAS12-47 | GRAIN_QUALITY | 131 |
| GRAIN_PERFORMANCE_MAS12-47 | GRAIN_QUALITY | 263 |
| GRAIN_PERFORMANCE_MAS12-47 | GRAIN_QUALITY | 263 |
| GRAIN_PERFORMANCE_MAS12-47 | GRAIN_QUALITY | 237 |
| GRAIN_PERFORMANCE_MAS12-58 | GRAIN_QUALITY | 291 |
| GRAIN_PERFORMANCE_MAS12-58 | GRAIN_QUALITY | 273 |
| GRAIN_PERFORMANCE_MAS12-58 | GRAIN_QUALITY | 25 |
| GRAIN_PERFORMANCE_MAS12-58 | GRAIN_QUALITY | 273 |
| GRAIN_PERFORMANCE_MAS12-58 | GRAIN_QUALITY | 291 |
| GRAIN_PERFORMANCE_MAS12-58 | GRAIN_QUALITY | 273 |
| GRAIN_PERFORMANCE_MAS12-58 | GRAIN_QUALITY | 25 |
| GRAIN_PERFORMANCE_MAS12-58 | GRAIN_QUALITY | 57 |
| GRAIN_PERFORMANCE_MAS12-58 | GRAIN_QUALITY | 291 |
| GRAIN_PERFORMANCE_MAS12-58 | GRAIN_QUALITY | 273 |
| GRAIN_PERFORMANCE_MAS12-58 | GRAIN_QUALITY | 33 |
| GRAIN_PERFORMANCE_MAS13-41 | GRAIN_QUALITY | 319 |
| GRAIN_PERFORMANCE_MAS13-43 | GRAIN_QUALITY | 131 |
| GRAIN_PERFORMANCE_MAS13-43 | GRAIN_QUALITY | 87 |
| GRAIN_PERFORMANCE_MAS13-43 | GRAIN_QUALITY | 331 |
| GRAIN_PERFORMANCE_MAS13-43 | GRAIN_QUALITY | 131 |
| GRAIN_PERFORMANCE_MAS13-43 | GRAIN_QUALITY | 319 |
| GRAIN_PERFORMANCE_MAS13-44 | GRAIN_QUALITY | 319 |
| GRAIN_PERFORMANCE_MAS13-56 | GRAIN_QUALITY | 99 |
| GRAIN_PERFORMANCE_MAS13-56 | GRAIN_QUALITY | 331 |
| GRAIN_PERFORMANCE_MAS16-1 | GRAIN_QUALITY | 319 |
| GRAIN_PERFORMANCE_MAS16-10 | GRAIN_QUALITY | 87 |
| GRAIN_PERFORMANCE_MAS16-10 | GRAIN_QUALITY | 217 |
| GRAIN_PERFORMANCE_MAS16-11 | GRAIN_QUALITY | 281 |
| GRAIN_PERFORMANCE_MAS16-12 | GRAIN_QUALITY | 59 |
| GRAIN_PERFORMANCE_MAS16-13 | GRAIN_QUALITY | 87 |
| GRAIN_PERFORMANCE_MAS16-4 | GRAIN_QUALITY | 87 |
| GRAIN_PERFORMANCE_MAS16-6 | GRAIN_QUALITY | 321 |
| GRAIN_PERFORMANCE_MAS16-8 | GRAIN_QUALITY | 281 |
| GRAIN_PERFORMANCE_MAS19-1 | GRAIN_QUALITY | 319 |
| GRAIN_PERFORMANCE_MAS19-10 | GRAIN_QUALITY | 291 |
| GRAIN_PERFORMANCE_MAS19-10 | GRAIN_QUALITY | 87 |
| GRAIN_PERFORMANCE_MAS19-10 | GRAIN_QUALITY | 291 |
| GRAIN_PERFORMANCE_MAS19-10 | GRAIN_QUALITY | 291 |
| GRAIN_PERFORMANCE_MAS19-13 | GRAIN_QUALITY | 281 |
| GRAIN_PERFORMANCE_MAS19-2 | GRAIN_QUALITY | 87 |
| GRAIN_PERFORMANCE_MAS19-2 | GRAIN_QUALITY | 217 |
| GRAIN_PERFORMANCE_MAS19-5 | GRAIN_QUALITY | 87 |
| GRAIN_PERFORMANCE_MAS19-6 | GRAIN_QUALITY | 87 |
| GRAIN_PERFORMANCE_MAS19-7 | GRAIN_QUALITY | 87 |
| GRAIN_PERFORMANCE_MAS19-7 | GRAIN_QUALITY | 331 |
| GRAIN_PERFORMANCE_MAS19-7 | GRAIN_QUALITY | 57 |
| GRAIN_PERFORMANCE_MAS19-7 | GRAIN_QUALITY | 217 |
| GRAIN_PERFORMANCE_MAS19-8 | GRAIN_QUALITY | 87 |
| GRAIN_PERFORMANCE_MAS19-8 | GRAIN_QUALITY | 331 |
| GRAIN_PERFORMANCE_MAS19-8 | GRAIN_QUALITY | 57 |
| GRAIN_PERFORMANCE_MAS19-8 | GRAIN_QUALITY | 217 |
| GRAIN_PERFORMANCE_MAS19-9 | GRAIN_QUALITY | 99 |
| GRAIN_PERFORMANCE_MAS19-9 | GRAIN_QUALITY | 331 |
| GRAIN_PERFORMANCE_MAS20-1 | GRAIN_QUALITY | 321 |
| GRAIN_PERFORMANCE_MAS20-2 | GRAIN_QUALITY | 359 |
| GRAIN_PERFORMANCE_MAS20-2 | GRAIN_QUALITY | 233 |
| GRAIN_PERFORMANCE_MAS20-2 | GRAIN_QUALITY | 281 |
| GRAIN_PERFORMANCE_MAS20-2 | GRAIN_QUALITY | 233 |
| GRAIN_PERFORMANCE_MAS20-2 | GRAIN_QUALITY | 33 |
| GRAIN_PERFORMANCE_MAS20-24 | GRAIN_QUALITY | 87 |
| GRAIN_PERFORMANCE_MAS20-3 | GRAIN_QUALITY | 57 |
| GRAIN_PERFORMANCE_MAS20-4 | GRAIN_QUALITY | 57 |
| GRAIN_PERFORMANCE_MAS20-5 | GRAIN_QUALITY | 87 |
| GRAIN_PERFORMANCE_MAS20-5 | GRAIN_QUALITY | 217 |
| GRAIN_PERFORMANCE_MAS20-6 | GRAIN_QUALITY | 87 |
| GRAIN_PERFORMANCE_MAS20-6 | GRAIN_QUALITY | 217 |
| GRAIN_PERFORMANCE_MAS20-7 | GRAIN_QUALITY | 131 |
| GRAIN_PERFORMANCE_MAS20-7 | GRAIN_QUALITY | 87 |
| GRAIN_PERFORMANCE_MAS20-7 | GRAIN_QUALITY | 331 |
| GRAIN_PERFORMANCE_MAS20-7 | GRAIN_QUALITY | 131 |
| GRAIN_PERFORMANCE_MAS20-7 | GRAIN_QUALITY | 237 |
| GRAIN_PERFORMANCE_MAS20-7 | GRAIN_QUALITY | 319 |
| GRAIN_PERFORMANCE_MAS20-8 | GRAIN_QUALITY | 319 |
| GRAIN_PERFORMANCE_MAS20-9 | GRAIN_QUALITY | 217 |
| GRAIN_PERFORMANCE_MAS21-1 | GRAIN_QUALITY | 87 |
| GRAIN_PERFORMANCE_MAS21-12 | GRAIN_QUALITY | 87 |
| GRAIN_PERFORMANCE_MAS21-12 | GRAIN_QUALITY | 217 |
| GRAIN_PERFORMANCE_MAS21-2 | GRAIN_QUALITY | 87 |
| GRAIN_PERFORMANCE_MAS21-4 | GRAIN_QUALITY | 87 |
| GRAIN_PERFORMANCE_MAS21-4 | GRAIN_QUALITY | 217 |
| GRAIN_PERFORMANCE_MAS21-5 | GRAIN_QUALITY | 281 |
| GRAIN_PERFORMANCE_MAS21-6 | GRAIN_QUALITY | 291 |
| GRAIN_PERFORMANCE_MAS21-6 | GRAIN_QUALITY | 291 |
| GRAIN_PERFORMANCE_MAS21-6 | GRAIN_QUALITY | 291 |
| GRAIN_PERFORMANCE_MAS21-7 | GRAIN_QUALITY | 291 |
| GRAIN_PERFORMANCE_MAS21-7 | GRAIN_QUALITY | 291 |
| GRAIN_PERFORMANCE_MAS21-7 | GRAIN_QUALITY | 291 |
| GRAIN_PERFORMANCE_MAS21-9 | GRAIN_QUALITY | 25 |
| GRAIN_PERFORMANCE_MAS21-9 | GRAIN_QUALITY | 25 |
| GRAIN_PERFORMANCE_MAS22-1 | GRAIN_QUALITY | 131 |
| GRAIN_PERFORMANCE_MAS22-1 | GRAIN_QUALITY | 263 |
| GRAIN_PERFORMANCE_MAS22-1 | GRAIN_QUALITY | 131 |
| GRAIN_PERFORMANCE_MAS22-1 | GRAIN_QUALITY | 263 |
| GRAIN_PERFORMANCE_MAS22-1 | GRAIN_QUALITY | 263 |
| GRAIN_PERFORMANCE_MAS22-1 | GRAIN_QUALITY | 237 |
| GRAIN_PERFORMANCE_MAS22-17 | GRAIN_QUALITY | 87 |
| GRAIN_PERFORMANCE_MAS22-18 | GRAIN_QUALITY | 87 |
| GRAIN_PERFORMANCE_MAS22-19 | GRAIN_QUALITY | 87 |
| GRAIN_PERFORMANCE_MAS22-2 | GRAIN_QUALITY | 291 |
| GRAIN_PERFORMANCE_MAS22-2 | GRAIN_QUALITY | 273 |
| GRAIN_PERFORMANCE_MAS22-2 | GRAIN_QUALITY | 273 |
| GRAIN_PERFORMANCE_MAS22-2 | GRAIN_QUALITY | 291 |
| GRAIN_PERFORMANCE_MAS22-2 | GRAIN_QUALITY | 273 |
| GRAIN_PERFORMANCE_MAS22-2 | GRAIN_QUALITY | 57 |
| GRAIN_PERFORMANCE_MAS22-2 | GRAIN_QUALITY | 291 |
| GRAIN_PERFORMANCE_MAS22-2 | GRAIN_QUALITY | 273 |
| GRAIN_PERFORMANCE_MAS22-20 | GRAIN_QUALITY | 291 |
| GRAIN_PERFORMANCE_MAS22-20 | GRAIN_QUALITY | 291 |
| GRAIN_PERFORMANCE_MAS22-20 | GRAIN_QUALITY | 291 |
| GRAIN_PERFORMANCE_MAS22-21 | GRAIN_QUALITY | 291 |
| GRAIN_PERFORMANCE_MAS22-21 | GRAIN_QUALITY | 291 |
| GRAIN_PERFORMANCE_MAS22-21 | GRAIN_QUALITY | 291 |
| GRAIN_PERFORMANCE_MAS22-3 | GRAIN_QUALITY | 291 |
| GRAIN_PERFORMANCE_MAS22-3 | GRAIN_QUALITY | 273 |
| GRAIN_PERFORMANCE_MAS22-3 | GRAIN_QUALITY | 273 |
| GRAIN_PERFORMANCE_MAS22-3 | GRAIN_QUALITY | 291 |
| GRAIN_PERFORMANCE_MAS22-3 | GRAIN_QUALITY | 273 |
| GRAIN_PERFORMANCE_MAS22-3 | GRAIN_QUALITY | 57 |
| GRAIN_PERFORMANCE_MAS22-3 | GRAIN_QUALITY | 291 |
| GRAIN_PERFORMANCE_MAS22-3 | GRAIN_QUALITY | 273 |
| GRAIN_PERFORMANCE_MAS22-4 | GRAIN_QUALITY | 291 |
| GRAIN_PERFORMANCE_MAS22-4 | GRAIN_QUALITY | 273 |
| GRAIN_PERFORMANCE_MAS22-4 | GRAIN_QUALITY | 273 |
| GRAIN_PERFORMANCE_MAS22-4 | GRAIN_QUALITY | 291 |
| GRAIN_PERFORMANCE_MAS22-4 | GRAIN_QUALITY | 273 |
| GRAIN_PERFORMANCE_MAS22-4 | GRAIN_QUALITY | 57 |
| GRAIN_PERFORMANCE_MAS22-4 | GRAIN_QUALITY | 291 |
| GRAIN_PERFORMANCE_MAS22-4 | GRAIN_QUALITY | 273 |
| GRAIN_PERFORMANCE_MAS22-5 | GRAIN_QUALITY | 319 |
| GRAIN_PERFORMANCE_MAS22-6 | GRAIN_QUALITY | 319 |
| GRAIN_PERFORMANCE_MAS22-7 | GRAIN_QUALITY | 131 |
| GRAIN_PERFORMANCE_MAS22-7 | GRAIN_QUALITY | 263 |
| GRAIN_PERFORMANCE_MAS22-7 | GRAIN_QUALITY | 131 |
| GRAIN_PERFORMANCE_MAS22-7 | GRAIN_QUALITY | 257 |
| GRAIN_PERFORMANCE_MAS22-7 | GRAIN_QUALITY | 263 |
| GRAIN_PERFORMANCE_MAS22-7 | GRAIN_QUALITY | 321 |
| GRAIN_PERFORMANCE_MAS22-7 | GRAIN_QUALITY | 263 |
| GRAIN_PERFORMANCE_MAS22-7 | GRAIN_QUALITY | 237 |
| GRAIN_PERFORMANCE_MAS22-8 | GRAIN_QUALITY | 131 |
| GRAIN_PERFORMANCE_MAS22-8 | GRAIN_QUALITY | 263 |
| GRAIN_PERFORMANCE_MAS22-8 | GRAIN_QUALITY | 131 |
| GRAIN_PERFORMANCE_MAS22-8 | GRAIN_QUALITY | 257 |
| GRAIN_PERFORMANCE_MAS22-8 | GRAIN_QUALITY | 263 |
| GRAIN_PERFORMANCE_MAS22-8 | GRAIN_QUALITY | 321 |
| GRAIN_PERFORMANCE_MAS22-8 | GRAIN_QUALITY | 263 |
| GRAIN_PERFORMANCE_MAS22-8 | GRAIN_QUALITY | 237 |
| GRAIN_PERFORMANCE_MAS22-9 | GRAIN_QUALITY | 131 |
| GRAIN_PERFORMANCE_MAS22-9 | GRAIN_QUALITY | 131 |
| GRAIN_PERFORMANCE_MAS22-9 | GRAIN_QUALITY | 237 |
| GRAIN_PERFORMANCE_MAS23-11 | GRAIN_QUALITY | 263 |
| GRAIN_PERFORMANCE_MAS23-11 | GRAIN_QUALITY | 331 |
| GRAIN_PERFORMANCE_MAS23-11 | GRAIN_QUALITY | 263 |
| GRAIN_PERFORMANCE_MAS23-11 | GRAIN_QUALITY | 263 |
| GRAIN_PERFORMANCE_MAS23-12 | GRAIN_QUALITY | 319 |
| GRAIN_PERFORMANCE_MAS23-20 | GRAIN_QUALITY | 319 |
| GRAIN_PERFORMANCE_MAS23-21 | GRAIN_QUALITY | 319 |
| GRAIN_PERFORMANCE_MAS23-22 | GRAIN_QUALITY | 319 |
| GRAIN_PERFORMANCE_MAS23-4 | GRAIN_QUALITY | 87 |
| GRAIN_PERFORMANCE_MAS23-4 | GRAIN_QUALITY | 331 |
| GRAIN_PERFORMANCE_MAS23-4 | GRAIN_QUALITY | 57 |
| GRAIN_PERFORMANCE_MAS23-4 | GRAIN_QUALITY | 217 |
| GRAIN_PERFORMANCE_MAS23-5 | GRAIN_QUALITY | 291 |
| GRAIN_PERFORMANCE_MAS23-5 | GRAIN_QUALITY | 291 |
| GRAIN_PERFORMANCE_MAS23-5 | GRAIN_QUALITY | 291 |
| GRAIN_PERFORMANCE_MAS23-6 | GRAIN_QUALITY | 281 |
| GRAIN_PERFORMANCE_MAS23-7 | GRAIN_QUALITY | 87 |
| GRAIN_PERFORMANCE_MAS23-8 | GRAIN_QUALITY | 87 |
| GRAIN_PERFORMANCE_MAS24-23 | GRAIN_QUALITY | 319 |
| GRAIN_PERFORMANCE_MAS24-24 | GRAIN_QUALITY | 319 |
| GRAIN_PERFORMANCE_MAS24-25 | GRAIN_QUALITY | 131 |
| GRAIN_PERFORMANCE_MAS24-25 | GRAIN_QUALITY | 263 |
| GRAIN_PERFORMANCE_MAS24-25 | GRAIN_QUALITY | 131 |
| GRAIN_PERFORMANCE_MAS24-25 | GRAIN_QUALITY | 257 |
| GRAIN_PERFORMANCE_MAS24-25 | GRAIN_QUALITY | 263 |
| GRAIN_PERFORMANCE_MAS24-25 | GRAIN_QUALITY | 263 |
| GRAIN_PERFORMANCE_MAS24-25 | GRAIN_QUALITY | 237 |
| GRAIN_PERFORMANCE_MAS24-26 | GRAIN_QUALITY | 87 |
| GRAIN_PERFORMANCE_MAS25-1 | GRAIN_QUALITY | 263 |
| GRAIN_PERFORMANCE_MAS25-1 | GRAIN_QUALITY | 331 |
| GRAIN_PERFORMANCE_MAS25-1 | GRAIN_QUALITY | 263 |
| GRAIN_PERFORMANCE_MAS25-1 | GRAIN_QUALITY | 263 |
| GRAIN_PERFORMANCE_MAS25-13 | GRAIN_QUALITY | 57 |
| GRAIN_PERFORMANCE_MAS25-14 | GRAIN_QUALITY | 57 |
| GRAIN_PERFORMANCE_MAS25-17 | GRAIN_QUALITY | 87 |
| GRAIN_PERFORMANCE_MAS25-19 | GRAIN_QUALITY | 319 |
| GRAIN_PERFORMANCE_MAS25-2 | GRAIN_QUALITY | 291 |
| GRAIN_PERFORMANCE_MAS25-2 | GRAIN_QUALITY | 291 |
| GRAIN_PERFORMANCE_MAS25-2 | GRAIN_QUALITY | 291 |
| GRAIN_PERFORMANCE_MAS25-20 | GRAIN_QUALITY | 319 |
| GRAIN_PERFORMANCE_MAS25-21 | GRAIN_QUALITY | 281 |
| GRAIN_PERFORMANCE_MAS25-22 | GRAIN_QUALITY | 87 |
| GRAIN_PERFORMANCE_MAS25-23 | GRAIN_QUALITY | 131 |
| GRAIN_PERFORMANCE_MAS25-23 | GRAIN_QUALITY | 25 |
| GRAIN_PERFORMANCE_MAS25-23 | GRAIN_QUALITY | 131 |
| GRAIN_PERFORMANCE_MAS25-23 | GRAIN_QUALITY | 25 |
| GRAIN_PERFORMANCE_MAS25-31 | GRAIN_QUALITY | 131 |
| GRAIN_PERFORMANCE_MAS25-31 | GRAIN_QUALITY | 263 |
| GRAIN_PERFORMANCE_MAS25-31 | GRAIN_QUALITY | 131 |
| GRAIN_PERFORMANCE_MAS25-31 | GRAIN_QUALITY | 257 |
| GRAIN_PERFORMANCE_MAS25-31 | GRAIN_QUALITY | 263 |
| GRAIN_PERFORMANCE_MAS25-31 | GRAIN_QUALITY | 321 |
| GRAIN_PERFORMANCE_MAS25-31 | GRAIN_QUALITY | 263 |
| GRAIN_PERFORMANCE_MAS25-31 | GRAIN_QUALITY | 237 |
| GRAIN_PERFORMANCE_MAS25-34 | GRAIN_QUALITY | 99 |
| GRAIN_PERFORMANCE_MAS25-34 | GRAIN_QUALITY | 331 |
| GRAIN_PERFORMANCE_MAS25-36 | GRAIN_QUALITY | 291 |
| GRAIN_PERFORMANCE_MAS25-36 | GRAIN_QUALITY | 291 |
| GRAIN_PERFORMANCE_MAS25-36 | GRAIN_QUALITY | 291 |
| GRAIN_PERFORMANCE_MAS25-37 | GRAIN_QUALITY | 57 |
| GRAIN_PERFORMANCE_MAS25-39 | GRAIN_QUALITY | 57 |
| GRAIN_PERFORMANCE_MAS25-4 | GRAIN_QUALITY | 25 |
| GRAIN_PERFORMANCE_MAS25-4 | GRAIN_QUALITY | 25 |
| GRAIN_PERFORMANCE_MAS25-43 | GRAIN_QUALITY | 257 |
| GRAIN_PERFORMANCE_MAS25-48 | GRAIN_QUALITY | 319 |
| GRAIN_PERFORMANCE_MAS25-5 | GRAIN_QUALITY | 131 |
| GRAIN_PERFORMANCE_MAS25-5 | GRAIN_QUALITY | 87 |
| GRAIN_PERFORMANCE_MAS25-5 | GRAIN_QUALITY | 331 |
| GRAIN_PERFORMANCE_MAS25-5 | GRAIN_QUALITY | 131 |
| GRAIN_PERFORMANCE_MAS25-5 | GRAIN_QUALITY | 319 |
| GRAIN_PERFORMANCE_MAS25-6 | GRAIN_QUALITY | 87 |
| GRAIN_PERFORMANCE_MAS25-7 | GRAIN_QUALITY | 319 |
| GRAIN_PERFORMANCE_MAS25-9 | GRAIN_QUALITY | 87 |
| GRAIN_PERFORMANCE_MAS27-1 | GRAIN_QUALITY | 57 |
| GRAIN_PERFORMANCE_MAS27-11 | GRAIN_QUALITY | 291 |
| GRAIN_PERFORMANCE_MAS27-11 | GRAIN_QUALITY | 87 |
| GRAIN_PERFORMANCE_MAS27-11 | GRAIN_QUALITY | 291 |
| GRAIN_PERFORMANCE_MAS27-11 | GRAIN_QUALITY | 291 |
| GRAIN_PERFORMANCE_MAS27-12 | GRAIN_QUALITY | 257 |
| GRAIN_PERFORMANCE_MAS27-14 | GRAIN_QUALITY | 87 |
| GRAIN_PERFORMANCE_MAS27-16 | GRAIN_QUALITY | 87 |
| GRAIN_PERFORMANCE_MAS27-19 | GRAIN_QUALITY | 57 |
| GRAIN_PERFORMANCE_MAS27-2 | GRAIN_QUALITY | 87 |
| GRAIN_PERFORMANCE_MAS27-6 | GRAIN_QUALITY | 131 |
| GRAIN_PERFORMANCE_MAS27-6 | GRAIN_QUALITY | 263 |
| GRAIN_PERFORMANCE_MAS27-6 | GRAIN_QUALITY | 131 |
| GRAIN_PERFORMANCE_MAS27-6 | GRAIN_QUALITY | 257 |
| GRAIN_PERFORMANCE_MAS27-6 | GRAIN_QUALITY | 263 |
| GRAIN_PERFORMANCE_MAS27-6 | GRAIN_QUALITY | 263 |
| GRAIN_PERFORMANCE_MAS27-6 | GRAIN_QUALITY | 237 |
| GRAIN_PERFORMANCE_MAS27-7 | GRAIN_QUALITY | 131 |
| GRAIN_PERFORMANCE_MAS27-7 | GRAIN_QUALITY | 87 |
| GRAIN_PERFORMANCE_MAS27-7 | GRAIN_QUALITY | 331 |
| GRAIN_PERFORMANCE_MAS27-7 | GRAIN_QUALITY | 131 |
| GRAIN_PERFORMANCE_MAS27-7 | GRAIN_QUALITY | 319 |
| GRAIN_PERFORMANCE_MAS27-8 | GRAIN_QUALITY | 291 |
| GRAIN_PERFORMANCE_MAS27-8 | GRAIN_QUALITY | 291 |
| GRAIN_PERFORMANCE_MAS27-8 | GRAIN_QUALITY | 291 |
| GRAIN_PERFORMANCE_MAS27-9 | GRAIN_QUALITY | 87 |
| GRAIN_PERFORMANCE_MAS27-9 | GRAIN_QUALITY | 331 |
| GRAIN_PERFORMANCE_MAS27-9 | GRAIN_QUALITY | 57 |
| GRAIN_PERFORMANCE_MAS27-9 | GRAIN_QUALITY | 217 |
| GRAIN_PERFORMANCE_SMS015-1 | GRAIN_QUALITY | 257 |
| GRAIN_PERFORMANCE_SMS015-2 | GRAIN_QUALITY | 57 |
| GRAIN_PERFORMANCE_SMS015-37 | GRAIN_QUALITY | 87 |
| GRAIN_PERFORMANCE_SMS015-37 | GRAIN_QUALITY | 217 |
| GRAIN_PERFORMANCE_SMS015-38 | GRAIN_QUALITY | 263 |
| GRAIN_PERFORMANCE_SMS015-38 | GRAIN_QUALITY | 263 |
| GRAIN_PERFORMANCE_SMS015-38 | GRAIN_QUALITY | 263 |
| GRAIN_PERFORMANCE_SMS015-39 | GRAIN_QUALITY | 281 |
| GRAIN_PERFORMANCE_SMS015-41 | GRAIN_QUALITY | 291 |
| GRAIN_PERFORMANCE_SMS015-41 | GRAIN_QUALITY | 273 |
| GRAIN_PERFORMANCE_SMS015-41 | GRAIN_QUALITY | 25 |
| GRAIN_PERFORMANCE_SMS015-41 | GRAIN_QUALITY | 273 |
| GRAIN_PERFORMANCE_SMS015-41 | GRAIN_QUALITY | 291 |
| GRAIN_PERFORMANCE_SMS015-41 | GRAIN_QUALITY | 273 |
| GRAIN_PERFORMANCE_SMS015-41 | GRAIN_QUALITY | 25 |
| GRAIN_PERFORMANCE_SMS015-41 | GRAIN_QUALITY | 57 |
| GRAIN_PERFORMANCE_SMS015-41 | GRAIN_QUALITY | 291 |
| GRAIN_PERFORMANCE_SMS015-41 | GRAIN_QUALITY | 273 |
| GRAIN_PERFORMANCE_SMS015-41 | GRAIN_QUALITY | 33 |
| GRAIN_PERFORMANCE_SMS015-42 | GRAIN_QUALITY | 263 |
| GRAIN_PERFORMANCE_SMS015-42 | GRAIN_QUALITY | 331 |
| GRAIN_PERFORMANCE_SMS015-42 | GRAIN_QUALITY | 263 |
| GRAIN_PERFORMANCE_SMS015-42 | GRAIN_QUALITY | 263 |
| GRAIN_PERFORMANCE_SMS015-51 | GRAIN_QUALITY | 87 |
| GRAIN_PERFORMANCE_SMS021-21 | GRAIN_QUALITY | 359 |
| GRAIN_PERFORMANCE_SMS021-21 | GRAIN_QUALITY | 233 |
| GRAIN_PERFORMANCE_SMS021-21 | GRAIN_QUALITY | 281 |
| GRAIN_PERFORMANCE_SMS021-21 | GRAIN_QUALITY | 233 |
| GRAIN_PERFORMANCE_SMS021-21 | GRAIN_QUALITY | 33 |
| GRAIN_PERFORMANCE_SMS021-38 | GRAIN_QUALITY | 217 |
| GRAIN_PERFORMANCE_SMS021-39 | GRAIN_QUALITY | 39 |
| GRAIN_PERFORMANCE_SMS021-41 | GRAIN_QUALITY | 131 |
| GRAIN_PERFORMANCE_SMS021-41 | GRAIN_QUALITY | 263 |
| GRAIN_PERFORMANCE_SMS021-41 | GRAIN_QUALITY | 131 |
| GRAIN_PERFORMANCE_SMS021-41 | GRAIN_QUALITY | 263 |
| GRAIN_PERFORMANCE_SMS021-41 | GRAIN_QUALITY | 263 |
| GRAIN_PERFORMANCE_SMS021-41 | GRAIN_QUALITY | 237 |
| GRAIN_PERFORMANCE_SMS021-42 | GRAIN_QUALITY | 87 |
| GRAIN_PERFORMANCE_SMS021-43 | GRAIN_QUALITY | 39 |
| GRAIN_PERFORMANCE_SMS021-44 | GRAIN_QUALITY | 321 |
| GRAIN_PERFORMANCE_SMS021-45 | GRAIN_QUALITY | 131 |
| GRAIN_PERFORMANCE_SMS021-45 | GRAIN_QUALITY | 131 |
| GRAIN_PERFORMANCE_SMS021-45 | GRAIN_QUALITY | 257 |
| GRAIN_PERFORMANCE_SMS021-45 | GRAIN_QUALITY | 319 |
| GRAIN_PERFORMANCE_SMS021-46 | GRAIN_QUALITY | 87 |
| GRAIN_PERFORMANCE_SMS021-46 | GRAIN_QUALITY | 217 |
| GRAIN_PERFORMANCE_SMS021-48 | GRAIN_QUALITY | 87 |
| GRAIN_PERFORMANCE_SMS021-50 | GRAIN_QUALITY | 25 |
| GRAIN_PERFORMANCE_SMS021-50 | GRAIN_QUALITY | 25 |
| GRAIN_PERFORMANCE_SMS021-69 | GRAIN_QUALITY | 359 |
| GRAIN_PERFORMANCE_SMS021-69 | GRAIN_QUALITY | 233 |
| GRAIN_PERFORMANCE_SMS021-69 | GRAIN_QUALITY | 281 |
| GRAIN_PERFORMANCE_SMS021-69 | GRAIN_QUALITY | 233 |
| GRAIN_PERFORMANCE_SMS021-69 | GRAIN_QUALITY | 33 |
| GRAIN_PERFORMANCE_SMS021-70 | GRAIN_QUALITY | 57 |
| GRAIN_PERFORMANCE_SMS021-71 | GRAIN_QUALITY | 25 |
| GRAIN_PERFORMANCE_SMS021-71 | GRAIN_QUALITY | 25 |
| GRAIN_PERFORMANCE_SMS021-72 | GRAIN_QUALITY | 281 |
| GRAIN_PERFORMANCE_SMS021-82 | GRAIN_QUALITY | 319 |
| GRAIN_PERFORMANCE_SMS021-83 | GRAIN_QUALITY | 319 |
| GRAIN_QUALITY_MAS12-18 | GRAIN_QUALITY | 131 |
| GRAIN_QUALITY_MAS12-18 | GRAIN_QUALITY | 263 |
| GRAIN_QUALITY_MAS12-18 | GRAIN_QUALITY | 131 |
| GRAIN_QUALITY_MAS12-18 | GRAIN_QUALITY | 263 |
| GRAIN_QUALITY_MAS12-18 | GRAIN_QUALITY | 263 |
| GRAIN_QUALITY_MAS12-18 | GRAIN_QUALITY | 237 |
| GRAIN_QUALITY_MAS13-22 | GRAIN_QUALITY | 87 |
| GRAIN_QUALITY_MAS13-24 | GRAIN_QUALITY | 257 |
| GRAIN_QUALITY_MAS13-31 | GRAIN_QUALITY | 87 |
| GRAIN_QUALITY_MAS13-32 | GRAIN_QUALITY | 87 |
| GRAIN_QUALITY_MAS13-32 | GRAIN_QUALITY | 217 |
| GRAIN_QUALITY_MAS19-14 | GRAIN_QUALITY | 319 |
| GRAIN_QUALITY_MAS24-16 | GRAIN_QUALITY | 131 |
| GRAIN_QUALITY_MAS24-16 | GRAIN_QUALITY | 263 |
| GRAIN_QUALITY_MAS24-16 | GRAIN_QUALITY | 131 |
| GRAIN_QUALITY_MAS24-16 | GRAIN_QUALITY | 263 |
| GRAIN_QUALITY_MAS24-16 | GRAIN_QUALITY | 263 |
| GRAIN_QUALITY_MAS24-16 | GRAIN_QUALITY | 237 |
| GRAIN_QUALITY_MAS24-2 | GRAIN_QUALITY | 87 |
| GRAIN_QUALITY_MAS24-2 | GRAIN_QUALITY | 331 |
| GRAIN_QUALITY_MAS24-2 | GRAIN_QUALITY | 57 |
| GRAIN_QUALITY_MAS24-2 | GRAIN_QUALITY | 217 |
| GRAIN_QUALITY_MAS24-21 | GRAIN_QUALITY | 25 |
| GRAIN_QUALITY_MAS24-21 | GRAIN_QUALITY | 25 |
| GRAIN_QUALITY_MAS24-21 | GRAIN_QUALITY | 321 |
| GRAIN_QUALITY_MAS24-28 | GRAIN_QUALITY | 291 |
| GRAIN_QUALITY_MAS24-28 | GRAIN_QUALITY | 291 |
| GRAIN_QUALITY_MAS24-28 | GRAIN_QUALITY | 291 |
| GRAIN_QUALITY_MAS24-3 | GRAIN_QUALITY | 217 |
| GRAIN_QUALITY_SMS015-16 | GRAIN_QUALITY | 131 |
| GRAIN_QUALITY_SMS015-16 | GRAIN_QUALITY | 263 |
| GRAIN_QUALITY_SMS015-16 | GRAIN_QUALITY | 131 |
| GRAIN_QUALITY_SMS015-16 | GRAIN_QUALITY | 257 |
| GRAIN_QUALITY_SMS015-16 | GRAIN_QUALITY | 263 |
| GRAIN_QUALITY_SMS015-16 | GRAIN_QUALITY | 321 |
| GRAIN_QUALITY_SMS015-16 | GRAIN_QUALITY | 263 |
| GRAIN_QUALITY_SMS015-16 | GRAIN_QUALITY | 237 |
| GRAIN_QUALITY_SMS015-23 | GRAIN_QUALITY | 87 |
| GRAIN_QUALITY_SMS015-9 | GRAIN_QUALITY | 291 |
| GRAIN_QUALITY_SMS015-9 | GRAIN_QUALITY | 273 |
| GRAIN_QUALITY_SMS015-9 | GRAIN_QUALITY | 273 |
| GRAIN_QUALITY_SMS015-9 | GRAIN_QUALITY | 291 |
| GRAIN_QUALITY_SMS015-9 | GRAIN_QUALITY | 273 |
| GRAIN_QUALITY_SMS015-9 | GRAIN_QUALITY | 57 |
| GRAIN_QUALITY_SMS015-9 | GRAIN_QUALITY | 291 |
| GRAIN_QUALITY_SMS015-9 | GRAIN_QUALITY | 273 |
| GRAIN_QUALITY_SMS021-80 | GRAIN_QUALITY | 291 |
| GRAIN_QUALITY_SMS021-80 | GRAIN_QUALITY | 291 |
| GRAIN_QUALITY_SMS021-80 | GRAIN_QUALITY | 291 |
| GRAIN_QUALITY_SMS021-81 | GRAIN_QUALITY | 263 |
| GRAIN_QUALITY_SMS021-81 | GRAIN_QUALITY | 257 |
| GRAIN_QUALITY_SMS021-81 | GRAIN_QUALITY | 263 |
| GRAIN_QUALITY_SMS021-81 | GRAIN_QUALITY | 263 |
| MATURITY_SMS024-20 | MATURITY | 291 |
| MATURITY_SMS024-51 | MATURITY | 291 |
| MATURITY_ZM-DTA-4-1 | MATURITY | 291 |
| MATURITY_ZM-DTA-4-1 | MATURITY | 273 |
| MATURITY_ZM-SILK-4-1 | MATURITY | 291 |
| MATURITY_ZM-SILK-4-1 | MATURITY | 273 |
| PATHOGEN_RESISTANCE_ZM-ADCNLB-3-1 | PATHOGEN RESISTANCE | 87 |
| PATHOGEN_RESISTANCE_ZM-ADCNLB-3-2 | PATHOGEN RESISTANCE | 87 |
| PATHOGEN_RESISTANCE_ZM-ADCNLB-5-1 | PATHOGEN RESISTANCE | 25 |
| PATHOGEN_RESISTANCE_ZM-ADCNLB-5-2 | PATHOGEN RESISTANCE | 25 |
| PATHOGEN_RESISTANCE_ZM-ADCNLB-9-1 | PATHOGEN RESISTANCE | 309 |
| PATHOGEN_RESISTANCE_ZM-ADCNLB-9-1 | PATHOGEN RESISTANCE | 257 |
| PATHOGEN_RESISTANCE_ZM-ADCNLB-9-1 | PATHOGEN RESISTANCE | 319 |
| PATHOGEN_RESISTANCE_ZM-ANMT-1-1 | PATHOGEN RESISTANCE | 257 |
| PATHOGEN_RESISTANCE_ZM-ANMT-1-2 | PATHOGEN RESISTANCE | 257 |
| PATHOGEN_RESISTANCE_ZM-ANMT-10-1 | PATHOGEN RESISTANCE | 87 |
| PATHOGEN_RESISTANCE_ZM-ANMT-10-2 | PATHOGEN RESISTANCE | 87 |
| PATHOGEN_RESISTANCE_ZM-ANMT-2-1 | PATHOGEN RESISTANCE | 87 |
| PATHOGEN_RESISTANCE_ZM-ANMT-5-1 | PATHOGEN RESISTANCE | 263 |
| PATHOGEN_RESISTANCE_ZM-ANMT-5-1 | PATHOGEN RESISTANCE | 263 |
| PATHOGEN_RESISTANCE_ZM-APIT-1-1 | PATHOGEN RESISTANCE | 257 |
| PATHOGEN_RESISTANCE_ZM-APIT-1-2 | PATHOGEN RESISTANCE | 257 |
| PATHOGEN_RESISTANCE_ZM-APIT-10-1 | PATHOGEN RESISTANCE | 87 |
| PATHOGEN_RESISTANCE_ZM-APIT-10-2 | PATHOGEN RESISTANCE | 87 |
| PATHOGEN_RESISTANCE_ZM-APIT-2-1 | PATHOGEN RESISTANCE | 359 |
| PATHOGEN_RESISTANCE_ZM-APIT-2-1 | PATHOGEN RESISTANCE | 33 |
| PATHOGEN_RESISTANCE_ZM-APIT-3-1 | PATHOGEN RESISTANCE | 349 |
| PATHOGEN_RESISTANCE_ZM-APIT-3-1 | PATHOGEN RESISTANCE | 57 |
| PATHOGEN_RESISTANCE_ZM-APIT-3-2 | PATHOGEN RESISTANCE | 257 |
| PATHOGEN_RESISTANCE_ZM-APIT-6-1 | PATHOGEN RESISTANCE | 257 |
| PATHOGEN_RESISTANCE_ZM-APIT-6-1 | PATHOGEN RESISTANCE | 263 |
| PATHOGEN_RESISTANCE_ZM-APIT-6-1 | PATHOGEN RESISTANCE | 349 |
| PATHOGEN_RESISTANCE_ZM-APIT-6-1 | PATHOGEN RESISTANCE | 263 |
| PATHOGEN_RESISTANCE_ZM-APIT-6-1 | PATHOGEN RESISTANCE | 237 |
| PATHOGEN_RESISTANCE_ZM-APIT-9-1 | PATHOGEN RESISTANCE | 87 |
| PATHOGEN_RESISTANCE_ZM-AUT-1-1 | PATHOGEN RESISTANCE | 257 |
| PATHOGEN_RESISTANCE_ZM-AUT-1-2 | PATHOGEN RESISTANCE | 257 |
| PATHOGEN_RESISTANCE_ZM-AUT-10-1 | PATHOGEN RESISTANCE | 87 |
| PATHOGEN_RESISTANCE_ZM-AUT-10-2 | PATHOGEN RESISTANCE | 87 |
| PATHOGEN_RESISTANCE_ZM-AUT-3-1 | PATHOGEN RESISTANCE | 257 |
| PATHOGEN_RESISTANCE_ZM-AUT-6-1 | PATHOGEN RESISTANCE | 257 |
| PATHOGEN_RESISTANCE_ZM-AUT-6-1 | PATHOGEN RESISTANCE | 263 |
| PATHOGEN_RESISTANCE_ZM-AUT-6-1 | PATHOGEN RESISTANCE | 349 |
| PATHOGEN_RESISTANCE_ZM-AUT-6-1 | PATHOGEN RESISTANCE | 263 |
| PATHOGEN_RESISTANCE_ZM-AUT-6-1 | PATHOGEN RESISTANCE | 237 |
| PATHOGEN_RESISTANCE_ZM-CELW-5-1 | PATHOGEN RESISTANCE | 291 |
| PATHOGEN_RESISTANCE_ZM-CELW-6-1 | PATHOGEN RESISTANCE | 87 |
| PATHOGEN_RESISTANCE_ZM-CELW-6-1 | PATHOGEN RESISTANCE | 349 |
| PATHOGEN_RESISTANCE_ZM-CELW-6-1 | PATHOGEN RESISTANCE | 319 |
| PATHOGEN_RESISTANCE_ZM-CRR-1-10 | PATHOGEN RESISTANCE | 87 |
| PATHOGEN_RESISTANCE_ZM-CRR-1-11 | PATHOGEN RESISTANCE | 257 |
| PATHOGEN_RESISTANCE_ZM-CRR-1-12 | PATHOGEN RESISTANCE | 87 |
| PATHOGEN_RESISTANCE_ZM-CRR-1-13 | PATHOGEN RESISTANCE | 257 |
| PATHOGEN_RESISTANCE_ZM-CRR-1-14 | PATHOGEN RESISTANCE | 71 |
| PATHOGEN_RESISTANCE_ZM-CRR-1-14 | PATHOGEN RESISTANCE | 319 |
| PATHOGEN_RESISTANCE_ZM-CRR-1-15 | PATHOGEN RESISTANCE | 257 |
| PATHOGEN_RESISTANCE_ZM-CRR-2-11 | PATHOGEN RESISTANCE | 359 |
| PATHOGEN_RESISTANCE_ZM-CRR-2-11 | PATHOGEN RESISTANCE | 33 |
| PATHOGEN_RESISTANCE_ZM-CRR-3-6 | PATHOGEN RESISTANCE | 349 |
| PATHOGEN_RESISTANCE_ZM-CRR-3-6 | PATHOGEN RESISTANCE | 57 |
| PATHOGEN_RESISTANCE_ZM-CRR-3-7 | PATHOGEN RESISTANCE | 87 |
| PATHOGEN_RESISTANCE_ZM-CRR-3-8 | PATHOGEN RESISTANCE | 87 |
| PATHOGEN_RESISTANCE_ZM-CRR-3-9 | PATHOGEN RESISTANCE | 87 |
| PATHOGEN_RESISTANCE_ZM-CRR-3-9 | PATHOGEN RESISTANCE | 257 |
| PATHOGEN_RESISTANCE_ZM-CRR-4-3 | PATHOGEN RESISTANCE | 59 |
| PATHOGEN_RESISTANCE_ZM-CRR-4-3 | PATHOGEN RESISTANCE | 25 |
| PATHOGEN_RESISTANCE_ZM-CRR-4-4 | PATHOGEN RESISTANCE | 291 |
| PATHOGEN_RESISTANCE_ZM-CRR-4-4 | PATHOGEN RESISTANCE | 71 |
| PATHOGEN_RESISTANCE_ZM-CRR-4-4 | PATHOGEN RESISTANCE | 59 |
| PATHOGEN_RESISTANCE_ZM-CRR-4-4 | PATHOGEN RESISTANCE | 273 |
| PATHOGEN_RESISTANCE_ZM-CRR-4-4 | PATHOGEN RESISTANCE | 273 |
| PATHOGEN_RESISTANCE_ZM-CRR-4-4 | PATHOGEN RESISTANCE | 25 |
| PATHOGEN_RESISTANCE_ZM-CRR-4-4 | PATHOGEN RESISTANCE | 57 |
| PATHOGEN_RESISTANCE_ZM-CRR-4-4 | PATHOGEN RESISTANCE | 33 |
| PATHOGEN_RESISTANCE_ZM-CRR-4-5 | PATHOGEN RESISTANCE | 99 |
| PATHOGEN_RESISTANCE_ZM-CRR-4-5 | PATHOGEN RESISTANCE | 25 |
| PATHOGEN_RESISTANCE_ZM-CRR-4-6 | PATHOGEN RESISTANCE | 291 |
| PATHOGEN_RESISTANCE_ZM-CRR-4-6 | PATHOGEN RESISTANCE | 71 |
| PATHOGEN_RESISTANCE_ZM-CRR-4-6 | PATHOGEN RESISTANCE | 59 |
| PATHOGEN_RESISTANCE_ZM-CRR-4-6 | PATHOGEN RESISTANCE | 273 |
| PATHOGEN_RESISTANCE_ZM-CRR-4-6 | PATHOGEN RESISTANCE | 273 |
| PATHOGEN_RESISTANCE_ZM-CRR-4-6 | PATHOGEN RESISTANCE | 25 |
| PATHOGEN_RESISTANCE_ZM-CRR-4-6 | PATHOGEN RESISTANCE | 57 |
| PATHOGEN_RESISTANCE_ZM-CRR-4-6 | PATHOGEN RESISTANCE | 33 |
| PATHOGEN_RESISTANCE_ZM-CRR-5-6 | PATHOGEN RESISTANCE | 263 |
| PATHOGEN_RESISTANCE_ZM-CRR-5-6 | PATHOGEN RESISTANCE | 263 |
| PATHOGEN_RESISTANCE_ZM-CRR-5-7 | PATHOGEN RESISTANCE | 291 |
| PATHOGEN_RESISTANCE_ZM-CRR-5-7 | PATHOGEN RESISTANCE | 25 |
| PATHOGEN_RESISTANCE_ZM-CRR-5-9 | PATHOGEN RESISTANCE | 291 |
| PATHOGEN_RESISTANCE_ZM-CRR-6-4 | PATHOGEN RESISTANCE | 257 |
| PATHOGEN_RESISTANCE_ZM-CRR-6-4 | PATHOGEN RESISTANCE | 263 |
| PATHOGEN_RESISTANCE_ZM-CRR-6-4 | PATHOGEN RESISTANCE | 263 |
| PATHOGEN_RESISTANCE_ZM-CRR-6-5 | PATHOGEN RESISTANCE | 263 |
| PATHOGEN_RESISTANCE_ZM-CRR-6-5 | PATHOGEN RESISTANCE | 349 |
| PATHOGEN_RESISTANCE_ZM-CRR-6-5 | PATHOGEN RESISTANCE | 263 |
| PATHOGEN_RESISTANCE_ZM-CRR-6-5 | PATHOGEN RESISTANCE | 237 |
| PATHOGEN_RESISTANCE_ZM-CRR-6-6 | PATHOGEN RESISTANCE | 349 |
| PATHOGEN_RESISTANCE_ZM-CRR-7-5 | PATHOGEN RESISTANCE | 87 |
| PATHOGEN_RESISTANCE_ZM-CRR-8-6 | PATHOGEN RESISTANCE | 71 |
| PATHOGEN_RESISTANCE_ZM-CRR-8-6 | PATHOGEN RESISTANCE | 263 |
| PATHOGEN_RESISTANCE_ZM-CRR-8-6 | PATHOGEN RESISTANCE | 349 |
| PATHOGEN_RESISTANCE_ZM-CRR-8-6 | PATHOGEN RESISTANCE | 263 |
| PATHOGEN_RESISTANCE_ZM-CRR-8-7 | PATHOGEN RESISTANCE | 71 |
| PATHOGEN_RESISTANCE_ZM-CRR-8-7 | PATHOGEN RESISTANCE | 263 |
| PATHOGEN_RESISTANCE_ZM-CRR-8-7 | PATHOGEN RESISTANCE | 349 |
| PATHOGEN_RESISTANCE_ZM-CRR-8-7 | PATHOGEN RESISTANCE | 263 |
| PATHOGEN_RESISTANCE_ZM-CRR-9-2 | PATHOGEN RESISTANCE | 309 |
| PATHOGEN_RESISTANCE_ZM-CRR-9-2 | PATHOGEN RESISTANCE | 257 |
| PATHOGEN_RESISTANCE_ZM-CRR-9-2 | PATHOGEN RESISTANCE | 319 |
| PATHOGEN_RESISTANCE_ZM-CRR-9-3 | PATHOGEN RESISTANCE | 309 |
| PATHOGEN_RESISTANCE_ZM-CRR-9-3 | PATHOGEN RESISTANCE | 257 |
| PATHOGEN_RESISTANCE_ZM-CRR-9-3 | PATHOGEN RESISTANCE | 319 |
| PATHOGEN_RESISTANCE_ZM-CRR-9-4 | PATHOGEN RESISTANCE | 87 |
| PATHOGEN_RESISTANCE_ZM-CRR-9-5 | PATHOGEN RESISTANCE | 99 |
| PATHOGEN_RESISTANCE_ZM-CRR-9-5 | PATHOGEN RESISTANCE | 309 |
| PATHOGEN_RESISTANCE_ZM-CRR-9-5 | PATHOGEN RESISTANCE | 257 |
| PATHOGEN_RESISTANCE_ZM-CRR-9-5 | PATHOGEN RESISTANCE | 319 |
| PATHOGEN_RESISTANCE_ZM-CSVEG-1-1 | PATHOGEN RESISTANCE | 87 |
| PATHOGEN_RESISTANCE_ZM-CSVEG-4-1 | PATHOGEN RESISTANCE | 291 |
| PATHOGEN_RESISTANCE_ZM-CSVEG-4-1 | PATHOGEN RESISTANCE | 71 |
| PATHOGEN_RESISTANCE_ZM-CSVEG-4-1 | PATHOGEN RESISTANCE | 59 |
| PATHOGEN_RESISTANCE_ZM-CSVEG-4-1 | PATHOGEN RESISTANCE | 273 |
| PATHOGEN_RESISTANCE_ZM-CSVEG-4-1 | PATHOGEN RESISTANCE | 273 |
| PATHOGEN_RESISTANCE_ZM-CSVEG-4-1 | PATHOGEN RESISTANCE | 25 |
| PATHOGEN_RESISTANCE_ZM-CSVEG-4-1 | PATHOGEN RESISTANCE | 57 |
| PATHOGEN_RESISTANCE_ZM-CSVEG-4-1 | PATHOGEN RESISTANCE | 33 |
| PATHOGEN_RESISTANCE_ZM-CSVEG-4-2 | PATHOGEN RESISTANCE | 99 |
| PATHOGEN_RESISTANCE_ZM-CSVEG-9-1 | PATHOGEN RESISTANCE | 309 |
| PATHOGEN_RESISTANCE_ZM-CSVEG-9-1 | PATHOGEN RESISTANCE | 257 |
| PATHOGEN_RESISTANCE_ZM-CSVEG-9-1 | PATHOGEN RESISTANCE | 319 |
| PATHOGEN_RESISTANCE_ZM-CSVSG-1-1 | PATHOGEN RESISTANCE | 319 |
| PATHOGEN_RESISTANCE_ZM-CSVSG-4-1 | PATHOGEN RESISTANCE | 99 |
| PATHOGEN_RESISTANCE_ZM-DMS-1-1 | PATHOGEN RESISTANCE | 319 |
| PATHOGEN_RESISTANCE_ZM-DMS-1-2 | PATHOGEN RESISTANCE | 257 |
| PATHOGEN_RESISTANCE_ZM-DMS-9-1 | PATHOGEN RESISTANCE | 87 |
| PATHOGEN_RESISTANCE_ZM-ETURI-2-1 | PATHOGEN RESISTANCE | 291 |
| PATHOGEN_RESISTANCE_ZM-ETURI-2-1 | PATHOGEN RESISTANCE | 87 |
| PATHOGEN_RESISTANCE_ZM-ETURI-5-1 | PATHOGEN RESISTANCE | 25 |
| PATHOGEN_RESISTANCE_ZM-HMAYI-3-1 | PATHOGEN RESISTANCE | 349 |
| PATHOGEN_RESISTANCE_ZM-HMAYI-3-1 | PATHOGEN RESISTANCE | 57 |
| PATHOGEN_RESISTANCE_ZM-HPVI-3-1 | PATHOGEN RESISTANCE | 349 |
| PATHOGEN_RESISTANCE_ZM-HPVI-3-1 | PATHOGEN RESISTANCE | 57 |
| PATHOGEN_RESISTANCE_ZM-HPVI-6-1 | PATHOGEN RESISTANCE | 87 |
| PATHOGEN_RESISTANCE_ZM-HPVI-6-1 | PATHOGEN RESISTANCE | 349 |
| PATHOGEN_RESISTANCE_ZM-HPVI-6-1 | PATHOGEN RESISTANCE | 319 |
| PATHOGEN_RESISTANCE_ZM-LT-1-1 | PATHOGEN RESISTANCE | 257 |
| PATHOGEN_RESISTANCE_ZM-LT-1-2 | PATHOGEN RESISTANCE | 87 |
| PATHOGEN_RESISTANCE_ZM-LT-4-1 | PATHOGEN RESISTANCE | 59 |
| PATHOGEN_RESISTANCE_ZM-LT-5-1 | PATHOGEN RESISTANCE | 25 |
| PATHOGEN_RESISTANCE_ZM-LVI-4-1 | PATHOGEN RESISTANCE | 25 |
| PATHOGEN_RESISTANCE_ZM-LVI-5-1 | PATHOGEN RESISTANCE | 25 |
| PATHOGEN_RESISTANCE_ZM-LVI-6-1 | PATHOGEN RESISTANCE | 87 |
| PATHOGEN_RESISTANCE_ZM-LVI-6-1 | PATHOGEN RESISTANCE | 349 |
| PATHOGEN_RESISTANCE_ZM-LVI-6-1 | PATHOGEN RESISTANCE | 319 |
| PATHOGEN_RESISTANCE_ZM-MSVI-1-1 | PATHOGEN RESISTANCE | 257 |
| PATHOGEN_RESISTANCE_ZM-MSVI-1-2 | PATHOGEN RESISTANCE | 87 |
| PATHOGEN_RESISTANCE_ZM-MSVI-1-2 | PATHOGEN RESISTANCE | 33 |
| PATHOGEN_RESISTANCE_ZM-MSVI-10-1 | PATHOGEN RESISTANCE | 87 |
| PATHOGEN_RESISTANCE_ZM-MSVI-2-1 | PATHOGEN RESISTANCE | 87 |
| PATHOGEN_RESISTANCE_ZM-MSVI-3-1 | PATHOGEN RESISTANCE | 257 |
| PATHOGEN_RESISTANCE_ZM-MSVI-3-2 | PATHOGEN RESISTANCE | 349 |
| PATHOGEN_RESISTANCE_ZM-MSVI-3-2 | PATHOGEN RESISTANCE | 57 |
| PATHOGEN_RESISTANCE_ZM-MSVI-9-1 | PATHOGEN RESISTANCE | 87 |
| PATHOGEN_RESISTANCE_ZM-NLBDS-1-1 | PATHOGEN RESISTANCE | 257 |
| PATHOGEN_RESISTANCE_ZM-NLBDS-3-2 | PATHOGEN RESISTANCE | 87 |
| PATHOGEN_RESISTANCE_ZM-NLBDS-3-5 | PATHOGEN RESISTANCE | 87 |
| PATHOGEN_RESISTANCE_ZM-NLBDS-4-1 | PATHOGEN RESISTANCE | 59 |
| PATHOGEN_RESISTANCE_ZM-NLBDS-4-2 | PATHOGEN RESISTANCE | 291 |
| PATHOGEN_RESISTANCE_ZM-NLBDS-4-2 | PATHOGEN RESISTANCE | 71 |
| PATHOGEN_RESISTANCE_ZM-NLBDS-4-2 | PATHOGEN RESISTANCE | 59 |
| PATHOGEN_RESISTANCE_ZM-NLBDS-4-2 | PATHOGEN RESISTANCE | 273 |
| PATHOGEN_RESISTANCE_ZM-NLBDS-4-2 | PATHOGEN RESISTANCE | 273 |
| PATHOGEN_RESISTANCE_ZM-NLBDS-4-2 | PATHOGEN RESISTANCE | 25 |
| PATHOGEN_RESISTANCE_ZM-NLBDS-4-2 | PATHOGEN RESISTANCE | 57 |
| PATHOGEN_RESISTANCE_ZM-NLBDS-4-2 | PATHOGEN RESISTANCE | 33 |
| PATHOGEN_RESISTANCE_ZM-NLBDS-5-1 | PATHOGEN RESISTANCE | 25 |
| PATHOGEN_RESISTANCE_ZM-NLBDS-5-2 | PATHOGEN RESISTANCE | 25 |
| PATHOGEN_RESISTANCE_ZM-NLBDS-5-5 | PATHOGEN RESISTANCE | 291 |
| PATHOGEN_RESISTANCE_ZM-NLBDS-6-1 | PATHOGEN RESISTANCE | 257 |
| PATHOGEN_RESISTANCE_ZM-NLBDS-6-1 | PATHOGEN RESISTANCE | 263 |
| PATHOGEN_RESISTANCE_ZM-NLBDS-6-1 | PATHOGEN RESISTANCE | 349 |
| PATHOGEN_RESISTANCE_ZM-NLBDS-6-1 | PATHOGEN RESISTANCE | 263 |
| PATHOGEN_RESISTANCE_ZM-NLBDS-6-1 | PATHOGEN RESISTANCE | 237 |
| PATHOGEN_RESISTANCE_ZM-NLBDS-8-1 | PATHOGEN RESISTANCE | 263 |
| PATHOGEN_RESISTANCE_ZM-NLBDS-8-1 | PATHOGEN RESISTANCE | 263 |
| PATHOGEN_RESISTANCE_ZM-NLBDS-8-2 | PATHOGEN RESISTANCE | 71 |
| PATHOGEN_RESISTANCE_ZM-NLBDS-8-2 | PATHOGEN RESISTANCE | 349 |
| PATHOGEN_RESISTANCE_ZM-NLBDS-9-1 | PATHOGEN RESISTANCE | 309 |
| PATHOGEN_RESISTANCE_ZM-NLBDS-9-1 | PATHOGEN RESISTANCE | 257 |
| PATHOGEN_RESISTANCE_ZM-NLBDS-9-1 | PATHOGEN RESISTANCE | 319 |
| PATHOGEN_RESISTANCE_ZM-NLBDS-9-2 | PATHOGEN RESISTANCE | 87 |
| PATHOGEN_RESISTANCE_ZM-NLBDS-9-2 | PATHOGEN RESISTANCE | 57 |
| PATHOGEN_RESISTANCE_ZM-NLBDS-9-2 | PATHOGEN RESISTANCE | 319 |
| PATHOGEN_RESISTANCE_ZM-NLBIP-3-1 | PATHOGEN RESISTANCE | 87 |
| PATHOGEN_RESISTANCE_ZM-NLBIP-4-1 | PATHOGEN RESISTANCE | 291 |
| PATHOGEN_RESISTANCE_ZM-NLBIP-4-1 | PATHOGEN RESISTANCE | 71 |
| PATHOGEN_RESISTANCE_ZM-NLBIP-4-1 | PATHOGEN RESISTANCE | 273 |
| PATHOGEN_RESISTANCE_ZM-NLBIP-4-1 | PATHOGEN RESISTANCE | 273 |
| PATHOGEN_RESISTANCE_ZM-NLBIP-4-1 | PATHOGEN RESISTANCE | 25 |
| PATHOGEN_RESISTANCE_ZM-NLBIP-4-1 | PATHOGEN RESISTANCE | 57 |
| PATHOGEN_RESISTANCE_ZM-NLBIP-4-1 | PATHOGEN RESISTANCE | 33 |
| PATHOGEN_RESISTANCE_ZM-NLBIP-5-1 | PATHOGEN RESISTANCE | 25 |
| PATHOGEN_RESISTANCE_ZM-NLBIP-8-1 | PATHOGEN RESISTANCE | 263 |
| PATHOGEN_RESISTANCE_ZM-NLBIP-8-1 | PATHOGEN RESISTANCE | 263 |
| PATHOGEN_RESISTANCE_ZM-NLBIP-9-1 | PATHOGEN RESISTANCE | 309 |
| PATHOGEN_RESISTANCE_ZM-NLBIP-9-1 | PATHOGEN RESISTANCE | 257 |
| PATHOGEN_RESISTANCE_ZM-NLBIP-9-1 | PATHOGEN RESISTANCE | 319 |
| PATHOGEN_RESISTANCE_ZM-PCSI-1-1 | PATHOGEN RESISTANCE | 71 |
| PATHOGEN_RESISTANCE_ZM-PCSI-1-1 | PATHOGEN RESISTANCE | 319 |
| PATHOGEN_RESISTANCE_ZM-PCSI-1-2 | PATHOGEN RESISTANCE | 257 |
| PATHOGEN_RESISTANCE_ZM-PCSI-1-4 | PATHOGEN RESISTANCE | 87 |
| PATHOGEN_RESISTANCE_ZM-PCSI-1-5 | PATHOGEN RESISTANCE | 319 |
| PATHOGEN_RESISTANCE_ZM-PCSI-1-6 | PATHOGEN RESISTANCE | 87 |
| PATHOGEN_RESISTANCE_ZM-PCSI-1-7 | PATHOGEN RESISTANCE | 257 |
| PATHOGEN_RESISTANCE_ZM-PCSI-2-1 | PATHOGEN RESISTANCE | 291 |
| PATHOGEN_RESISTANCE_ZM-PCSI-2-1 | PATHOGEN RESISTANCE | 87 |
| PATHOGEN_RESISTANCE_ZM-PCSI-2-4 | PATHOGEN RESISTANCE | 233 |
| PATHOGEN_RESISTANCE_ZM-PCSI-2-4 | PATHOGEN RESISTANCE | 359 |
| PATHOGEN_RESISTANCE_ZM-PCSI-2-4 | PATHOGEN RESISTANCE | 233 |
| PATHOGEN_RESISTANCE_ZM-PCSI-2-4 | PATHOGEN RESISTANCE | 233 |
| PATHOGEN_RESISTANCE_ZM-PCSI-2-4 | PATHOGEN RESISTANCE | 33 |
| PATHOGEN_RESISTANCE_ZM-PCSI-2-5 | PATHOGEN RESISTANCE | 291 |
| PATHOGEN_RESISTANCE_ZM-PCSI-2-5 | PATHOGEN RESISTANCE | 87 |
| PATHOGEN_RESISTANCE_ZM-PCSI-3-1 | PATHOGEN RESISTANCE | 349 |
| PATHOGEN_RESISTANCE_ZM-PCSI-3-1 | PATHOGEN RESISTANCE | 57 |
| PATHOGEN_RESISTANCE_ZM-PCSI-3-2 | PATHOGEN RESISTANCE | 87 |
| PATHOGEN_RESISTANCE_ZM-PCSI-3-4 | PATHOGEN RESISTANCE | 87 |
| PATHOGEN_RESISTANCE_ZM-PCSI-3-4 | PATHOGEN RESISTANCE | 257 |
| PATHOGEN_RESISTANCE_ZM-PCSI-3-5 | PATHOGEN RESISTANCE | 87 |
| PATHOGEN_RESISTANCE_ZM-PCSI-3-6 | PATHOGEN RESISTANCE | 349 |
| PATHOGEN_RESISTANCE_ZM-PCSI-3-6 | PATHOGEN RESISTANCE | 57 |
| PATHOGEN_RESISTANCE_ZM-PCSI-4-1 | PATHOGEN RESISTANCE | 59 |
| PATHOGEN_RESISTANCE_ZM-PCSI-4-2 | PATHOGEN RESISTANCE | 291 |
| PATHOGEN_RESISTANCE_ZM-PCSI-4-2 | PATHOGEN RESISTANCE | 71 |
| PATHOGEN_RESISTANCE_ZM-PCSI-4-2 | PATHOGEN RESISTANCE | 59 |
| PATHOGEN_RESISTANCE_ZM-PCSI-4-2 | PATHOGEN RESISTANCE | 273 |
| PATHOGEN_RESISTANCE_ZM-PCSI-4-2 | PATHOGEN RESISTANCE | 273 |
| PATHOGEN_RESISTANCE_ZM-PCSI-4-2 | PATHOGEN RESISTANCE | 25 |
| PATHOGEN_RESISTANCE_ZM-PCSI-4-2 | PATHOGEN RESISTANCE | 57 |
| PATHOGEN_RESISTANCE_ZM-PCSI-4-2 | PATHOGEN RESISTANCE | 33 |
| PATHOGEN_RESISTANCE_ZM-PCSI-4-3 | PATHOGEN RESISTANCE | 99 |
| PATHOGEN_RESISTANCE_ZM-PCSI-4-5 | PATHOGEN RESISTANCE | 99 |
| PATHOGEN_RESISTANCE_ZM-PCSI-4-6 | PATHOGEN RESISTANCE | 25 |
| PATHOGEN_RESISTANCE_ZM-PCSI-5-1 | PATHOGEN RESISTANCE | 263 |
| PATHOGEN_RESISTANCE_ZM-PCSI-5-1 | PATHOGEN RESISTANCE | 263 |
| PATHOGEN_RESISTANCE_ZM-PCSI-5-2 | PATHOGEN RESISTANCE | 263 |
| PATHOGEN_RESISTANCE_ZM-PCSI-5-2 | PATHOGEN RESISTANCE | 263 |
| PATHOGEN_RESISTANCE_ZM-PCSI-5-3 | PATHOGEN RESISTANCE | 263 |
| PATHOGEN_RESISTANCE_ZM-PCSI-5-3 | PATHOGEN RESISTANCE | 263 |
| PATHOGEN_RESISTANCE_ZM-PCSI-5-5 | PATHOGEN RESISTANCE | 263 |
| PATHOGEN_RESISTANCE_ZM-PCSI-5-5 | PATHOGEN RESISTANCE | 263 |
| PATHOGEN_RESISTANCE_ZM-PCSI-5-7 | PATHOGEN RESISTANCE | 25 |
| PATHOGEN_RESISTANCE_ZM-PCSI-6-1 | PATHOGEN ERSISTANCE | 257 |
| PATHOGEN_RESISTANCE_ZM-PCSI-6-1 | PATHOGEN RESISTANCE | 263 |
| PATHOGEN_RESISTANCE_ZM-PCSI-6-1 | PATHOGEN RESISTANCE | 263 |
| PATHOGEN_RESISTANCE_ZM-PCSI-9-1 | PATHOGEN RESISTANCE | 87 |
| PATHOGEN_RESISTANCE_ZM-PCSI-9-2 | PATHOGEN RESISTANCE | 309 |
| PATHOGEN_RESISTANCE_ZM-PCSI-9-2 | PATHOGEN RESISTANCE | 257 |
| PATHOGEN_RESISTANCE_ZM-PCSI-9-2 | PATHOGEN RESISTANCE | 319 |
| PATHOGEN_RESISTANCE_ZM-PCSI-9-3 | PATHOGEN RESISTANCE | 309 |
| PATHOGEN_RESISTANCE_ZM-PCSI-9-3 | PATHOGEN RESISTANCE | 257 |
| PATHOGEN_RESISTANCE_ZM-PCSI-9-3 | PATHOGEN RESISTANCE | 319 |
| PATHOGEN_RESISTANCE_ZM-PHSI-4-1 | PATHOGEN RESISTANCE | 25 |
| PATHOGEN_RESISTANCE_ZM-PHSI-5-1 | PATHOGEN RESISTANCE | 291 |
| PATHOGEN_RESISTANCE_ZM-PHSI-6-1 | PATHOGEN RESISTANCE | 257 |
| PATHOGEN_RESISTANCE_ZM-PHSI-6-1 | PATHOGEN RESISTANCE | 263 |
| PATHOGEN_RESISTANCE_ZM-PHSI-6-1 | PATHOGEN RESISTANCE | 263 |
| PATHOGEN_RESISTANCE_ZM-PHSI-6-2 | PATHOGEN RESISTANCE | 87 |
| PATHOGEN_RESISTANCE_ZM-PHSI-6-2 | PATHOGEN RESISTANCE | 349 |
| PATHOGEN_RESISTANCE_ZM-PHSI-6-2 | PATHOGEN RESISTANCE | 319 |
| PATHOGEN_RESISTANCE_ZM-PHSI-6-3 | PATHOGEN RESISTANCE | 263 |
| PATHOGEN_RESISTANCE_ZM-PHSI-6-3 | PATHOGEN RESISTANCE | 263 |
| PATHOGEN_RESISTANCE_ZM-PHSI-8-1 | PATHOGEN RESISTANCE | 71 |
| PATHOGEN_RESISTANCE_ZM-PHSI-8-1 | PATHOGEN RESISTANCE | 349 |
| PATHOGEN_RESISTANCE_ZM-PHSI-9-1 | PATHOGEN RESISTANCE | 319 |
| PATHOGEN_RESISTANCE_ZM-SCBR-1-1 | PATHOGEN RESISTANCE | 319 |
| PATHOGEN_RESISTANCE_ZM-SCBR-1-2 | PATHOGEN RESISTANCE | 257 |
| PATHOGEN_RESISTANCE_ZM-SCBR-5-1 | PATHOGEN RESISTANCE | 25 |
| PATHOGEN_RESISTANCE_ZM-SCBR-8-1 | PATHOGEN RESISTANCE | 263 |
| PATHOGEN_RESISTANCE_ZM-SCBR-8-1 | PATHOGEN RESISTANCE | 263 |
| PATHOGEN_RESISTANCE_ZM-SCBR-9-1 | PATHOGEN RESISTANCE | 319 |
| PATHOGEN_RESISTANCE_ZM-SWCBR-1-1 | PATHOGEN RESISTANCE | 257 |
| PATHOGEN_RESISTANCE_ZM-SWCBR-1-2 | PATHOGEN RESISTANCE | 71 |
| PATHOGEN_RESISTANCE_ZM-SWCBR-1-2 | PATHOGEN RESISTANCE | 319 |
| PATHOGEN_RESISTANCE_ZM-SWCBR-1-3 | PATHOGEN RESISTANCE | 319 |
| PATHOGEN_RESISTANCE_ZM-SWCBR-1-4 | PATHOGEN RESISTANCE | 257 |
| PATHOGEN_RESISTANCE_ZM-SWCBR-1-5 | PATHOGEN RESISTANCE | 71 |
| PATHOGEN_RESISTANCE_ZM-SWCBR-1-6 | PATHOGEN RESISTANCE | 319 |
| PATHOGEN_RESISTANCE_ZM-SWCBR-3-1 | PATHOGEN RESISTANCE | 257 |
| PATHOGEN_RESISTANCE_ZM-SWCBR-3-2 | PATHOGEN RESISTANCE | 257 |
| PATHOGEN_RESISTANCE_ZM-SWCBR-5-1 | PATHOGEN RESISTANCE | 291 |
| PATHOGEN_RESISTANCE_ZM-SWCBR-5-1 | PATHOGEN RESISTANCE | 25 |
| PATHOGEN_RESISTANCE_ZM-SWCBR-5-2 | PATHOGEN RESISTANCE | 25 |
| PATHOGEN_RESISTANCE_ZM-SWCBR-5-3 | PATHOGEN RESISTANCE | 25 |
| PATHOGEN_RESISTANCE_ZM-SWCBR-6-1 | PATHOGEN RESISTANCE | 87 |
| PATHOGEN_RESISTANCE_ZM-SWCBR-6-1 | PATHOGEN RESISTANCE | 349 |
| PATHOGEN_RESISTANCE_ZM-SWCBR-6-1 | PATHOGEN RESISTANCE | 319 |
| PATHOGEN_RESISTANCE_ZM-SWCBR-6-2 | PATHOGEN RESISTANCE | 263 |
| PATHOGEN_RESISTANCE_ZM-SWCBR-6-2 | PATHOGEN RESISTANCE | 349 |
| PATHOGEN_RESISTANCE_ZM-SWCBR-6-2 | PATHOGEN RESISTANCE | 263 |
| PATHOGEN_RESISTANCE_ZM-SWCBR-6-2 | PATHOGEN RESISTANCE | 237 |
| PATHOGEN_RESISTANCE_ZM-SWCBR-6-3 | PATHOGEN RESISTANCE | 263 |
| PATHOGEN_RESISTANCE_ZM-SWCBR-6-3 | PATHOGEN RESISTANCE | 263 |
| PATHOGEN_RESISTANCE_ZM-SWCBR-8-1 | PATHOGEN RESISTANCE | 71 |
| PATHOGEN_RESISTANCE_ZM-SWCBR-8-1 | PATHOGEN RESISTANCE | 349 |
| PATHOGEN_RESISTANCE_ZM-SWCBR-8-2 | PATHOGEN RESISTANCE | 263 |
| PATHOGEN_RESISTANCE_ZM-SWCBR-8-2 | PATHOGEN RESISTANCE | 263 |
| PATHOGEN_RESISTANCE_ZM-SWCBR-9-1 | PATHOGEN RESISTANCE | 309 |
| PATHOGEN_RESISTANCE_ZM-SWCBR-9-1 | PATHOGEN RESISTANCE | 257 |
| PATHOGEN_RESISTANCE_ZM-SWCBR-9-1 | PATHOGEN RESISTANCE | 319 |
| PATHOGEN_RESISTANCE_ZM-SWCBR-9-2 | PATHOGEN RESISTANCE | 87 |
| PATHOGEN_RESISTANCE_ZM-SWCBR-9-2 | PATHOGEN RESISTANCE | 57 |
| PATHOGEN_RESISTANCE_ZM-SWCBR-9-2 | PATHOGEN RESISTANCE | 319 |
| PATHOGEN_RESISTANCE_ZM-SWCBR-9-3 | PATHOGEN RESISTANCE | 309 |
| PATHOGEN_RESISTANCE_ZM-SWCBR-9-3 | PATHOGEN RESISTANCE | 257 |
| PATHOGEN_RESISTANCE_ZM-SWCBR-9-3 | PATHOGEN RESISTANCE | 319 |
| PATHOGEN_RESISTANCE_ZM-SWCBR-9-4 | PATHOGEN RESISTANCE | 309 |
| PATHOGEN_RESISTANCE_ZM-SWCBR-9-4 | PATHOGEN RESISTANCE | 257 |
| PATHOGEN_RESISTANCE_ZM-SWCBR-9-4 | PATHOGEN RESISTANCE | 319 |
| QUALITY_ZM-CPC-1-2 | GRAIN_QUALITY | 319 |
| QUALITY_ZM-CPC-1-3 | GRAIN_QUALITY | 257 |
| QUALITY_ZM-CPC-1-4 | GRAIN_QUALITY | 87 |
| QUALITY_ZM-CPC-1-4 | GRAIN_QUALITY | 217 |
| QUALITY_ZM-CPC-1-5 | GRAIN_QUALITY | 257 |
| QUALITY_ZM-CPC-1-6 | GRAIN_QUALITY | 87 |
| QUALITY_ZM-CPC-1-6 | GRAIN_QUALITY | 217 |
| QUALITY_ZM-CPC-10-1 | GRAIN_QUALITY | 217 |
| QUALITY_ZM-CPC-3-1 | GRAIN_QUALITY | 57 |
| QUALITY_ZM-CPC-3-2 | GRAIN_QUALITY | 87 |
| QUALITY_ZM-CPC-3-3 | GRAIN_QUALITY | 57 |
| QUALITY_ZM-CPC-6-1 | GRAIN_QUALITY | 131 |
| QUALITY_ZM-CPC-6-1 | GRAIN_QUALITY | 131 |
| QUALITY_ZM-CPC-6-2 | GRAIN_QUALITY | 131 |
| QUALITY_ZM-CPC-6-2 | GRAIN_QUALITY | 87 |
| QUALITY_ZM-CPC-6-2 | GRAIN_QUALITY | 331 |
| QUALITY_ZM-CPC-6-2 | GRAIN_QUALITY | 131 |
| QUALITY_ZM-CPC-6-2 | GRAIN_QUALITY | 319 |
| QUALITY_ZM-CPC-7-1 | GRAIN_QUALITY | 281 |
| QUALITY_ZM-CPC-7-2 | GRAIN_QUALITY | 87 |
| QUALITY_ZM-CPC-7-3 | GRAIN_QUALITY | 281 |
| QUALITY_ZM-IVDOM-1-1 | GRAIN_QUALITY | 319 |
| QUALITY_ZM-IVDOM-1-2 | GRAIN_QUALITY | 257 |
| QUALITY_ZM-IVDOM-1-4 | GRAIN_QUALITY | 257 |
| QUALITY_ZM-IVDOM-10-1 | GRAIN_QUALITY | 217 |
| QUALITY_ZM-IVDOM-10-2 | GRAIN_QUALITY | 217 |
| QUALITY_ZM-IVDOM-3-1 | GRAIN_QUALITY | 57 |
| QUALITY_ZM-IVDOM-3-3 | GRAIN_QUALITY | 57 |
| QUALITY_ZM-IVDOM-5-2 | GRAIN_QUALITY | 291 |
| QUALITY_ZM-IVDOM-5-2 | GRAIN_QUALITY | 291 |
| QUALITY_ZM-IVDOM-5-2 | GRAIN_QUALITY | 291 |
| QUALITY_ZM-IVDOM-9-1 | GRAIN_QUALITY | 131 |
| QUALITY_ZM-IVDOM-9-1 | GRAIN_QUALITY | 131 |
| QUALITY_ZM-IVDOM-9-1 | GRAIN_QUALITY | 257 |
| QUALITY_ZM-IVDOM-9-1 | GRAIN_QUALITY | 319 |
| QUALITY_ZM-IVDOM-9-2 | GRAIN_QUALITY | 131 |
| QUALITY_ZM-IVDOM-9-2 | GRAIN_QUALITY | 131 |
| QUALITY_ZM-IVDOM-9-2 | GRAIN_QUALITY | 257 |
| QUALITY_ZM-IVDOM-9-2 | GRAIN_QUALITY | 319 |
| QUALITY_ZM-PC-1-1 | GRAIN_QUALITY | 257 |
| QUALITY_ZM-PC-5-1 | GRAIN_QUALITY | 25 |
| QUALITY_ZM-PC-5-1 | GRAIN_QUALITY | 25 |
| QUALITY_ZM-PC-8-1 | GRAIN_QUALITY | 131 |
| QUALITY_ZM-PC-8-1 | GRAIN_QUALITY | 263 |
| QUALITY_ZM-PC-8-1 | GRAIN_QUALITY | 131 |
| QUALITY_ZM-PC-8-1 | GRAIN_QUALITY | 257 |
| QUALITY_ZM-PC-8-1 | GRAIN_QUALITY | 263 |
| QUALITY_ZM-PC-8-1 | GRAIN_QUALITY | 321 |
| QUALITY_ZM-PC-8-1 | GRAIN_QUALITY | 263 |
| QUALITY_ZM-PC-8-1 | GRAIN_QUALITY | 237 |
| QUALITY_ZM-PC-9-1 | GRAIN_QUALITY | 87 |
| QUALITY_ZM-PC-9-1 | GRAIN_QUALITY | 331 |
| QUALITY_ZM-PC-9-1 | GRAIN_QUALITY | 57 |
| QUALITY_ZM-PC-9-1 | GRAIN_QUALITY | 217 |
| QUALITY_ZM-PC-9-1 | GRAIN_QUALITY | 319 |
| QUALITY_ZM-STC-10-2 | GRAIN_QUALITY | 217 |
| QUALITY_ZM-STC-2-2 | GRAIN_QUALITY | 359 |
| QUALITY_ZM-STC-2-2 | GRAIN_QUALITY | 217 |
| QUALITY_ZM-STC-2-2 | GRAIN_QUALITY | 33 |
| QUALITY_ZM-STC-5-1 | GRAIN_QUALITY | 25 |
| QUALITY_ZM-STC-5-1 | GRAIN_QUALITY | 25 |
| QUALITY_ZM-STC-6-1 | GRAIN_QUALITY | 131 |
| QUALITY_ZM-STC-6-1 | GRAIN_QUALITY | 263 |
| QUALITY_ZM-STC-6-1 | GRAIN_QUALITY | 131 |
| QUALITY_ZM-STC-6-1 | GRAIN_QUALITY | 263 |
| QUALITY_ZM-STC-6-1 | GRAIN_QUALITY | 263 |
| QUALITY_ZM-STC-6-1 | GRAIN_QUALITY | 237 |
| QUALITY_ZM-STC-7-1 | GRAIN_QUALITY | 39 |
| QUALITY_ZM-STC-7-2 | GRAIN_QUALITY | 281 |
| QUALITY_ZM-STC-8-1 | GRAIN_QUALITY | 131 |
| QUALITY_ZM-STC-8-1 | GRAIN_QUALITY | 263 |
| QUALITY_ZM-STC-8-1 | GRAIN_QUALITY | 131 |
| QUALITY_ZM-STC-8-1 | GRAIN_QUALITY | 257 |
| QUALITY_ZM-STC-8-1 | GRAIN_QUALITY | 263 |
| QUALITY_ZM-STC-8-1 | GRAIN_QUALITY | 321 |
| QUALITY_ZM-STC-8-1 | GRAIN_QUALITY | 263 |
| QUALITY_ZM-STC-8-1 | GRAIN_QUALITY | 237 |
| YIELD_ZM-BIOM-3-1 | YIELD | 257 |
| YIELD_ZM-BIOM-8-1 | YIELD | 263 |
| YIELD_ZM-BIOM-8-1 | YIELD | 263 |
| YIELD_ZM-BIOM-8-1 | YIELD | 263 |
| YIELD_ZM-DMC-1-1 | YIELD | 257 |
| YIELD_ZM-DMC-5-1 | YIELD | 25 |
| YIELD_ZM-DMC-6-1 | YIELD | 319 |
| YIELD_ZM-DMC-6-2 | YIELD | 319 |
| YIELD_ZM-DMY-1-4 | YIELD | 257 |
| YIELD_ZM-DMY-2-3 | YIELD | 359 |
| YIELD_ZM-DMY-2-3 | YIELD | 233 |
| YIELD_ZM-DMY-2-3 | YIELD | 359 |
| YIELD_ZM-DMY-2-3 | YIELD | 233 |
| YIELD_ZM-DMY-2-3 | YIELD | 233 |
| YIELD_ZM-DMY-2-3 | YIELD | 233 |
| YIELD_ZM-DMY-2-3 | YIELD | 33 |
| YIELD_ZM-DMY-2-4 | YIELD | 359 |
| YIELD_ZM-DMY-2-4 | YIELD | 359 |
| YIELD_ZM-DMY-2-4 | YIELD | 33 |
| YIELD_ZM-DMY-3-2 | YIELD | 57 |
| YIELD_ZM-DMY-3-3 | YIELD | 57 |
| YIELD_ZM-DMY-4-2 | YIELD | 25 |
| YIELD_ZM-DMY-4-3 | YIELD | 291 |
| YIELD_ZM-DMY-4-3 | YIELD | 273 |
| YIELD_ZM-DMY-4-3 | YIELD | 71 |
| YIELD_ZM-DMY-4-3 | YIELD | 273 |
| YIELD_ZM-DMY-4-3 | YIELD | 291 |
| YIELD_ZM-DMY-4-3 | YIELD | 273 |
| YIELD_ZM-DMY-4-3 | YIELD | 25 |
| YIELD_ZM-DMY-4-3 | YIELD | 57 |
| YIELD_ZM-DMY-4-3 | YIELD | 33 |
| YIELD_ZM-DMY-4-4 | YIELD | 25 |
| YIELD_ZM-DMY-5-1 | YIELD | 291 |
| YIELD_ZM-DMY-5-1 | YIELD | 291 |
| YIELD_ZM-DMY-8-1 | YIELD | 71 |
| YIELD_ZM-DMY-8-1 | YIELD | 305 |
| YIELD_ZM-DMY-8-1 | YIELD | 263 |
| YIELD_ZM-DMY-8-1 | YIELD | 257 |
| YIELD_ZM-DMY-8-1 | YIELD | 263 |
| YIELD_ZM-DMY-8-1 | YIELD | 321 |
| YIELD_ZM-DMY-8-1 | YIELD | 263 |
| YIELD_ZM-DMY-8-2 | YIELD | 263 |
| YIELD_ZM-DMY-8-2 | YIELD | 263 |
| YIELD_ZM-DMY-8-2 | YIELD | 263 |
| YIELD_ZM-DMY-9-1 | YIELD | 257 |
| YIELD_ZM-DMY-9-1 | YIELD | 319 |
| YIELD_ZM-EWT-4-2 | YIELD | 25 |
| YIELD_ZM-GWM2-3-1 | YIELD | 57 |
| YIELD_ZM-GWM2-3-2 | YIELD | 257 |
| YIELD_ZM-GYHA-1-2 | YIELD | 71 |
| YIELD_ZM-GYHA-1-3 | YIELD | 319 |
| YIELD_ZM-GYHA-1-4 | YIELD | 319 |
| YIELD_ZM-GYHA-5-1 | YIELD | 263 |
| YIELD_ZM-GYHA-5-1 | YIELD | 263 |
| YIELD_ZM-GYHA-5-1 | YIELD | 263 |
| YIELD_ZM-GYHA-6-1 | YIELD | 263 |
| YIELD_ZM-GYHA-6-1 | YIELD | 263 |
| YIELD_ZM-GYHA-6-1 | YIELD | 263 |
| YIELD_ZM-GYLD-6-1 | YIELD | 319 |
| YIELD_ZM-HI-1-1 | YIELD | 71 |
| YIELD_ZM-HI-1-1 | YIELD | 319 |
| YIELD_ZM-HI-3-1 | YIELD | 57 |
| YIELD_ZM-HI-4-1 | YIELD | 305 |
| YIELD_ZM-HI-4-1 | YIELD | 25 |
| YIELD_ZM-HI-4-1 | YIELD | 321 |
| YIELD_ZM-HI-8-1 | YIELD | 263 |
| YIELD_ZM-HI-8-1 | YIELD | 263 |
| YIELD_ZM-HI-8-1 | YIELD | 263 |
| YIELD_ZM-KNE-4-1 | YIELD | 25 |
| YIELD_ZM-KW100-1-2 | YIELD | 319 |
| YIELD_ZM-KW100-9-1 | YIELD | 99 |
| YIELD_ZM-KW100-9-1 | YIELD | 257 |
| YIELD_ZM-KW100-9-1 | YIELD | 319 |
| YIELD_ZM-KW300-1-2 | YIELD | 257 |
| YIELD_ZM-KW300-3-2 | YIELD | 57 |
| YIELD_ZM-KW300-4-2 | YIELD | 25 |
| YIELD_ZM-KW300-6-2 | YIELD | 263 |
| YIELD_ZM-KW300-6-2 | YIELD | 263 |
| YIELD_ZM-KW300-6-2 | YIELD | 263 |
| YIELD_ZM-KW300-8-1 | YIELD | 71 |
| YIELD_ZM-KW300-8-1 | YIELD | 305 |
| YIELD_ZM-KW300-8-1 | YIELD | 321 |
| YIELD_ZM-KW300-9-1 | YIELD | 57 |
| YIELD_ZM-KW300-9-2 | YIELD | 99 |
| YIELD_ZM-KW300-9-2 | YIELD | 57 |
| YIELD_ZM-KWE-4-1 | YIELD | 25 |

TABLE 5B
References for the listed quantitative
trait loci (QTLs) listed in Table 5A

| QTL | REFERENCE |
|---|---|
| ZM-LABA-1-1 | THEOR APPL GENET (1998) 97: 744-755 |
| ZM-LABA-1-2 | THEOR APPL GENET (1998) 97: 744-755 |
| ZM-LABA-2-4 | THEOR APPL GENET (1998) 97: 744-755 |
| ZM-LABA-2-3 | THEOR APPL GENET (1998) 97: 744-755 |
| ZM-LABA-2-1 | THEOR APPL GENET (1998) 97: 744-755 |
| ZM-LABA-3-1 | THEOR APPL GENET (1998) 97: 744-755 |
| ZM-LABA-4-2 | THEOR APPL GENET (1998) 97: 744-755 |
| ZM-LABA-4-1 | THEOR APPL GENET (1998) 97: 744-755 |
| ZM-LABA-7-1 | THEOR APPL GENET (1998) 97: 744-755 |
| ZM-LABA-7-2 | THEOR APPL GENET (1998) 97: 744-755 |
| ZM-LABA-9-1 | THEOR APPL GENET (1998) 97: 744-755 |
| ZM-LABA-10-2 | THEOR APPL GENET (1998) 97: 744-755 |
| ZM-CELW-5-1 | GENETICS (1998) 149: 1997-2006 |
| ZM-CELW-6-1 | GENETICS (1998) 149: 1997-2006 |
| ZM-CRR-1-10 | PHYTOPATHOLOGY (1998) 88: 1324-1329 |
| ZM-CRR-6-4 | PHYTOPATHOLOGY (1998) 88: 1324-1329 |
| ZM-CRR-9-2 | PHYTOPATHOLOGY (1998) 88: 1324-1329 |
| ZM-CRR-1-12 | PHYTOPATHOLOGY (1998) 88: 1324-1329 |
| ZM-CRR-1-11 | PHYTOPATHOLOGY (1998) 88: 1324-1329 |
| ZM-CRR-4-3 | PHYTOPATHOLOGY (1998) 88: 1324-1329 |
| ZM-CRR-7-5 | PHYTOPATHOLOGY (1998) 88: 1324-1329 |
| ZM-CRR-8-6 | PHYTOPATHOLOGY (1998) 88: 1324-1329 |
| ZM-CRR-9-3 | PHYTOPATHOLOGY (1998) 88: 1324-1329 |
| ZM-CRR-1-13 | PHYTOPATHOLOGY (1998) 88: 1324-1329 |
| ZM-CRR-2-11 | PHYTOPATHOLOGY (1998) 88: 1324-1329 |
| ZM-CRR-3-7 | PHYTOPATHOLOGY (1998) 88: 1324-1329 |
| ZM-CRR-3-6 | PHYTOPATHOLOGY (1998) 88: 1324-1329 |
| ZM-CRR-4-5 | PHYTOPATHOLOGY (1998) 88: 1324-1329 |
| ZM-CRR-4-4 | PHYTOPATHOLOGY (1998) 88: 1324-1329 |
| ZM-CRR-5-6 | PHYTOPATHOLOGY (1998) 88: 1324-1329 |
| ZM-CRR-5-7 | PHYTOPATHOLOGY (1998) 88: 1324-1329 |
| ZM-CRR-6-5 | PHYTOPATHOLOGY (1998) 88: 1324-1329 |
| ZM-CRR-8-7 | PHYTOPATHOLOGY (1998) 88: 1324-1329 |
| ZM-CRR-9-4 | PHYTOPATHOLOGY (1998) 88: 1324-1329 |
| ZM-PCSI-1-1 | THEOR APPL GENET (1998) 97: 1321-1330 |
| ZM-PCSI-2-1 | THEOR APPL GENET (1998) 97: 1321-1330 |
| ZM-PCSI-3-1 | THEOR APPL GENET (1998) 97: 1321-1330 |
| ZM-PCSI-3-2 | THEOR APPL GENET (1998) 97: 1321-1330 |
| ZM-PCSI-4-1 | THEOR APPL GENET (1998) 97: 1321-1330 |
| ZM-PCSI-5-1 | THEOR APPL GENET (1998) 97: 1321-1330 |
| ZM-PCSI-9-2 | THEOR APPL GENET (1998) 97: 1321-1330 |
| ZM-PCSI-9-1 | THEOR APPL GENET (1998) 97: 1321-1330 |
| ZM-PCSI-1-2 | THEOR APPL GENET (1998) 97: 1321-1330 |
| ZM-PCSI-5-2 | THEOR APPL GENET (1998) 97: 1321-1330 |
| ZM-PCSI-6-1 | THEOR APPL GENET (1998) 97: 1321-1330 |
| ZM-PCSI-1-4 | THEOR APPL GENET (1998) 97: 1321-1330 |
| ZM-PCSI-2-4 | THEOR APPL GENET (1998) 97: 1321-1330 |
| ZM-PCSI-3-4 | THEOR APPL GENET (1998) 97: 1321-1330 |
| ZM-PCSI-5-3 | THEOR APPL GENET (1998) 97: 1321-1330 |
| ZM-PCSI-1-5 | THEOR APPL GENET (1998) 97: 1321-1330 |
| ZM-PCSI-1-6 | THEOR APPL GENET (1998) 97: 1321-1330 |
| ZM-PCSI-2-5 | THEOR APPL GENET (1998) 97: 1321-1330 |
| ZM-PCSI-3-5 | THEOR APPL GENET (1998) 97: 1321-1330 |
| ZM-PCSI-4-2 | THEOR APPL GENET (1998) 97: 1321-1330 |
| ZM-PCSI-4-3 | THEOR APPL GENET (1998) 97: 1321-1330 |
| ZM-PCSI-4-5 | THEOR APPL GENET (1998) 97: 1321-1330 |
| ZM-PCSI-5-5 | THEOR APPL GENET (1998) 97: 1321-1330 |
| ZM-PCSI-9-3 | THEOR APPL GENET (1998) 97: 1321-1330 |
| ZM-CSVEG-1-1 | THEOR APPL GENET (1998) 97: 1321-1330 |
| ZM-CSVEG-4-1 | THEOR APPL GENET (1998) 97: 1321-1330 |
| ZM-CSVEG-4-2 | THEOR APPL GENET (1998) 97: 1321-1330 |
| ZM-CSVEG-9-1 | THEOR APPL GENET (1998) 97: 1321-1330 |
| ZM-CSVSG-1-1 | THEOR APPL GENET (1998) 97: 1321-1330 |
| ZM-CSVSG-4-1 | THEOR APPL GENET (1998) 97: 1321-1330 |
| ZM-LT-1-1 | CROP SCI (1998) 38: 1062-1072 |
| ZM-LT-1-2 | CROP SCI (1998) 38: 1062-1072 |
| ZM-LT-4-1 | CROP SCI (1998) 38: 1062-1072 |
| ZM-PC-1-1 | CROP SCI (1998) 38: 1062-1072 |
| ZM-PC-5-1 | CROP SCI (1998) 38: 1062-1072 |
| ZM-PC-8-1 | CROP SCI (1998) 38: 1062-1072 |
| ZM-PC-9-1 | CROP SCI (1998) 38: 1062-1072 |
| ZM-SWCBR-1-4 | CROP SCI (1998) 38: 1062-1072 |
| ZM-SWCBR-1-3 | CROP SCI (1998) 38: 1062-1072 |
| ZM-SWCBR-1-2 | CROP SCI (1998) 38: 1062-1072 |
| ZM-SWCBR-1-5 | CROP SCI (1998) 38: 1062-1072 |
| ZM-SWCBR-5-3 | CROP SCI (1998) 38: 1062-1072 |
| ZM-SWCBR-8-2 | CROP SCI (1998) 38: 1062-1072 |
| ZM-SWCBR-9-2 | CROP SCI (1998) 38: 1062-1072 |
| ZM-SCBR-1-2 | CROP SCI (1998) 38: 1062-1072 |
| ZM-SCBR-1-1 | CROP SCI (1998) 38: 1062-1072 |
| ZM-SCBR-5-1 | CROP SCI (1998) 38: 1062-1072 |
| ZM-SCBR-8-1 | CROP SCI (1998) 38: 1062-1072 |
| ZM-SCBR-9-1 | CROP SCI (1998) 38: 1062-1072 |
| ZM-LT-5-1 | CROP SCI (1998) 38: 1062-1072 |
| ZM-SWCBR-1-6 | CROP SCI (1998) 38: 1062-1072 |
| ZM-SWCBR-6-3 | CROP SCI (1998) 38: 1062-1072 |
| ZM-SWCBR-9-3 | CROP SCI (1998) 38: 1062-1072 |
| ZM-SWCBR-9-4 | CROP SCI (1998) 38: 1062-1072 |
| ZM-ASI-1-1 | PLANT BREEDING (1998) 117: 309-318 |
| ZM-ASI-3-1 | PLANT BREEDING (1998) 117: 309-318 |
| ZM-ASI-5-1 | PLANT BREEDING (1998) 117: 309-318 |
| ZM-ASI-6-1 | PLANT BREEDING (1998) 117: 309-318 |
| ZM-ASI-6-2 | PLANT BREEDING (1998) 117: 309-318 |
| ZM-ASI-7-1 | PLANT BREEDING (1998) 117: 309-318 |
| ZM-ASI-8-1 | PLANT BREEDING (1998) 117: 309-318 |
| ZM-ASI-9-1 | PLANT BREEDING (1998) 117: 309-318 |
| ZM-ASI-10-1 | PLANT BREEDING (1998) 117: 309-318 |
| ZM-SILK-1-2 | PLANT BREEDING (1998) 117: 309-318 |
| ZM-SILK-1-1 | PLANT BREEDING (1998) 117: 309-318 |
| ZM-SILK-2-2 | PLANT BREEDING (1998) 117: 309-318 |
| ZM-SILK-2-1 | PLANT BREEDING (1998) 117: 309-318 |
| ZM-SILK-3-1 | PLANT BREEDING (1998) 117: 309-318 |
| ZM-SILK-4-1 | PLANT BREEDING (1998) 117: 309-318 |
| ZM-SILK-5-1 | PLANT BREEDING (1998) 117: 309-318 |
| ZM-SILK-5-2 | PLANT BREEDING (1998) 117: 309-318 |
| ZM-SILK-6-1 | PLANT BREEDING (1998) 117: 309-318 |
| ZM-SILK-7-1 | PLANT BREEDING (1998) 117: 309-318 |
| ZM-SILK-9-2 | PLANT BREEDING (1998) 117: 309-318 |
| ZM-SILK-9-1 | PLANT BREEDING (1998) 117: 309-318 |
| ZM-SILK-10-1 | PLANT BREEDING (1998) 117: 309-318 |
| ZM-SWCBR-3-1 | PLANT BREEDING (1998) 117: 309-318 |
| ZM-SWCBR-5-1 | PLANT BREEDING (1998) 117: 309-318 |
| ZM-SWCBR-5-2 | PLANT BREEDING (1998) 117: 309-318 |
| ZM-SWCBR-6-1 | PLANT BREEDING (1998) 117: 309-318 |
| ZM-SWCBR-6-2 | PLANT BREEDING (1998) 117: 309-318 |
| ZM-SWCBR-8-1 | PLANT BREEDING (1998) 117: 309-318 |
| ZM-SWCBR-9-1 | PLANT BREEDING (1998) 117: 309-318 |
| ZM-ASI-1-3 | PLANT BREEDING (1998) 117: 193-202 |
| ZM-ASI-2-1 | PLANT BREEDING (1998) 117: 193-202 |
| ZM-ASI-3-2 | PLANT BREEDING (1998) 117: 193-202 |
| ZM-ASI-4-1 | PLANT BREEDING (1998) 117: 193-202 |
| ZM-ASI-5-2 | PLANT BREEDING (1998) 117: 193-202 |
| ZM-ASI-8-2 | PLANT BREEDING (1998) 117: 193-202 |
| ZM-SILK-3-2 | PLANT BREEDING (1998) 117: 193-202 |
| ZM-SILK-3-3 | PLANT BREEDING (1998) 117: 193-202 |
| ZM-SILK-6-2 | PLANT BREEDING (1998) 117: 193-202 |
| ZM-SILK-7-2 | PLANT BREEDING (1998) 117: 193-202 |
| ZM-SILK-8-1 | PLANT BREEDING (1998) 117: 193-202 |
| ZM-SILK-9-3 | PLANT BREEDING (1998) 117: 193-202 |
| ZM-SWCBR-1-1 | PLANT BREEDING (1998) 117: 193-202 |
| ZM-SWCBR-3-2 | PLANT BREEDING (1998) 117: 193-202 |
| ZM-ASI-1-4 | PLANT BREEDING (1998) 117: 193-202 |
| ZM-ASI-1-5 | PLANT BREEDING (1998) 117: 193-202 |
| ZM-ASI-3-4 | PLANT BREEDING (1998) 117: 193-202 |
| ZM-ASI-3-3 | PLANT BREEDING (1998) 117: 193-202 |
| ZM-ASI-5-3 | PLANT BREEDING (1998) 117: 193-202 |
| ZM-ASI-10-2 | PLANT BREEDING (1998) 117: 193-202 |
| ZM-GYLD-6-1 | PLANT BREEDING (1998) 117: 193-202 |
| ZM-SILK-3-5 | PLANT BREEDING (1998) 117: 193-202 |
| ZM-SILK-3-4 | PLANT BREEDING (1998) 117: 193-202 |
| ZM-SILK-9-4 | PLANT BREEDING (1998) 117: 193-202 |
| ZM-CPC-7-1 | CROP SCI (1998) 38: 1278-1289 |
| ZM-DMC-5-1 | CROP SCI (1998) 38: 1278-1289 |
| ZM-DMC-6-2 | CROP SCI (1998) 38: 1278-1289 |
| ZM-DMC-6-1 | CROP SCI (1998) 38: 1278-1289 |
| ZM-DMY-4-2 | CROP SCI (1998) 38: 1278-1289 |
| ZM-DMY-8-1 | CROP SCI (1998) 38: 1278-1289 |
| ZM-DMY-9-1 | CROP SCI (1998) 38: 1278-1289 |
| ZM-IVDOM-3-1 | CROP SCI (1998) 38: 1278-1289 |
| ZM-IVDOM-5-2 | CROP SCI (1998) 38: 1278-1289 |
| ZM-STC-5-1 | CROP SCI (1998) 38: 1278-1289 |
| ZM-STC-7-1 | CROP SCI (1998) 38: 1278-1289 |
| ZM-STC-8-1 | CROP SCI (1998) 38: 1278-1289 |
| ZM-CPC-1-4 | CROP SCI (1998) 38: 1278-1289 |
| ZM-CPC-1-3 | CROP SCI (1998) 38: 1278-1289 |
| ZM-CPC-1-2 | CROP SCI (1998) 38: 1278-1289 |
| ZM-CPC-3-2 | CROP SCI (1998) 38: 1278-1289 |
| ZM-CPC-3-1 | CROP SCI (1998) 38: 1278-1289 |
| ZM-CPC-6-1 | CROP SCI (1998) 38: 1278-1289 |
| ZM-CPC-7-3 | CROP SCI (1998) 38: 1278-1289 |
| ZM-CPC-7-2 | CROP SCI (1998) 38: 1278-1289 |
| ZM-DMC-1-1 | CROP SCI (1998) 38: 1278-1289 |
| ZM-DMY-2-4 | CROP SCI (1998) 38: 1278-1289 |
| ZM-DMY-2-3 | CROP SCI (1998) 38: 1278-1289 |
| ZM-DMY-3-2 | CROP SCI (1998) 38: 1278-1289 |
| ZM-DMY-5-1 | CROP SCI (1998) 38: 1278-1289 |
| ZM-DMY-8-2 | CROP SCI (1998) 38: 1278-1289 |
| ZM-IVDOM-1-2 | CROP SCI (1998) 38: 1278-1289 |
| ZM-IVDOM-1-1 | CROP SCI (1998) 38: 1278-1289 |
| ZM-IVDOM-10-1 | CROP SCI (1998) 38: 1278-1289 |
| ZM-STC-6-1 | CROP SCI (1998) 38: 1278-1289 |
| ZM-CPC-1-5 | CROP SCI (1998) 38: 1278-1289 |
| ZM-CPC-1-6 | CROP SCI (1998) 38: 1278-1289 |
| ZM-CPC-3-3 | CROP SCI (1998) 38: 1278-1289 |
| ZM-CPC-6-2 | CROP SCI (1998) 38: 1278-1289 |
| ZM-CPC-10-1 | CROP SCI (1998) 38: 1278-1289 |
| ZM-DMY-1-4 | CROP SCI (1998) 38: 1278-1289 |
| ZM-DMY-3-3 | CROP SCI (1998) 38: 1278-1289 |
| ZM-DMY-4-4 | CROP SCI (1998) 38: 1278-1289 |
| ZM-DMY-4-3 | CROP SCI (1998) 38: 1278-1289 |
| ZM-IVDOM-1-4 | CROP SCI (1998) 38: 1278-1289 |
| ZM-IVDOM-3-3 | CROP SCI (1998) 38: 1278-1289 |
| ZM-IVDOM-9-2 | CROP SCI (1998) 38: 1278-1289 |
| ZM-IVDOM-9-1 | CROP SCI (1998) 38: 1278-1289 |
| ZM-IVDOM-10-2 | CROP SCI (1998) 38: 1278-1289 |
| ZM-STC-2-2 | CROP SCI (1998) 38: 1278-1289 |
| ZM-STC-7-2 | CROP SCI (1998) 38: 1278-1289 |
| ZM-STC-10-2 | CROP SCI (1998) 38: 1278-1289 |
| ZM-GYHA-1-2 | CROP SCI (1998) 38: 1296-1308 |
| ZM-GYHA-1-4 | CROP SCI (1998) 38: 1296-1308 |
| ZM-GYHA-1-3 | CROP SCI (1998) 38: 1296-1308 |
| ZM-GYHA-5-1 | CROP SCI (1998) 38: 1296-1308 |
| ZM-GYHA-6-1 | CROP SCI (1998) 38: 1296-1308 |
| ZM-KW300-1-2 | CROP SCI (1998) 38: 1296-1308 |
| ZM-KW300-3-2 | CROP SCI (1998) 38: 1296-1308 |
| ZM-KW300-4-2 | CROP SCI (1998) 38: 1296-1308 |
| ZM-KW300-6-2 | CROP SCI (1998) 38: 1296-1308 |
| ZM-KW300-8-1 | CROP SCI (1998) 38: 1296-1308 |
| ZM-KW300-9-1 | CROP SCI (1998) 38: 1296-1308 |
| ZM-KW300-9-2 | CROP SCI (1998) 38: 1296-1308 |
| ZM-DMS-1-2 | THEOR APPL GENET (1999) 99: 519-523 |
| ZM-DMS-1-1 | THEOR APPL GENET (1999) 99: 519-523 |
| ZM-DMS-9-1 | THEOR APPL GENET (1999) 99: 519-523 |
| ZM-ASI-1-6 | THEOR APPL GENET (1999) 99: 289-295 |
| ZM-ASI-2-2 | THEOR APPL GENET (1999) 99: 289-295 |
| ZM-ASI-5-4 | THEOR APPL GENET (1999) 99: 289-295 |
| ZM-ASI-7-2 | THEOR APPL GENET (1999) 99: 289-295 |
| ZM-ASI-7-3 | THEOR APPL GENET (1999) 99: 289-295 |
| ZM-ASI-7-4 | THEOR APPL GENET (1999) 99: 289-295 |
| ZM-ASI-8-3 | THEOR APPL GENET (1999) 99: 289-295 |
| ZM-ASI-8-4 | THEOR APPL GENET (1999) 99: 289-295 |
| ZM-ASI-9-3 | THEOR APPL GENET (1999) 99: 289-295 |
| ZM-ASI-9-2 | THEOR APPL GENET (1999) 99: 289-295 |
| ZM-DTA-1-3 | THEOR APPL GENET (1999) 99: 289-295 |
| ZM-DTA-2-3 | THEOR APPL GENET (1999) 99: 289-295 |
| ZM-DTA-2-2 | THEOR APPL GENET (1999) 99: 289-295 |
| ZM-DTA-7-1 | THEOR APPL GENET (1999) 99: 289-295 |
| ZM-DTA-8-3 | THEOR APPL GENET (1999) 99: 289-295 |
| ZM-DTOL-4-3 | THEOR APPL GENET (1999) 99: 289-295 |
| ZM-SILK-7-3 | THEOR APPL GENET (1999) 99: 289-295 |
| ZM-SILK-9-5 | THEOR APPL GENET (1999) 99: 289-295 |
| ZM-DTOL-4-1 | THEOR APPL GENET (1999) 99: 280-288 |
| ZM-DTOL-9-1 | THEOR APPL GENET (1999) 99: 280-288 |
| ZM-EWT-4-2 | THEOR APPL GENET (1999) 99: 280-288 |
| ZM-KNE-4-1 | THEOR APPL GENET (1999) 99: 280-288 |
| ZM-KWE-4-1 | THEOR APPL GENET (1999) 99: 280-288 |
| ZM-CRR-1-14 | THEOR APPL GENET (1999) 99: 593-598 |
| ZM-CRR-1-15 | THEOR APPL GENET (1999) 99: 593-598 |
| ZM-CRR-3-8 | THEOR APPL GENET (1999) 99: 593-598 |
| ZM-CRR-5-9 | THEOR APPL GENET (1999) 99: 593-598 |
| ZM-CRR-6-6 | THEOR APPL GENET (1999) 99: 593-598 |
| ZM-PCSI-1-7 | THEOR APPL GENET (1999) 99: 593-598 |
| ZM-PCSI-3-6 | THEOR APPL GENET (1999) 99: 593-598 |
| ZM-PCSI-4-6 | THEOR APPL GENET (1999) 99: 593-598 |
| ZM-PCSI-5-7 | THEOR APPL GENET (1999) 99: 593-598 |
| ZM-PHSI-4-1 | THEOR APPL GENET (1999) 99: 593-598 |
| ZM-PHSI-5-1 | THEOR APPL GENET (1999) 99: 593-598 |
| ZM-PHSI-6-2 | THEOR APPL GENET (1999) 99: 593-598 |
| ZM-PHSI-6-3 | THEOR APPL GENET (1999) 99: 593-598 |
| ZM-PHSI-6-1 | THEOR APPL GENET (1999) 99: 593-598 |
| ZM-PHSI-8-1 | THEOR APPL GENET (1999) 99: 593-598 |
| ZM-PHSI-9-1 | THEOR APPL GENET (1999) 99: 593-598 |
| ZM-HPVI-3-1 | CROP SCI (1999) 39: 1171-1177 |
| ZM-HPVI-6-1 | CROP SCI (1999) 39: 1171-1177 |
| ZM-LVI-4-1 | CROP SCI (1999) 39: 1171-1177 |
| ZM-LVI-5-1 | CROP SCI (1999) 39: 1171-1177 |
| ZM-LVI-6-1 | CROP SCI (1999) 39: 1171-1177 |
| ZM-TRANS-1-1 | GENETICS (1999) 153: 993-1007 |
| ZM-TRANS-8-1 | GENETICS (1999) 153: 993-1007 |
| ZM-TRANS-8-2 | GENETICS (1999) 153: 993-1007 |
| ZM-ADCNLB-3-1 | CROP SCI (1999) 39: 514-523 |
| ZM-ADCNLB-3-2 | CROP SCI (1999) 39: 514-523 |
| ZM-ADCNLB-5-2 | CROP SCI (1999) 39: 514-523 |
| ZM-ADCNLB-5-1 | CROP SCI (1999) 39: 514-523 |
| ZM-ADCNLB-9-1 | CROP SCI (1999) 39: 514-523 |
| ZM-DTA-1-1 | CROP SCI (1999) 39: 514-523 |
| ZM-DTA-1-2 | CROP SCI (1999) 39: 514-523 |
| ZM-DTA-2-1 | CROP SCI (1999) 39: 514-523 |
| ZM-DTA-3-2 | CROP SCI (1999) 39: 514-523 |
| ZM-DTA-3-1 | CROP SCI (1999) 39: 514-523 |
| ZM-DTA-4-1 | CROP SCI (1999) 39: 514-523 |
| ZM-DTA-5-1 | CROP SCI (1999) 39: 514-523 |
| ZM-DTA-6-1 | CROP SCI (1999) 39: 514-523 |
| ZM-DTA-8-1 | CROP SCI (1999) 39: 514-523 |
| ZM-DTA-10-1 | CROP SCI (1999) 39: 514-523 |
| ZM-NLBIP-3-1 | CROP SCI (1999) 39: 514-523 |
| ZM-NLBIP-4-1 | CROP SCI (1999) 39: 514-523 |
| ZM-NLBIP-5-1 | CROP SCI (1999) 39: 514-523 |
| ZM-NLBIP-8-1 | CROP SCI (1999) 39: 514-523 |
| ZM-NLBIP-9-1 | CROP SCI (1999) 39: 514-523 |
| ZM-NLBDS-3-2 | THEOR APPL GENET (1999) 98: 1036-1045 |
| ZM-NLBDS-5-2 | THEOR APPL GENET (1999) 98: 1036-1045 |
| ZM-NLBDS-5-1 | THEOR APPL GENET (1999) 98: 1036-1045 |
| ZM-NLBDS-8-1 | THEOR APPL GENET (1999) 98: 1036-1045 |
| ZM-NLBDS-9-1 | THEOR APPL GENET (1999) 98: 1036-1045 |
| ZM-NLBDS-1-1 | THEOR APPL GENET (1999) 99: 649-655 |
| ZM-NLBDS-3-5 | THEOR APPL GENET (1999) 99: 649-655 |
| ZM-NLBDS-4-2 | THEOR APPL GENET (1999) 99: 649-655 |
| ZM-NLBDS-4-1 | THEOR APPL GENET (1999) 99: 649-655 |
| ZM-NLBDS-5-5 | THEOR APPL GENET (1999) 99: 649-655 |
| ZM-NLBDS-6-1 | THEOR APPL GENET (1999) 99: 649-655 |
| ZM-NLBDS-8-2 | THEOR APPL GENET (1999) 99: 649-655 |
| ZM-NLBDS-9-2 | THEOR APPL GENET (1999) 99: 649-655 |
| ZM-AUT-1-1 | THEOR APPL GENET (1999) 99: 524-539 |
| ZM-AUT-10-1 | THEOR APPL GENET (1999) 99: 524-539 |
| ZM-APIT-1-1 | THEOR APPL GENET (1999) 99: 524-539 |
| ZM-APIT-2-1 | THEOR APPL GENET (1999) 99: 524-539 |
| ZM-APIT-3-1 | THEOR APPL GENET (1999) 99: 524-539 |
| ZM-APIT-9-1 | THEOR APPL GENET (1999) 99: 524-539 |
| ZM-APIT-10-1 | THEOR APPL GENET (1999) 99: 524-539 |
| ZM-ANMT-1-1 | THEOR APPL GENET (1999) 99: 524-539 |
| ZM-ANMT-2-1 | THEOR APPL GENET (1999) 99: 524-539 |
| ZM-ANMT-10-1 | THEOR APPL GENET (1999) 99: 524-539 |
| ZM-MSVI-1-1 | THEOR APPL GENET (1999) 99: 524-539 |
| ZM-MSVI-1-2 | THEOR APPL GENET (1999) 99: 524-539 |
| ZM-MSVI-2-1 | THEOR APPL GENET (1999) 99: 524-539 |
| ZM-MSVI-3-2 | THEOR APPL GENET (1999) 99: 524-539 |
| ZM-MSVI-3-1 | THEOR APPL GENET (1999) 99: 524-539 |
| ZM-MSVI-9-1 | THEOR APPL GENET (1999) 99: 524-539 |
| ZM-MSVI-10- | THEOR APPL GENET (1999) 99: 524-539 |
| ZM-AUT-1-2 | THEOR APPL GENET (1999) 99: 540-553 |
| ZM-AUT-3-1 | THEOR APPL GENET (1999) 99: 540-553 |
| ZM-AUT-6-1 | THEOR APPL GENET (1999) 99: 540-553 |
| ZM-AUT-10-2 | THEOR APPL GENET (1999) 99: 540-553 |
| ZM-APIT-1-2 | THEOR APPL GENET (1999) 99: 540-553 |
| ZM-APIT-3-2 | THEOR APPL GENET (1999) 99: 540-553 |
| ZM-APIT-6-1 | THEOR APPL GENET (1999) 99: 540-553 |
| ZM-APIT-10-2 | THEOR APPL GENET (1999) 99: 540-553 |
| ZM-ANMT-1-2 | THEOR APPL GENET (1999) 99: 540-553 |
| ZM-ANMT-5-1 | THEOR APPL GENET (1999) 99: 540-553 |
| ZM-ANMT-10-2 | THEOR APPL GENET (1999) 99: 540-553 |
| ZM-ASI-1-7 | THEOR APPL GENET (1999) 99: 1106-1119 |
| ZM-ASI-2-3 | THEOR APPL GENET (1999) 99: 1106-1119 |
| ZM-ASI-4-2 | THEOR APPL GENET (1999) 99: 1106-1119 |
| ZM-ASI-7-5 | THEOR APPL GENET (1999) 99: 1106-1119 |
| ZM-ASI-9-4 | THEOR APPL GENET (1999) 99: 1106-1119 |
| ZM-ASI-10-3 | THEOR APPL GENET (1999) 99: 1106-1119 |
| ZM-BIOM-3-1 | THEOR APPL GENET (1999) 99: 1106-1119 |
| ZM-BIOM-8-1 | THEOR APPL GENET (1999) 99: 1106-1119 |
| ZM-CRR-3-9 | THEOR APPL GENET (1999) 99: 1106-1119 |
| ZM-CRR-4-6 | THEOR APPL GENET (1999) 99: 1106-1119 |
| ZM-CRR-9-5 | THEOR APPL GENET (1999) 99: 1106-1119 |
| ZM-DTA-1-4 | THEOR APPL GENET (1999) 99: 1106-1119 |
| ZM-DTA-4-2 | THEOR APPL GENET (1999) 99: 1106-1119 |
| ZM-DTA-5-2 | THEOR APPL GENET (1999) 99: 1106-1119 |
| ZM-DTA-7-2 | THEOR APPL GENET (1999) 99: 1106-1119 |
| ZM-DTA-8-4 | THEOR APPL GENET (1999) 99: 1106-1119 |
| ZM-DTA-8-2 | THEOR APPL GENET (1999) 99: 1106-1119 |
| ZM-DTA-9-1 | THEOR APPL GENET (1999) 99: 1106-1119 |
| ZM-DTA-10-2 | THEOR APPL GENET (1999) 99: 1106-1119 |
| ZM-ETURI-2-1 | THEOR APPL GENET (1999) 99: 1106-1119 |
| ZM-ETURI-5-1 | THEOR APPL GENET (1999) 99: 1106-1119 |
| ZM-HMAYI-3-1 | THEOR APPL GENET (1999) 99: 1106-1119 |
| ZM-HI-1-1 | THEOR APPL GENET (1999) 99: 1106-1119 |
| ZM-HI-3-1 | THEOR APPL GENET (1999) 99: 1106-1119 |
| ZM-HI-4-1 | THEOR APPL GENET (1999) 99: 1106-1119 |
| ZM-HI-8-1 | THEOR APPL GENET (1999) 99: 1106-1119 |
| ZM-KW100-1-2 | THEOR APPL GENET (1999) 99: 1106-1119 |
| ZM-KW100-9-1 | THEOR APPL GENET (1999) 99: 1106-1119 |
| ZM-SILK-1-3 | THEOR APPL GENET (1999) 99: 1106-1119 |
| ZM-SILK-5-3 | THEOR APPL GENET (1999) 99: 1106-1119 |
| ZM-SILK-7-4 | THEOR APPL GENET (1999) 99: 1106-1119 |
| ZM-SILK-8-2 | THEOR APPL GENET (1999) 99: 1106-1119 |
| ZM-SILK-9-6 | THEOR APPL GENET (1999) 99: 1106-1119 |
| ZM-SILK-10-2 | THEOR APPL GENET (1999) 99: 1106-1119 |
| ZM-GWM2-3-1 | THEOR APPL GENET (1999) 99: 1106-1119 |
| ZM-GWM2-3-2 | THEOR APPL GENET (1999) 99: 1106-1119 |
| ZM-DA-1-1 | THEOR APPL GENET (2000) 101: 907-917 |
| ZM-DA-1-2 | THEOR APPL GENET (2000) 101: 907-917 |
| ZM-DA-2-1 | THEOR APPL GENET (2000) 101: 907-917 |
| ZM-DA-3-1 | THEOR APPL GENET (2000) 101: 907-917 |
| ZM-DA-3-2 | THEOR APPL GENET (2000) 101: 907-917 |
| ZM-DA-4-1 | THEOR APPL GENET (2000) 101: 907-917 |
| ZM-DA-4-2 | THEOR APPL GENET (2000) 101: 907-917 |
| ZM-DA-5-1 | THEOR APPL GENET (2000) 101: 907-917 |
| ZM-DA-6-1 | THEOR APPL GENET (2000) 101: 907-917 |
| ZM-DA-6-2 | THEOR APPL GENET (2000) 101: 907-917 |
| ZM-DA-8-1 | THEOR APPL GENET (2000) 101: 907-917 |
| ZM-DA-10-1 | THEOR APPL GENET (2000) 101: 907-917 |
| ZM-CPC-1-2 | CROP SCI (2001) 41: 690-697 |
| ZM-CPC-6-1 | CROP SCI (2001) 41: 690-697 |
| ZM-CPC-7-1 | CROP SCI (2001) 41: 690-697 |
| ZM-DMY-8-1 | CROP SCI (2001) 41: 690-697 |
| ZM-DMC-5-1 | CROP SCI (2001) 41: 690-697 |
| ZM-SILK-1-1 | CROP SCI (2001) 41: 690-697 |
| ZM-SILK-5-1 | CROP SCI (2001) 41: 690-697 |
| ZM-SILK-8-1 | CROP SCI (2001) 41: 690-697 |
| ZM-STC-5-1 | CROP SCI (2001) 41: 690-697 |
| ZM-STC-8-1 | CROP SCI (2001) 41: 690-697 |
| ZM-DMC-1-1 | THEOR APPL GENET (2001) 102: 230-243 |
| ZM-DMC-5-1 | THEOR APPL GENET (2001) 102: 230-243 |
| ZM-DMC-6-1 | THEOR APPL GENET (2001) 102: 230-243 |
| ZM-AD-1-1 | CROP SCI (2001) 41: 835-845 |
| ZM-AD-1-2 | CROP SCI (2001) 41: 835-845 |
| ZM-AD-2-1 | CROP SCI (2001) 41: 835-845 |
| ZM-AD-3-1 | CROP SCI (2001) 41: 835-845 |
| ZM-AD-3-2 | CROP SCI (2001) 41: 835-845 |
| ZM-AD-4-1 | CROP SCI (2001) 41: 835-845 |
| ZM-AD-8-1 | CROP SCI (2001) 41: 835-845 |
| ZM-AD-9-1 | CROP SCI (2001) 41: 835-845 |
| ZM-ANT-1-1 | THEOR APPL GENET (2001) 102: 163-176 |
| ZM-ANT-1-2 | THEOR APPL GENET (2001) 102: 163-176 |
| ZM-ANT-1-3 | THEOR APPL GENET (2001) 102: 163-176 |
| ZM-ANT-1-4 | THEOR APPL GENET (2001) 102: 163-176 |
| ZM-ANT-1-5 | THEOR APPL GENET (2001) 102: 163-176 |
| ZM-ANT-2-1 | THEOR APPL GENET (2001) 102: 163-176 |
| ZM-ANT-2-2 | THEOR APPL GENET (2001) 102: 163-176 |
| ZM-ANT-3-1 | THEOR APPL GENET (2001) 102: 163-176 |
| ZM-ANT-4-1 | THEOR APPL GENET (2001) 102: 163-176 |
| ZM-ANT-4-2 | THEOR APPL GENET (2001) 102: 163-176 |
| ZM-ANT-4-3 | THEOR APPL GENET (2001) 102: 163-176 |
| ZM-ANT-4-4 | THEOR APPL GENET (2001) 102: 163-176 |
| ZM-ANT-5-1 | THEOR APPL GENET (2001) 102: 163-176 |
| ZM-ANT-6-1 | THEOR APPL GENET (2001) 102: 163-176 |
| ZM-ANT-6-2 | THEOR APPL GENET (2001) 102: 163-176 |
| ZM-ANT-7-1 | THEOR APPL GENET (2001) 102: 163-176 |
| ZM-ANT-7-2 | THEOR APPL GENET (2001) 102: 163-176 |
| ZM-ANT-8-1 | THEOR APPL GENET (2001) 102: 163-176 |
| ZM-ANT-8-2 | THEOR APPL GENET (2001) 102: 163-176 |
| ZM-ANT-9-1 | THEOR APPL GENET (2001) 102: 163-176 |
| ZM-ANT-9-2 | THEOR APPL GENET (2001) 102: 163-176 |
| ZM-ANT-9-3 | THEOR APPL GENET (2001) 102: 163-176 |
| ZM-SILK-1-1 | THEOR APPL GENET (2001) 102: 163-176 |
| ZM-SILK-1-2 | THEOR APPL GENET (2001) 102: 163-176 |
| ZM-SILK-1-3 | THEOR APPL GENET (2001) 102: 163-176 |
| ZM-SILK-1-4 | THEOR APPL GENET (2001) 102: 163-176 |
| ZM-SILK-1-5 | THEOR APPL GENET (2001) 102: 163-176 |
| ZM-SILK-2-1 | THEOR APPL GENET (2001) 102: 163-176 |
| ZM-SILK-2-2 | THEOR APPL GENET (2001) 102: 163-176 |
| ZM-SILK-2-3 | THEOR APPL GENET (2001) 102: 163-176 |
| ZM-SILK-3-1 | THEOR APPL GENET (2001) 102: 163-176 |
| ZM-SILK-4-1 | THEOR APPL GENET (2001) 102: 163-176 |
| ZM-SILK-4-2 | THEOR APPL GENET (2001) 102: 163-176 |
| ZM-SILK-6-1 | THEOR APPL GENET (2001) 102: 163-176 |
| ZM-SILK-6-2 | THEOR APPL GENET (2001) 102: 163-176 |
| ZM-SILK-6-3 | THEOR APPL GENET (2001) 102: 163-176 |
| ZM-SILK-7-1 | THEOR APPL GENET (2001) 102: 163-176 |
| ZM-SILK-7-2 | THEOR APPL GENET (2001) 102: 163-176 |
| ZM-SILK-7-3 | THEOR APPL GENET (2001) 102: 163-176 |
| ZM-SILK-8-1 | THEOR APPL GENET (2001) 102: 163-176 |
| ZM-SILK-9-1 | THEOR APPL GENET (2001) 102: 163-176 |
| ZM-SILK-9-2 | THEOR APPL GENET (2001) 102: 163-176 |
| ZM-SILK-10-1 | THEOR APPL GENET (2001) 102: 163-176 |
| ZM-ASI-1-1 | THEOR APPL GENET (2001) 102: 163-176 |
| ZM-ASI-1-2 | THEOR APPL GENET (2001) 102: 163-176 |
| ZM-ASI-1-3 | THEOR APPL GENET (2001) 102: 163-176 |
| ZM-ASI-2-1 | THEOR APPL GENET (2001) 102: 163-176 |
| ZM-ASI-2-2 | THEOR APPL GENET (2001) 102: 163-176 |
| ZM-ASI-3-1 | THEOR APPL GENET (2001) 102: 163-176 |
| ZM-ASI-4-1 | THEOR APPL GENET (2001) 102: 163-176 |
| ZM-ASI-5-1 | THEOR APPL GENET (2001) 102: 163-176 |
| ZM-ASI-7-1 | THEOR APPL GENET (2001) 102: 163-176 |
| ZM-ASI-7-2 | THEOR APPL GENET (2001) 102: 163-176 |
| ZM-ASI-8-1 | THEOR APPL GENET (2001) 102: 163-176 |
| ZM-ASI-8-2 | THEOR APPL GENET (2001) 102: 163-176 |
| ZM-ASI-9-1 | THEOR APPL GENET (2001) 102: 163-176 |

TABLE 6A
Sequences associated with the listed maize quantitative trait loci (QTLs) and showing
at least a 2-fold alteration in expression associated with the listed trait.

| QTL | TRAIT | SEQ ID |
|---|---|---|
| ABIOTIC_STRESS_ZM-DTOL-1-1 | ABIOTIC STRESS | 71 |
| ABIOTIC_STRESS_ZM-DTOL-1-1 | ABIOTIC STRESS | 217 |
| ABIOTIC_STRESS_ZM-DTOL-1-1 | ABIOTIC STRESS | 217 |
| ABIOTIC_STRESS_ZM-DTOL-4-1 | ABIOTIC STRESS | 131 |
| ABIOTIC_STRESS_ZM-DTOL-4-1 | ABIOTIC STRESS | 197 |
| ABIOTIC_STRESS_ZM-DTOL-4-1 | ABIOTIC STRESS | 25 |
| ABIOTIC_STRESS_ZM-DTOL-4-1 | ABIOTIC STRESS | 197 |
| ABIOTIC_STRESS_ZM-DTOL-4-1 | ABIOTIC STRESS | 131 |
| ABIOTIC_STRESS_ZM-DTOL-4-1 | ABIOTIC STRESS | 131 |
| ABIOTIC_STRESS_ZM-DTOL-4-1 | ABIOTIC STRESS | 25 |
| ABIOTIC_STRESS_ZM-DTOL-4-3 | ABIOTIC STRESS | 131 |
| ABIOTIC_STRESS_ZM-DTOL-4-3 | ABIOTIC STRESS | 197 |
| ABIOTIC_STRESS_ZM-DTOL-4-3 | ABIOTIC STRESS | 25 |
| ABIOTIC_STRESS_ZM-DTOL-4-3 | ABIOTIC STRESS | 197 |
| ABIOTIC_STRESS_ZM-DTOL-4-3 | ABIOTIC STRESS | 131 |
| ABIOTIC_STRESS_ZM-DTOL-4-3 | ABIOTIC STRESS | 131 |
| ABIOTIC_STRESS_ZM-DTOL-4-3 | ABIOTIC STRESS | 25 |
| ABIOTIC_STRESS_ZM-DTOL-9-1 | ABIOTIC STRESS | 99 |
| ABIOTIC_STRESS_ZM-DTOL-9-1 | ABIOTIC STRESS | 99 |
| ABIOTIC_STRESS_ZM-DTOL-9-1 | ABIOTIC STRESS | 331 |
| ABIOTIC_STRESS_ZM-DTOL-9-1 | ABIOTIC STRESS | 331 |
| ABIOTIC_STRESS_ZM-LABA-1-1 | ABIOTIC STRESS | 319 |
| ABIOTIC_STRESS_ZM-LABA-1-1 | ABIOTIC STRESS | 319 |
| ABIOTIC_STRESS_ZM-LABA-1-1 | ABIOTIC STRESS | 319 |
| ABIOTIC_STRESS_ZM-LABA-1-2 | ABIOTIC STRESS | 285 |
| ABIOTIC_STRESS_ZM-LABA-1-2 | ABIOTIC STRESS | 87 |
| ABIOTIC_STRESS_ZM-LABA-1-2 | ABIOTIC STRESS | 285 |
| ABIOTIC_STRESS_ZM-LABA-1-2 | ABIOTIC STRESS | 87 |
| ABIOTIC_STRESS_ZM-LABA-1-2 | ABIOTIC STRESS | 217 |
| ABIOTIC_STRESS_ZM-LABA-1-2 | ABIOTIC STRESS | 285 |
| ABIOTIC_STRESS_ZM-LABA-1-2 | ABIOTIC STRESS | 87 |
| ABIOTIC_STRESS_ZM-LABA-1-2 | ABIOTIC STRESS | 217 |
| ABIOTIC_STRESS_ZM-LABA-10-2 | ABIOTIC STRESS | 87 |
| ABIOTIC_STRESS_ZM-LABA-10-2 | ABIOTIC STRESS | 87 |
| ABIOTIC_STRESS_ZM-LABA-10-2 | ABIOTIC STRESS | 217 |
| ABIOTIC_STRESS_ZM-LABA-10-2 | ABIOTIC STRESS | 87 |
| ABIOTIC_STRESS_ZM-LABA-10-2 | ABIOTIC STRESS | 217 |
| ABIOTIC_STRESS_ZM-LABA-2-1 | ABIOTIC STRESS | 291 |
| ABIOTIC_STRESS_ZM-LABA-2-1 | ABIOTIC STRESS | 87 |
| ABIOTIC_STRESS_ZM-LABA-2-1 | ABIOTIC STRESS | 291 |
| ABIOTIC_STRESS_ZM-LABA-2-1 | ABIOTIC STRESS | 87 |
| ABIOTIC_STRESS_ZM-LABA-2-1 | ABIOTIC STRESS | 87 |
| ABIOTIC_STRESS_ZM-LABA-2-3 | ABIOTIC STRESS | 233 |
| ABIOTIC_STRESS_ZM-LABA-2-3 | ABIOTIC STRESS | 285 |
| ABIOTIC_STRESS_ZM-LABA-2-3 | ABIOTIC STRESS | 233 |
| ABIOTIC_STRESS_ZM-LABA-2-3 | ABIOTIC STRESS | 33 |
| ABIOTIC_STRESS_ZM-LABA-2-3 | ABIOTIC STRESS | 233 |
| ABIOTIC_STRESS_ZM-LABA-2-3 | ABIOTIC STRESS | 359 |
| ABIOTIC_STRESS_ZM-LABA-2-3 | ABIOTIC STRESS | 233 |
| ABIOTIC_STRESS_ZM-LABA-2-3 | ABIOTIC STRESS | 359 |
| ABIOTIC_STRESS_ZM-LABA-2-3 | ABIOTIC STRESS | 285 |
| ABIOTIC_STRESS_ZM-LABA-2-3 | ABIOTIC STRESS | 281 |
| ABIOTIC_STRESS_ZM-LABA-2-3 | ABIOTIC STRESS | 281 |
| ABIOTIC_STRESS_ZM-LABA-2-3 | ABIOTIC STRESS | 33 |
| ABIOTIC_STRESS_ZM-LABA-2-3 | ABIOTIC STRESS | 359 |
| ABIOTIC_STRESS_ZM-LABA-2-3 | ABIOTIC STRESS | 285 |
| ABIOTIC_STRESS_ZM-LABA-2-3 | ABIOTIC STRESS | 233 |
| ABIOTIC_STRESS_ZM-LABA-2-3 | ABIOTIC STRESS | 33 |
| ABIOTIC_STRESS_ZM-LABA-2-4 | ABIOTIC STRESS | 33 |
| ABIOTIC_STRESS_ZM-LABA-2-4 | ABIOTIC STRESS | 359 |
| ABIOTIC_STRESS_ZM-LABA-2-4 | ABIOTIC STRESS | 359 |
| ABIOTIC_STRESS_ZM-LABA-2-4 | ABIOTIC STRESS | 33 |
| ABIOTIC_STRESS_ZM-LABA-2-4 | ABIOTIC STRESS | 217 |
| ABIOTIC_STRESS_ZM-LABA-2-4 | ABIOTIC STRESS | 359 |
| ABIOTIC_STRESS_ZM-LABA-2-4 | ABIOTIC STRESS | 217 |
| ABIOTIC_STRESS_ZM-LABA-2-4 | ABIOTIC STRESS | 33 |
| ABIOTIC_STRESS_ZM-LABA-3-1 | ABIOTIC STRESS | 285 |
| ABIOTIC_STRESS_ZM-LABA-3-1 | ABIOTIC STRESS | 285 |
| ABIOTIC_STRESS_ZM-LABA-3-1 | ABIOTIC STRESS | 257 |
| ABIOTIC_STRESS_ZM-LABA-3-1 | ABIOTIC STRESS | 257 |
| ABIOTIC_STRESS_ZM-LABA-3-1 | ABIOTIC STRESS | 285 |
| ABIOTIC_STRESS_ZM-LABA-4-1 | ABIOTIC STRESS | 291 |
| ABIOTIC_STRESS_ZM-LABA-4-1 | ABIOTIC STRESS | 33 |
| ABIOTIC_STRESS_ZM-LABA-4-1 | ABIOTIC STRESS | 273 |
| ABIOTIC_STRESS_ZM-LABA-4-1 | ABIOTIC STRESS | 291 |
| ABIOTIC_STRESS_ZM-LABA-4-1 | ABIOTIC STRESS | 25 |
| ABIOTIC_STRESS_ZM-LABA-4-1 | ABIOTIC STRESS | 273 |
| ABIOTIC_STRESS_ZM-LABA-4-1 | ABIOTIC STRESS | 273 |
| ABIOTIC_STRESS_ZM-LABA-4-1 | ABIOTIC STRESS | 71 |
| ABIOTIC_STRESS_ZM-LABA-4-1 | ABIOTIC STRESS | 57 |
| ABIOTIC_STRESS_ZM-LABA-4-1 | ABIOTIC STRESS | 273 |
| ABIOTIC_STRESS_ZM-LABA-4-1 | ABIOTIC STRESS | 273 |
| ABIOTIC_STRESS_ZM-LABA-4-1 | ABIOTIC STRESS | 33 |
| ABIOTIC_STRESS_ZM-LABA-4-1 | ABIOTIC STRESS | 25 |
| ABIOTIC_STRESS_ZM-LABA-4-1 | ABIOTIC STRESS | 57 |
| ABIOTIC_STRESS_ZM-LABA-4-1 | ABIOTIC STRESS | 273 |
| ABIOTIC_STRESS_ZM-LABA-4-1 | ABIOTIC STRESS | 33 |
| ABIOTIC_STRESS_ZM-LABA-4-2 | ABIOTIC STRESS | 291 |
| ABIOTIC_STRESS_ZM-LABA-4-2 | ABIOTIC STRESS | 33 |
| ABIOTIC_STRESS_ZM-LABA-4-2 | ABIOTIC STRESS | 273 |
| ABIOTIC_STRESS_ZM-LABA-4-2 | ABIOTIC STRESS | 291 |
| ABIOTIC_STRESS_ZM-LABA-4-2 | ABIOTIC STRESS | 25 |
| ABIOTIC_STRESS_ZM-LABA-4-2 | ABIOTIC STRESS | 273 |
| ABIOTIC_STRESS_ZM-LABA-4-2 | ABIOTIC STRESS | 273 |
| ABIOTIC_STRESS_ZM-LABA-4-2 | ABIOTIC STRESS | 71 |
| ABIOTIC_STRESS_ZM-LABA-4-2 | ABIOTIC STRESS | 57 |
| ABIOTIC_STRESS_ZM-LABA-4-2 | ABIOTIC STRESS | 273 |
| ABIOTIC_STRESS_ZM-LABA-4-2 | ABIOTIC STRESS | 273 |
| ABIOTIC_STRESS_ZM-LABA-4-2 | ABIOTIC STRESS | 33 |
| ABIOTIC_STRESS_ZM-LABA-4-2 | ABIOTIC STRESS | 25 |
| ABIOTIC_STRESS_ZM-LABA-4-2 | ABIOTIC STRESS | 57 |
| ABIOTIC_STRESS_ZM-LABA-4-2 | ABIOTIC STRESS | 273 |
| ABIOTIC_STRESS_ZM-LABA-4-2 | ABIOTIC STRESS | 33 |
| ABIOTIC_STRESS_ZM-LABA-7-1 | ABIOTIC STRESS | 87 |
| ABIOTIC_STRESS_ZM-LABA-7-1 | ABIOTIC STRESS | 87 |
| ABIOTIC_STRESS_ZM-LABA-7-1 | ABIOTIC STRESS | 87 |
| ABIOTIC_STRESS_ZM-LABA-7-2 | ABIOTIC STRESS | 87 |
| ABIOTIC_STRESS_ZM-LABA-7-2 | ABIOTIC STRESS | 87 |
| ABIOTIC_STRESS_ZM-LABA-7-2 | ABIOTIC STRESS | 87 |
| ABIOTIC_STRESS_ZM-LABA-9-1 | ABIOTIC STRESS | 131 |
| ABIOTIC_STRESS_ZM-LABA-9-1 | ABIOTIC STRESS | 319 |
| ABIOTIC_STRESS_ZM-LABA-9-1 | ABIOTIC STRESS | 309 |
| ABIOTIC_STRESS_ZM-LABA-9-1 | ABIOTIC STRESS | 197 |
| ABIOTIC_STRESS_ZM-LABA-9-1 | ABIOTIC STRESS | 197 |
| ABIOTIC_STRESS_ZM-LABA-9-1 | ABIOTIC STRESS | 257 |
| ABIOTIC_STRESS_ZM-LABA-9-1 | ABIOTIC STRESS | 319 |
| ABIOTIC_STRESS_ZM-LABA-9-1 | ABIOTIC STRESS | 131 |
| ABIOTIC_STRESS_ZM-LABA-9-1 | ABIOTIC STRESS | 131 |
| ABIOTIC_STRESS_ZM-LABA-9-1 | ABIOTIC STRESS | 257 |
| ABIOTIC_STRESS_ZM-LABA-9-1 | ABIOTIC STRESS | 319 |
| FLOWERING_MAS13-15 | FLOWERING | 131 |
| FLOWERING_MAS13-3 | FLOWERING | 131 |
| FLOWERING_SMS021-12 | FLOWERING | 131 |
| FLOWERING_SMS024-46 | FLOWERING | 131 |
| FLOWERING_SMS024-50 | FLOWERING | 131 |
| GRAIN_PERFORMANCE_MAS12-22 | GRAIN_QUALITY | 25 |
| GRAIN_PERFORMANCE_MAS12-22 | GRAIN_QUALITY | 25 |
| GRAIN_PERFORMANCE_MAS12-23 | GRAIN_QUALITY | 285 |
| GRAIN_PERFORMANCE_MAS12-23 | GRAIN_QUALITY | 131 |
| GRAIN_PERFORMANCE_MAS12-23 | GRAIN_QUALITY | 349 |
| GRAIN_PERFORMANCE_MAS12-23 | GRAIN_QUALITY | 197 |
| GRAIN_PERFORMANCE_MAS12-23 | GRAIN_QUALITY | 263 |
| GRAIN_PERFORMANCE_MAS12-23 | GRAIN_QUALITY | 131 |
| GRAIN_PERFORMANCE_MAS12-23 | GRAIN_QUALITY | 263 |
| GRAIN_PERFORMANCE_MAS12-23 | GRAIN_QUALITY | 349 |
| GRAIN_PERFORMANCE_MAS12-23 | GRAIN_QUALITY | 285 |
| GRAIN_PERFORMANCE_MAS12-23 | GRAIN_QUALITY | 263 |
| GRAIN_PERFORMANCE_MAS12-23 | GRAIN_QUALITY | 237 |
| GRAIN_PERFORMANCE_MAS12-24 | GRAIN_QUALITY | 263 |
| GRAIN_PERFORMANCE_MAS12-24 | GRAIN_QUALITY | 257 |
| GRAIN_PERFORMANCE_MAS12-24 | GRAIN_QUALITY | 263 |
| GRAIN_PERFORMANCE_MAS12-24 | GRAIN_QUALITY | 263 |
| GRAIN_PERFORMANCE_MAS12-29 | GRAIN_QUALITY | 281 |
| GRAIN_PERFORMANCE_MAS12-32 | GRAIN_QUALITY | 285 |
| GRAIN_PERFORMANCE_MAS12-32 | GRAIN_QUALITY | 359 |
| GRAIN_PERFORMANCE_MAS12-32 | GRAIN_QUALITY | 233 |
| GRAIN_PERFORMANCE_MAS12-32 | GRAIN_QUALITY | 359 |
| GRAIN_PERFORMANCE_MAS12-32 | GRAIN_QUALITY | 233 |
| GRAIN_PERFORMANCE_MAS12-32 | GRAIN_QUALITY | 281 |
| GRAIN_PERFORMANCE_MAS12-32 | GRAIN_QUALITY | 233 |
| GRAIN_PERFORMANCE_MAS12-32 | GRAIN_QUALITY | 285 |
| GRAIN_PERFORMANCE_MAS12-32 | GRAIN_QUALITY | 233 |
| GRAIN_PERFORMANCE_MAS12-32 | GRAIN_QUALITY | 33 |
| GRAIN_PERFORMANCE_MAS12-33 | GRAIN_QUALITY | 285 |
| GRAIN_PERFORMANCE_MAS12-33 | GRAIN_QUALITY | 87 |
| GRAIN_PERFORMANCE_MAS12-33 | GRAIN_QUALITY | 87 |
| GRAIN_PERFORMANCE_MAS12-33 | GRAIN_QUALITY | 285 |
| GRAIN_PERFORMANCE_MAS12-34 | GRAIN_QUALITY | 291 |
| GRAIN_PERFORMANCE_MAS12-34 | GRAIN_QUALITY | 273 |
| GRAIN_PERFORMANCE_MAS12-34 | GRAIN_QUALITY | 291 |
| GRAIN_PERFORMANCE_MAS12-34 | GRAIN_QUALITY | 273 |
| GRAIN_PERFORMANCE_MAS12-34 | GRAIN_QUALITY | 291 |
| GRAIN_PERFORMANCE_MAS12-34 | GRAIN_QUALITY | 291 |
| GRAIN_PERFORMANCE_MAS12-34 | GRAIN_QUALITY | 273 |
| GRAIN_PERFORMANCE_MAS12-34 | GRAIN_QUALITY | 57 |
| GRAIN_PERFORMANCE_MAS12-34 | GRAIN_QUALITY | 291 |
| GRAIN_PERFORMANCE_MAS12-34 | GRAIN_QUALITY | 273 |
| GRAIN_PERFORMANCE_MAS12-35 | GRAIN_QUALITY | 25 |
| GRAIN_PERFORMANCE_MAS12-35 | GRAIN_QUALITY | 25 |
| GRAIN_PERFORMANCE_MAS12-35 | GRAIN_QUALITY | 321 |
| GRAIN_PERFORMANCE_MAS12-37 | GRAIN_QUALITY | 25 |
| GRAIN_PERFORMANCE_MAS12-37 | GRAIN_QUALITY | 25 |
| GRAIN_PERFORMANCE_MAS12-38 | GRAIN_QUALITY | 281 |
| GRAIN_PERFORMANCE_MAS12-40 | GRAIN_QUALITY | 87 |
| GRAIN_PERFORMANCE_MAS12-40 | GRAIN_QUALITY | 87 |
| GRAIN_PERFORMANCE_MAS12-40 | GRAIN_QUALITY | 217 |
| GRAIN_PERFORMANCE_MAS12-40 | GRAIN_QUALITY | 33 |
| GRAIN_PERFORMANCE_MAS12-41 | GRAIN_QUALITY | 285 |
| GRAIN_PERFORMANCE_MAS12-41 | GRAIN_QUALITY | 359 |
| GRAIN_PERFORMANCE_MAS12-41 | GRAIN_QUALITY | 233 |
| GRAIN_PERFORMANCE_MAS12-41 | GRAIN_QUALITY | 359 |
| GRAIN_PERFORMANCE_MAS12-41 | GRAIN_QUALITY | 233 |
| GRAIN_PERFORMANCE_MAS12-41 | GRAIN_QUALITY | 281 |
| GRAIN_PERFORMANCE_MAS12-41 | GRAIN_QUALITY | 233 |
| GRAIN_PERFORMANCE_MAS12-41 | GRAIN_QUALITY | 285 |
| GRAIN_PERFORMANCE_MAS12-41 | GRAIN_QUALITY | 233 |
| GRAIN_PERFORMANCE_MAS12-41 | GRAIN_QUALITY | 33 |
| GRAIN_PERFORMANCE_MAS12-42 | GRAIN_QUALITY | 285 |
| GRAIN_PERFORMANCE_MAS12-42 | GRAIN_QUALITY | 349 |
| GRAIN_PERFORMANCE_MAS12-42 | GRAIN_QUALITY | 349 |
| GRAIN_PERFORMANCE_MAS12-42 | GRAIN_QUALITY | 57 |
| GRAIN_PERFORMANCE_MAS12-42 | GRAIN_QUALITY | 285 |
| GRAIN_PERFORMANCE_MAS12-43 | GRAIN_QUALITY | 131 |
| GRAIN_PERFORMANCE_MAS12-43 | GRAIN_QUALITY | 349 |
| GRAIN_PERFORMANCE_MAS12-43 | GRAIN_QUALITY | 197 |
| GRAIN_PERFORMANCE_MAS12-43 | GRAIN_QUALITY | 131 |
| GRAIN_PERFORMANCE_MAS12-43 | GRAIN_QUALITY | 349 |
| GRAIN_PERFORMANCE_MAS12-44 | GRAIN_QUALITY | 71 |
| GRAIN_PERFORMANCE_MAS12-44 | GRAIN_QUALITY | 349 |
| GRAIN_PERFORMANCE_MAS12-44 | GRAIN_QUALITY | 349 |
| GRAIN_PERFORMANCE_MAS12-44 | GRAIN_QUALITY | 321 |
| GRAIN_PERFORMANCE_MAS12-47 | GRAIN_QUALITY | 285 |
| GRAIN_PERFORMANCE_MAS12-47 | GRAIN_QUALITY | 131 |
| GRAIN_PERFORMANCE_MAS12-47 | GRAIN_QUALITY | 349 |
| GRAIN_PERFORMANCE_MAS12-47 | GRAIN_QUALITY | 197 |
| GRAIN_PERFORMANCE_MAS12-47 | GRAIN_QUALITY | 263 |
| GRAIN_PERFORMANCE_MAS12-47 | GRAIN_QUALITY | 131 |
| GRAIN_PERFORMANCE_MAS12-47 | GRAIN_QUALITY | 263 |
| GRAIN_PERFORMANCE_MAS12-47 | GRAIN_QUALITY | 349 |
| GRAIN_PERFORMANCE_MAS12-47 | GRAIN_QUALITY | 285 |
| GRAIN_PERFORMANCE_MAS12-47 | GRAIN_QUALITY | 263 |
| GRAIN_PERFORMANCE_MAS12-47 | GRAIN_QUALITY | 237 |
| GRAIN_PERFORMANCE_MAS12-58 | GRAIN_QUALITY | 291 |
| GRAIN_PERFORMANCE_MAS12-58 | GRAIN_QUALITY | 273 |
| GRAIN_PERFORMANCE_MAS12-58 | GRAIN_QUALITY | 71 |
| GRAIN_PERFORMANCE_MAS12-58 | GRAIN_QUALITY | 291 |
| GRAIN_PERFORMANCE_MAS12-58 | GRAIN_QUALITY | 25 |
| GRAIN_PERFORMANCE_MAS12-58 | GRAIN_QUALITY | 273 |
| GRAIN_PERFORMANCE_MAS12-58 | GRAIN_QUALITY | 291 |
| GRAIN_PERFORMANCE_MAS12-58 | GRAIN_QUALITY | 291 |
| GRAIN_PERFORMANCE_MAS12-58 | GRAIN_QUALITY | 273 |
| GRAIN_PERFORMANCE_MAS12-58 | GRAIN_QUALITY | 25 |
| GRAIN_PERFORMANCE_MAS12-58 | GRAIN_QUALITY | 57 |
| GRAIN_PERFORMANCE_MAS12-58 | GRAIN_QUALITY | 291 |
| GRAIN_PERFORMANCE_MAS12-58 | GRAIN_QUALITY | 273 |
| GRAIN_PERFORMANCE_MAS12-58 | GRAIN_QUALITY | 33 |
| GRAIN_PERFORMANCE_MAS13-41 | GRAIN_QUALITY | 319 |
| GRAIN_PERFORMANCE_MAS13-43 | GRAIN_QUALITY | 131 |
| GRAIN_PERFORMANCE_MAS13-43 | GRAIN_QUALITY | 87 |
| GRAIN_PERFORMANCE_MAS13-43 | GRAIN_QUALITY | 349 |
| GRAIN_PERFORMANCE_MAS13-43 | GRAIN_QUALITY | 197 |
| GRAIN_PERFORMANCE_MAS13-43 | GRAIN_QUALITY | 331 |
| GRAIN_PERFORMANCE_MAS13-43 | GRAIN_QUALITY | 131 |
| GRAIN_PERFORMANCE_MAS13-43 | GRAIN_QUALITY | 349 |
| GRAIN_PERFORMANCE_MAS13-43 | GRAIN_QUALITY | 331 |
| GRAIN_PERFORMANCE_MAS13-43 | GRAIN_QUALITY | 87 |
| GRAIN_PERFORMANCE_MAS13-43 | GRAIN_QUALITY | 319 |
| GRAIN_PERFORMANCE_MAS13-44 | GRAIN_QUALITY | 319 |
| GRAIN_PERFORMANCE_MAS13-56 | GRAIN_QUALITY | 99 |
| GRAIN_PERFORMANCE_MAS13-56 | GRAIN_QUALITY | 331 |
| GRAIN_PERFORMANCE_MAS13-56 | GRAIN_QUALITY | 331 |
| GRAIN_PERFORMANCE_MAS16-1 | GRAIN_QUALITY | 319 |
| GRAIN_PERFORMANCE_MAS16-10 | GRAIN_QUALITY | 285 |
| GRAIN_PERFORMANCE_MAS16-10 | GRAIN_QUALITY | 87 |
| GRAIN_PERFORMANCE_MAS16-10 | GRAIN_QUALITY | 87 |
| GRAIN_PERFORMANCE_MAS16-10 | GRAIN_QUALITY | 285 |
| GRAIN_PERFORMANCE_MAS16-10 | GRAIN_QUALITY | 217 |
| GRAIN_PERFORMANCE_MAS16-11 | GRAIN_QUALITY | 281 |
| GRAIN_PERFORMANCE_MAS16-12 | GRAIN_QUALITY | 59 |
| GRAIN_PERFORMANCE_MAS16-13 | GRAIN_QUALITY | 285 |
| GRAIN_PERFORMANCE_MAS16-13 | GRAIN_QUALITY | 87 |
| GRAIN_PERFORMANCE_MAS16-13 | GRAIN_QUALITY | 87 |
| GRAIN_PERFORMANCE_MAS16-13 | GRAIN_QUALITY | 285 |
| GRAIN_PERFORMANCE_MAS16-4 | GRAIN_QUALITY | 285 |
| GRAIN_PERFORMANCE_MAS16-4 | GRAIN_QUALITY | 87 |
| GRAIN_PERFORMANCE_MAS16-4 | GRAIN_QUALITY | 87 |
| GRAIN_PERFORMANCE_MAS16-4 | GRAIN_QUALITY | 285 |
| GRAIN_PERFORMANCE_MAS16-6 | GRAIN_QUALITY | 71 |
| GRAIN_PERFORMANCE_MAS16-6 | GRAIN_QUALITY | 349 |
| GRAIN_PERFORMANCE_MAS16-6 | GRAIN_QUALITY | 349 |
| GRAIN_PERFORMANCE_MAS16-6 | GRAIN_QUALITY | 321 |
| GRAIN_PERFORMANCE_MAS16-8 | GRAIN_QUALITY | 281 |
| GRAIN_PERFORMANCE_MAS19-1 | GRAIN_QUALITY | 319 |
| GRAIN_PERFORMANCE_MAS19-10 | GRAIN_QUALITY | 291 |
| GRAIN_PERFORMANCE_MAS19-10 | GRAIN_QUALITY | 291 |
| GRAIN_PERFORMANCE_MAS19-10 | GRAIN_QUALITY | 87 |
| GRAIN_PERFORMANCE_MAS19-10 | GRAIN_QUALITY | 291 |
| GRAIN_PERFORMANCE_MAS19-10 | GRAIN_QUALITY | 291 |
| GRAIN_PERFORMANCE_MAS19-10 | GRAIN_QUALITY | 87 |
| GRAIN_PERFORMANCE_MAS19-10 | GRAIN_QUALITY | 291 |
| GRAIN_PERFORMANCE_MAS19-13 | GRAIN_QUALITY | 281 |
| GRAIN_PERFORMANCE_MAS19-2 | GRAIN_QUALITY | 285 |
| GRAIN_PERFORMANCE_MAS19-2 | GRAIN_QUALITY | 87 |
| GRAIN_PERFORMANCE_MAS19-2 | GRAIN_QUALITY | 87 |
| GRAIN_PERFORMANCE_MAS19-2 | GRAIN_QUALITY | 285 |
| GRAIN_PERFORMANCE_MAS19-2 | GRAIN_QUALITY | 217 |
| GRAIN_PERFORMANCE_MAS19-5 | GRAIN_QUALITY | 87 |
| GRAIN_PERFORMANCE_MAS19-5 | GRAIN_QUALITY | 87 |
| GRAIN_PERFORMANCE_MAS19-6 | GRAIN_QUALITY | 87 |
| GRAIN_PERFORMANCE_MAS19-6 | GRAIN_QUALITY | 87 |
| GRAIN_PERFORMANCE_MAS19-7 | GRAIN_QUALITY | 285 |
| GRAIN_PERFORMANCE_MAS19-7 | GRAIN_QUALITY | 87 |
| GRAIN_PERFORMANCE_MAS19-7 | GRAIN_QUALITY | 331 |
| GRAIN_PERFORMANCE_MAS19-7 | GRAIN_QUALITY | 331 |
| GRAIN_PERFORMANCE_MAS19-7 | GRAIN_QUALITY | 87 |
| GRAIN_PERFORMANCE_MAS19-7 | GRAIN_QUALITY | 57 |
| GRAIN_PERFORMANCE_MAS19-7 | GRAIN_QUALITY | 285 |
| GRAIN_PERFORMANCE_MAS19-7 | GRAIN_QUALITY | 217 |
| GRAIN_PERFORMANCE_MAS19-8 | GRAIN_QUALITY | 285 |
| GRAIN_PERFORMANCE_MAS19-8 | GRAIN_QUALITY | 87 |
| GRAIN_PERFORMANCE_MAS19-8 | GRAIN_QUALITY | 331 |
| GRAIN_PERFORMANCE_MAS19-8 | GRAIN_QUALITY | 331 |
| GRAIN_PERFORMANCE_MAS19-8 | GRAIN_QUALITY | 87 |
| GRAIN_PERFORMANCE_MAS19-8 | GRAIN_QUALITY | 57 |
| GRAIN_PERFORMANCE_MAS19-8 | GRAIN_QUALITY | 285 |
| GRAIN_PERFORMANCE_MAS19-8 | GRAIN_QUALITY | 217 |
| GRAIN_PERFORMANCE_MAS19-9 | GRAIN_QUALITY | 99 |
| GRAIN_PERFORMANCE_MAS19-9 | GRAIN_QUALITY | 331 |
| GRAIN_PERFORMANCE_MAS19-9 | GRAIN_QUALITY | 331 |
| GRAIN_PERFORMANCE_MAS20-1 | GRAIN_QUALITY | 71 |
| GRAIN_PERFORMANCE_MAS20-1 | GRAIN_QUALITY | 349 |
| GRAIN_PERFORMANCE_MAS20-1 | GRAIN_QUALITY | 349 |
| GRAIN_PERFORMANCE_MAS20-1 | GRAIN_QUALITY | 321 |
| GRAIN_PERFORMANCE_MAS20-2 | GRAIN_QUALITY | 285 |
| GRAIN_PERFORMANCE_MAS20-2 | GRAIN_QUALITY | 359 |
| GRAIN_PERFORMANCE_MAS20-2 | GRAIN_QUALITY | 233 |
| GRAIN_PERFORMANCE_MAS20-2 | GRAIN_QUALITY | 359 |
| GRAIN_PERFORMANCE_MAS20-2 | GRAIN_QUALITY | 233 |
| GRAIN_PERFORMANCE_MAS20-2 | GRAIN_QUALITY | 281 |
| GRAIN_PERFORMANCE_MAS20-2 | GRAIN_QUALITY | 233 |
| GRAIN_PERFORMANCE_MAS20-2 | GRAIN_QUALITY | 285 |
| GRAIN_PERFORMANCE_MAS20-2 | GRAIN_QUALITY | 233 |
| GRAIN_PERFORMANCE_MAS20-2 | GRAIN_QUALITY | 33 |
| GRAIN_PERFORMANCE_MAS20-24 | GRAIN_QUALITY | 87 |
| GRAIN_PERFORMANCE_MAS20-24 | GRAIN_QUALITY | 87 |
| GRAIN_PERFORMANCE_MAS20-3 | GRAIN_QUALITY | 285 |
| GRAIN_PERFORMANCE_MAS20-3 | GRAIN_QUALITY | 349 |
| GRAIN_PERFORMANCE_MAS20-3 | GRAIN_QUALITY | 349 |
| GRAIN_PERFORMANCE_MAS20-3 | GRAIN_QUALITY | 57 |
| GRAIN_PERFORMANCE_MAS20-3 | GRAIN_QUALITY | 285 |
| GRAIN_PERFORMANCE_MAS20-4 | GRAIN_QUALITY | 285 |
| GRAIN_PERFORMANCE_MAS20-4 | GRAIN_QUALITY | 349 |
| GRAIN_PERFORMANCE_MAS20-4 | GRAIN_QUALITY | 349 |
| GRAIN_PERFORMANCE_MAS20-4 | GRAIN_QUALITY | 57 |
| GRAIN_PERFORMANCE_MAS20-4 | GRAIN_QUALITY | 285 |
| GRAIN_PERFORMANCE_MAS20-5 | GRAIN_QUALITY | 285 |
| GRAIN_PERFORMANCE_MAS20-5 | GRAIN_QUALITY | 87 |
| GRAIN_PERFORMANCE_MAS20-5 | GRAIN_QUALITY | 87 |
| GRAIN_PERFORMANCE_MAS20-5 | GRAIN_QUALITY | 285 |
| GRAIN_PERFORMANCE_MAS20-5 | GRAIN_QUALITY | 217 |
| GRAIN_PERFORMANCE_MAS20-6 | GRAIN_QUALITY | 285 |
| GRAIN_PERFORMANCE_MAS20-6 | GRAIN_QUALITY | 87 |
| GRAIN_PERFORMANCE_MAS20-6 | GRAIN_QUALITY | 87 |
| GRAIN_PERFORMANCE_MAS20-6 | GRAIN_QUALITY | 285 |
| GRAIN_PERFORMANCE_MAS20-6 | GRAIN_QUALITY | 217 |
| GRAIN_PERFORMANCE_MAS20-7 | GRAIN_QUALITY | 131 |
| GRAIN_PERFORMANCE_MAS20-7 | GRAIN_QUALITY | 87 |
| GRAIN_PERFORMANCE_MAS20-7 | GRAIN_QUALITY | 197 |
| GRAIN_PERFORMANCE_MAS20-7 | GRAIN_QUALITY | 331 |
| GRAIN_PERFORMANCE_MAS20-7 | GRAIN_QUALITY | 131 |
| GRAIN_PERFORMANCE_MAS20-7 | GRAIN_QUALITY | 331 |
| GRAIN_PERFORMANCE_MAS20-7 | GRAIN_QUALITY | 87 |
| GRAIN_PERFORMANCE_MAS20-7 | GRAIN_QUALITY | 237 |
| GRAIN_PERFORMANCE_MAS20-7 | GRAIN_QUALITY | 319 |
| GRAIN_PERFORMANCE_MAS20-8 | GRAIN_QUALITY | 319 |
| GRAIN_PERFORMANCE_MAS20-9 | GRAIN_QUALITY | 71 |
| GRAIN_PERFORMANCE_MAS20-9 | GRAIN_QUALITY | 217 |
| GRAIN_PERFORMANCE_MAS21-1 | GRAIN_QUALITY | 87 |
| GRAIN_PERFORMANCE_MAS21-1 | GRAIN_QUALITY | 87 |
| GRAIN_PERFORMANCE_MAS21-12 | GRAIN_QUALITY | 87 |
| GRAIN_PERFORMANCE_MAS21-12 | GRAIN_QUALITY | 87 |
| GRAIN_PERFORMANCE_MAS21-12 | GRAIN_QUALITY | 217 |
| GRAIN_PERFORMANCE_MAS21-2 | GRAIN_QUALITY | 87 |
| GRAIN_PERFORMANCE_MAS21-2 | GRAIN_QUALITY | 87 |
| GRAIN_PERFORMANCE_MAS21-4 | GRAIN_QUALITY | 87 |
| GRAIN_PERFORMANCE_MAS21-4 | GRAIN_QUALITY | 87 |
| GRAIN_PERFORMANCE_MAS21-4 | GRAIN_QUALITY | 217 |
| GRAIN_PERFORMANCE_MAS21-5 | GRAIN_QUALITY | 281 |
| GRAIN_PERFORMANCE_MAS21-6 | GRAIN_QUALITY | 291 |
| GRAIN_PERFORMANCE_MAS21-6 | GRAIN_QUALITY | 291 |
| GRAIN_PERFORMANCE_MAS21-6 | GRAIN_QUALITY | 291 |
| GRAIN_PERFORMANCE_MAS21-6 | GRAIN_QUALITY | 291 |
| GRAIN_PERFORMANCE_MAS21-6 | GRAIN_QUALITY | 291 |
| GRAIN_PERFORMANCE_MAS21-7 | GRAIN_QUALITY | 291 |
| GRAIN_PERFORMANCE_MAS21-7 | GRAIN_QUALITY | 291 |
| GRAIN_PERFORMANCE_MAS21-7 | GRAIN_QUALITY | 291 |
| GRAIN_PERFORMANCE_MAS21-7 | GRAIN_QUALITY | 291 |
| GRAIN_PERFORMANCE_MAS21-7 | GRAIN_QUALITY | 291 |
| GRAIN_PERFORMANCE_MAS21-9 | GRAIN_QUALITY | 25 |
| GRAIN_PERFORMANCE_MAS21-9 | GRAIN_QUALITY | 25 |
| GRAIN_PERFORMANCE_MAS22-1 | GRAIN_QUALITY | 285 |
| GRAIN_PERFORMANCE_MAS22-1 | GRAIN_QUALITY | 131 |
| GRAIN_PERFORMANCE_MAS22-1 | GRAIN_QUALITY | 349 |
| GRAIN_PERFORMANCE_MAS22-1 | GRAIN_QUALITY | 197 |
| GRAIN_PERFORMANCE_MAS22-1 | GRAIN_QUALITY | 263 |
| GRAIN_PERFORMANCE_MAS22-1 | GRAIN_QUALITY | 131 |
| GRAIN_PERFORMANCE_MAS22-1 | GRAIN_QUALITY | 263 |
| GRAIN_PERFORMANCE_MAS22-1 | GRAIN_QUALITY | 349 |
| GRAIN_PERFORMANCE_MAS22-1 | GRAIN_QUALITY | 285 |
| GRAIN_PERFORMANCE_MAS22-1 | GRAIN_QUALITY | 263 |
| GRAIN_PERFORMANCE_MAS22-1 | GRAIN_QUALITY | 237 |
| GRAIN_PERFORMANCE_MAS22-17 | GRAIN_QUALITY | 285 |
| GRAIN_PERFORMANCE_MAS22-17 | GRAIN_QUALITY | 87 |
| GRAIN_PERFORMANCE_MAS22-17 | GRAIN_QUALITY | 87 |
| GRAIN_PERFORMANCE_MAS22-17 | GRAIN_QUALITY | 285 |
| GRAIN_PERFORMANCE_MAS22-18 | GRAIN_QUALITY | 285 |
| GRAIN_PERFORMANCE_MAS22-18 | GRAIN_QUALITY | 87 |
| GRAIN_PERFORMANCE_MAS22-18 | GRAIN_QUALITY | 87 |
| GRAIN_PERFORMANCE_MAS22-18 | GRAIN_QUALITY | 285 |
| GRAIN_PERFORMANCE_MAS22-19 | GRAIN_QUALITY | 285 |
| GRAIN_PERFORMANCE_MAS22-19 | GRAIN_QUALITY | 87 |
| GRAIN_PERFORMANCE_MAS22-19 | GRAIN_QUALITY | 87 |
| GRAIN_PERFORMANCE_MAS22-19 | GRAIN_QUALITY | 285 |
| GRAIN_PERFORMANCE_MAS22-2 | GRAIN_QUALITY | 291 |
| GRAIN_PERFORMANCE_MAS22-2 | GRAIN_QUALITY | 273 |
| GRAIN_PERFORMANCE_MAS22-2 | GRAIN_QUALITY | 291 |
| GRAIN_PERFORMANCE_MAS22-2 | GRAIN_QUALITY | 273 |
| GRAIN_PERFORMANCE_MAS22-2 | GRAIN_QUALITY | 291 |
| GRAIN_PERFORMANCE_MAS22-2 | GRAIN_QUALITY | 291 |
| GRAIN_PERFORMANCE_MAS22-2 | GRAIN_QUALITY | 273 |
| GRAIN_PERFORMANCE_MAS22-2 | GRAIN_QUALITY | 57 |
| GRAIN_PERFORMANCE_MAS22-2 | GRAIN_QUALITY | 291 |
| GRAIN_PERFORMANCE_MAS22-2 | GRAIN_QUALITY | 273 |
| GRAIN_PERFORMANCE_MAS22-20 | GRAIN_QUALITY | 291 |
| GRAIN_PERFORMANCE_MAS22-20 | GRAIN_QUALITY | 291 |
| GRAIN_PERFORMANCE_MAS22-20 | GRAIN_QUALITY | 291 |
| GRAIN_PERFORMANCE_MAS22-20 | GRAIN_QUALITY | 291 |
| GRAIN_PERFORMANCE_MAS22-20 | GRAIN_QUALITY | 291 |
| GRAIN_PERFORMANCE_MAS22-21 | GRAIN_QUALITY | 291 |
| GRAIN_PERFORMANCE_MAS22-21 | GRAIN_QUALITY | 291 |
| GRAIN_PERFORMANCE_MAS22-21 | GRAIN_QUALITY | 291 |
| GRAIN_PERFORMANCE_MAS22-21 | GRAIN_QUALITY | 291 |
| GRAIN_PERFORMANCE_MAS22-21 | GRAIN_QUALITY | 291 |
| GRAIN_PERFORMANCE_MAS22-3 | GRAIN_QUALITY | 291 |
| GRAIN_PERFORMANCE_MAS22-3 | GRAIN_QUALITY | 273 |
| GRAIN_PERFORMANCE_MAS22-3 | GRAIN_QUALITY | 291 |
| GRAIN_PERFORMANCE_MAS22-3 | GRAIN_QUALITY | 273 |
| GRAIN_PERFORMANCE_MAS22-3 | GRAIN_QUALITY | 291 |
| GRAIN_PERFORMANCE_MAS22-3 | GRAIN_QUALITY | 291 |
| GRAIN_PERFORMANCE_MAS22-3 | GRAIN_QUALITY | 273 |
| GRAIN_PERFORMANCE_MAS22-3 | GRAIN_QUALITY | 57 |
| GRAIN_PERFORMANCE_MAS22-3 | GRAIN_QUALITY | 291 |
| GRAIN_PERFORMANCE_MAS22-3 | GRAIN_QUALITY | 273 |
| GRAIN_PERFORMANCE_MAS22-4 | GRAIN_QUALITY | 291 |
| GRAIN_PERFORMANCE_MAS22-4 | GRAIN_QUALITY | 273 |
| GRAIN_PERFORMANCE_MAS22-4 | GRAIN_QUALITY | 291 |
| GRAIN_PERFORMANCE_MAS22-4 | GRAIN_QUALITY | 273 |
| GRAIN_PERFORMANCE_MAS22-4 | GRAIN_QUALITY | 291 |
| GRAIN_PERFORMANCE_MAS22-4 | GRAIN_QUALITY | 291 |
| GRAIN_PERFORMANCE_MAS22-4 | GRAIN_QUALITY | 273 |
| GRAIN_PERFORMANCE_MAS22-4 | GRAIN_QUALITY | 57 |
| GRAIN_PERFORMANCE_MAS22-4 | GRAIN_QUALITY | 291 |
| GRAIN_PERFORMANCE_MAS22-4 | GRAIN_QUALITY | 273 |
| GRAIN_PERFORMANCE_MAS22-5 | GRAIN_QUALITY | 319 |
| GRAIN_PERFORMANCE_MAS22-6 | GRAIN_QUALITY | 319 |
| GRAIN_PERFORMANCE_MAS22-7 | GRAIN_QUALITY | 71 |
| GRAIN_PERFORMANCE_MAS22-7 | GRAIN_QUALITY | 131 |
| GRAIN_PERFORMANCE_MAS22-7 | GRAIN_QUALITY | 349 |
| GRAIN_PERFORMANCE_MAS22-7 | GRAIN_QUALITY | 197 |
| GRAIN_PERFORMANCE_MAS22-7 | GRAIN_QUALITY | 263 |
| GRAIN_PERFORMANCE_MAS22-7 | GRAIN_QUALITY | 131 |
| GRAIN_PERFORMANCE_MAS22-7 | GRAIN_QUALITY | 257 |
| GRAIN_PERFORMANCE_MAS22-7 | GRAIN_QUALITY | 263 |
| GRAIN_PERFORMANCE_MAS22-7 | GRAIN_QUALITY | 349 |
| GRAIN_PERFORMANCE_MAS22-7 | GRAIN_QUALITY | 321 |
| GRAIN_PERFORMANCE_MAS22-7 | GRAIN_QUALITY | 263 |
| GRAIN_PERFORMANCE_MAS22-7 | GRAIN_QUALITY | 237 |
| GRAIN_PERFORMANCE_MAS22-8 | GRAIN_QUALITY | 71 |
| GRAIN_PERFORMANCE_MAS22-8 | GRAIN_QUALITY | 131 |
| GRAIN_PERFORMANCE_MAS22-8 | GRAIN_QUALITY | 349 |
| GRAIN_PERFORMANCE_MAS22-8 | GRAIN_QUALITY | 197 |
| GRAIN_PERFORMANCE_MAS22-8 | GRAIN_QUALITY | 263 |
| GRAIN_PERFORMANCE_MAS22-8 | GRAIN_QUALITY | 131 |
| GRAIN_PERFORMANCE_MAS22-8 | GRAIN_QUALITY | 257 |
| GRAIN_PERFORMANCE_MAS22-8 | GRAIN_QUALITY | 263 |
| GRAIN_PERFORMANCE_MAS22-8 | GRAIN_QUALITY | 349 |
| GRAIN_PERFORMANCE_MAS22-8 | GRAIN_QUALITY | 321 |
| GRAIN_PERFORMANCE_MAS22-8 | GRAIN_QUALITY | 263 |
| GRAIN_PERFORMANCE_MAS22-8 | GRAIN_QUALITY | 237 |
| GRAIN_PERFORMANCE_MAS22-9 | GRAIN_QUALITY | 131 |
| GRAIN_PERFORMANCE_MAS22-9 | GRAIN_QUALITY | 197 |
| GRAIN_PERFORMANCE_MAS22-9 | GRAIN_QUALITY | 131 |
| GRAIN_PERFORMANCE_MAS22-9 | GRAIN_QUALITY | 237 |
| GRAIN_PERFORMANCE_MAS23-11 | GRAIN_QUALITY | 263 |
| GRAIN_PERFORMANCE_MAS23-11 | GRAIN_QUALITY | 331 |
| GRAIN_PERFORMANCE_MAS23-11 | GRAIN_QUALITY | 263 |
| GRAIN_PERFORMANCE_MAS23-11 | GRAIN_QUALITY | 331 |
| GRAIN_PERFORMANCE_MAS23-11 | GRAIN_QUALITY | 263 |
| GRAIN_PERFORMANCE_MAS23-12 | GRAIN_QUALITY | 319 |
| GRAIN_PERFORMANCE_MAS23-20 | GRAIN_QUALITY | 319 |
| GRAIN_PERFORMANCE_MAS23-21 | GRAIN_QUALITY | 319 |
| GRAIN_PERFORMANCE_MAS23-22 | GRAIN_QUALITY | 319 |
| GRAIN_PERFORMANCE_MAS23-4 | GRAIN_QUALITY | 285 |
| GRAIN_PERFORMANCE_MAS23-4 | GRAIN_QUALITY | 87 |
| GRAIN_PERFORMANCE_MAS23-4 | GRAIN_QUALITY | 331 |
| GRAIN_PERFORMANCE_MAS23-4 | GRAIN_QUALITY | 331 |
| GRAIN_PERFORMANCE_MAS23-4 | GRAIN_QUALITY | 87 |
| GRAIN_PERFORMANCE_MAS23-4 | GRAIN_QUALITY | 57 |
| GRAIN_PERFORMANCE_MAS23-4 | GRAIN_QUALITY | 285 |
| GRAIN_PERFORMANCE_MAS23-4 | GRAIN_QUALITY | 217 |
| GRAIN_PERFORMANCE_MAS23-5 | GRAIN_QUALITY | 291 |
| GRAIN_PERFORMANCE_MAS23-5 | GRAIN_QUALITY | 291 |
| GRAIN_PERFORMANCE_MAS23-5 | GRAIN_QUALITY | 291 |
| GRAIN_PERFORMANCE_MAS23-5 | GRAIN_QUALITY | 291 |
| GRAIN_PERFORMANCE_MAS23-5 | GRAIN_QUALITY | 291 |
| GRAIN_PERFORMANCE_MAS23-6 | GRAIN_QUALITY | 281 |
| GRAIN_PERFORMANCE_MAS23-7 | GRAIN_QUALITY | 285 |
| GRAIN_PERFORMANCE_MAS23-7 | GRAIN_QUALITY | 87 |
| GRAIN_PERFORMANCE_MAS23-7 | GRAIN_QUALITY | 87 |
| GRAIN_PERFORMANCE_MAS23-7 | GRAIN_QUALITY | 285 |
| GRAIN_PERFORMANCE_MAS23-8 | GRAIN_QUALITY | 285 |
| GRAIN_PERFORMANCE_MAS23-8 | GRAIN_QUALITY | 87 |
| GRAIN_PERFORMANCE_MAS23-8 | GRAIN_QUALITY | 87 |
| GRAIN_PERFORMANCE_MAS23-8 | GRAIN_QUALITY | 285 |
| GRAIN_PERFORMANCE_MAS24-23 | GRAIN_QUALITY | 319 |
| GRAIN_PERFORMANCE_MAS24-24 | GRAIN_QUALITY | 319 |
| GRAIN_PERFORMANCE_MAS24-25 | GRAIN_QUALITY | 131 |
| GRAIN_PERFORMANCE_MAS24-25 | GRAIN_QUALITY | 197 |
| GRAIN_PERFORMANCE_MAS24-25 | GRAIN_QUALITY | 263 |
| GRAIN_PERFORMANCE_MAS24-25 | GRAIN_QUALITY | 131 |
| GRAIN_PERFORMANCE_MAS24-25 | GRAIN_QUALITY | 257 |
| GRAIN_PERFORMANCE_MAS24-25 | GRAIN_QUALITY | 263 |
| GRAIN_PERFORMANCE_MAS24-25 | GRAIN_QUALITY | 263 |
| GRAIN_PERFORMANCE_MAS24-25 | GRAIN_QUALITY | 237 |
| GRAIN_PERFORMANCE_MAS24-26 | GRAIN_QUALITY | 87 |
| GRAIN_PERFORMANCE_MAS24-26 | GRAIN_QUALITY | 87 |
| GRAIN_PERFORMANCE_MAS25-1 | GRAIN_QUALITY | 285 |
| GRAIN_PERFORMANCE_MAS25-1 | GRAIN_QUALITY | 263 |
| GRAIN_PERFORMANCE_MAS25-1 | GRAIN_QUALITY | 331 |
| GRAIN_PERFORMANCE_MAS25-1 | GRAIN_QUALITY | 263 |
| GRAIN_PERFORMANCE_MAS25-1 | GRAIN_QUALITY | 331 |
| GRAIN_PERFORMANCE_MAS25-1 | GRAIN_QUALITY | 285 |
| GRAIN_PERFORMANCE_MAS25-1 | GRAIN_QUALITY | 263 |
| GRAIN_PERFORMANCE_MAS25-13 | GRAIN_QUALITY | 349 |
| GRAIN_PERFORMANCE_MAS25-13 | GRAIN_QUALITY | 349 |
| GRAIN_PERFORMANCE_MAS25-13 | GRAIN_QUALITY | 57 |
| GRAIN_PERFORMANCE_MAS25-14 | GRAIN_QUALITY | 349 |
| GRAIN_PERFORMANCE_MAS25-14 | GRAIN_QUALITY | 349 |
| GRAIN_PERFORMANCE_MAS25-14 | GRAIN_QUALITY | 57 |
| GRAIN_PERFORMANCE_MAS25-17 | GRAIN_QUALITY | 87 |
| GRAIN_PERFORMANCE_MAS25-17 | GRAIN_QUALITY | 87 |
| GRAIN_PERFORMANCE_MAS25-19 | GRAIN_QUALITY | 319 |
| GRAIN_PERFORMANCE_MAS25-2 | GRAIN_QUALITY | 291 |
| GRAIN_PERFORMANCE_MAS25-2 | GRAIN_QUALITY | 291 |
| GRAIN_PERFORMANCE_MAS25-2 | GRAIN_QUALITY | 291 |
| GRAIN_PERFORMANCE_MAS25-2 | GRAIN_QUALITY | 291 |
| GRAIN_PERFORMANCE_MAS25-2 | GRAIN_QUALITY | 291 |
| GRAIN_PERFORMANCE_MAS25-20 | GRAIN_QUALITY | 319 |
| GRAIN_PERFORMANCE_MAS25-21 | GRAIN_QUALITY | 281 |
| GRAIN_PERFORMANCE_MAS25-22 | GRAIN_QUALITY | 87 |
| GRAIN_PERFORMANCE_MAS25-22 | GRAIN_QUALITY | 87 |
| GRAIN_PERFORMANCE_MAS25-23 | GRAIN_QUALITY | 131 |
| GRAIN_PERFORMANCE_MAS25-23 | GRAIN_QUALITY | 25 |
| GRAIN_PERFORMANCE_MAS25-23 | GRAIN_QUALITY | 197 |
| GRAIN_PERFORMANCE_MAS25-23 | GRAIN_QUALITY | 131 |
| GRAIN_PERFORMANCE_MAS25-23 | GRAIN_QUALITY | 25 |
| GRAIN_PERFORMANCE_MAS25-31 | GRAIN_QUALITY | 71 |
| GRAIN_PERFORMANCE_MAS25-31 | GRAIN_QUALITY | 131 |
| GRAIN_PERFORMANCE_MAS25-31 | GRAIN_QUALITY | 349 |
| GRAIN_PERFORMANCE_MAS25-31 | GRAIN_QUALITY | 197 |
| GRAIN_PERFORMANCE_MAS25-31 | GRAIN_QUALITY | 263 |
| GRAIN_PERFORMANCE_MAS25-31 | GRAIN_QUALITY | 131 |
| GRAIN_PERFORMANCE_MAS25-31 | GRAIN_QUALITY | 257 |
| GRAIN_PERFORMANCE_MAS25-31 | GRAIN_QUALITY | 263 |
| GRAIN_PERFORMANCE_MAS25-31 | GRAIN_QUALITY | 349 |
| GRAIN_PERFORMANCE_MAS25-31 | GRAIN_QUALITY | 321 |
| GRAIN_PERFORMANCE_MAS25-31 | GRAIN_QUALITY | 263 |
| GRAIN_PERFORMANCE_MAS25-31 | GRAIN_QUALITY | 237 |
| GRAIN_PERFORMANCE_MAS25-34 | GRAIN_QUALITY | 99 |
| GRAIN_PERFORMANCE_MAS25-34 | GRAIN_QUALITY | 331 |
| GRAIN_PERFORMANCE_MAS25-34 | GRAIN_QUALITY | 331 |
| GRAIN_PERFORMANCE_MAS25-36 | GRAIN_QUALITY | 291 |
| GRAIN_PERFORMANCE_MAS25-36 | GRAIN_QUALITY | 291 |
| GRAIN_PERFORMANCE_MAS25-36 | GRAIN_QUALITY | 291 |
| GRAIN_PERFORMANCE_MAS25-36 | GRAIN_QUALITY | 291 |
| GRAIN_PERFORMANCE_MAS25-36 | GRAIN_QUALITY | 291 |
| GRAIN_PERFORMANCE_MAS25-37 | GRAIN_QUALITY | 285 |
| GRAIN_PERFORMANCE_MAS25-37 | GRAIN_QUALITY | 349 |
| GRAIN_PERFORMANCE_MAS25-37 | GRAIN_QUALITY | 349 |
| GRAIN_PERFORMANCE_MAS25-37 | GRAIN_QUALITY | 57 |
| GRAIN_PERFORMANCE_MAS25-37 | GRAIN_QUALITY | 285 |
| GRAIN_PERFORMANCE_MAS25-39 | GRAIN_QUALITY | 349 |
| GRAIN_PERFORMANCE_MAS25-39 | GRAIN_QUALITY | 349 |
| GRAIN_PERFORMANCE_MAS25-39 | GRAIN_QUALITY | 57 |
| GRAIN_PERFORMANCE_MAS25-4 | GRAIN_QUALITY | 25 |
| GRAIN_PERFORMANCE_MAS25-4 | GRAIN_QUALITY | 25 |
| GRAIN_PERFORMANCE_MAS25-43 | GRAIN_QUALITY | 257 |
| GRAIN_PERFORMANCE_MAS25-48 | GRAIN_QUALITY | 319 |
| GRAIN_PERFORMANCE_MAS25-5 | GRAIN_QUALITY | 131 |
| GRAIN_PERFORMANCE_MAS25-5 | GRAIN_QUALITY | 87 |
| GRAIN_PERFORMANCE_MAS25-5 | GRAIN_QUALITY | 349 |
| GRAIN_PERFORMANCE_MAS25-5 | GRAIN_QUALITY | 197 |
| GRAIN_PERFORMANCE_MAS25-5 | GRAIN_QUALITY | 331 |
| GRAIN_PERFORMANCE_MAS25-5 | GRAIN_QUALITY | 131 |
| GRAIN_PERFORMANCE_MAS25-5 | GRAIN_QUALITY | 349 |
| GRAIN_PERFORMANCE_MAS25-5 | GRAIN_QUALITY | 331 |
| GRAIN_PERFORMANCE_MAS25-5 | GRAIN_QUALITY | 87 |
| GRAIN_PERFORMANCE_MAS25-5 | GRAIN_QUALITY | 319 |
| GRAIN_PERFORMANCE_MAS25-6 | GRAIN_QUALITY | 87 |
| GRAIN_PERFORMANCE_MAS25-6 | GRAIN_QUALITY | 87 |
| GRAIN_PERFORMANCE_MAS25-7 | GRAIN_QUALITY | 319 |
| GRAIN_PERFORMANCE_MAS25-9 | GRAIN_QUALITY | 285 |
| GRAIN_PERFORMANCE_MAS25-9 | GRAIN_QUALITY | 87 |
| GRAIN_PERFORMANCE_MAS25-9 | GRAIN_QUALITY | 87 |
| GRAIN_PERFORMANCE_MAS25-9 | GRAIN_QUALITY | 285 |
| GRAIN_PERFORMANCE_MAS27-1 | GRAIN_QUALITY | 285 |
| GRAIN_PERFORMANCE_MAS27-1 | GRAIN_QUALITY | 349 |
| GRAIN_PERFORMANCE_MAS27-1 | GRAIN_QUALITY | 349 |
| GRAIN_PERFORMANCE_MAS27-1 | GRAIN_QUALITY | 57 |
| GRAIN_PERFORMANCE_MAS27-1 | GRAIN_QUALITY | 285 |
| GRAIN_PERFORMANCE_MAS27-11 | GRAIN_QUALITY | 291 |
| GRAIN_PERFORMANCE_MAS27-11 | GRAIN_QUALITY | 291 |
| GRAIN_PERFORMANCE_MAS27-11 | GRAIN_QUALITY | 87 |
| GRAIN_PERFORMANCE_MAS27-11 | GRAIN_QUALITY | 291 |
| GRAIN_PERFORMANCE_MAS27-11 | GRAIN_QUALITY | 291 |
| GRAIN_PERFORMANCE_MAS27-11 | GRAIN_QUALITY | 87 |
| GRAIN_PERFORMANCE_MAS27-11 | GRAIN_QUALITY | 291 |
| GRAIN_PERFORMANCE_MAS27-12 | GRAIN_QUALITY | 257 |
| GRAIN_PERFORMANCE_MAS27-14 | GRAIN_QUALITY | 87 |
| GRAIN_PERFORMANCE_MAS27-14 | GRAIN_QUALITY | 87 |
| GRAIN_PERFORMANCE_MAS27-16 | GRAIN_QUALITY | 87 |
| GRAIN_PERFORMANCE_MAS27-16 | GRAIN_QUALITY | 87 |
| GRAIN_PERFORMANCE_MAS27-19 | GRAIN_QUALITY | 285 |
| GRAIN_PERFORMANCE_MAS27-19 | GRAIN_QUALITY | 349 |
| GRAIN_PERFORMANCE_MAS27-19 | GRAIN_QUALITY | 349 |
| GRAIN_PERFORMANCE_MAS27-19 | GRAIN_QUALITY | 57 |
| GRAIN_PERFORMANCE_MAS27-19 | GRAIN_QUALITY | 285 |
| GRAIN_PERFORMANCE_MAS27-2 | GRAIN_QUALITY | 87 |
| GRAIN_PERFORMANCE_MAS27-2 | GRAIN_QUALITY | 87 |
| GRAIN_PERFORMANCE_MAS27-6 | GRAIN_QUALITY | 131 |
| GRAIN_PERFORMANCE_MAS27-6 | GRAIN_QUALITY | 197 |
| GRAIN_PERFORMANCE_MAS27-6 | GRAIN_QUALITY | 263 |
| GRAIN_PERFORMANCE_MAS27-6 | GRAIN_QUALITY | 131 |
| GRAIN_PERFORMANCE_MAS27-6 | GRAIN_QUALITY | 257 |
| GRAIN_PERFORMANCE_MAS27-6 | GRAIN_QUALITY | 263 |
| GRAIN_PERFORMANCE_MAS27-6 | GRAIN_QUALITY | 263 |
| GRAIN_PERFORMANCE_MAS27-6 | GRAIN_QUALITY | 237 |
| GRAIN_PERFORMANCE_MAS27-7 | GRAIN_QUALITY | 131 |
| GRAIN_PERFORMANCE_MAS27-7 | GRAIN_QUALITY | 87 |
| GRAIN_PERFORMANCE_MAS27-7 | GRAIN_QUALITY | 349 |
| GRAIN_PERFORMANCE_MAS27-7 | GRAIN_QUALITY | 197 |
| GRAIN_PERFORMANCE_MAS27-7 | GRAIN_QUALITY | 331 |
| GRAIN_PERFORMANCE_MAS27-7 | GRAIN_QUALITY | 131 |
| GRAIN_PERFORMANCE_MAS27-7 | GRAIN_QUALITY | 349 |
| GRAIN_PERFORMANCE_MAS27-7 | GRAIN_QUALITY | 331 |
| GRAIN_PERFORMANCE_MAS27-7 | GRAIN_QUALITY | 87 |
| GRAIN_PERFORMANCE_MAS27-7 | GRAIN_QUALITY | 319 |
| GRAIN_PERFORMANCE_MAS27-8 | GRAIN_QUALITY | 291 |
| GRAIN_PERFORMANCE_MAS27-8 | GRAIN_QUALITY | 291 |
| GRAIN_PERFORMANCE_MAS27-8 | GRAIN_QUALITY | 291 |
| GRAIN_PERFORMANCE_MAS27-8 | GRAIN_QUALITY | 291 |
| GRAIN_PERFORMANCE_MAS27-8 | GRAIN_QUALITY | 291 |
| GRAIN_PERFORMANCE_MAS27-9 | GRAIN_QUALITY | 285 |
| GRAIN_PERFORMANCE_MAS27-9 | GRAIN_QUALITY | 87 |
| GRAIN_PERFORMANCE_MAS27-9 | GRAIN_QUALITY | 331 |
| GRAIN_PERFORMANCE_MAS27-9 | GRAIN_QUALITY | 331 |
| GRAIN_PERFORMANCE_MAS27-9 | GRAIN_QUALITY | 87 |
| GRAIN_PERFORMANCE_MAS27-9 | GRAIN_QUALITY | 57 |
| GRAIN_PERFORMANCE_MAS27-9 | GRAIN_QUALITY | 285 |
| GRAIN_PERFORMANCE_MAS27-9 | GRAIN_QUALITY | 217 |
| GRAIN_PERFORMANCE_SMS015-1 | GRAIN_QUALITY | 257 |
| GRAIN_PERFORMANCE_SMS015-2 | GRAIN_QUALITY | 349 |
| GRAIN_PERFORMANCE_SMS015-2 | GRAIN_QUALITY | 349 |
| GRAIN_PERFORMANCE_SMS015-2 | GRAIN_QUALITY | 57 |
| GRAIN_PERFORMANCE_SMS015-37 | GRAIN_QUALITY | 285 |
| GRAIN_PERFORMANCE_SMS015-37 | GRAIN_QUALITY | 87 |
| GRAIN_PERFORMANCE_SMS015-37 | GRAIN_QUALITY | 87 |
| GRAIN_PERFORMANCE_SMS015-37 | GRAIN_QUALITY | 285 |
| GRAIN_PERFORMANCE_SMS015-37 | GRAIN_QUALITY | 217 |
| GRAIN_PERFORMANCE_SMS015-38 | GRAIN_QUALITY | 263 |
| GRAIN_PERFORMANCE_SMS015-38 | GRAIN_QUALITY | 263 |
| GRAIN_PERFORMANCE_SMS015-38 | GRAIN_QUALITY | 263 |
| GRAIN_PERFORMANCE_SMS015-39 | GRAIN_QUALITY | 281 |
| GRAIN_PERFORMANCE_SMS015-41 | GRAIN_QUALITY | 291 |
| GRAIN_PERFORMANCE_SMS015-41 | GRAIN_QUALITY | 273 |
| GRAIN_PERFORMANCE_SMS015-41 | GRAIN_QUALITY | 71 |
| GRAIN_PERFORMANCE_SMS015-41 | GRAIN_QUALITY | 291 |
| GRAIN_PERFORMANCE_SMS015-41 | GRAIN_QUALITY | 25 |
| GRAIN_PERFORMANCE_SMS015-41 | GRAIN_QUALITY | 273 |
| GRAIN_PERFORMANCE_SMS015-41 | GRAIN_QUALITY | 291 |
| GRAIN_PERFORMANCE_SMS015-41 | GRAIN_QUALITY | 291 |
| GRAIN_PERFORMANCE_SMS015-41 | GRAIN_QUALITY | 273 |
| GRAIN_PERFORMANCE_SMS015-41 | GRAIN_QUALITY | 25 |
| GRAIN_PERFORMANCE_SMS015-41 | GRAIN_QUALITY | 57 |
| GRAIN_PERFORMANCE_SMS015-41 | GRAIN_QUALITY | 291 |
| GRAIN_PERFORMANCE_SMS015-41 | GRAIN_QUALITY | 273 |
| GRAIN_PERFORMANCE_SMS015-41 | GRAIN_QUALITY | 33 |
| GRAIN_PERFORMANCE_SMS015-42 | GRAIN_QUALITY | 285 |
| GRAIN_PERFORMANCE_SMS015-42 | GRAIN_QUALITY | 263 |
| GRAIN_PERFORMANCE_SMS015-42 | GRAIN_QUALITY | 331 |
| GRAIN_PERFORMANCE_SMS015-42 | GRAIN_QUALITY | 263 |
| GRAIN_PERFORMANCE_SMS015-42 | GRAIN_QUALITY | 331 |
| GRAIN_PERFORMANCE_SMS015-42 | GRAIN_QUALITY | 285 |
| GRAIN_PERFORMANCE_SMS015-42 | GRAIN_QUALITY | 263 |
| GRAIN_PERFORMANCE_SMS015-51 | GRAIN_QUALITY | 285 |
| GRAIN_PERFORMANCE_SMS015-51 | GRAIN_QUALITY | 87 |
| GRAIN_PERFORMANCE_SMS015-51 | GRAIN_QUALITY | 87 |
| GRAIN_PERFORMANCE_SMS015-51 | GRAIN_QUALITY | 285 |
| GRAIN_PERFORMANCE_SMS021-21 | GRAIN_QUALITY | 285 |
| GRAIN_PERFORMANCE_SMS021-21 | GRAIN_QUALITY | 359 |
| GRAIN_PERFORMANCE_SMS021-21 | GRAIN_QUALITY | 233 |
| GRAIN_PERFORMANCE_SMS021-21 | GRAIN_QUALITY | 359 |
| GRAIN_PERFORMANCE_SMS021-21 | GRAIN_QUALITY | 233 |
| GRAIN_PERFORMANCE_SMS021-21 | GRAIN_QUALITY | 281 |
| GRAIN_PERFORMANCE_SMS021-21 | GRAIN_QUALITY | 233 |
| GRAIN_PERFORMANCE_SMS021-21 | GRAIN_QUALITY | 285 |
| GRAIN_PERFORMANCE_SMS021-21 | GRAIN_QUALITY | 233 |
| GRAIN_PERFORMANCE_SMS021-21 | GRAIN_QUALITY | 33 |
| GRAIN_PERFORMANCE_SMS021-38 | GRAIN_QUALITY | 71 |
| GRAIN_PERFORMANCE_SMS021-38 | GRAIN_QUALITY | 217 |
| GRAIN_PERFORMANCE_SMS021-39 | GRAIN_QUALITY | 39 |
| GRAIN_PERFORMANCE_SMS021-41 | GRAIN_QUALITY | 285 |
| GRAIN_PERFORMANCE_SMS021-41 | GRAIN_QUALITY | 131 |
| GRAIN_PERFORMANCE_SMS021-41 | GRAIN_QUALITY | 349 |
| GRAIN_PERFORMANCE_SMS021-41 | GRAIN_QUALITY | 197 |
| GRAIN_PERFORMANCE_SMS021-41 | GRAIN_QUALITY | 263 |
| GRAIN_PERFORMANCE_SMS021-41 | GRAIN_QUALITY | 131 |
| GRAIN_PERFORMANCE_SMS021-41 | GRAIN_QUALITY | 263 |
| GRAIN_PERFORMANCE_SMS021-41 | GRAIN_QUALITY | 349 |
| GRAIN_PERFORMANCE_SMS021-41 | GRAIN_QUALITY | 285 |
| GRAIN_PERFORMANCE_SMS021-41 | GRAIN_QUALITY | 263 |
| GRAIN_PERFORMANCE_SMS021-41 | GRAIN_QUALITY | 237 |
| GRAIN_PERFORMANCE_SMS021-42 | GRAIN_QUALITY | 87 |
| GRAIN_PERFORMANCE_SMS021-42 | GRAIN_QUALITY | 87 |
| GRAIN_PERFORMANCE_SMS021-43 | GRAIN_QUALITY | 39 |
| GRAIN_PERFORMANCE_SMS021-44 | GRAIN_QUALITY | 71 |
| GRAIN_PERFORMANCE_SMS021-44 | GRAIN_QUALITY | 349 |
| GRAIN_PERFORMANCE_SMS021-44 | GRAIN_QUALITY | 349 |
| GRAIN_PERFORMANCE_SMS021-44 | GRAIN_QUALITY | 321 |
| GRAIN_PERFORMANCE_SMS021-45 | GRAIN_QUALITY | 131 |
| GRAIN_PERFORMANCE_SMS021-45 | GRAIN_QUALITY | 197 |
| GRAIN_PERFORMANCE_SMS021-45 | GRAIN_QUALITY | 131 |
| GRAIN_PERFORMANCE_SMS021-45 | GRAIN_QUALITY | 257 |
| GRAIN_PERFORMANCE_SMS021-45 | GRAIN_QUALITY | 319 |
| GRAIN_PERFORMANCE_SMS021-46 | GRAIN_QUALITY | 87 |
| GRAIN_PERFORMANCE_SMS021-46 | GRAIN_QUALITY | 87 |
| GRAIN_PERFORMANCE_SMS021-46 | GRAIN_QUALITY | 217 |
| GRAIN_PERFORMANCE_SMS021-48 | GRAIN_QUALITY | 87 |
| GRAIN_PERFORMANCE_SMS021-48 | GRAIN_QUALITY | 87 |
| GRAIN_PERFORMANCE_SMS021-50 | GRAIN_QUALITY | 25 |
| GRAIN_PERFORMANCE_SMS021-50 | GRAIN_QUALITY | 25 |
| GRAIN_PERFORMANCE_SMS021-69 | GRAIN_QUALITY | 285 |
| GRAIN_PERFORMANCE_SMS021-69 | GRAIN_QUALITY | 359 |
| GRAIN_PERFORMANCE_SMS021-69 | GRAIN_QUALITY | 233 |
| GRAIN_PERFORMANCE_SMS021-69 | GRAIN_QUALITY | 359 |
| GRAIN_PERFORMANCE_SMS021-69 | GRAIN_QUALITY | 233 |
| GRAIN_PERFORMANCE_SMS021-69 | GRAIN_QUALITY | 281 |
| GRAIN_PERFORMANCE_SMS021-69 | GRAIN_QUALITY | 233 |
| GRAIN_PERFORMANCE_SMS021-69 | GRAIN_QUALITY | 285 |
| GRAIN_PERFORMANCE_SMS021-69 | GRAIN_QUALITY | 233 |
| GRAIN_PERFORMANCE_SMS021-69 | GRAIN_QUALITY | 33 |
| GRAIN_PERFORMANCE_SMS021-70 | GRAIN_QUALITY | 349 |
| GRAIN_PERFORMANCE_SMS021-70 | GRAIN_QUALITY | 349 |
| GRAIN_PERFORMANCE_SMS021-70 | GRAIN_QUALITY | 57 |
| GRAIN_PERFORMANCE_SMS021-71 | GRAIN_QUALITY | 25 |
| GRAIN_PERFORMANCE_SMS021-71 | GRAIN_QUALITY | 25 |
| GRAIN_PERFORMANCE_SMS021-72 | GRAIN_QUALITY | 281 |
| GRAIN_PERFORMANCE_SMS021-82 | GRAIN_QUALITY | 319 |
| GRAIN_PERFORMANCE_SMS021-83 | GRAIN_QUALITY | 319 |
| GRAIN_QUALITY_MAS12-18 | GRAIN_QUALITY | 285 |
| GRAIN_QUALITY_MAS12-18 | GRAIN_QUALITY | 131 |
| GRAIN_QUALITY_MAS12-18 | GRAIN_QUALITY | 349 |
| GRAIN_QUALITY_MAS12-18 | GRAIN_QUALITY | 197 |
| GRAIN_QUALITY_MAS12-18 | GRAIN_QUALITY | 263 |
| GRAIN_QUALITY_MAS12-18 | GRAIN_QUALITY | 131 |
| GRAIN_QUALITY_MAS12-18 | GRAIN_QUALITY | 263 |
| GRAIN_QUALITY_MAS12-18 | GRAIN_QUALITY | 349 |
| GRAIN_QUALITY_MAS12-18 | GRAIN_QUALITY | 285 |
| GRAIN_QUALITY_MAS12-18 | GRAIN_QUALITY | 263 |
| GRAIN_QUALITY_MAS12-18 | GRAIN_QUALITY | 237 |
| GRAIN_QUALITY_MAS13-22 | GRAIN_QUALITY | 285 |
| GRAIN_QUALITY_MAS13-22 | GRAIN_QUALITY | 87 |
| GRAIN_QUALITY_MAS13-22 | GRAIN_QUALITY | 87 |
| GRAIN_QUALITY_MAS13-22 | GRAIN_QUALITY | 285 |
| GRAIN_QUALITY_MAS13-24 | GRAIN_QUALITY | 257 |
| GRAIN_QUALITY_MAS13-31 | GRAIN_QUALITY | 87 |
| GRAIN_QUALITY_MAS13-31 | GRAIN_QUALITY | 87 |
| GRAIN_QUALITY_MAS13-32 | GRAIN_QUALITY | 87 |
| GRAIN_QUALITY_MAS13-32 | GRAIN_QUALITY | 87 |
| GRAIN_QUALITY_MAS13-32 | GRAIN_QUALITY | 217 |
| GRAIN_QUALITY_MAS19-14 | GRAIN_QUALITY | 319 |
| GRAIN_QUALITY_MAS24-16 | GRAIN_QUALITY | 285 |
| GRAIN_QUALITY_MAS24-16 | GRAIN_QUALITY | 131 |
| GRAIN_QUALITY_MAS24-16 | GRAIN_QUALITY | 349 |
| GRAIN_QUALITY_MAS24-16 | GRAIN_QUALITY | 197 |
| GRAIN_QUALITY_MAS24-16 | GRAIN_QUALITY | 263 |
| GRAIN_QUALITY_MAS24-16 | GRAIN_QUALITY | 131 |
| GRAIN_QUALITY_MAS24-16 | GRAIN_QUALITY | 263 |
| GRAIN_QUALITY_MAS24-16 | GRAIN_QUALITY | 349 |
| GRAIN_QUALITY_MAS24-16 | GRAIN_QUALITY | 285 |
| GRAIN_QUALITY_MAS24-16 | GRAIN_QUALITY | 263 |
| GRAIN_QUALITY_MAS24-16 | GRAIN_QUALITY | 237 |
| GRAIN_QUALITY_MAS24-2 | GRAIN_QUALITY | 285 |
| GRAIN_QUALITY_MAS24-2 | GRAIN_QUALITY | 87 |
| GRAIN_QUALITY_MAS24-2 | GRAIN_QUALITY | 331 |
| GRAIN_QUALITY_MAS24-2 | GRAIN_QUALITY | 331 |
| GRAIN_QUALITY_MAS24-2 | GRAIN_QUALITY | 87 |
| GRAIN_QUALITY_MAS24-2 | GRAIN_QUALITY | 57 |
| GRAIN_QUALITY_MAS24-2 | GRAIN_QUALITY | 285 |
| GRAIN_QUALITY_MAS24-2 | GRAIN_QUALITY | 217 |
| GRAIN_QUALITY_MAS24-21 | GRAIN_QUALITY | 25 |
| GRAIN_QUALITY_MAS24-21 | GRAIN_QUALITY | 25 |
| GRAIN_QUALITY_MAS24-21 | GRAIN_QUALITY | 321 |
| GRAIN_QUALITY_MAS24-28 | GRAIN_QUALITY | 291 |
| GRAIN_QUALITY_MAS24-28 | GRAIN_QUALITY | 291 |
| MATURITY_MAS25-42 | MATURITY | 87 |
| MATURITY_SMS015-10 | MATURITY | 321 |
| MATURITY_SMS024-1 | MATURITY | 87 |
| MATURITY_SMS024-13 | MATURITY | 87 |
| MATURITY_SMS024-20 | MATURITY | 291 |
| MATURITY_SMS024-20 | MATURITY | 87 |
| MATURITY_SMS024-20 | MATURITY | 291 |
| MATURITY_SMS024-21 | MATURITY | 87 |
| MATURITY_SMS024-22 | MATURITY | 87 |
| MATURITY_SMS024-3 | MATURITY | 87 |
| MATURITY_SMS024-42 | MATURITY | 87 |
| MATURITY_SMS024-44 | MATURITY | 87 |
| MATURITY_SMS024-5 | MATURITY | 87 |
| MATURITY_SMS024-51 | MATURITY | 291 |
| MATURITY_SMS024-51 | MATURITY | 87 |
| MATURITY_SMS024-51 | MATURITY | 291 |
| MATURITY_SMS024-6 | MATURITY | 87 |
| MATURITY_SMS024-60 | MATURITY | 321 |
| MATURITY_SMS024-62 | MATURITY | 321 |
| MATURITY_SMS024-66 | MATURITY | 87 |
| MATURITY_SMS024-72 | MATURITY | 87 |
| MATURITY_SMS024-74 | MATURITY | 87 |
| MATURITY_ZM-ASI-1-3 | MATURITY | 87 |
| MATURITY_ZM-ASI-1-5 | MATURITY | 87 |
| MATURITY_ZM-ASI-10-2 | MATURITY | 87 |
| MATURITY_ZM-ASI-3-4 | MATURITY | 87 |
| MATURITY_ZM-ASI-4-2 | MATURITY | 321 |
| MATURITY_ZM-ASI-5-3 | MATURITY | 321 |
| MATURITY_ZM-ASI-7-2 | MATURITY | 87 |
| MATURITY_ZM-ASI-7-5 | MATURITY | 87 |
| MATURITY_ZM-ASI-8-1 | MATURITY | 321 |
| MATURITY_ZM-DTA-3-1 | MATURITY | 87 |
| MATURITY_ZM-DTA-3-2 | MATURITY | 87 |
| MATURITY_ZM-DTA-4-1 | MATURITY | 273 |
| MATURITY_ZM-DTA-4-1 | MATURITY | 291 |
| MATURITY_ZM-DTA-4-1 | MATURITY | 291 |
| MATURITY_ZM-DTA-4-1 | MATURITY | 273 |
| MATURITY_ZM-DTA-4-2 | MATURITY | 321 |
| MATURITY_ZM-DTA-8-4 | MATURITY | 321 |
| MATURITY_ZM-SILK-3-1 | MATURITY | 87 |
| MATURITY_ZM-SILK-3-2 | MATURITY | 87 |
| MATURITY_ZM-SILK-3-5 | MATURITY | 87 |
| MATURITY_ZM-SILK-4-1 | MATURITY | 273 |
| MATURITY_ZM-SILK-4-1 | MATURITY | 291 |
| MATURITY_ZM-SILK-4-1 | MATURITY | 291 |
| MATURITY_ZM-SILK-4-1 | MATURITY | 273 |
| MATURITY_ZM-SILK-6-2 | MATURITY | 87 |
| MATURITY_ZM-SILK-7-2 | MATURITY | 87 |
| MATURITY_ZM-SILK-8-1 | MATURITY | 321 |
| MATURITY_ZM-SILK-9-3 | MATURITY | 87 |
| PATHOGEN_RESISTANCE_ZM-ADCNLB-3-1 | PATHOGEN RESISTANCE | 285 |
| PATHOGEN_RESISTANCE_ZM-ADCNLB-3-1 | PATHOGEN RESISTANCE | 87 |
| PATHOGEN_RESISTANCE_ZM-ADCNLB-3-1 | PATHOGEN RESISTANCE | 285 |
| PATHOGEN_RESISTANCE_ZM-ADCNLB-3-2 | PATHOGEN RESISTANCE | 285 |
| PATHOGEN_RESISTANCE_ZM-ADCNLB-3-2 | PATHOGEN RESISTANCE | 87 |
| PATHOGEN_RESISTANCE_ZM-ADCNLB-3-2 | PATHOGEN RESISTANCE | 285 |
| PATHOGEN_RESISTANCE_ZM-ADCNLB-5-1 | PATHOGEN RESISTANCE | 25 |
| PATHOGEN_RESISTANCE_ZM-ADCNLB-5-1 | PATHOGEN RESISTANCE | 25 |
| PATHOGEN_RESISTANCE_ZM-ADCNLB-5-2 | PATHOGEN RESISTANCE | 25 |
| PATHOGEN_RESISTANCE_ZM-ADCNLB-5-2 | PATHOGEN RESISTANCE | 25 |
| PATHOGEN_RESISTANCE_ZM-ADCNLB-5-3 | PATHOGEN RESISTANCE | 305 |
| PATHOGEN_RESISTANCE_ZM-ADCNLB-5-3 | PATHOGEN RESISTANCE | 321 |
| PATHOGEN_RESISTANCE_ZM-ADCNLB-9-1 | PATHOGEN RESISTANCE | 309 |
| PATHOGEN_RESISTANCE_ZM-ADCNLB-9-1 | PATHOGEN RESISTANCE | 131 |
| PATHOGEN_RESISTANCE_ZM-ADCNLB-9-1 | PATHOGEN RESISTANCE | 257 |
| PATHOGEN_RESISTANCE_ZM-ADCNLB-9-1 | PATHOGEN RESISTANCE | 319 |
| PATHOGEN_RESISTANCE_ZM-ANMT-1-1 | PATHOGEN RESISTANCE | 257 |
| PATHOGEN_RESISTANCE_ZM-ANMT-1-2 | PATHOGEN RESISTANCE | 257 |
| PATHOGEN_RESISTANCE_ZM-ANMT-10-1 | PATHOGEN RESISTANCE | 87 |
| PATHOGEN_RESISTANCE_ZM-ANMT-10-1 | PATHOGEN RESISTANCE | 217 |
| PATHOGEN_RESISTANCE_ZM-ANMT-10-2 | PATHOGEN RESISTANCE | 87 |
| PATHOGEN_RESISTANCE_ZM-ANMT-10-2 | PATHOGEN RESISTANCE | 217 |
| PATHOGEN_RESISTANCE_ZM-ANMT-2-1 | PATHOGEN RESISTANCE | 87 |
| PATHOGEN_RESISTANCE_ZM-ANMT-2-1 | PATHOGEN RESISTANCE | 217 |
| PATHOGEN_RESISTANCE_ZM-ANMT-5-1 | PATHOGEN RESISTANCE | 263 |
| PATHOGEN_RESISTANCE_ZM-ANMT-5-1 | PATHOGEN RESISTANCE | 331 |
| PATHOGEN_RESISTANCE_ZM-ANMT-5-1 | PATHOGEN RESISTANCE | 263 |
| PATHOGEN_RESISTANCE_ZM-ANMT-5-1 | PATHOGEN RESISTANCE | 263 |
| PATHOGEN_RESISTANCE_ZM-APIT-1-1 | PATHOGEN RESISTANCE | 257 |
| PATHOGEN_RESISTANCE_ZM-APIT-1-2 | PATHOGEN RESISTANCE | 257 |
| PATHOGEN_RESISTANCE_ZM-APIT-10-1 | PATHOGEN RESISTANCE | 87 |
| PATHOGEN_RESISTANCE_ZM-APIT-10-1 | PATHOGEN RESISTANCE | 217 |
| PATHOGEN_RESISTANCE_ZM-APIT-10-2 | PATHOGEN RESISTANCE | 87 |
| PATHOGEN_RESISTANCE_ZM-APIT-10-2 | PATHOGEN RESISTANCE | 217 |
| PATHOGEN_RESISTANCE_ZM-APIT-2-1 | PATHOGEN RESISTANCE | 359 |
| PATHOGEN_RESISTANCE_ZM-APIT-2-1 | PATHOGEN RESISTANCE | 359 |
| PATHOGEN_RESISTANCE_ZM-APIT-2-1 | PATHOGEN RESISTANCE | 217 |
| PATHOGEN_RESISTANCE_ZM-APIT-2-1 | PATHOGEN RESISTANCE | 33 |
| PATHOGEN_RESISTANCE_ZM-APIT-3-1 | PATHOGEN RESISTANCE | 349 |
| PATHOGEN_RESISTANCE_ZM-APIT-3-1 | PATHOGEN RESISTANCE | 349 |
| PATHOGEN_RESISTANCE_ZM-APIT-3-1 | PATHOGEN RESISTANCE | 57 |
| PATHOGEN_RESISTANCE_ZM-APIT-3-2 | PATHOGEN RESISTANCE | 285 |
| PATHOGEN_RESISTANCE_ZM-APIT-3-2 | PATHOGEN RESISTANCE | 257 |
| PATHOGEN_RESISTANCE_ZM-APIT-3-2 | PATHOGEN RESISTANCE | 285 |
| PATHOGEN_RESISTANCE_ZM-APIT-6-1 | PATHOGEN RESISTANCE | 285 |
| PATHOGEN_RESISTANCE_ZM-APIT-6-1 | PATHOGEN RESISTANCE | 349 |
| PATHOGEN_RESISTANCE_ZM-APIT-6-1 | PATHOGEN RESISTANCE | 263 |
| PATHOGEN_RESISTANCE_ZM-APIT-6-1 | PATHOGEN RESISTANCE | 131 |
| PATHOGEN_RESISTANCE_ZM-APIT-6-1 | PATHOGEN RESISTANCE | 257 |
| PATHOGEN_RESISTANCE_ZM-APIT-6-1 | PATHOGEN RESISTANCE | 263 |
| PATHOGEN_RESISTANCE_ZM-APIT-6-1 | PATHOGEN RESISTANCE | 349 |
| PATHOGEN_RESISTANCE_ZM-APIT-6-1 | PATHOGEN RESISTANCE | 285 |
| PATHOGEN_RESISTANCE_ZM-APIT-6-1 | PATHOGEN RESISTANCE | 263 |
| PATHOGEN_RESISTANCE_ZM-APIT-6-1 | PATHOGEN RESISTANCE | 237 |
| PATHOGEN_RESISTANCE_ZM-APIT-9-1 | PATHOGEN RESISTANCE | 285 |
| PATHOGEN_RESISTANCE_ZM-APIT-9-1 | PATHOGEN RESISTANCE | 87 |
| PATHOGEN_RESISTANCE_ZM-APIT-9-1 | PATHOGEN RESISTANCE | 285 |
| PATHOGEN_RESISTANCE_ZM-APIT-9-1 | PATHOGEN RESISTANCE | 217 |
| PATHOGEN_RESISTANCE_ZM-AUT-1-1 | PATHOGEN RESISTANCE | 257 |
| PATHOGEN_RESISTANCE_ZM-AUT-1-2 | PATHOGEN RESISTANCE | 257 |
| PATHOGEN_RESISTANCE_ZM-AUT-10-1 | PATHOGEN RESISTANCE | 87 |
| PATHOGEN_RESISTANCE_ZM-AUT-10-1 | PATHOGEN RESISTANCE | 217 |
| PATHOGEN_RESISTANCE_ZM-AUT-10-2 | PATHOGEN RESISTANCE | 87 |
| PATHOGEN_RESISTANCE_ZM-AUT-10-2 | PATHOGEN RESISTANCE | 217 |
| PATHOGEN_RESISTANCE_ZM-AUT-3-1 | PATHOGEN RESISTANCE | 285 |
| PATHOGEN_RESISTANCE_ZM-AUT-3-1 | PATHOGEN RESISTANCE | 257 |
| PATHOGEN_RESISTANCE_ZM-AUT-3-1 | PATHOGEN RESISTANCE | 285 |
| PATHOGEN_RESISTANCE_ZM-AUT-6-1 | PATHOGEN RESISTANCE | 285 |
| PATHOGEN_RESISTANCE_ZM-AUT-6-1 | PATHOGEN RESISTANCE | 349 |
| PATHOGEN_RESISTANCE_ZM-AUT-6-1 | PATHOGEN RESISTANCE | 263 |
| PATHOGEN_RESISTANCE_ZM-AUT-6-1 | PATHOGEN RESISTANCE | 131 |
| PATHOGEN_RESISTANCE_ZM-AUT-6-1 | PATHOGEN RESISTANCE | 257 |
| PATHOGEN_RESISTANCE_ZM-AUT-6-1 | PATHOGEN RESISTANCE | 263 |
| PATHOGEN_RESISTANCE_ZM-AUT-6-1 | PATHOGEN RESISTANCE | 349 |
| PATHOGEN_RESISTANCE_ZM-AUT-6-1 | PATHOGEN RESISTANCE | 285 |
| PATHOGEN_RESISTANCE_ZM-AUT-6-1 | PATHOGEN RESISTANCE | 263 |
| PATHOGEN_RESISTANCE_ZM-AUT-6-1 | PATHOGEN RESISTANCE | 237 |
| PATHOGEN_RESISTANCE_ZM-CELW-5-1 | PATHOGEN RESISTANCE | 291 |
| PATHOGEN_RESISTANCE_ZM-CELW-6-1 | PATHOGEN RESISTANCE | 87 |
| PATHOGEN_RESISTANCE_ZM-CELW-6-1 | PATHOGEN RESISTANCE | 349 |
| PATHOGEN_RESISTANCE_ZM-CELW-6-1 | PATHOGEN RESISTANCE | 331 |
| PATHOGEN_RESISTANCE_ZM-CELW-6-1 | PATHOGEN RESISTANCE | 131 |
| PATHOGEN_RESISTANCE_ZM-CELW-6-1 | PATHOGEN RESISTANCE | 349 |
| PATHOGEN_RESISTANCE_ZM-CELW-6-1 | PATHOGEN RESISTANCE | 319 |
| PATHOGEN_RESISTANCE_ZM-CRR-1-10 | PATHOGEN RESISTANCE | 285 |
| PATHOGEN_RESISTANCE_ZM-CRR-1-10 | PATHOGEN RESISTANCE | 87 |
| PATHOGEN_RESISTANCE_ZM-CRR-1-10 | PATHOGEN RESISTANCE | 285 |
| PATHOGEN_RESISTANCE_ZM-CRR-1-10 | PATHOGEN RESISTANCE | 217 |
| PATHOGEN_RESISTANCE_ZM-CRR-1-11 | PATHOGEN RESISTANCE | 257 |
| PATHOGEN_RESISTANCE_ZM-CRR-1-12 | PATHOGEN RESISTANCE | 285 |
| PATHOGEN_RESISTANCE_ZM-CRR-1-12 | PATHOGEN RESISTANCE | 87 |
| PATHOGEN_RESISTANCE_ZM-CRR-1-12 | PATHOGEN RESISTANCE | 285 |
| PATHOGEN_RESISTANCE_ZM-CRR-1-12 | PATHOGEN RESISTANCE | 217 |
| PATHOGEN_RESISTANCE_ZM-CRR-1-13 | PATHOGEN RESISTANCE | 257 |
| PATHOGEN_RESISTANCE_ZM-CRR-1-14 | PATHOGEN RESISTANCE | 71 |
| PATHOGEN_RESISTANCE_ZM-CRR-1-14 | PATHOGEN RESISTANCE | 217 |
| PATHOGEN_RESISTANCE_ZM-CRR-1-14 | PATHOGEN RESISTANCE | 319 |
| PATHOGEN_RESISTANCE_ZM-CRR-1-15 | PATHOGEN RESISTANCE | 257 |
| PATHOGEN_RESISTANCE_ZM-CRR-2-11 | PATHOGEN RESISTANCE | 359 |
| PATHOGEN_RESISTANCE_ZM-CRR-2-11 | PATHOGEN RESISTANCE | 359 |
| PATHOGEN_RESISTANCE_ZM-CRR-2-11 | PATHOGEN RESISTANCE | 217 |
| PATHOGEN_RESISTANCE_ZM-CRR-2-11 | PATHOGEN RESISTANCE | 33 |
| PATHOGEN_RESISTANCE_ZM-CRR-3-6 | PATHOGEN RESISTANCE | 349 |
| PATHOGEN_RESISTANCE_ZM-CRR-3-6 | PATHOGEN RESISTANCE | 349 |
| PATHOGEN_RESISTANCE_ZM-CRR-3-6 | PATHOGEN RESISTANCE | 57 |
| PATHOGEN_RESISTANCE_ZM-CRR-3-7 | PATHOGEN RESISTANCE | 285 |
| PATHOGEN_RESISTANCE_ZM-CRR-3-7 | PATHOGEN RESISTANCE | 87 |
| PATHOGEN_RESISTANCE_ZM-CRR-3-7 | PATHOGEN RESISTANCE | 285 |
| PATHOGEN_RESISTANCE_ZM-CRR-3-8 | PATHOGEN RESISTANCE | 285 |
| PATHOGEN_RESISTANCE_ZM-CRR-3-8 | PATHOGEN RESISTANCE | 87 |
| PATHOGEN_RESISTANCE_ZM-CRR-3-8 | PATHOGEN RESISTANCE | 285 |
| PATHOGEN_RESISTANCE_ZM-CRR-3-9 | PATHOGEN RESISTANCE | 285 |
| PATHOGEN_RESISTANCE_ZM-CRR-3-9 | PATHOGEN RESISTANCE | 87 |
| PATHOGEN_RESISTANCE_ZM-CRR-3-9 | PATHOGEN RESISTANCE | 257 |
| PATHOGEN_RESISTANCE_ZM-CRR-3-9 | PATHOGEN RESISTANCE | 285 |
| PATHOGEN_RESISTANCE_ZM-CRR-4-3 | PATHOGEN RESISTANCE | 25 |
| PATHOGEN_RESISTANCE_ZM-CRR-4-3 | PATHOGEN RESISTANCE | 59 |
| PATHOGEN_RESISTANCE_ZM-CRR-4-3 | PATHOGEN RESISTANCE | 131 |
| PATHOGEN_RESISTANCE_ZM-CRR-4-3 | PATHOGEN RESISTANCE | 25 |
| PATHOGEN_RESISTANCE_ZM-CRR-4-4 | PATHOGEN RESISTANCE | 291 |
| PATHOGEN_RESISTANCE_ZM-CRR-4-4 | PATHOGEN RESISTANCE | 71 |
| PATHOGEN_RESISTANCE_ZM-CRR-4-4 | PATHOGEN RESISTANCE | 25 |
| PATHOGEN_RESISTANCE_ZM-CRR-4-4 | PATHOGEN RESISTANCE | 59 |
| PATHOGEN_RESISTANCE_ZM-CRR-4-4 | PATHOGEN RESISTANCE | 273 |
| PATHOGEN_RESISTANCE_ZM-CRR-4-4 | PATHOGEN EESISTANCE | 273 |
| PATHOGEN_RESISTANCE_ZM-CRR-4-4 | PATHOGEN RESISTANCE | 25 |
| PATHOGEN_RESISTANCE_ZM-CRR-4-4 | PATHOGEN RESISTANCE | 57 |
| PATHOGEN_RESISTANCE_ZM-CRR-4-4 | PATHOGEN RESISTANCE | 273 |
| PATHOGEN_RESISTANCE_ZM-CRR-4-4 | PATHOGEN RESISTANCE | 33 |
| PATHOGEN_RESISTANCE_ZM-CRR-4-5 | PATHOGEN RESISTANCE | 99 |
| PATHOGEN_RESISTANCE_ZM-CRR-4-5 | PATHOGEN RESISTANCE | 25 |
| PATHOGEN_RESISTANCE_ZM-CRR-4-5 | PATHOGEN RESISTANCE | 131 |
| PATHOGEN_RESISTANCE_ZM-CRR-4-5 | PATHOGEN RESISTANCE | 25 |
| PATHOGEN_RESISTANCE_ZM-CRR-4-6 | PATHOGEN RESISTANCE | 291 |
| PATHOGEN_RESISTANCE_ZM-CRR-4-6 | PATHOGEN RESISTANCE | 71 |
| PATHOGEN_RESISTANCE_ZM-CRR-4-6 | PATHOGEN RESISTANCE | 25 |
| PATHOGEN_RESISTANCE_ZM-CRR-4-6 | PATHOGEN RESISTANCE | 59 |
| PATHOGEN_RESISTANCE_ZM-CRR-4-6 | PATHOGEN RESISTANCE | 273 |
| PATHOGEN_RESISTANCE_ZM-CRR-4-6 | PATHOGEN RESISTANCE | 273 |
| PATHOGEN_RESISTANCE_ZM-CRR-4-6 | PATHOGEN RESISTANCE | 25 |
| PATHOGEN_RESISTANCE_ZM-CRR-4-6 | PATHOGEN RESISTANCE | 57 |
| PATHOGEN_RESISTANCE_ZM-CRR-4-6 | PATHOGEN RESISTANCE | 273 |
| PATHOGEN_RESISTANCE_ZM-CRR-4-6 | PATHOGEN RESISTANCE | 33 |
| PATHOGEN_RESISTANCE_ZM-CRR-5-6 | PATHOGEN RESISTANCE | 263 |
| PATHOGEN_RESISTANCE_ZM-CRR-5-6 | PATHOGEN RESISTANCE | 331 |
| PATHOGEN_RESISTANCE_ZM-CRR-5-6 | PATHOGEN RESISTANCE | 263 |
| PATHOGEN_RESISTANCE_ZM-CRR-5-6 | PATHOGEN RESISTANCE | 263 |
| PATHOGEN_RESISTANCE_ZM-CRR-5-7 | PATHOGEN RESISTANCE | 291 |
| PATHOGEN_RESISTANCE_ZM-CRR-5-7 | PATHOGEN RESISTANCE | 25 |
| PATHOGEN_RESISTANCE_ZM-CRR-5-7 | PATHOGEN RESISTANCE | 25 |
| PATHOGEN_RESISTANCE_ZM-CRR-5-9 | PATHOGEN RESISTANCE | 291 |
| PATHOGEN_RESISTANCE_ZM-CRR-6-4 | PATHOGEN RESISTANCE | 263 |
| PATHOGEN_RESISTANCE_ZM-CRR-6-4 | PATHOGEN RESISTANCE | 257 |
| PATHOGEN_RESISTANCE_ZM-CRR-6-4 | PATHOGEN RESISTANCE | 263 |
| PATHOGEN_RESISTANCE_ZM-CRR-6-4 | PATHOGEN RESISTANCE | 263 |
| PATHOGEN_RESISTANCE_ZM-CRR-6-5 | PATHOGEN RESISTANCE | 285 |
| PATHOGEN_RESISTANCE_ZM-CRR-6-5 | PATHOGEN RESISTANCE | 349 |
| PATHOGEN_RESISTANCE_ZM-CRR-6-5 | PATHOGEN RESISTANCE | 263 |
| PATHOGEN_RESISTANCE_ZM-CRR-6-5 | PATHOGEN RESISTANCE | 131 |
| PATHOGEN_RESISTANCE_ZM-CRR-6-5 | PATHOGEN RESISTANCE | 263 |
| PATHOGEN_RESISTANCE_ZM-CRR-6-5 | PATHOGEN RESISTANCE | 349 |
| PATHOGEN_RESISTANCE_ZM-CRR-6-5 | PATHOGEN RESISTANCE | 285 |
| PATHOGEN_RESISTANCE_ZM-CRR-6-5 | PATHOGEN RESISTANCE | 263 |
| PATHOGEN_RESISTANCE_ZM-CRR-6-5 | PATHOGEN RESISTANCE | 237 |
| PATHOGEN_RESISTANCE_ZM-CRR-6-6 | PATHOGEN RESISTANCE | 349 |
| PATHOGEN_RESISTANCE_ZM-CRR-6-6 | PATHOGEN RESISTANCE | 131 |
| PATHOGEN_RESISTANCE_ZM-CRR-6-6 | PATHOGEN RESISTANCE | 349 |
| PATHOGEN_RESISTANCE_ZM-CRR-7-5 | PATHOGEN RESISTANCE | 87 |
| PATHOGEN_RESISTANCE_ZM-CRR-7-6 | PATHOGEN RESISTANCE | 281 |
| PATHOGEN_RESISTANCE_ZM-CRR-8-6 | PATHOGEN RESISTANCE | 71 |
| PATHOGEN_RESISTANCE_ZM-CRR-8-6 | PATHOGEN RESISTANCE | 305 |
| PATHOGEN_RESISTANCE_ZM-CRR-8-6 | PATHOGEN RESISTANCE | 349 |
| PATHOGEN_RESISTANCE_ZM-CRR-8-6 | PATHOGEN RESISTANCE | 263 |
| PATHOGEN_RESISTANCE_ZM-CRR-8-6 | PATHOGEN RESISTANCE | 263 |
| PATHOGEN_RESISTANCE_ZM-CRR-8-6 | PATHOGEN RESISTANCE | 349 |
| PATHOGEN_RESISTANCE_ZM-CRR-8-6 | PATHOGEN RESISTANCE | 321 |
| PATHOGEN_RESISTANCE_ZM-CRR-8-6 | PATHOGEN RESISTANCE | 263 |
| PATHOGEN_RESISTANCE_ZM-CRR-8-7 | PATHOGEN RESISTANCE | 71 |
| PATHOGEN_RESISTANCE_ZM-CRR-8-7 | PATHOGEN RESISTANCE | 305 |
| PATHOGEN_RESISTANCE_ZM-CRR-8-7 | PATHOGEN RESISTANCE | 349 |
| PATHOGEN_RESISTANCE_ZM-CRR-8-7 | PATHOGEN RESISTANCE | 263 |
| PATHOGEN_RESISTANCE_ZM-CRR-8-7 | PATHOGEN RESISTANCE | 263 |
| PATHOGEN_RESISTANCE_ZM-CRR-8-7 | PATHOGEN RESISTANCE | 349 |
| PATHOGEN_RESISTANCE_ZM-CRR-8-7 | PATHOGEN RESISTANCE | 321 |
| PATHOGEN_RESISTANCE_ZM-CRR-8-7 | PATHOGEN RESISTANCE | 263 |
| PATHOGEN_RESISTANCE_ZM-CRR-9-2 | PATHOGEN RESISTANCE | 309 |
| PATHOGEN_RESISTANCE_ZM-CRR-9-2 | PATHOGEN RESISTANCE | 131 |
| PATHOGEN_RESISTANCE_ZM-CRR-9-2 | PATHOGEN RESISTANCE | 257 |
| PATHOGEN_RESISTANCE_ZM-CRR-9-2 | PATHOGEN RESISTANCE | 319 |
| PATHOGEN_RESISTANCE_ZM-CRR-9-3 | PATHOGEN RESISTANCE | 309 |
| PATHOGEN_RESISTANCE_ZM-CRR-9-3 | PATHOGEN RESISTANCE | 131 |
| PATHOGEN_RESISTANCE_ZM-CRR-9-3 | PATHOGEN RESISTANCE | 257 |
| PATHOGEN_RESISTANCE_ZM-CRR-9-3 | PATHOGEN RESISTANCE | 319 |
| PATHOGEN_RESISTANCE_ZM-CRR-9-4 | PATHOGEN RESISTANCE | 285 |
| PATHOGEN_RESISTANCE_ZM-CRR-9-4 | PATHOGEN RESISTANCE | 87 |
| PATHOGEN_RESISTANCE_ZM-CRR-9-4 | PATHOGEN RESISTANCE | 285 |
| PATHOGEN_RESISTANCE_ZM-CRR-9-4 | PATHOGEN RESISTANCE | 217 |
| PATHOGEN_RESISTANCE_ZM-CRR-9-5 | PATHOGEN RESISTANCE | 99 |
| PATHOGEN_RESISTANCE_ZM-CRR-9-5 | PATHOGEN RESISTANCE | 309 |
| PATHOGEN_RESISTANCE_ZM-CRR-9-5 | PATHOGEN RESISTANCE | 331 |
| PATHOGEN_RESISTANCE_ZM-CRR-9-5 | PATHOGEN RESISTANCE | 131 |
| PATHOGEN_RESISTANCE_ZM-CRR-9-5 | PATHOGEN RESISTANCE | 257 |
| PATHOGEN_RESISTANCE_ZM-CRR-9-5 | PATHOGEN RESISTANCE | 319 |
| PATHOGEN_RESISTANCE_ZM-CSVEG-1-1 | PATHOGEN RESISTANCE | 285 |
| PATHOGEN_RESISTANCE_ZM-CSVEG-1-1 | PATHOGEN RESISTANCE | 87 |
| PATHOGEN_RESISTANCE_ZM-CSVEG-1-1 | PATHOGEN RESISTANCE | 285 |
| PATHOGEN_RESISTANCE_ZM-CSVEG-1-1 | PATHOGEN RESISTANCE | 217 |
| PATHOGEN_RESISTANCE_ZM-CSVEG-4-1 | PATHOGEN RESISTANCE | 291 |
| PATHOGEN_RESISTANCE_ZM-CSVEG-4-1 | PATHOGEN RESISTANCE | 71 |
| PATHOGEN_RESISTANCE_ZM-CSVEG-4-1 | PATHOGEN RESISTANCE | 25 |
| PATHOGEN_RESISTANCE_ZM-CSVEG-4-1 | PATHOGEN RESISTANCE | 59 |
| PATHOGEN_RESISTANCE_ZM-CSVEG-4-1 | PATHOGEN RESISTANCE | 273 |
| PATHOGEN_RESISTANCE_ZM-CSVEG-4-1 | PATHOGEN RESISTANCE | 273 |
| PATHOGEN_RESISTANCE_ZM-CSVEG-4-1 | PATHOGEN RESISTANCE | 25 |
| PATHOGEN_RESISTANCE_ZM-CSVEG-4-1 | PATHOGEN RESISTANCE | 57 |
| PATHOGEN_RESISTANCE_ZM-CSVEG-4-1 | PATHOGEN RESISTANCE | 273 |
| PATHOGEN_RESISTANCE_ZM-CSVEG-4-1 | PATHOGEN RESISTANCE | 33 |
| PATHOGEN_RESISTANCE_ZM-CSVEG-4-2 | PATHOGEN RESISTANCE | 99 |
| PATHOGEN_RESISTANCE_ZM-CSVEG-4-2 | PATHOGEN RESISTANCE | 131 |
| PATHOGEN_RESISTANCE_ZM-CSVEG-7-1 | PATHOGEN RESISTANCE | 281 |
| PATHOGEN_RESISTANCE_ZM-CSVEG-9-1 | PATHOGEN RESISTANCE | 309 |
| PATHOGEN_RESISTANCE_ZM-CSVEG-9-1 | PATHOGEN RESISTANCE | 131 |
| PATHOGEN_RESISTANCE_ZM-CSVEG-9-1 | PATHOGEN RESISTANCE | 257 |
| PATHOGEN_RESISTANCE_ZM-CSVEG-9-1 | PATHOGEN RESISTANCE | 319 |
| PATHOGEN_RESISTANCE_ZM-CSVSG-1-1 | PATHOGEN RESISTANCE | 319 |
| PATHOGEN_RESISTANCE_ZM-CSVSG-4-1 | PATHOGEN RESISTANCE | 99 |
| PATHOGEN_RESISTANCE_ZM-CSVSG-4-1 | PATHOGEN RESISTANCE | 131 |
| PATHOGEN_RESISTANCE_ZM-DMS-1-1 | PATHOGEN RESISTANCE | 319 |
| PATHOGEN_RESISTANCE_ZM-DMS-1-2 | PATHOGEN RESISTANCE | 257 |
| PATHOGEN_RESISTANCE_ZM-DMS-9-1 | PATHOGEN RESISTANCE | 285 |
| PATHOGEN_RESISTANCE_ZM-DMS-9-1 | PATHOGEN RESISTANCE | 87 |
| PATHOGEN_RESISTANCE_ZM-DMS-9-1 | PATHOGEN RESISTANCE | 285 |
| PATHOGEN_RESISTANCE_ZM-DMS-9-1 | PATHOGEN RESISTANCE | 217 |
| PATHOGEN_RESISTANCE_ZM-ETURI-2-1 | PATHOGEN RESISTANCE | 291 |
| PATHOGEN_RESISTANCE_ZM-ETURI-2-1 | PATHOGEN RESISTANCE | 87 |
| PATHOGEN_RESISTANCE_ZM-ETURI-5-1 | PATHOGEN RESISTANCE | 25 |
| PATHOGEN_RESISTANCE_ZM-ETURI-5-1 | PATHOGEN RESISTANCE | 25 |
| PATHOGEN_RESISTANCE_ZM-HMAYI-3-1 | PATHOGEN RESISTANCE | 349 |
| PATHOGEN_RESISTANCE_ZM-HMAYI-3-1 | PATHOGEN RESISTANCE | 349 |
| PATHOGEN_RESISTANCE_ZM-HMAYI-3-1 | PATHOGEN RESISTANCE | 57 |
| PATHOGEN_RESISTANCE_ZM-HPVI-3-1 | PATHOGEN RESISTANCE | 349 |
| PATHOGEN_RESISTANCE_ZM-HPVI-3-1 | PATHOGEN RESISTANCE | 349 |
| PATHOGEN_RESISTANCE_ZM-HPVI-3-1 | PATHOGEN RESISTANCE | 57 |
| PATHOGEN_RESISTANCE_ZM-HPVI-6-1 | PATHOGEN RESISTANCE | 87 |
| PATHOGEN_RESISTANCE_ZM-HPVI-6-1 | PATHOGEN RESISTANCE | 349 |
| PATHOGEN_RESISTANCE_ZM-HPVI-6-1 | PATHOGEN RESISTANCE | 331 |
| PATHOGEN_RESISTANCE_ZM-HPVI-6-1 | PATHOGEN RESISTANCE | 131 |
| PATHOGEN_RESISTANCE_ZM-HPVI-6-1 | PATHOGEN RESISTANCE | 349 |
| PATHOGEN_RESISTANCE_ZM-HPVI-6-1 | PATHOGEN RESISTANCE | 319 |
| PATHOGEN_RESISTANCE_ZM-LT-1-1 | PATHOGEN RESISTANCE | 257 |
| PATHOGEN_RESISTANCE_ZM-LT-1-2 | PATHOGEN RESISTANCE | 285 |
| PATHOGEN_RESISTANCE_ZM-LT-1-2 | PATHOGEN RESISTANCE | 87 |
| PATHOGEN_RESISTANCE_ZM-LT-1-2 | PATHOGEN RESISTANCE | 285 |
| PATHOGEN_RESISTANCE_ZM-LT-1-2 | PATHOGEN RESISTANCE | 217 |
| PATHOGEN_RESISTANCE_ZM-LT-4-1 | PATHOGEN RESISTANCE | 59 |
| PATHOGEN_RESISTANCE_ZM-LT-5-1 | PATHOGEN RESISTANCE | 25 |
| PATHOGEN_RESISTANCE_ZM-LT-5-1 | PATHOGEN RESISTANCE | 25 |
| PATHOGEN_RESISTANCE_ZM-LT-7-1 | PATHOGEN RESISTANCE | 281 |
| PATHOGEN_RESISTANCE_ZM-LVI-4-1 | PATHOGEN RESISTANCE | 305 |
| PATHOGEN_RESISTANCE_ZM-LVI-4-1 | PATHOGEN RESISTANCE | 25 |
| PATHOGEN_RESISTANCE_ZM-LVI-4-1 | PATHOGEN RESISTANCE | 25 |
| PATHOGEN_RESISTANCE_ZM-LVI-4-1 | PATHOGEN RESISTANCE | 321 |
| PATHOGEN_RESISTANCE_ZM-LVI-5-1 | PATHOGEN RESISTANCE | 25 |
| PATHOGEN_RESISTANCE_ZM-LVI-5-1 | PATHOGEN RESISTANCE | 25 |
| PATHOGEN_RESISTANCE_ZM-LVI-6-1 | PATHOGEN RESISTANCE | 87 |
| PATHOGEN_RESISTANCE_ZM-LVI-6-1 | PATHOGEN RESISTANCE | 349 |
| PATHOGEN_RESISTANCE_ZM-LVI-6-1 | PATHOGEN RESISTANCE | 331 |
| PATHOGEN_RESISTANCE_ZM-LVI-6-1 | PATHOGEN RESISTANCE | 131 |
| PATHOGEN_RESISTANCE_ZM-LVI-6-1 | PATHOGEN RESISTANCE | 349 |
| PATHOGEN_RESISTANCE_ZM-LVI-6-1 | PATHOGEN RESISTANCE | 319 |
| PATHOGEN_RESISTANCE_ZM-MSVI-1-1 | PATHOGEN RESISTANCE | 257 |
| PATHOGEN_RESISTANCE_ZM-MSVI-1-2 | PATHOGEN RESISTANCE | 87 |
| PATHOGEN_RESISTANCE_ZM-MSVI-1-2 | PATHOGEN RESISTANCE | 217 |
| PATHOGEN_RESISTANCE_ZM-MSVI-1-2 | PATHOGEN RESISTANCE | 33 |
| PATHOGEN_RESISTANCE_ZM-MSVI-10-1 | PATHOGEN RESISTANCE | 87 |
| PATHOGEN_RESISTANCE_ZM-MSVI-10-1 | PATHOGEN RESISTANCE | 217 |
| PATHOGEN_RESISTANCE_ZM-MSVI-2-1 | PATHOGEN RESISTANCE | 87 |
| PATHOGEN_RESISTANCE_ZM-MSVI-2-1 | PATHOGEN RESISTANCE | 217 |
| PATHOGEN_RESISTANCE_ZM-MSVI-3-1 | PATHOGEN RESISTANCE | 285 |
| PATHOGEN_RESISTANCE_ZM-MSVI-3-1 | PATHOGEN RESISTANCE | 257 |
| PATHOGEN_RESISTANCE_ZM-MSVI-3-1 | PATHOGEN RESISTANCE | 285 |
| PATHOGEN_RESISTANCE_ZM-MSVI-3-2 | PATHOGEN RESISTANCE | 349 |
| PATHOGEN_RESISTANCE_ZM-MSVI-3-2 | PATHOGEN RESISTANCE | 349 |
| PATHOGEN_RESISTANCE_ZM-MSVI-3-2 | PATHOGEN RESISTANCE | 57 |
| PATHOGEN_RESISTANCE_ZM-MSVI-9-1 | PATHOGEN RESISTANCE | 285 |
| PATHOGEN_RESISTANCE_ZM-MSVI-9-1 | PATHOGEN RESISTANCE | 87 |
| PATHOGEN_RESISTANCE_ZM-MSVI-9-1 | PATHOGEN RESISTANCE | 285 |
| PATHOGEN_RESISTANCE_ZM-MSVI-9-1 | PATHOGEN RESISTANCE | 217 |
| PATHOGEN_RESISTANCE_ZM-NLBDS-1-1 | PATHOGEN RESISTANCE | 257 |
| PATHOGEN_RESISTANCE_ZM-NLBDS-10-2 | PATHOGEN RESISTANCE | 217 |
| PATHOGEN_RESISTANCE_ZM-NLBDS-3-2 | PATHOGEN RESISTANCE | 285 |
| PATHOGEN_RESISTANCE_ZM-NLBDS-3-2 | PATHOGEN RESISTANCE | 87 |
| PATHOGEN_RESISTANCE_ZM-NLBDS-3-2 | PATHOGEN RESISTANCE | 285 |
| PATHOGEN_RESISTANCE_ZM-NLBDS-3-5 | PATHOGEN RESISTANCE | 285 |
| PATHOGEN_RESISTANCE_ZM-NLBDS-3-5 | PATHOGEN RESISTANCE | 87 |
| PATHOGEN_RESISTANCE_ZM-NLBDS-3-5 | PATHOGEN RESISTANCE | 285 |
| PATHOGEN_RESISTANCE_ZM-NLBDS-4-1 | PATHOGEN RESISTANCE | 59 |
| PATHOGEN_RESISTANCE_ZM-NLBDS-4-2 | PATHOGEN RESISTANCE | 291 |
| PATHOGEN_RESISTANCE_ZM-NLBDS-4-2 | PATHOGEN RESISTANCE | 71 |
| PATHOGEN_RESISTANCE_ZM-NLBDS-4-2 | PATHOGEN RESISTANCE | 305 |
| PATHOGEN_RESISTANCE_ZM-NLBDS-4-2 | PATHOGEN RESISTANCE | 25 |
| PATHOGEN_RESISTANCE_ZM-NLBDS-4-2 | PATHOGEN RESISTANCE | 59 |
| PATHOGEN_RESISTANCE_ZM-NLBDS-4-2 | PATHOGEN RESISTANCE | 273 |
| PATHOGEN_RESISTANCE_ZM-NLBDS-4-2 | PATHOGEN RESISTANCE | 273 |
| PATHOGEN_RESISTANCE_ZM-NLBDS-4-2 | PATHOGEN RESISTANCE | 25 |
| PATHOGEN_RESISTANCE_ZM-NLBDS-4-2 | PATHOGEN RESISTANCE | 321 |
| PATHOGEN_RESISTANCE_ZM-NLBDS-4-2 | PATHOGEN RESISTANCE | 57 |
| PATHOGEN_RESISTANCE_ZM-NLBDS-4-2 | PATHOGEN RESISTANCE | 273 |
| PATHOGEN_RESISTANCE_ZM-NLBDS-4-2 | PATHOGEN RESISTANCE | 33 |
| PATHOGEN_RESISTANCE_ZM-NLBDS-5-1 | PATHOGEN RESISTANCE | 25 |
| PATHOGEN_RESISTANCE_ZM-NLBDS-5-1 | PATHOGEN RESISTANCE | 25 |
| PATHOGEN_RESISTANCE_ZM-NLBDS-5-2 | PATHOGEN RESISTANCE | 25 |
| PATHOGEN_RESISTANCE_ZM-NLBDS-5-2 | PATHOGEN RESISTANCE | 25 |
| PATHOGEN_RESISTANCE_ZM-NLBDS-5-3 | PATHOGEN RESISTANCE | 305 |
| PATHOGEN_RESISTANCE_ZM-NLBDS-5-3 | PATHOGEN RESISTANCE | 321 |
| PATHOGEN_RESISTANCE_ZM-NLBDS-5-5 | PATHOGEN RESISTANCE | 291 |
| PATHOGEN_RESISTANCE_ZM-NLBDS-6-1 | PATHOGEN RESISTANCE | 285 |
| PATHOGEN_RESISTANCE_ZM-NLBDS-6-1 | PATHOGEN RESISTANCE | 349 |
| PATHOGEN_RESISTANCE_ZM-NLBDS-6-1 | PATHOGEN RESISTANCE | 263 |
| PATHOGEN_RESISTANCE_ZM-NLBDS-6-1 | PATHOGEN RESISTANCE | 131 |
| PATHOGEN_RESISTANCE_ZM-NLBDS-6-1 | PATHOGEN RESISTANCE | 257 |
| PATHOGEN_RESISTANCE_ZM-NLBDS-6-1 | PATHOGEN RESISTANCE | 263 |
| PATHOGEN_RESISTANCE_ZM-NLBDS-6-1 | PATHOGEN RESISTANCE | 349 |
| PATHOGEN_RESISTANCE_ZM-NLBDS-6-1 | PATHOGEN RESISTANCE | 285 |
| PATHOGEN_RESISTANCE_ZM-NLBDS-6-1 | PATHOGEN RESISTANCE | 263 |
| PATHOGEN_RESISTANCE_ZM-NLBDS-6-1 | PATHOGEN RESISTANCE | 237 |
| PATHOGEN_RESISTANCE_ZM-NLBDS-8-1 | PATHOGEN RESISTANCE | 263 |
| PATHOGEN_RESISTANCE_ZM-NLBDS-8-1 | PATHOGEN RESISTANCE | 263 |
| PATHOGEN_RESISTANCE_ZM-NLBDS-8-1 | PATHOGEN RESISTANCE | 263 |
| PATHOGEN_RESISTANCE_ZM-NLBDS-8-2 | PATHOGEN RESISTANCE | 71 |
| PATHOGEN_RESISTANCE_ZM-NLBDS-8-2 | PATHOGEN RESISTANCE | 305 |
| PATHOGEN_RESISTANCE_ZM-NLBDS-8-2 | PATHOGEN RESISTANCE | 349 |
| PATHOGEN_RESISTANCE_ZM-NLBDS-8-2 | PATHOGEN RESISTANCE | 349 |
| PATHOGEN_RESISTANCE_ZM-NLBDS-8-2 | PATHOGEN RESISTANCE | 321 |
| PATHOGEN_RESISTANCE_ZM-NLBDS-9-1 | PATHOGEN RESISTANCE | 309 |
| PATHOGEN_RESISTANCE_ZM-NLBDS-9-1 | PATHOGEN RESISTANCE | 131 |
| PATHOGEN_RESISTANCE_ZM-NLBDS-9-1 | PATHOGEN RESISTANCE | 257 |
| PATHOGEN_RESISTANCE_ZM-NLBDS-9-1 | PATHOGEN RESISTANCE | 319 |
| PATHOGEN_RESISTANCE_ZM-NLBDS-9-2 | PATHOGEN RESISTANCE | 285 |
| PATHOGEN_RESISTANCE_ZM-NLBDS-9-2 | PATHOGEN RESISTANCE | 87 |
| PATHOGEN_RESISTANCE_ZM-NLBDS-9-2 | PATHOGEN RESISTANCE | 331 |
| PATHOGEN_RESISTANCE_ZM-NLBDS-9-2 | PATHOGEN RESISTANCE | 57 |
| PATHOGEN_RESISTANCE_ZM-NLBDS-9-2 | PATHOGEN RESISTANCE | 285 |
| PATHOGEN_RESISTANCE_ZM-NLBDS-9-2 | PATHOGEN RESISTANCE | 217 |
| PATHOGEN_RESISTANCE_ZM-NLBDS-9-2 | PATHOGEN RESISTANCE | 319 |
| PATHOGEN_RESISTANCE_ZM-NLBIP-3-1 | PATHOGEN RESISTANCE | 285 |
| PATHOGEN_RESISTANCE_ZM-NLBIP-3-1 | PATHOGEN RESISTANCE | 87 |
| PATHOGEN_RESISTANCE_ZM-NLBIP-3-1 | PATHOGEN RESISTANCE | 285 |
| PATHOGEN_RESISTANCE_ZM-NLBIP-4-1 | PATHOGEN RESISTANCE | 291 |
| PATHOGEN_RESISTANCE_ZM-NLBIP-4-1 | PATHOGEN RESISTANCE | 71 |
| PATHOGEN_RESISTANCE_ZM-NLBIP-4-1 | PATHOGEN RESISTANCE | 25 |
| PATHOGEN_RESISTANCE_ZM-NLBIP-4-1 | PATHOGEN RESISTANCE | 273 |
| PATHOGEN_RESISTANCE_ZM-NLBIP-4-1 | PATHOGEN RESISTANCE | 273 |
| PATHOGEN_RESISTANCE_ZM-NLBIP-4-1 | PATHOGEN RESISTANCE | 25 |
| PATHOGEN_RESISTANCE_ZM-NLBIP-4-1 | PATHOGEN RESISTANCE | 57 |
| PATHOGEN_RESISTANCE_ZM-NLBIP-4-1 | PATHOGEN RESISTANCE | 273 |
| PATHOGEN_RESISTANCE_ZM-NLBIP-4-1 | PATHOGEN RESISTANCE | 33 |
| PATHOGEN_RESISTANCE_ZM-NLBIP-5-1 | PATHOGEN RESISTANCE | 25 |
| PATHOGEN_RESISTANCE_ZM-NLBIP-5-1 | PATHOGEN RESISTANCE | 25 |
| PATHOGEN_RESISTANCE_ZM-NLBIP-8-1 | PATHOGEN RESISTANCE | 263 |
| PATHOGEN_RESISTANCE_ZM-NLBIP-8-1 | PATHOGEN RESISTANCE | 263 |
| PATHOGEN_RESISTANCE_ZM-NLBIP-8-1 | PATHOGEN RESISTANCE | 263 |
| PATHOGEN_RESISTANCE_ZM-NLBIP-9-1 | PATHOGEN RESISTANCE | 309 |
| PATHOGEN_RESISTANCE_ZM-NLBIP-9-1 | PATHOGEN RESISTANCE | 131 |
| PATHOGEN_RESISTANCE_ZM-NLBIP-9-1 | PATHOGEN RESISTANCE | 257 |
| PATHOGEN_RESISTANCE_ZM-NLBIP-9-1 | PATHOGEN RESISTANCE | 319 |
| PATHOGEN_RESISTANCE_ZM-PCSI-1-1 | PATHOGEN RESISTANCE | 71 |
| PATHOGEN_RESISTANCE_ZM-PCSI-1-1 | PATHOGEN RESISTANCE | 217 |
| PATHOGEN_RESISTANCE_ZM-PCSI-1-1 | PATHOGEN RESISTANCE | 319 |
| PATHOGEN_RESISTANCE_ZM-PCSI-1-2 | PATHOGEN RESISTANCE | 257 |
| PATHOGEN_RESISTANCE_ZM-PCSI-1-4 | PATHOGEN RESISTANCE | 285 |
| PATHOGEN_RESISTANCE_ZM-PCSI-1-4 | PATHOGEN RESISTANCE | 87 |
| PATHOGEN_RESISTANCE_ZM-PCSI-1-4 | PATHOGEN RESISTANCE | 285 |
| PATHOGEN_RESISTANCE_ZM-PCSI-1-4 | PATHOGEN RESISTANCE | 217 |
| PATHOGEN_RESISTANCE_ZM-PCSI-1-5 | PATHOGEN RESISTANCE | 319 |
| PATHOGEN_RESISTANCE_ZM-PCSI-1-6 | PATHOGEN RESISTANCE | 285 |
| PATHOGEN_RESISTANCE_ZM-PCSI-1-6 | PATHOGEN RESISTANCE | 87 |
| PATHOGEN_RESISTANCE_ZM-PCSI-1-6 | PATHOGEN RESISTANCE | 285 |
| PATHOGEN_RESISTANCE_ZM-PCSI-1-6 | PATHOGEN RESISTANCE | 217 |
| PATHOGEN_RESISTANCE_ZM-PCSI-1-7 | PATHOGEN RESISTANCE | 257 |
| PATHOGEN_RESISTANCE_ZM-PCSI-2-1 | PATHOGEN RESISTANCE | 291 |
| PATHOGEN_RESISTANCE_ZM-PCSI-2-1 | PATHOGEN RESISTANCE | 87 |
| PATHOGEN_RESISTANCE_ZM-PCSI-2-4 | PATHOGEN RESISTANCE | 285 |
| PATHOGEN_RESISTANCE_ZM-PCSI-2-4 | PATHOGEN RESISTANCE | 359 |
| PATHOGEN_RESISTANCE_ZM-PCSI-2-4 | PATHOGEN RESISTANCE | 233 |
| PATHOGEN_RESISTANCE_ZM-PCSI-2-4 | PATHOGEN RESISTANCE | 359 |
| PATHOGEN_RESISTANCE_ZM-PCSI-2-4 | PATHOGEN RESISTANCE | 233 |
| PATHOGEN_RESISTANCE_ZM-PCSI-2-4 | PATHOGEN RESISTANCE | 281 |
| PATHOGEN_RESISTANCE_ZM-PCSI-2-4 | PATHOGEN RESISTANCE | 233 |
| PATHOGEN_RESISTANCE_ZM-PCSI-2-4 | PATHOGEN RESISTANCE | 285 |
| PATHOGEN_RESISTANCE_ZM-PCSI-2-4 | PATHOGEN RESISTANCE | 233 |
| PATHOGEN_RESISTANCE_ZM-PCSI-2-4 | PATHOGEN RESISTANCE | 33 |
| PATHOGEN_RESISTANCE_ZM-PCSI-2-5 | PATHOGEN RESISTANCE | 291 |
| PATHOGEN_RESISTANCE_ZM-PCSI-2-5 | PATHOGEN RESISTANCE | 87 |
| PATHOGEN_RESISTANCE_ZM-PCSI-3-1 | PATHOGEN RESISTANCE | 285 |
| PATHOGEN_RESISTANCE_ZM-PCSI-3-1 | PATHOGEN RESISTANCE | 349 |
| PATHOGEN_RESISTANCE_ZM-PCSI-3-1 | PATHOGEN RESISTANCE | 349 |
| PATHOGEN_RESISTANCE_ZM-PCSI-3-1 | PATHOGEN RESISTANCE | 57 |
| PATHOGEN_RESISTANCE_ZM-PCSI-3-1 | PATHOGEN RESISTANCE | 285 |
| PATHOGEN_RESISTANCE_ZM-PCSI-3-2 | PATHOGEN RESISTANCE | 285 |
| PATHOGEN_RESISTANCE_ZM-PCSI-3-2 | PATHOGEN RESISTANCE | 87 |
| PATHOGEN_RESISTANCE_ZM-PCSI-3-2 | PATHOGEN RESISTANCE | 285 |
| PATHOGEN_RESISTANCE_ZM-PCSI-3-3 | PATHOGEN RESISTANCE | 285 |
| PATHOGEN_RESISTANCE_ZM-PCSI-3-3 | PATHOGEN RESISTANCE | 285 |
| PATHOGEN_RESISTANCE_ZM-PCSI-3-4 | PATHOGEN RESISTANCE | 285 |
| PATHOGEN_RESISTANCE_ZM-PCSI-3-4 | PATHOGEN RESISTANCE | 87 |
| PATHOGEN_RESISTANCE_ZM-PCSI-3-4 | PATHOGEN RESISTANCE | 257 |
| PATHOGEN_RESISTANCE_ZM-PCSI-3-4 | PATHOGEN RESISTANCE | 285 |
| PATHOGEN_RESISTANCE_ZM-PCSI-3-5 | PATHOGEN RESISTANCE | 285 |
| PATHOGEN_RESISTANCE_ZM-PCSI-3-5 | PATHOGEN RESISTANCE | 87 |
| PATHOGEN_RESISTANCE_ZM-PCSI-3-5 | PATHOGEN RESISTANCE | 285 |
| PATHOGEN_RESISTANCE_ZM-PCSI-3-6 | PATHOGEN RESISTANCE | 285 |
| PATHOGEN_RESISTANCE_ZM-PCSI-3-6 | PATHOGEN RESISTANCE | 349 |
| PATHOGEN_RESISTANCE_ZM-PCSI-3-6 | PATHOGEN RESISTANCE | 349 |
| PATHOGEN_RESISTANCE_ZM-PCSI-3-6 | PATHOGEN RESISTANCE | 57 |
| PATHOGEN_RESISTANCE_ZM-PCSI-3-6 | PATHOGEN RESISTANCE | 285 |
| PATHOGEN_RESISTANCE_ZM-PCSI-4-1 | PATHOGEN RESISTANCE | 59 |
| PATHOGEN_RESISTANCE_ZM-PCSI-4-2 | PATHOGEN RESISTANCE | 291 |
| PATHOGEN_RESISTANCE_ZM-PCSI-4-2 | PATHOGEN RESISTANCE | 71 |
| PATHOGEN_RESISTANCE_ZM-PCSI-4-2 | PATHOGEN RESISTANCE | 25 |
| PATHOGEN_RESISTANCE_ZM-PCSI-4-2 | PATHOGEN RESISTANCE | 59 |
| PATHOGEN_RESISTANCE_ZM-PCSI-4-2 | PATHOGEN RESISTANCE | 273 |
| PATHOGEN_RESISTANCE_ZM-PCSI-4-2 | PATHOGEN RESISTANCE | 273 |
| PATHOGEN_RESISTANCE_ZM-PCSI-4-2 | PATHOGEN RESISTANCE | 25 |
| PATHOGEN_RESISTANCE_ZM-PCSI-4-2 | PATHOGEN RESISTANCE | 57 |
| PATHOGEN_RESISTANCE_ZM-PCSI-4-2 | PATHOGEN RESISTANCE | 273 |
| PATHOGEN_RESISTANCE_ZM-PCSI-4-2 | PATHOGEN RESISTANCE | 33 |
| PATHOGEN_RESISTANCE_ZM-PCSI-4-3 | PATHOGEN RESISTANCE | 99 |
| PATHOGEN_RESISTANCE_ZM-PCSI-4-3 | PATHOGEN RESISTANCE | 131 |
| PATHOGEN_RESISTANCE_ZM-PCSI-4-5 | PATHOGEN RESISTANCE | 99 |
| PATHOGEN_RESISTANCE_ZM-PCSI-4-5 | PATHOGEN RESISTANCE | 131 |
| PATHOGEN_RESISTANCE_ZM-PCSI-4-6 | PATHOGEN RESISTANCE | 305 |
| PATHOGEN_RESISTANCE_ZM-PCSI-4-6 | PATHOGEN RESISTANCE | 25 |
| PATHOGEN_RESISTANCE_ZM-PCSI-4-6 | PATHOGEN RESISTANCE | 25 |
| PATHOGEN_RESISTANCE_ZM-PCSI-4-6 | PATHOGEN RESISTANCE | 321 |
| PATHOGEN_RESISTANCE_ZM-PCSI-5-1 | PATHOGEN RESISTANCE | 263 |
| PATHOGEN_RESISTANCE_ZM-PCSI-5-1 | PATHOGEN RESISTANCE | 331 |
| PATHOGEN_RESISTANCE_ZM-PCSI-5-1 | PATHOGEN RESISTANCE | 263 |
| PATHOGEN_RESISTANCE_ZM-PCSI-5-1 | PATHOGEN RESISTANCE | 263 |
| PATHOGEN_RESISTANCE_ZM-PCSI-5-2 | PATHOGEN RESISTANCE | 263 |
| PATHOGEN_RESISTANCE_ZM-PCSI-5-2 | PATHOGEN RESISTANCE | 331 |
| PATHOGEN_RESISTANCE_ZM-PCSI-5-2 | PATHOGEN RESISTANCE | 263 |
| PATHOGEN_RESISTANCE_ZM-PCSI-5-2 | PATHOGEN RESISTANCE | 263 |
| PATHOGEN_RESISTANCE_ZM-PCSI-5-3 | PATHOGEN RESISTANCE | 263 |
| PATHOGEN_RESISTANCE_ZM-PCSI-5-3 | PATHOGEN RESISTANCE | 331 |
| PATHOGEN_RESISTANCE_ZM-PCSI-5-3 | PATHOGEN RESISTANCE | 263 |
| PATHOGEN_RESISTANCE_ZM-PCSI-5-3 | PATHOGEN RESISTANCE | 263 |
| PATHOGEN_RESISTANCE_ZM-PCSI-5-5 | PATHOGEN RESISTANCE | 263 |
| PATHOGEN_RESISTANCE_ZM-PCSI-5-5 | PATHOGEN RESISTANCE | 331 |
| PATHOGEN_RESISTANCE_ZM-PCSI-5-5 | PATHOGEN RESISTANCE | 263 |
| PATHOGEN_RESISTANCE_ZM-PCSI-5-5 | PATHOGEN RESISTANCE | 263 |
| PATHOGEN_RESISTANCE_ZM-PCSI-5-7 | PATHOGEN RESISTANCE | 25 |
| PATHOGEN_RESISTANCE_ZM-PCSI-5-7 | PATHOGEN RESISTANCE | 25 |
| PATHOGEN_RESISTANCE_ZM-PCSI-6-1 | PATHOGEN RESISTANCE | 263 |
| PATHOGEN_RESISTANCE_ZM-PCSI-6-1 | PATHOGEN RESISTANCE | 257 |
| PATHOGEN_RESISTANCE_ZM-PCSI-6-1 | PATHOGEN RESISTANCE | 263 |
| PATHOGEN_RESISTANCE_ZM-PCSI-6-1 | PATHOGEN RESISTANCE | 263 |
| PATHOGEN_RESISTANCE_ZM-PCSI-7-1 | PATHOGEN RESISTANCE | 281 |
| PATHOGEN_RESISTANCE_ZM-PCSI-7-2 | PATHOGEN RESISTANCE | 281 |
| PATHOGEN_RESISTANCE_ZM-PCSI-7-3 | PATHOGEN RESISTANCE | 281 |
| PATHOGEN_RESISTANCE_ZM-PCSI-9-1 | PATHOGEN RESISTANCE | 285 |
| PATHOGEN_RESISTANCE_ZM-PCSI-9-1 | PATHOGEN RESISTANCE | 87 |
| PATHOGEN_RESISTANCE_ZM-PCSI-9-1 | PATHOGEN RESISTANCE | 285 |
| PATHOGEN_RESISTANCE_ZM-PCSI-9-1 | PATHOGEN RESISTANCE | 217 |
| PATHOGEN_RESISTANCE_ZM-PCSI-9-2 | PATHOGEN RESISTANCE | 309 |
| PATHOGEN_RESISTANCE_ZM-PCSI-9-2 | PATHOGEN RESISTANCE | 131 |
| PATHOGEN_RESISTANCE_ZM-PCSI-9-2 | PATHOGEN RESISTANCE | 257 |
| PATHOGEN_RESISTANCE_ZM-PCSI-9-2 | PATHOGEN RESISTANCE | 319 |
| PATHOGEN_RESISTANCE_ZM-PCSI-9-3 | PATHOGEN RESISTANCE | 309 |
| PATHOGEN_RESISTANCE_ZM-PCSI-9-3 | PATHOGEN RESISTANCE | 131 |
| PATHOGEN_RESISTANCE_ZM-PCSI-9-3 | PATHOGEN RESISTANCE | 257 |
| PATHOGEN_RESISTANCE_ZM-PCSI-9-3 | PATHOGEN RESISTANCE | 319 |
| PATHOGEN_RESISTANCE_ZM-PHSI-4-1 | PATHOGEN RESISTANCE | 305 |
| PATHOGEN_RESISTANCE_ZM-PHSI-4-1 | PATHOGEN RESISTANCE | 25 |
| PATHOGEN_RESISTANCE_ZM-PHSI-4-1 | PATHOGEN RESISTANCE | 25 |
| PATHOGEN_RESISTANCE_ZM-PHSI-4-1 | PATHOGEN RESISTANCE | 321 |
| PATHOGEN_RESISTANCE_ZM-PHSI-5-1 | PATHOGEN RESISTANCE | 291 |
| PATHOGEN_RESISTANCE_ZM-PHSI-6-1 | PATHOGEN RESISTANCE | 263 |
| PATHOGEN_RESISTANCE_ZM-PHSI-6-1 | PATHOGEN RESISTANCE | 257 |
| PATHOGEN_RESISTANCE_ZM-PHSI-6-1 | PATHOGEN RESISTANCE | 263 |
| PATHOGEN_RESISTANCE_ZM-PHSI-6-1 | PATHOGEN RESISTANCE | 263 |
| PATHOGEN_RESISTANCE_ZM-PHSI-6-2 | PATHOGEN RESISTANCE | 87 |
| PATHOGEN_RESISTANCE_ZM-PHSI-6-2 | PATHOGEN RESISTANCE | 349 |
| PATHOGEN_RESISTANCE_ZM-PHSI-6-2 | PATHOGEN RESISTANCE | 331 |
| PATHOGEN_RESISTANCE_ZM-PHSI-6-2 | PATHOGEN RESISTANCE | 131 |
| PATHOGEN_RESISTANCE_ZM-PHSI-6-2 | PATHOGEN RESISTANCE | 349 |
| PATHOGEN_RESISTANCE_ZM-PHSI-6-2 | PATHOGEN RESISTANCE | 319 |
| PATHOGEN_RESISTANCE_ZM-PHSI-6-3 | PATHOGEN RESISTANCE | 285 |
| PATHOGEN_RESISTANCE_ZM-PHSI-6-3 | PATHOGEN RESISTANCE | 263 |
| PATHOGEN_RESISTANCE_ZM-PHSI-6-3 | PATHOGEN RESISTANCE | 131 |
| PATHOGEN_RESISTANCE_ZM-PHSI-6-3 | PATHOGEN RESISTANCE | 263 |
| PATHOGEN_RESISTANCE_ZM-PHSI-6-3 | PATHOGEN RESISTANCE | 285 |
| PATHOGEN_RESISTANCE_ZM-PHSI-6-3 | PATHOGEN RESISTANCE | 263 |
| PATHOGEN_RESISTANCE_ZM-PHSI-8-1 | PATHOGEN RESISTANCE | 71 |
| PATHOGEN_RESISTANCE_ZM-PHSI-8-1 | PATHOGEN RESISTANCE | 305 |
| PATHOGEN_RESISTANCE_ZM-PHSI-8-1 | PATHOGEN RESISTANCE | 349 |
| PATHOGEN_RESISTANCE_ZM-PHSI-8-1 | PATHOGEN RESISTANCE | 349 |
| PATHOGEN_RESISTANCE_ZM-PHSI-8-1 | PATHOGEN RESISTANCE | 321 |
| PATHOGEN_RESISTANCE_ZM-PHSI-9-1 | PATHOGEN RESISTANCE | 319 |
| PATHOGEN_RESISTANCE_ZM-SCBR-1-1 | PATHOGEN RESISTANCE | 319 |
| PATHOGEN_RESISTANCE_ZM-SCBR-1-2 | PATHOGEN RESISTANCE | 257 |
| PATHOGEN_RESISTANCE_ZM-SCBR-5-1 | PATHOGEN RESISTANCE | 25 |
| PATHOGEN_RESISTANCE_ZM-SCBR-5-1 | PATHOGEN RESISTANCE | 25 |
| PATHOGEN_RESISTANCE_ZM-SCBR-7-1 | PATHOGEN RESISTANCE | 281 |
| PATHOGEN_RESISTANCE_ZM-SCBR-8-1 | PATHOGEN RESISTANCE | 263 |
| PATHOGEN_RESISTANCE_ZM-SCBR-8-1 | PATHOGEN RESISTANCE | 263 |
| PATHOGEN_RESISTANCE_ZM-SCBR-8-1 | PATHOGEN RESISTANCE | 263 |
| PATHOGEN_RESISTANCE_ZM-SCBR-9-1 | PATHOGEN RESISTANCE | 319 |
| PATHOGEN_RESISTANCE_ZM-SWCBR-1-1 | PATHOGEN RESISTANCE | 257 |
| PATHOGEN_RESISTANCE_ZM-SWCBR-1-2 | PATHOGEN RESISTANCE | 71 |
| PATHOGEN_RESISTANCE_ZM-SWCBR-1-2 | PATHOGEN RESISTANCE | 217 |
| PATHOGEN_RESISTANCE_ZM-SWCBR-1-2 | PATHOGEN RESISTANCE | 319 |
| PATHOGEN_RESISTANCE_ZM-SWCBR-1-3 | PATHOGEN RESISTANCE | 319 |
| PATHOGEN_RESISTANCE_ZM-SWCBR-1-4 | PATHOGEN RESISTANCE | 257 |
| PATHOGEN_RESISTANCE_ZM-SWCBR-1-5 | PATHOGEN RESISTANCE | 71 |
| PATHOGEN_RESISTANCE_ZM-SWCBR-1-5 | PATHOGEN RESISTANCE | 217 |
| PATHOGEN_RESISTANCE_ZM-SWCBR-1-6 | PATHOGEN RESISTANCE | 319 |
| PATHOGEN_RESISTANCE_ZM-SWCBR-3-1 | PATHOGEN RESISTANCE | 285 |
| PATHOGEN_RESISTANCE_ZM-SWCBR-3-1 | PATHOGEN RESISTANCE | 257 |
| PATHOGEN_RESISTANCE_ZM-SWCBR-3-1 | PATHOGEN RESISTANCE | 285 |
| PATHOGEN_RESISTANCE_ZM-SWCBR-3-2 | PATHOGEN RESISTANCE | 285 |
| PATHOGEN_RESISTANCE_ZM-SWCBR-3-2 | PATHOGEN RESISTANCE | 257 |
| PATHOGEN_RESISTANCE_ZM-SWCBR-3-2 | PATHOGEN RESISTANCE | 285 |
| PATHOGEN_RESISTANCE_ZM-SWCBR-5-1 | PATHOGEN RESISTANCE | 291 |
| PATHOGEN_RESISTANCE_ZM-SWCBR-5-1 | PATHOGEN RESISTANCE | 25 |
| PATHOGEN_RESISTANCE_ZM-SWCBR-5-1 | PATHOGEN RESISTANCE | 25 |
| PATHOGEN_RESISTANCE_ZM-SWCBR-5-2 | PATHOGEN RESISTANCE | 25 |
| PATHOGEN_RESISTANCE_ZM-SWCBR-5-2 | PATHOGEN RESISTANCE | 25 |
| PATHOGEN_RESISTANCE_ZM-SWCBR-5-3 | PATHOGEN RESISTANCE | 25 |
| PATHOGEN_RESISTANCE_ZM-SWCBR-5-3 | PATHOGEN RESISTANCE | 25 |
| PATHOGEN_RESISTANCE_ZM-SWCBR-6-1 | PATHOGEN RESISTANCE | 87 |
| PATHOGEN_RESISTANCE_ZM-SWCBR-6-1 | PATHOGEN RESISTANCE | 349 |
| PATHOGEN_RESISTANCE_ZM-SWCBR-6-1 | PATHOGEN RESISTANCE | 331 |
| PATHOGEN_RESISTANCE_ZM-SWCBR-6-1 | PATHOGEN RESISTANCE | 131 |
| PATHOGEN_RESISTANCE_ZM-SWCBR-6-1 | PATHOGEN RESISTANCE | 349 |
| PATHOGEN_RESISTANCE_ZM-SWCBR-6-1 | PATHOGEN RESISTANCE | 319 |
| PATHOGEN_RESISTANCE_ZM-SWCBR-6-2 | PATHOGEN RESISTANCE | 285 |
| PATHOGEN_RESISTANCE_ZM-SWCBR-6-2 | PATHOGEN RESISTANCE | 349 |
| PATHOGEN_RESISTANCE_ZM-SWCBR-6-2 | PATHOGEN RESISTANCE | 263 |
| PATHOGEN_RESISTANCE_ZM-SWCBR-6-2 | PATHOGEN RESISTANCE | 131 |
| PATHOGEN_RESISTANCE_ZM-SWCBR-6-2 | PATHOGEN RESISTANCE | 263 |
| PATHOGEN_RESISTANCE_ZM-SWCBR-6-2 | PATHOGEN RESISTANCE | 349 |
| PATHOGEN_RESISTANCE_ZM-SWCBR-6-2 | PATHOGEN RESISTANCE | 285 |
| PATHOGEN_RESISTANCE_ZM-SWCBR-6-2 | PATHOGEN RESISTANCE | 263 |
| PATHOGEN_RESISTANCE_ZM-SWCBR-6-2 | PATHOGEN RESISTANCE | 237 |
| PATHOGEN_RESISTANCE_ZM-SWCBR-6-3 | PATHOGEN RESISTANCE | 285 |
| PATHOGEN_RESISTANCE_ZM-SWCBR-6-3 | PATHOGEN RESISTANCE | 263 |
| PATHOGEN_RESISTANCE_ZM-SWCBR-6-3 | PATHOGEN RESISTANCE | 131 |
| PATHOGEN_RESISTANCE_ZM-SWCBR-6-3 | PATHOGEN RESISTANCE | 263 |
| PATHOGEN_RESISTANCE_ZM-SWCBR-6-3 | PATHOGEN RESISTANCE | 285 |
| PATHOGEN_RESISTANCE_ZM-SWCBR-6-3 | PATHOGEN RESISTANCE | 263 |
| PATHOGEN_RESISTANCE_ZM-SWCBR-7-1 | PATHOGEN RESISTANCE | 281 |
| PATHOGEN_RESISTANCE_ZM-SWCBR-7-2 | PATHOGEN RESISTANCE | 281 |
| PATHOGEN_RESISTANCE_ZM-SWCBR-7-3 | PATHOGEN RESISTANCE | 281 |
| PATHOGEN_RESISTANCE_ZM-SWCBR-8-1 | PATHOGEN RESISTANCE | 71 |
| PATHOGEN_RESISTANCE_ZM-SWCBR-8-1 | PATHOGEN RESISTANCE | 305 |
| PATHOGEN_RESISTANCE_ZM-SWCBR-8-1 | PATHOGEN RESISTANCE | 349 |
| PATHOGEN_RESISTANCE_ZM-SWCBR-8-1 | PATHOGEN RESISTANCE | 349 |
| PATHOGEN_RESISTANCE_ZM-SWCBR-8-1 | PATHOGEN RESISTANCE | 321 |
| PATHOGEN_RESISTANCE_ZM-SWCBR-8-2 | PATHOGEN RESISTANCE | 263 |
| PATHOGEN_RESISTANCE_ZM-SWCBR-8-2 | PATHOGEN RESISTANCE | 263 |
| PATHOGEN_RESISTANCE_ZM-SWCBR-8-2 | PATHOGEN RESISTANCE | 263 |
| PATHOGEN_RESISTANCE_ZM-SWCBR-9-1 | PATHOGEN RESISTANCE | 309 |
| PATHOGEN_RESISTANCE_ZM-SWCBR-9-1 | PATHOGEN RESISTANCE | 131 |
| PATHOGEN_RESISTANCE_ZM-SWCBR-9-1 | PATHOGEN RESISTANCE | 257 |
| PATHOGEN_RESISTANCE_ZM-SWCBR-9-1 | PATHOGEN RESISTANCE | 319 |
| PATHOGEN_RESISTANCE_ZM-SWCBR-9-2 | PATHOGEN RESISTANCE | 285 |
| PATHOGEN_RESISTANCE_ZM-SWCBR-9-2 | PATHOGEN RESISTANCE | 87 |
| PATHOGEN_RESISTANCE_ZM-SWCBR-9-2 | PATHOGEN RESISTANCE | 331 |
| PATHOGEN_RESISTANCE_ZM-SWCBR-9-2 | PATHOGEN RESISTANCE | 57 |
| PATHOGEN_RESISTANCE_ZM-SWCBR-9-2 | PATHOGEN RESISTANCE | 285 |
| PATHOGEN_RESISTANCE_ZM-SWCBR-9-2 | PATHOGEN RESISTANCE | 217 |
| PATHOGEN_RESISTANCE_ZM-SWCBR-9-2 | PATHOGEN RESISTANCE | 319 |
| PATHOGEN_RESISTANCE_ZM-SWCBR-9-3 | PATHOGEN RESISTANCE | 309 |
| PATHOGEN_RESISTANCE_ZM-SWCBR-9-3 | PATHOGEN RESISTANCE | 257 |
| PATHOGEN_RESISTANCE_ZM-SWCBR-9-3 | PATHOGEN RESISTANCE | 319 |
| PATHOGEN_RESISTANCE_ZM-SWCBR-9-4 | PATHOGEN RESISTANCE | 309 |
| PATHOGEN_RESISTANCE_ZM-SWCBR-9-4 | PATHOGEN RESISTANCE | 131 |
| PATHOGEN_RESISTANCE_ZM-SWCBR-9-4 | PATHOGEN RESISTANCE | 257 |
| PATHOGEN_RESISTANCE_ZM-SWCBR-9-4 | PATHOGEN RESISTANCE | 319 |
| QUALITY_ZM-CPC-1-2 | GRAIN_QUALITY | 319 |
| QUALITY_ZM-CPC-1-3 | GRAIN_QUALITY | 257 |
| QUALITY_ZM-CPC-1-4 | GRAIN_QUALITY | 285 |
| QUALITY_ZM-CPC-1-4 | GRAIN_QUALITY | 87 |
| QUALITY_ZM-CPC-1-4 | GRAIN_QUALITY | 87 |
| QUALITY_ZM-CPC-1-4 | GRAIN_QUALITY | 285 |
| QUALITY_ZM-CPC-1-4 | GRAIN_QUALITY | 217 |
| QUALITY_ZM-CPC-1-5 | GRAIN_QUALITY | 257 |
| QUALITY_ZM-CPC-1-6 | GRAIN_QUALITY | 285 |
| QUALITY_ZM-CPC-1-6 | GRAIN_QUALITY | 87 |
| QUALITY_ZM-CPC-1-6 | GRAIN_QUALITY | 87 |
| QUALITY_ZM-CPC-1-6 | GRAIN_QUALITY | 285 |
| QUALITY_ZM-CPC-1-6 | GRAIN_QUALITY | 217 |
| QUALITY_ZM-CPC-10-1 | GRAIN_QUALITY | 217 |
| QUALITY_ZM-CPC-3-1 | GRAIN_QUALITY | 349 |
| QUALITY_ZM-CPC-3-1 | GRAIN_QUALITY | 349 |
| QUALITY_ZM-CPC-3-1 | GRAIN_QUALITY | 57 |
| QUALITY_ZM-CPC-3-2 | GRAIN_QUALITY | 285 |
| QUALITY_ZM-CPC-3-2 | GRAIN_QUALITY | 87 |
| QUALITY_ZM-CPC-3-2 | GRAIN_QUALITY | 87 |
| QUALITY_ZM-CPC-3-2 | GRAIN_QUALITY | 285 |
| QUALITY_ZM-CPC-3-3 | GRAIN_QUALITY | 349 |
| QUALITY_ZM-CPC-3-3 | GRAIN_QUALITY | 349 |
| QUALITY_ZM-CPC-3-3 | GRAIN_QUALITY | 57 |
| QUALITY_ZM-CPC-6-1 | GRAIN_QUALITY | 131 |
| QUALITY_ZM-CPC-6-1 | GRAIN_QUALITY | 349 |
| QUALITY_ZM-CPC-6-1 | GRAIN_QUALITY | 197 |
| QUALITY_ZM-CPC-6-1 | GRAIN_QUALITY | 131 |
| QUALITY_ZM-CPC-6-1 | GRAIN_QUALITY | 349 |
| QUALITY_ZM-CPC-6-2 | GRAIN_QUALITY | 131 |
| QUALITY_ZM-CPC-6-2 | GRAIN_QUALITY | 87 |
| QUALITY_ZM-CPC-6-2 | GRAIN_QUALITY | 349 |
| QUALITY_ZM-CPC-6-2 | GRAIN_QUALITY | 197 |
| QUALITY_ZM-CPC-6-2 | GRAIN_QUALITY | 331 |
| QUALITY_ZM-CPC-6-2 | GRAIN_QUALITY | 131 |
| QUALITY_ZM-CPC-6-2 | GRAIN_QUALITY | 349 |
| QUALITY_ZM-CPC-6-2 | GRAIN_QUALITY | 331 |
| QUALITY_ZM-CPC-6-2 | GRAIN_QUALITY | 87 |
| QUALITY_ZM-CPC-6-2 | GRAIN_QUALITY | 319 |
| QUALITY_ZM-CPC-7-1 | GRAIN_QUALITY | 281 |
| QUALITY_ZM-CPC-7-2 | GRAIN_QUALITY | 87 |
| QUALITY_ZM-CPC-7-2 | GRAIN_QUALITY | 87 |
| QUALITY_ZM-CPC-7-3 | GRAIN_QUALITY | 281 |
| QUALITY_ZM-IVDOM-1-1 | GRAIN_QUALITY | 319 |
| QUALITY_ZM-IVDOM-1-2 | GRAIN_QUALITY | 257 |
| QUALITY_ZM-IVDOM-1-4 | GRAIN_QUALITY | 257 |
| QUALITY_ZM-IVDOM-10-1 | GRAIN_QUALITY | 217 |
| QUALITY_ZM-IVDOM-10-2 | GRAIN_QUALITY | 217 |
| QUALITY_ZM-IVDOM-3-1 | GRAIN_QUALITY | 349 |
| QUALITY_ZM-IVDOM-3-1 | GRAIN_QUALITY | 349 |
| QUALITY_ZM-IVDOM-3-1 | GRAIN_QUALITY | 57 |
| QUALITY_ZM-IVDOM-3-3 | GRAIN_QUALITY | 349 |
| QUALITY_ZM-IVDOM-3-3 | GRAIN_QUALITY | 349 |
| QUALITY_ZM-IVDOM-3-3 | GRAIN_QUALITY | 57 |
| QUALITY_ZM-IVDOM-5-2 | GRAIN_QUALITY | 291 |
| QUALITY_ZM-IVDOM-5-2 | GRAIN_QUALITY | 291 |
| QUALITY_ZM-IVDOM-5-2 | GRAIN_QUALITY | 291 |
| QUALITY_ZM-IVDOM-5-2 | GRAIN_QUALITY | 291 |
| QUALITY_ZM-IVDOM-5-2 | GRAIN_QUALITY | 291 |
| QUALITY_ZM-IVDOM-9-1 | GRAIN_QUALITY | 131 |
| QUALITY_ZM-IVDOM-9-1 | GRAIN_QUALITY | 197 |
| QUALITY_ZM-IVDOM-9-1 | GRAIN_QUALITY | 131 |
| QUALITY_ZM-IVDOM-9-1 | GRAIN_QUALITY | 257 |
| QUALITY_ZM-IVDOM-9-1 | GRAIN_QUALITY | 319 |
| QUALITY_ZM-IVDOM-9-2 | GRAIN_QUALITY | 131 |
| QUALITY_ZM-IVDOM-9-2 | GRAIN_QUALITY | 197 |
| QUALITY_ZM-IVDOM-9-2 | GRAIN_QUALITY | 131 |
| QUALITY_ZM-IVDOM-9-2 | GRAIN_QUALITY | 257 |
| QUALITY_ZM-IVDOM-9-2 | GRAIN_QUALITY | 319 |
| QUALITY_ZM-PC-1-1 | GRAIN_QUALITY | 257 |
| QUALITY_ZM-PC-5-1 | GRAIN_QUALITY | 25 |
| QUALITY_ZM-PC-5-1 | GRAIN_QUALITY | 25 |
| QUALITY_ZM-PC-8-1 | GRAIN_QUALITY | 71 |
| QUALITY_ZM-PC-8-1 | GRAIN_QUALITY | 131 |
| QUALITY_ZM-PC-8-1 | GRAIN_QUALITY | 349 |
| QUALITY_ZM-PC-8-1 | GRAIN_QUALITY | 197 |
| QUALITY_ZM-PC-8-1 | GRAIN_QUALITY | 263 |
| QUALITY_ZM-PC-8-1 | GRAIN_QUALITY | 131 |
| QUALITY_ZM-PC-8-1 | GRAIN_QUALITY | 257 |
| QUALITY_ZM-PC-8-1 | GRAIN_QUALITY | 263 |
| QUALITY_ZM-PC-8-1 | GRAIN_QUALITY | 349 |
| QUALITY_ZM-PC-8-1 | GRAIN_QUALITY | 321 |
| QUALITY_ZM-PC-8-1 | GRAIN_QUALITY | 263 |
| QUALITY_ZM-PC-8-1 | GRAIN_QUALITY | 237 |
| QUALITY_ZM-PC-9-1 | GRAIN_QUALITY | 285 |
| QUALITY_ZM-PC-9-1 | GRAIN_QUALITY | 87 |
| QUALITY_ZM-PC-9-1 | GRAIN_QUALITY | 331 |
| QUALITY_ZM-PC-9-1 | GRAIN_QUALITY | 331 |
| QUALITY_ZM-PC-9-1 | GRAIN_QUALITY | 87 |
| QUALITY_ZM-PC-9-1 | GRAIN_QUALITY | 57 |
| QUALITY_ZM-PC-9-1 | GRAIN_QUALITY | 285 |
| QUALITY_ZM-PC-9-1 | GRAIN_QUALITY | 217 |
| QUALITY_ZM-PC-9-1 | GRAIN_QUALITY | 319 |
| QUALITY_ZM-STC-10-2 | GRAIN_QUALITY | 217 |
| QUALITY_ZM-STC-2-2 | GRAIN_QUALITY | 359 |
| QUALITY_ZM-STC-2-2 | GRAIN_QUALITY | 359 |
| QUALITY_ZM-STC-2-2 | GRAIN_QUALITY | 217 |
| QUALITY_ZM-STC-2-2 | GRAIN_QUALITY | 33 |
| QUALITY_ZM-STC-5-1 | GRAIN_QUALITY | 25 |
| QUALITY_ZM-STC-5-1 | GRAIN_QUALITY | 25 |
| QUALITY_ZM-STC-6-1 | GRAIN_QUALITY | 285 |
| QUALITY_ZM-STC-6-1 | GRAIN_QUALITY | 131 |
| QUALITY_ZM-STC-6-1 | GRAIN_QUALITY | 349 |
| QUALITY_ZM-STC-6-1 | GRAIN_QUALITY | 197 |
| QUALITY_ZM-STC-6-1 | GRAIN_QUALITY | 263 |
| QUALITY_ZM-STC-6-1 | GRAIN_QUALITY | 131 |
| QUALITY_ZM-STC-6-1 | GRAIN_QUALITY | 263 |
| QUALITY_ZM-STC-6-1 | GRAIN_QUALITY | 349 |
| QUALITY_ZM-STC-6-1 | GRAIN_QUALITY | 285 |
| QUALITY_ZM-STC-6-1 | GRAIN_QUALITY | 263 |
| QUALITY_ZM-STC-6-1 | GRAIN_QUALITY | 237 |
| QUALITY_ZM-STC-7-1 | GRAIN_QUALITY | 39 |
| QUALITY_ZM-STC-7-2 | GRAIN_QUALITY | 281 |
| QUALITY_ZM-STC-8-1 | GRAIN_QUALITY | 71 |
| QUALITY_ZM-STC-8-1 | GRAIN_QUALITY | 131 |
| QUALITY_ZM-STC-8-1 | GRAIN_QUALITY | 349 |
| QUALITY_ZM-STC-8-1 | GRAIN_QUALITY | 197 |
| QUALITY_ZM-STC-8-1 | GRAIN_QUALITY | 263 |
| QUALITY_ZM-STC-8-1 | GRAIN_QUALITY | 131 |
| QUALITY_ZM-STC-8-1 | GRAIN_QUALITY | 257 |
| QUALITY_ZM-STC-8-1 | GRAIN_QUALITY | 263 |
| QUALITY_ZM-STC-8-1 | GRAIN_QUALITY | 349 |
| QUALITY_ZM-STC-8-1 | GRAIN_QUALITY | 321 |
| QUALITY_ZM-STC-8-1 | GRAIN_QUALITY | 263 |
| QUALITY_ZM-STC-8-1 | GRAIN_QUALITY | 237 |
| YIELD_ZM-BIOM-2-1 | YIELD | 87 |
| YIELD_ZM-BIOM-2-1 | YIELD | 87 |
| YIELD_ZM-BIOM-3-1 | YIELD | 285 |
| YIELD_ZM-BIOM-3-1 | YIELD | 87 |
| YIELD_ZM-BIOM-3-1 | YIELD | 257 |
| YIELD_ZM-BIOM-3-1 | YIELD | 87 |
| YIELD_ZM-BIOM-7-1 | YIELD | 87 |
| YIELD_ZM-BIOM-7-1 | YIELD | 281 |
| YIELD_ZM-BIOM-7-1 | YIELD | 281 |
| YIELD_ZM-BIOM-7-1 | YIELD | 87 |
| YIELD_ZM-BIOM-8-1 | YIELD | 263 |
| YIELD_ZM-BIOM-8-1 | YIELD | 263 |
| YIELD_ZM-BIOM-8-1 | YIELD | 263 |
| YIELD_ZM-DMC-1-1 | YIELD | 257 |
| YIELD_ZM-DMC-1-2 | YIELD | 285 |
| YIELD_ZM-DMC-1-2 | YIELD | 87 |
| YIELD_ZM-DMC-1-2 | YIELD | 87 |
| YIELD_ZM-DMC-1-2 | YIELD | 217 |
| YIELD_ZM-DMC-5-1 | YIELD | 25 |
| YIELD_ZM-DMC-5-1 | YIELD | 25 |
| YIELD_ZM-DMC-6-1 | YIELD | 131 |
| YIELD_ZM-DMC-6-1 | YIELD | 87 |
| YIELD_ZM-DMC-6-1 | YIELD | 331 |
| YIELD_ZM-DMC-6-1 | YIELD | 349 |
| YIELD_ZM-DMC-6-1 | YIELD | 87 |
| YIELD_ZM-DMC-6-1 | YIELD | 319 |
| YIELD_ZM-DMC-6-2 | YIELD | 131 |
| YIELD_ZM-DMC-6-2 | YIELD | 87 |
| YIELD_ZM-DMC-6-2 | YIELD | 331 |
| YIELD_ZM-DMC-6-2 | YIELD | 87 |
| YIELD_ZM-DMC-6-2 | YIELD | 237 |
| YIELD_ZM-DMC-6-2 | YIELD | 319 |
| YIELD_ZM-DMC-6-3 | YIELD | 131 |
| YIELD_ZM-DMC-6-3 | YIELD | 349 |
| YIELD_ZM-DMY-1-3 | YIELD | 285 |
| YIELD_ZM-DMY-1-3 | YIELD | 87 |
| YIELD_ZM-DMY-1-3 | YIELD | 87 |
| YIELD_ZM-DMY-1-3 | YIELD | 217 |
| YIELD_ZM-DMY-1-4 | YIELD | 257 |
| YIELD_ZM-DMY-1-5 | YIELD | 285 |
| YIELD_ZM-DMY-1-5 | YIELD | 87 |
| YIELD_ZM-DMY-1-5 | YIELD | 87 |
| YIELD_ZM-DMY-1-5 | YIELD | 217 |
| YIELD_ZM-DMY-2-1 | YIELD | 87 |
| YIELD_ZM-DMY-2-1 | YIELD | 87 |
| YIELD_ZM-DMY-2-1 | YIELD | 217 |
| YIELD_ZM-DMY-2-3 | YIELD | 285 |
| YIELD_ZM-DMY-2-3 | YIELD | 359 |
| YIELD_ZM-DMY-2-3 | YIELD | 33 |
| YIELD_ZM-DMY-2-3 | YIELD | 233 |
| YIELD_ZM-DMY-2-3 | YIELD | 359 |
| YIELD_ZM-DMY-2-3 | YIELD | 233 |
| YIELD_ZM-DMY-2-3 | YIELD | 281 |
| YIELD_ZM-DMY-2-3 | YIELD | 281 |
| YIELD_ZM-DMY-2-3 | YIELD | 233 |
| YIELD_ZM-DMY-2-3 | YIELD | 233 |
| YIELD_ZM-DMY-2-3 | YIELD | 33 |
| YIELD_ZM-DMY-2-4 | YIELD | 359 |
| YIELD_ZM-DMY-2-4 | YIELD | 33 |
| YIELD_ZM-DMY-2-4 | YIELD | 359 |
| YIELD_ZM-DMY-2-4 | YIELD | 217 |
| YIELD_ZM-DMY-2-4 | YIELD | 33 |
| YIELD_ZM-DMY-3-1 | YIELD | 285 |
| YIELD_ZM-DMY-3-1 | YIELD | 87 |
| YIELD_ZM-DMY-3-1 | YIELD | 87 |
| YIELD_ZM-DMY-3-2 | YIELD | 349 |
| YIELD_ZM-DMY-3-2 | YIELD | 57 |
| YIELD_ZM-DMY-3-3 | YIELD | 349 |
| YIELD_ZM-DMY-3-3 | YIELD | 57 |
| YIELD_ZM-DMY-4-1 | YIELD | 59 |
| YIELD_ZM-DMY-4-2 | YIELD | 131 |
| YIELD_ZM-DMY-4-2 | YIELD | 25 |
| YIELD_ZM-DMY-4-2 | YIELD | 25 |
| YIELD_ZM-DMY-4-3 | YIELD | 291 |
| YIELD_ZM-DMY-4-3 | YIELD | 273 |
| YIELD_ZM-DMY-4-3 | YIELD | 71 |
| YIELD_ZM-DMY-4-3 | YIELD | 33 |
| YIELD_ZM-DMY-4-3 | YIELD | 291 |
| YIELD_ZM-DMY-4-3 | YIELD | 291 |
| YIELD_ZM-DMY-4-3 | YIELD | 25 |
| YIELD_ZM-DMY-4-3 | YIELD | 273 |
| YIELD_ZM-DMY-4-3 | YIELD | 291 |
| YIELD_ZM-DMY-4-3 | YIELD | 291 |
| YIELD_ZM-DMY-4-3 | YIELD | 273 |
| YIELD_ZM-DMY-4-3 | YIELD | 273 |
| YIELD_ZM-DMY-4-3 | YIELD | 25 |
| YIELD_ZM-DMY-4-3 | YIELD | 57 |
| YIELD_ZM-DMY-4-3 | YIELD | 273 |
| YIELD_ZM-DMY-4-3 | YIELD | 33 |
| YIELD_ZM-DMY-4-4 | YIELD | 131 |
| YIELD_ZM-DMY-4-4 | YIELD | 25 |
| YIELD_ZM-DMY-4-4 | YIELD | 25 |
| YIELD_ZM-DMY-5-1 | YIELD | 291 |
| YIELD_ZM-DMY-5-1 | YIELD | 291 |
| YIELD_ZM-DMY-5-1 | YIELD | 291 |
| YIELD_ZM-DMY-5-1 | YIELD | 291 |
| YIELD_ZM-DMY-5-1 | YIELD | 291 |
| YIELD_ZM-DMY-6-1 | YIELD | 131 |
| YIELD_ZM-DMY-6-1 | YIELD | 349 |
| YIELD_ZM-DMY-7-1 | YIELD | 281 |
| YIELD_ZM-DMY-7-1 | YIELD | 281 |
| YIELD_ZM-DMY-8-1 | YIELD | 71 |
| YIELD_ZM-DMY-8-1 | YIELD | 131 |
| YIELD_ZM-DMY-8-1 | YIELD | 305 |
| YIELD_ZM-DMY-8-1 | YIELD | 263 |
| YIELD_ZM-DMY-8-1 | YIELD | 257 |
| YIELD_ZM-DMY-8-1 | YIELD | 263 |
| YIELD_ZM-DMY-8-1 | YIELD | 349 |
| YIELD_ZM-DMY-8-1 | YIELD | 321 |
| YIELD_ZM-DMY-8-1 | YIELD | 263 |
| YIELD_ZM-DMY-8-1 | YIELD | 237 |
| YIELD_ZM-DMY-8-2 | YIELD | 263 |
| YIELD_ZM-DMY-8-2 | YIELD | 263 |
| YIELD_ZM-DMY-8-2 | YIELD | 263 |
| YIELD_ZM-DMY-9-1 | YIELD | 309 |
| YIELD_ZM-DMY-9-1 | YIELD | 257 |
| YIELD_ZM-DMY-9-1 | YIELD | 319 |
| YIELD_ZM-EWT-4-2 | YIELD | 131 |
| YIELD_ZM-EWT-4-2 | YIELD | 25 |
| YIELD_ZM-EWT-4-2 | YIELD | 25 |
| YIELD_ZM-GWM2-3-1 | YIELD | 349 |
| YIELD_ZM-GWM2-3-1 | YIELD | 57 |
| YIELD_ZM-GWM2-3-2 | YIELD | 285 |
| YIELD_ZM-GWM2-3-2 | YIELD | 87 |
| YIELD_ZM-GWM2-3-2 | YIELD | 257 |
| YIELD_ZM-GWM2-3-2 | YIELD | 87 |
| YIELD_ZM-GWM2-7-1 | YIELD | 87 |
| YIELD_ZM-GWM2-7-1 | YIELD | 87 |
| YIELD_ZM-GYHA-1-2 | YIELD | 71 |
| YIELD_ZM-GYHA-1-2 | YIELD | 217 |
| YIELD_ZM-GYHA-1-3 | YIELD | 319 |
| YIELD_ZM-GYHA-1-4 | YIELD | 319 |
| YIELD_ZM-GYHA-3-1 | YIELD | 285 |
| YIELD_ZM-GYHA-3-1 | YIELD | 87 |
| YIELD_ZM-GYHA-3-1 | YIELD | 87 |
| YIELD_ZM-GYHA-5-1 | YIELD | 263 |
| YIELD_ZM-GYHA-5-1 | YIELD | 331 |
| YIELD_ZM-GYHA-5-1 | YIELD | 263 |
| YIELD_ZM-GYHA-5-1 | YIELD | 263 |
| YIELD_ZM-GYHA-6-1 | YIELD | 285 |
| YIELD_ZM-GYHA-6-1 | YIELD | 131 |
| YIELD_ZM-GYHA-6-1 | YIELD | 263 |
| YIELD_ZM-GYHA-6-1 | YIELD | 263 |
| YIELD_ZM-GYHA-6-1 | YIELD | 263 |
| YIELD_ZM-GYLD-6-1 | YIELD | 131 |
| YIELD_ZM-GYLD-6-1 | YIELD | 87 |
| YIELD_ZM-GYLD-6-1 | YIELD | 331 |
| YIELD_ZM-GYLD-6-1 | YIELD | 349 |
| YIELD_ZM-GYLD-6-1 | YIELD | 87 |
| YIELD_ZM-GYLD-6-1 | YIELD | 319 |
| YIELD_ZM-GYLD-7-1 | YIELD | 87 |
| YIELD_ZM-GYLD-7-1 | YIELD | 87 |
| YIELD_ZM-HI-1-1 | YIELD | 71 |
| YIELD_ZM-HI-1-1 | YIELD | 217 |
| YIELD_ZM-HI-1-1 | YIELD | 319 |
| YIELD_ZM-HI-3-1 | YIELD | 349 |
| YIELD_ZM-HI-3-1 | YIELD | 57 |
| YIELD_ZM-HI-4-1 | YIELD | 305 |
| YIELD_ZM-HI-4-1 | YIELD | 25 |
| YIELD_ZM-HI-4-1 | YIELD | 25 |
| YIELD_ZM-HI-4-1 | YIELD | 321 |
| YIELD_ZM-HI-7-1 | YIELD | 87 |
| YIELD_ZM-HI-7-1 | YIELD | 87 |
| YIELD_ZM-HI-8-1 | YIELD | 263 |
| YIELD_ZM-HI-8-1 | YIELD | 263 |
| YIELD_ZM-HI-8-1 | YIELD | 263 |
| YIELD_ZM-KNE-4-1 | YIELD | 131 |
| YIELD_ZM-KNE-4-1 | YIELD | 25 |
| YIELD_ZM-KNE-4-1 | YIELD | 25 |
| YIELD_ZM-KW100-1-2 | YIELD | 319 |
| YIELD_ZM-KW100-3-1 | YIELD | 285 |
| YIELD_ZM-KW100-3-1 | YIELD | 87 |
| YIELD_ZM-KW100-3-1 | YIELD | 87 |
| YIELD_ZM-KW100-9-1 | YIELD | 99 |
| YIELD_ZM-KW100-9-1 | YIELD | 131 |
| YIELD_ZM-KW100-9-1 | YIELD | 309 |
| YIELD_ZM-KW100-9-1 | YIELD | 331 |
| YIELD_ZM-KW100-9-1 | YIELD | 257 |
| YIELD_ZM-KW100-9-1 | YIELD | 319 |
| YIELD_ZM-KW300-1-2 | YIELD | 257 |
| YIELD_ZM-KW300-3-2 | YIELD | 349 |
| YIELD_ZM-KW300-3-2 | YIELD | 57 |
| YIELD_ZM-KW300-3-3 | YIELD | 285 |
| YIELD_ZM-KW300-3-3 | YIELD | 87 |
| YIELD_ZM-KW300-3-3 | YIELD | 87 |
| YIELD_ZM-KW300-4-2 | YIELD | 131 |
| YIELD_ZM-KW300-4-2 | YIELD | 25 |
| YIELD_ZM-KW300-4-2 | YIELD | 25 |
| YIELD_ZM-KW300-6-1 | YIELD | 131 |
| YIELD_ZM-KW300-6-1 | YIELD | 349 |
| YIELD_ZM-KW300-6-2 | YIELD | 285 |
| YIELD_ZM-KW300-6-2 | YIELD | 131 |
| YIELD_ZM-KW300-6-2 | YIELD | 263 |
| YIELD_ZM-KW300-6-2 | YIELD | 263 |
| YIELD_ZM-KW300-6-2 | YIELD | 263 |
| YIELD_ZM-KW300-8-1 | YIELD | 71 |
| YIELD_ZM-KW300-8-1 | YIELD | 305 |
| YIELD_ZM-KW300-8-1 | YIELD | 349 |
| YIELD_ZM-KW300-8-1 | YIELD | 321 |
| YIELD_ZM-KW300-9-1 | YIELD | 285 |
| YIELD_ZM-KW300-9-1 | YIELD | 87 |
| YIELD_ZM-KW300-9-1 | YIELD | 331 |
| YIELD_ZM-KW300-9-1 | YIELD | 87 |
| YIELD_ZM-KW300-9-1 | YIELD | 57 |
| YIELD_ZM-KW300-9-1 | YIELD | 217 |
| YIELD_ZM-KW300-9-2 | YIELD | 285 |
| YIELD_ZM-KW300-9-2 | YIELD | 99 |
| YIELD_ZM-KW300-9-2 | YIELD | 87 |
| YIELD_ZM-KW300-9-2 | YIELD | 331 |
| YIELD_ZM-KW300-9-2 | YIELD | 87 |
| YIELD_ZM-KW300-9-2 | YIELD | 57 |
| YIELD_ZM-KW300-9-2 | YIELD | 217 |
| YIELD_ZM-KWE-4-1 | YIELD | 131 |
| YIELD_ZM-KWE-4-1 | YIELD | 25 |
| YIELD_ZM-KWE-4-1 | YIELD | 25 |

TABLE 6B
References for the listed quantitative trait loci (QTLs) listed
in Table 6A

| REFERENCE | QTL |
|---|---|
| THEOR APPL GENET (1998) 97:744-755 | ZM-LABA-1-1 |
| THEOR APPL GENET (1998) 97:744-755 | ZM-LABA-1-2 |
| THEOR APPL GENET (1998) 97:744-755 | ZM-LABA-2-4 |
| THEOR APPL GENET (1998) 97:744-755 | ZM-LABA-2-3 |
| THEOR APPL GENET (1998) 97:744-755 | ZM-LABA-2-1 |
| THEOR APPL GENET (1998) 97:744-755 | ZM-LABA-3-1 |
| THEOR APPL GENET (1998) 97:744-755 | ZM-LABA-4-2 |
| THEOR APPL GENET (1998) 97:744-755 | ZM-LABA-4-1 |
| THEOR APPL GENET (1998) 97:744-755 | ZM-LABA-7-1 |
| THEOR APPL GENET (1998) 97:744-755 | ZM-LABA-7-2 |
| THEOR APPL GENET (1998) 97:744-755 | ZM-LABA-9-1 |
| THEOR APPL GENET (1998) 97:744-755 | ZM-LABA-10-2 |
| GENETICS (1998) 149:1997-2006 | ZM-CELW-5-1 |
| GENETICS (1998) 149:1997-2006 | ZM-CELW-6-1 |
| PHYTOPATHOLOGY (1998) 88:1324-1329 | ZM-CRR-1-10 |
| PHYTOPATHOLOGY (1998) 88:1324-1329 | ZM-CRR-6-4 |
| PHYTOPATHOLOGY (1998) 88:1324-1329 | ZM-CRR-9-2 |
| PHYTOPATHOLOGY (1998) 88:1324-1329 | ZM-CRR-1-12 |
| PHYTOPATHOLOGY (1998) 88:1324-1329 | ZM-CRR-1-11 |
| PHYTOPATHOLOGY (1998) 88:1324-1329 | ZM-CRR-4-3 |
| PHYTOPATHOLOGY (1998) 88:1324-1329 | ZM-CRR-7-5 |
| PHYTOPATHOLOGY (1998) 88:1324-1329 | ZM-CRR-8-6 |
| PHYTOPATHOLOGY (1998) 88:1324-1329 | ZM-CRR-9-3 |
| PHYTOPATHOLOGY (1998) 88:1324-1329 | ZM-CRR-1-13 |
| PHYTOPATHOLOGY (1998) 88:1324-1329 | ZM-CRR-2-11 |
| PHYTOPATHOLOGY (1998) 88:1324-1329 | ZM-CRR-3-7 |
| PHYTOPATHOLOGY (1998) 88:1324-1329 | ZM-CRR-3-6 |
| PHYTOPATHOLOGY (1998) 88:1324-1329 | ZM-CRR-4-5 |
| PHYTOPATHOLOGY (1998) 88:1324-1329 | ZM-CRR-4-4 |
| PHYTOPATHOLOGY (1998) 88:1324-1329 | ZM-CRR-5-6 |
| PHYTOPATHOLOGY (1998) 88:1324-1329 | ZM-CRR-5-7 |
| PHYTOPATHOLOGY (1998) 88:1324-1329 | ZM-CRR-6-5 |
| PHYTOPATHOLOGY (1998) 88:1324-1329 | ZM-CRR-7-6 |
| PHYTOPATHOLOGY (1998) 88:1324-1329 | ZM-CRR-8-7 |
| PHYTOPATHOLOGY (1998) 88:1324-1329 | ZM-CRR-9-4 |
| THEOR APPL GENET (1998) 97:1321-1330 | ZM-PCSI-1-1 |
| THEOR APPL GENET (1998) 97:1321-1330 | ZM-PCSI-2-1 |
| THEOR APPL GENET (1998) 97:1321-1330 | ZM-PCSI-3-1 |
| THEOR APPL GENET (1998) 97:1321-1330 | ZM-PCSI-3-2 |
| THEOR APPL GENET (1998) 97:1321-1330 | ZM-PCSI-4-1 |
| THEOR APPL GENET (1998) 97:1321-1330 | ZM-PCSI-5-1 |
| THEOR APPL GENET (1998) 97:1321-1330 | ZM-PCSI-9-2 |
| THEOR APPL GENET (1998) 97:1321-1330 | ZM-PCSI-9-1 |
| THEOR APPL GENET (1998) 97:1321-1330 | ZM-PCSI-1-2 |
| THEOR APPL GENET (1998) 97:1321-1330 | ZM-PCSI-3-3 |
| THEOR APPL GENET (1998) 97:1321-1330 | ZM-PCSI-5-2 |
| THEOR APPL GENET (1998) 97:1321-1330 | ZM-PCSI-6-1 |
| THEOR APPL GENET (1998) 97:1321-1330 | ZM-PCSI-1-4 |
| THEOR APPL GENET (1998) 97:1321-1330 | ZM-PCSI-2-4 |
| THEOR APPL GENET (1998) 97:1321-1330 | ZM-PCSI-3-4 |
| THEOR APPL GENET (1998) 97:1321-1330 | ZM-PCSI-5-3 |
| THEOR APPL GENET (1998) 97:1321-1330 | ZM-PCSI-1-5 |
| THEOR APPL GENET (1998) 97:1321-1330 | ZM-PCSI-1-6 |
| THEOR APPL GENET (1998) 97:1321-1330 | ZM-PCSI-2-5 |
| THEOR APPL GENET (1998) 97:1321-1330 | ZM-PCSI-3-5 |
| THEOR APPL GENET (1998) 97:1321-1330 | ZM-PCSI-4-2 |
| THEOR APPL GENET (1998) 97:1321-1330 | ZM-PCSI-4-3 |
| THEOR APPL GENET (1998) 97:1321-1330 | ZM-PCSI-4-5 |
| THEOR APPL GENET (1998) 97:1321-1330 | ZM-PCSI-5-5 |
| THEOR APPL GENET (1998) 97:1321-1330 | ZM-PCSI-7-1 |
| THEOR APPL GENET (1998) 97:1321-1330 | ZM-PCSI-7-2 |
| THEOR APPL GENET (1998) 97:1321-1330 | ZM-PCSI-9-3 |
| THEOR APPL GENET (1998) 97:1321-1330 | ZM-CSVEG-1-1 |
| THEOR APPL GENET (1998) 97:1321-1330 | ZM-CSVEG-4-1 |
| THEOR APPL GENET (1998) 97:1321-1330 | ZM-CSVEG-4-2 |
| THEOR APPL GENET (1998) 97:1321-1330 | ZM-CSVEG-7-1 |
| THEOR APPL GENET (1998) 97:1321-1330 | ZM-CSVEG-9-1 |
| THEOR APPL GENET (1998) 97:1321-1330 | ZM-CSVSG-1-1 |
| THEOR APPL GENET (1998) 97:1321-1330 | ZM-CSVSG-4-1 |
| CROP SCI (1998) 38:1062-1072 | ZM-LT-1-1 |
| CROP SCI (1998) 38:1062-1072 | ZM-LT-1-2 |
| CROP SCI (1998) 38:1062-1072 | ZM-LT-4-1 |
| CROP SCI (1998) 38:1062-1072 | ZM-LT-7-1 |
| CROP SCI (1998) 38:1062-1072 | ZM-PC-1-1 |
| CROP SCI (1998) 38:1062-1072 | ZM-PC-5-1 |
| CROP SCI (1998) 38:1062-1072 | ZM-PC-8-1 |
| CROP SCI (1998) 38:1062-1072 | ZM-PC-9-1 |
| CROP SCI (1998) 38:1062-1072 | ZM-SWCBR-1-4 |
| CROP SCI (1998) 38:1062-1072 | ZM-SWCBR-1-3 |
| CROP SCI (1998) 38:1062-1072 | ZM-SWCBR-1-2 |
| CROP SCI (1998) 38:1062-1072 | ZM-SWCBR-1-5 |
| CROP SCI (1998) 38:1062-1072 | ZM-SWCBR-5-3 |
| CROP SCI (1998) 38:1062-1072 | ZM-SWCBR-7-3 |
| CROP SCI (1998) 38:1062-1072 | ZM-SWCBR-8-2 |
| CROP SCI (1998) 38:1062-1072 | ZM-SWCBR-9-2 |
| CROP SCI (1998) 38:1062-1072 | ZM-SCBR-1-2 |
| CROP SCI (1998) 38:1062-1072 | ZM-SCBR-1-1 |
| CROP SCI (1998) 38:1062-1072 | ZM-SCBR-5-1 |
| CROP SCI (1998) 38:1062-1072 | ZM-SCBR-7-1 |
| CROP SCI (1998) 38:1062-1072 | ZM-SCBR-8-1 |
| CROP SCI (1998) 38:1062-1072 | ZM-SCBR-9-1 |
| CROP SCI (1998) 38:1062-1072 | ZM-LT-5-1 |
| CROP SCI (1998) 38:1062-1072 | ZM-SWCBR-1-6 |
| CROP SCI (1998) 38:1062-1072 | ZM-SWCBR-6-3 |
| CROP SCI (1998) 38:1062-1072 | ZM-SWCBR-9-3 |
| CROP SCI (1998) 38:1062-1072 | ZM-SWCBR-9-4 |
| PLANT BREEDING (1998) 117:309-318 | ZM-ASI-8-1 |
| PLANT BREEDING (1998) 117:309-318 | ZM-SILK-3-1 |
| PLANT BREEDING (1998) 117:309-318 | ZM-SILK-4-1 |
| PLANT BREEDING (1998) 117:309-318 | ZM-SWCBR-3-1 |
| PLANT BREEDING (1998) 117:309-318 | ZM-SWCBR-5-1 |
| PLANT BREEDING (1998) 117:309-318 | ZM-SWCBR-5-2 |
| PLANT BREEDING (1998) 117:309-318 | ZM-SWCBR-6-1 |
| PLANT BREEDING (1998) 117:309-318 | ZM-SWCBR-6-2 |
| PLANT BREEDING (1998) 117:309-318 | ZM-SWCBR-8-1 |
| PLANT BREEDING (1998) 117:309-318 | ZM-SWCBR-9-1 |
| PLANT BREEDING (1998) 117:193-202 | ZM-ASI-1-3 |
| PLANT BREEDING (1998) 117:193-202 | ZM-GYLD-7-1 |
| PLANT BREEDING (1998) 117:193-202 | ZM-SILK-3-2 |
| PLANT BREEDING (1998) 117:193-202 | ZM-SILK-6-2 |
| PLANT BREEDING (1998) 117:193-202 | ZM-SILK-7-2 |
| PLANT BREEDING (1998) 117:193-202 | ZM-SILK-8-1 |
| PLANT BREEDING (1998) 117:193-202 | ZM-SILK-9-3 |
| PLANT BREEDING (1998) 117:193-202 | ZM-SWCBR-1-1 |
| PLANT BREEDING (1998) 117:193-202 | ZM-SWCBR-3-2 |
| PLANT BREEDING (1998) 117:193-202 | ZM-SWCBR-7-1 |
| PLANT BREEDING (1998) 117:193-202 | ZM-SWCBR-7-2 |
| PLANT BREEDING (1998) 117:193-202 | ZM-ASI-1-5 |
| PLANT BREEDING (1998) 117:193-202 | ZM-ASI-3-4 |
| PLANT BREEDING (1998) 117:193-202 | ZM-ASI-5-3 |
| PLANT BREEDING (1998) 117:193-202 | ZM-ASI-10-2 |
| PLANT BREEDING (1998) 117:193-202 | ZM-GYLD-6-1 |
| PLANT BREEDING (1998) 117:193-202 | ZM-SILK-3-5 |
| CROP SCI (1998) 38:1278-1289 | ZM-CPC-7-1 |
| CROP SCI (1998) 38:1278-1289 | ZM-DMC-5-1 |
| CROP SCI (1998) 38:1278-1289 | ZM-DMC-6-2 |
| CROP SCI (1998) 38:1278-1289 | ZM-DMC-6-1 |
| CROP SCI (1998) 38:1278-1289 | ZM-DMY-3-1 |
| CROP SCI (1998) 38:1278-1289 | ZM-DMY-4-2 |
| CROP SCI (1998) 38:1278-1289 | ZM-DMY-4-1 |
| CROP SCI (1998) 38:1278-1289 | ZM-DMY-7-1 |
| CROP SCI (1998) 38:1278-1289 | ZM-DMY-8-1 |
| CROP SCI (1998) 38:1278-1289 | ZM-DMY-9-1 |
| CROP SCI (1998) 38:1278-1289 | ZM-IVDOM-3-1 |
| CROP SCI (1998) 38:1278-1289 | ZM-IVDOM-5-2 |
| CROP SCI (1998) 38:1278-1289 | ZM-STC-5-1 |
| CROP SCI (1998) 38:1278-1289 | ZM-STC-7-1 |
| CROP SCI (1998) 38:1278-1289 | ZM-STC-8-1 |
| CROP SCI (1998) 38:1278-1289 | ZM-CPC-1-4 |
| CROP SCI (1998) 38:1278-1289 | ZM-CPC-1-3 |
| CROP SCI (1998) 38:1278-1289 | ZM-CPC-1-2 |
| CROP SCI (1998) 38:1278-1289 | ZM-CPC-3-2 |
| CROP SCI (1998) 38:1278-1289 | ZM-CPC-3-1 |
| CROP SCI (1998) 38:1278-1289 | ZM-CPC-6-1 |
| CROP SCI (1998) 38:1278-1289 | ZM-CPC-7-3 |
| CROP SCI (1998) 38:1278-1289 | ZM-CPC-7-2 |
| CROP SCI (1998) 38:1278-1289 | ZM-DMC-1-1 |
| CROP SCI (1998) 38:1278-1289 | ZM-DMY-1-3 |
| CROP SCI (1998) 38:1278-1289 | ZM-DMY-2-4 |
| CROP SCI (1998) 38:1278-1289 | ZM-DMY-2-3 |
| CROP SCI (1998) 38:1278-1289 | ZM-DMY-2-1 |
| CROP SCI (1998) 38:1278-1289 | ZM-DMY-3-2 |
| CROP SCI (1998) 38:1278-1289 | ZM-DMY-5-1 |
| CROP SCI (1998) 38:1278-1289 | ZM-DMY-6-1 |
| CROP SCI (1998) 38:1278-1289 | ZM-DMY-8-2 |
| CROP SCI (1998) 38:1278-1289 | ZM-IVDOM-1-2 |
| CROP SCI (1998) 38:1278-1289 | ZM-IVDOM-1-1 |
| CROP SCI (1998) 38:1278-1289 | ZM-IVDOM-10-1 |
| CROP SCI (1998) 38:1278-1289 | ZM-STC-6-1 |
| CROP SCI (1998) 38:1278-1289 | ZM-CPC-1-5 |
| CROP SCI (1998) 38:1278-1289 | ZM-CPC-1-6 |
| CROP SCI (1998) 38:1278-1289 | ZM-CPC-3-3 |
| CROP SCI (1998) 38:1278-1289 | ZM-CPC-6-2 |
| CROP SCI (1998) 38:1278-1289 | ZM-CPC-10-1 |
| CROP SCI (1998) 38:1278-1289 | ZM-DMC-1-2 |
| CROP SCI (1998) 38:1278-1289 | ZM-DMC-6-3 |
| CROP SCI (1998) 38:1278-1289 | ZM-DMY-1-4 |
| CROP SCI (1998) 38:1278-1289 | ZM-DMY-1-5 |
| CROP SCI (1998) 38:1278-1289 | ZM-DMY-3-3 |
| CROP SCI (1998) 38:1278-1289 | ZM-DMY-4-4 |
| CROP SCI (1998) 38:1278-1289 | ZM-DMY-4-3 |
| CROP SCI (1998) 38:1278-1289 | ZM-IVDOM-1-4 |
| CROP SCI (1998) 38:1278-1289 | ZM-IVDOM-3-3 |
| CROP SCI (1998) 38:1278-1289 | ZM-IVDOM-9-2 |
| CROP SCI (1998) 38:1278-1289 | ZM-IVDOM-9-1 |
| CROP SCI (1998) 38:1278-1289 | ZM-IVDOM-10-2 |
| CROP SCI (1998) 38:1278-1289 | ZM-STC-2-2 |
| CROP SCI (1998) 38:1278-1289 | ZM-STC-7-2 |
| CROP SCI (1998) 38:1278-1289 | ZM-STC-10-2 |
| CROP SCI (1998) 38:1296-1308 | ZM-GYHA-1-2 |
| CROP SCI (1998) 38:1296-1308 | ZM-GYHA-1-4 |
| CROP SCI (1998) 38:1296-1308 | ZM-GYHA-1-3 |
| CROP SCI (1998) 38:1296-1308 | ZM-GYHA-3-1 |
| CROP SCI (1998) 38:1296-1308 | ZM-GYHA-5-1 |
| CROP SCI (1998) 38:1296-1308 | ZM-GYHA-6-1 |
| CROP SCI (1998) 38:1296-1308 | ZM-KW300-1-2 |
| CROP SCI (1998) 38:1296-1308 | ZM-KW300-3-3 |
| CROP SCI (1998) 38:1296-1308 | ZM-KW300-3-2 |
| CROP SCI (1998) 38:1296-1308 | ZM-KW300-4-2 |
| CROP SCI (1998) 38:1296-1308 | ZM-KW300-6-2 |
| CROP SCI (1998) 38:1296-1308 | ZM-KW300-6-1 |
| CROP SCI (1998) 38:1296-1308 | ZM-KW300-8-1 |
| CROP SCI (1998) 38:1296-1308 | ZM-KW300-9-1 |
| CROP SCI (1998) 38:1296-1308 | ZM-KW300-9-2 |
| THEOR APPL GENET (1999) 99:519-523 | ZM-DMS-1-2 |
| THEOR APPL GENET (1999) 99:519-523 | ZM-DMS-1-1 |
| THEOR APPL GENET (1999) 99:519-523 | ZM-DMS-9-1 |
| THEOR APPL GENET (1999) 99:289-295 | ZM-ASI-7-2 |
| THEOR APPL GENET (1999) 99:289-295 | ZM-DTOL-4-3 |
| THEOR APPL GENET (1999) 99:280-288 | ZM-DTOL-1-1 |
| THEOR APPL GENET (1999) 99:280-288 | ZM-DTOL-4-1 |
| THEOR APPL GENET (1999) 99:280-288 | ZM-DTOL-9-1 |
| THEOR APPL GENET (1999) 99:280-288 | ZM-EWT-4-2 |
| THEOR APPL GENET (1999) 99:280-288 | ZM-KNE-4-1 |
| THEOR APPL GENET (1999) 99:280-288 | ZM-KWE-4-1 |
| THEOR APPL GENET (1999) 99:593-598 | ZM-CRR-1-14 |
| THEOR APPL GENET (1999) 99:593-598 | ZM-CRR-1-15 |
| THEOR APPL GENET (1999) 99:593-598 | ZM-CRR-3-8 |
| THEOR APPL GENET (1999) 99:593-598 | ZM-CRR-5-9 |
| THEOR APPL GENET (1999) 99:593-598 | ZM-CRR-6-6 |
| THEOR APPL GENET (1999) 99:593-598 | ZM-PCSI-1-7 |
| THEOR APPL GENET (1999) 99:593-598 | ZM-PCSI-3-6 |
| THEOR APPL GENET (1999) 99:593-598 | ZM-PCSI-4-6 |
| THEOR APPL GENET (1999) 99:593-598 | ZM-PCSI-5-7 |
| THEOR APPL GENET (1999) 99:593-598 | ZM-PCSI-7-3 |
| THEOR APPL GENET (1999) 99:593-598 | ZM-PHSI-4-1 |
| THEOR APPL GENET (1999) 99:593-598 | ZM-PHSI-5-1 |
| THEOR APPL GENET (1999) 99:593-598 | ZM-PHSI-6-2 |
| THEOR APPL GENET (1999) 99:593-598 | ZM-PHSI-6-3 |
| THEOR APPL GENET (1999) 99:593-598 | ZM-PHSI-6-1 |
| THEOR APPL GENET (1999) 99:593-598 | ZM-PHSI-8-1 |
| THEOR APPL GENET (1999) 99:593-598 | ZM-PHSI-9-1 |
| CROP SCI (1999) 39:1171-1177 | ZM-HPVI-3-1 |
| CROP SCI (1999) 39:1171-1177 | ZM-HPVI-6-1 |
| CROP SCI (1999) 39:1171-1177 | ZM-LVI-4-1 |
| CROP SCI (1999) 39:1171-1177 | ZM-LVI-5-1 |
| CROP SCI (1999) 39:1171-1177 | ZM-LVI-6-1 |
| CROP SCI (1999) 39:514-523 | ZM-ADCNLB-3-1 |
| CROP SCI (1999) 39:514-523 | ZM-ADCNLB-3-2 |
| CROP SCI (1999) 39:514-523 | ZM-ADCNLB-5-3 |
| CROP SCI (1999) 39:514-523 | ZM-ADCNLB-5-2 |
| CROP SCI (1999) 39:514-523 | ZM-ADCNLB-5-1 |
| CROP SCI (1999) 39:514-523 | ZM-ADCNLB-9-1 |
| CROP SCI (1999) 39:514-523 | ZM-DTA-3-2 |
| CROP SCI (1999) 39:514-523 | ZM-DTA-3-1 |
| CROP SCI (1999) 39:514-523 | ZM-DTA-4-1 |
| CROP SCI (1999) 39:514-523 | ZM-NLBIP-3-1 |
| CROP SCI (1999) 39:514-523 | ZM-NLBIP-4-1 |
| CROP SCI (1999) 39:514-523 | ZM-NLBIP-5-1 |
| CROP SCI (1999) 39:514-523 | ZM-NLBIP-8-1 |
| CROP SCI (1999) 39:514-523 | ZM-NLBIP-9-1 |
| THEOR APPL GENET (1999) 98:1036-1045 | ZM-NLBDS-3-2 |
| THEOR APPL GENET (1999) 98:1036-1045 | ZM-NLBDS-5-2 |
| THEOR APPL GENET (1999) 98:1036-1045 | ZM-NLBDS-5-1 |
| THEOR APPL GENET (1999) 98:1036-1045 | ZM-NLBDS-5-3 |
| THEOR APPL GENET (1999) 98:1036-1045 | ZM-NLBDS-8-1 |
| THEOR APPL GENET (1999) 98:1036-1045 | ZM-NLBDS-9-1 |
| THEOR APPL GENET (1999) 98:1036-1045 | ZM-NLBDS-10-2 |
| THEOR APPL GENET (1999) 99:649-655 | ZM-NLBDS-1-1 |
| THEOR APPL GENET (1999) 99:649-655 | ZM-NLBDS-3-5 |
| THEOR APPL GENET (1999) 99:649-655 | ZM-NLBDS-4-2 |
| THEOR APPL GENET (1999) 99:649-655 | ZM-NLBDS-4-1 |
| THEOR APPL GENET (1999) 99:649-655 | ZM-NLBDS-5-5 |
| THEOR APPL GENET (1999) 99:649-655 | ZM-NLBDS-6-1 |
| THEOR APPL GENET (1999) 99:649-655 | ZM-NLBDS-8-2 |
| THEOR APPL GENET (1999) 99:649-655 | ZM-NLBDS-9-2 |
| THEOR APPL GENET (1999) 99:524-539 | ZM-AUT-1-1 |
| THEOR APPL GENET (1999) 99:524-539 | ZM-AUT-10-1 |
| THEOR APPL GENET (1999) 99:524-539 | ZM-APIT-1-1 |
| THEOR APPL GENET (1999) 99:524-539 | ZM-APIT-2-1 |
| THEOR APPL GENET (1999) 99:524-539 | ZM-APIT-3-1 |
| THEOR APPL GENET (1999) 99:524-539 | ZM-APIT-9-1 |
| THEOR APPL GENET (1999) 99:524-539 | ZM-APIT-10-1 |
| THEOR APPL GENET (1999) 99:524-539 | ZM-ANMT-1-1 |
| THEOR APPL GENET (1999) 99:524-539 | ZM-ANMT-2-1 |
| THEOR APPL GENET (1999) 99:524-539 | ZM-ANMT-10-1 |
| THEOR APPL GENET (1999) 99:524-539 | ZM-MSVI-1-1 |
| THEOR APPL GENET (1999) 99:524-539 | ZM-MSVI-1-2 |
| THEOR APPL GENET (1999) 99:524-539 | ZM-MSVI-2-1 |
| THEOR APPL GENET (1999) 99:524-539 | ZM-MSVI-3-2 |
| THEOR APPL GENET (1999) 99:524-539 | ZM-MSVI-3-1 |
| THEOR APPL GENET (1999) 99:524-539 | ZM-MSVI-9-1 |
| THEOR APPL GENET (1999) 99:524-539 | ZM-MSVI-10-1 |
| THEOR APPL GENET (1999) 99:540-553 | ZM-AUT-1-2 |
| THEOR APPL GENET (1999) 99:540-553 | ZM-AUT-3-1 |
| THEOR APPL GENET (1999) 99:540-553 | ZM-AUT-6-1 |
| THEOR APPL GENET (1999) 99:540-553 | ZM-AUT-10-2 |
| THEOR APPL GENET (1999) 99:540-553 | ZM-APIT-1-2 |
| THEOR APPL GENET (1999) 99:540-553 | ZM-APIT-3-2 |
| THEOR APPL GENET (1999) 99:540-553 | ZM-APIT-6-1 |
| THEOR APPL GENET (1999) 99:540-553 | ZM-APIT-10-2 |
| THEOR APPL GENET (1999) 99:540-553 | ZM-ANMT-1-2 |
| THEOR APPL GENET (1999) 99:540-553 | ZM-ANMT-5-1 |
| THEOR APPL GENET (1999) 99:540-553 | ZM-ANMT-10-2 |
| THEOR APPL GENET (1999) 99:1106-1119 | ZM-ASI-4-2 |
| THEOR APPL GENET (1999) 99:1106-1119 | ZM-ASI-7-5 |
| THEOR APPL GENET (1999) 99:1106-1119 | ZM-BIOM-2-1 |
| THEOR APPL GENET (1999) 99:1106-1119 | ZM-BIOM-3-1 |
| THEOR APPL GENET (1999) 99:1106-1119 | ZM-BIOM-7-1 |
| THEOR APPL GENET (1999) 99:1106-1119 | ZM-BIOM-8-1 |
| THEOR APPL GENET (1999) 99:1106-1119 | ZM-CRR-3-9 |
| THEOR APPL GENET (1999) 99:1106-1119 | ZM-CRR-4-6 |
| THEOR APPL GENET (1999) 99:1106-1119 | ZM-CRR-9-5 |
| THEOR APPL GENET (1999) 99:1106-1119 | ZM-DTA-4-2 |
| THEOR APPL GENET (1999) 99:1106-1119 | ZM-DTA-8-4 |
| THEOR APPL GENET (1999) 99:1106-1119 | ZM-ETURI-2-1 |
| THEOR APPL GENET (1999) 99:1106-1119 | ZM-ETURI-5-1 |
| THEOR APPL GENET (1999) 99:1106-1119 | ZM-HMAYI-3-1 |
| THEOR APPL GENET (1999) 99:1106-1119 | ZM-HI-1-1 |
| THEOR APPL GENET (1999) 99:1106-1119 | ZM-HI-3-1 |
| THEOR APPL GENET (1999) 99:1106-1119 | ZM-HI-4-1 |
| THEOR APPL GENET (1999) 99:1106-1119 | ZM-HI-7-1 |
| THEOR APPL GENET (1999) 99:1106-1119 | ZM-HI-8-1 |
| THEOR APPL GENET (1999) 99:1106-1119 | ZM-KW100-1-2 |
| THEOR APPL GENET (1999) 99:1106-1119 | ZM-KW100-3-1 |
| THEOR APPL GENET (1999) 99:1106-1119 | ZM-KW100-9-1 |
| THEOR APPL GENET (1999) 99:1106-1119 | ZM-GWM2-3-1 |
| THEOR APPL GENET (1999) 99:1106-1119 | ZM-GWM2-3-2 |
| THEOR APPL GENET (1999) 99:1106-1119 | ZM-GWM2-7-1 |
| CROP SCI (2001) 41:690-697 | ZM-CPC-1-2 |
| CROP SCI (2001) 41:690-697 | ZM-CPC-6-1 |
| CROP SCI (2001) 41:690-697 | ZM-CPC-7-1 |
| CROP SCI (2001) 41:690-697 | ZM-DMY-6-1 |
| CROP SCI (2001) 41:690-697 | ZM-DMY-8-1 |
| CROP SCI (2001) 41:690-697 | ZM-DMC-5-1 |
| CROP SCI (2001) 41:690-697 | ZM-SILK-8-1 |
| CROP SCI (2001) 41:690-697 | ZM-STC-5-1 |
| CROP SCI (2001) 41:690-697 | ZM-STC-8-1 |
| THEOR APPL GENET (2001) 102:230-243 | ZM-DMC-1-1 |
| THEOR APPL GENET (2001) 102:230-243 | ZM-DMC-5-1 |
| THEOR APPL GENET (2001) 102:230-243 | ZM-DMC-6-1 |
| THEOR APPL GENET (2001) 102:163-176 | ZM-SILK-3-1 |
| THEOR APPL GENET (2001) 102:163-176 | ZM-SILK-4-1 |
| THEOR APPL GENET (2001) 102:163-176 | ZM-SILK-6-2 |
| THEOR APPL GENET (2001) 102:163-176 | ZM-SILK-7-2 |
| THEOR APPL GENET (2001) 102:163-176 | ZM-SILK-8-1 |
| THEOR APPL GENET (2001) 102:163-176 | ZM-ASI-1-3 |
| THEOR APPL GENET (2001) 102:163-176 | ZM-ASI-7-2 |
| THEOR APPL GENET (2001) 102:163-176 | ZM-ASI-8-1 |

TABLE 7
Swiss Prot Information

Seq ID: 1
Accession: P13983
Swissprot_id: EXTN_TOBAC
Gi_number: 119714
Description: Extensin precursor (Cell wall hydroxyproline-rich

glycoprotein)

Seq ID: 3
Accession: P52835
Swissprot_id: F3ST_FLABI
Gi_number: 1706738
Description: FLAVONOL 3-SULFOTRANSFERASE (F3-ST)
Seq ID: 7
Accession: P91428
Swissprot_id: COQ4_CAEEL
Gi_number: 3121872
Description: UBIQUINONE BIOSYNTHESIS PROTEIN COQ4 HOMOLOG
Seq ID: 13
Accession: P25087
Swissprot_id: ERG6_YEAST
Gi_number: 462024
Description: DELTA(24)-STEROL C-METHYLTRANSFERASE
Seq ID: 15
Accession: P52837
Swissprot_id: F4ST_FLACH
Gi_number: 1706740
Description: FLAVONOL 4′-SULFOTRANSFERASE (F4-ST)
Seq ID: 19
Accession: Q43207
Swissprot_id: FKB7_WHEAT
Gi_number: 3023751
Description: 70 kDa peptidylprolyl isomerase (Peptidylprolyl cis-trans

isomerase) (Cyclophilin) (PPiase)

Seq ID: 21
Accession: P51659
Swissprot_id: DHB4_HUMAN
Gi_number: 1706396
Description: Estradiol 17 beta-dehydrogenase 4 (17-beta-HSD 4)

(17-beta-hydroxysteroid dehydrogenase 4)

Seq ID: 23
Accession: Q92696
Swissprot_id: PGTA_HUMAN
Gi_number: 6093707
Description: RAB geranylgeranyltransferase alpha subunit (RAB

geranyl-geranyltransferase alpha subunit) (RAB GG
transferase alpha) (RAB GGTase alpha)

Seq ID: 25
Accession: P08547
Swissprot_id: LIN1_HUMAN
Gi_number: 126295
Description: LINE-1 REVERSE TRANSCRIPTASE HOMOLOG
Seq ID: 31
Accession: P23514
Swissprot_id: COPB_RAT
Gi_number: 116923
Description: COATOMER BETA SUBUNIT (BETA-COAT PROTEIN) (BETA-COP)
Seq ID: 37
Accession: P43293
Swissprot_id: NAK_ARATH
Gi_number: 1171642
Description: Probable serine/threonine-protem kinase NAK
Seq ID: 39
Accession: P19338
Swissprot_id: NUCL_HUMAN
Gi_number: 128841
Description: Nucleolin (Protein C23)
Seq ID: 41
Accession: Q24120
Swissprot_id: CAPU_DROME
Gi_number: 13124006
Description: CAPPUCCINO PROTEIN
Seq ID: 43
Accession: Q03363
Swissprot_id: DNJ1_ALLPO
Gi_number: 461942
Description: DnaJ protein homolog 1 (DNAJ-1)
Seq ID: 45
Accession: P04929
Swissprot_id: HRPX_PLALO
Gi_number: 123530
Description: HISTIDINE-RICH GLYCOPROTEIN PRECURSOR
Seq ID: 47
Accession: P13983
Swissprot_id: EXTN_TOBAC
Gi_number: 119714
Description: Extensin precursor (Cell wall hydroxyproline-rich

glycoprotem)

Seq ID: 55
Accession: P74457
Swissprot_id: PYRH_SYNY3
Gi_number: 2497492
Description: Uridylate kinase (UK) (Uridine monophosphate kinase) (UMP

kinase)

Seq ID: 59
Accession: P08640
Swissprot_id: AMYH_YEAST
Gi_number: 728850
Description: GLUCOAMYLASE S1/S2 PRECURSOR (GLUCAN

1,4-ALPHA-GLUCOSIDASE) (1,4-ALPHA-D-GLUCAN GLUCOHYDROLASE)

Seq ID: 63
Accession: Q16620
Swissprot_id: TRKB_HUMAN
Gi_number: 2497560
Description: BDNF/NT-3 GROWTH FACTORS RECEPTOR PRECURSOR (TRKB TYROSINE

KINASE) (GP145-TRKB) (TRK-B)

Seq ID: 65
Accession: P17840
Swissprot_id: SLS3_BRAOL
Gi_number: 134532
Description: S-locus-specific glycoprotein S13 precursor (SLSG-13)
Seq ID: 67
Accession: P08453
Swissprot_id: GDB2_WHEAT
Gi_number: 121101
Description: GAMMA-GLIADIN PRECURSOR
Seq ID: 69
Accession: Q9S1G2
Swissprot_id: DPO1_RHILE
Gi_number: 12229815
Description: DNA polymerase I (POL I)
Seq ID: 71
Accession: Q99728
Swissprot_id: BAR1_HUMAN
Gi_number: 13123980
Description: BRCA1-ASSOCIATED RING DOMAIN PROTEIN 1 (BARD-1)
Seq ID: 75
Accession: Q09052
Swissprot_id: ACC1_BRAJU
Gi_number: 728780
Description: 1-AMINOCYCLOPROPANE-1-CARBOXYLATE OXIDASE (ACC OXIDASE)

(ETHYLENE-FORMING ENZYME) (EFE)

Seq ID: 77
Accession: Q39760
Swissprot_id: DCS2_GOSAR
Gi_number: 3287834
Description: (+)-delta-cadinene synthase isozyme XC14 (D-cadinene

synthase)

Seq ID: 79
Accession: Q05963
Swissprot_id: FL3H_CALCH
Gi_number: 729503
Description: Naringenin,2-oxoglutarate 3-dioxygenase

(Flavonone-3-hydroxylase) (F3H) (FHT)

Seq ID: 83
Accession: P40267
Swissprot_id: H1_LYCPN
Gi_number: 729668
Description: HISTONE H1
Seq ID: 85
Accession: Q13439
Swissprot_id: GOG4_HUMAN
Gi_number: 12643718
Description: GOLGI AUTOANTIGEN, GOLGIN SUBFAMILY A 4 (TRANS-GOLGI P230)

(256 KDA GOLGIN) (GOLGIN-245) (72.1 PROTEIN)

Seq ID: 87
Accession: P38137
Swissprot_id: FAT2_YEAST
Gi_number: 586339
Description: Peroxisomal-coenzyme A synthetase
Seq ID: 89
Accession: Q9RW10
Swissprot_id: MENG_DEIRA
Gi_number: 17369587
Description: Probable S-adenosylmethionine:2-demethylmenaquinone

methyltransferase

Seq ID: 91
Accession: Q9ZT66
Swissprot_id: E134_MAIZE
Gi_number: 8928122
Description: Endo-1,3;1,4-beta-D-glucanase precursor
Seq ID: 95
Accession: Q15738
Swissprot_id: NSDL_HUMAN
Gi_number: 8488997
Description: AND(P)-DEPENDENT STEROID DEHYDROGENASE (H105E3 PROTEIN)
Seq ID: 99
Accession: O14727
Swissprot_id: APAF_HUMAN
Gi_number: 3023307
Description: Apoptotic protease activating factor 1 (Apaf-1)
Seq ID: 105
Accession: P26170
Swissprot_id: BCHG_RHOCA
Gi_number: 114850
Description: Bacteriochlorophyll synthase 33 kDa chain (Geranylgeranyl

bacteriochlorophyll synthase)

Seq ID: 107
Accession: O22340
Swissprot_id: TSD3_ABIGR
Gi_number: 17367918
Description: (−)-(45)-limonene synthase, chloroplast precursor
Seq ID: 109
Accession: Q05085
Swissprot_id: CHL1_ARATH
Gi_number: 544018
Description: Nitrate/chlorate transporter
Seq ID: 111
Accession: P46086
Swissprot_id: KIME_ARATH
Gi_number: 1170660
Description: Mevalonate kinase (MK)
Seq ID: 113
Accession: O22567
Swissprot_id: CLA1_ORYSA
Gi_number: 3913239
Description: Probable 1-deoxy-D-xylulose 5-phosphate synthase,

chloroplast (1-deoxyxylulose-5-phosphate synthase) (DXP
synthase) (DXPS)

Seq ID: 115
Accession: P48450
Swissprot_id: ERG7_RAT
Gi_number: 1352388
Description: Lanosterol synthase (Oxidosqualene--lanosterol cyclase)

(2,3-epoxysqualene--lanosterol cyclase) (OSC)

Seq ID: 117
Accession: Q06587
Swissprot_id: RNG1_HUMAN
Gi_number: 548733
Description: Polycomb complex protein RING1 (RNF1)
Seq ID: 119
Accession: P04929
Swissprot_id: HRPX_PLALO
Gi_number: 123530
Description: HISTIDINE-RICH GLYCOPROTEIN PRECURSOR
Seq ID: 131
Accession: P52839
Swissprot_id: FSTL_ARATH
Gi_number: 1706917
Description: Flavonol sulfotransferase-like (RaRO47)
Seq ID: 133
Accession: Q9XHL5
Swissprot_id: HMD3_ORYSA
Gi_number: 11133198
Description: 3-hydroxy-3-methylglutaryl-coenzyme A reductase 3 (HMG-CoA

reductase 3)

Seq ID: 135
Accession: Q55087
Swissprot_id: CHLP_SYNY3
Gi_number: 2493698
Description: GERANYLGERANYL HYDROGENASE
Seq ID: 139
Accession: P27806
Swissprot_id: H1_WHEAT
Gi_number: 14916992
Description: Histone H1
Seq ID: 141
Accession: Q96330
Swissprot_id: FLS1_ARATH
Gi_number: 6166164
Description: FLAVONOL SYNTHASE 1 (FLS 1)
Seq ID: 145
Accession: P04323
Swissprot_id: POL3_DROME
Gi_number: 130405
Description: Retrovirus-related Pol polyprotein from transposon 17.6

[Contains: Protease ; Reverse transcriptase ;
Endonuclease]

Seq ID: 147
Accession: Q9ZCV0
Swissprot_id: RL20_RICPR
Gi_number: 6225976
Description: 50S ribosomal protein L20
Seq ID: 149
Accession: O48674
Swissprot_id: HEM1_ORYSA
Gi_number: 3913811
Description: Glutamyl-tRNA reductase, chloroplast precursor (GluTR)
Seq ID: 151
Accession: P28968
Swissprot_id: VGLX_HSVEB
Gi_number: 138350
Description: GLYCOPROTEIN X PRECURSOR
Seq ID: 155
Accession: Q43147
Swissprot_id: CP85_LYCES
Gi_number: 5921933
Description: CYTOCHROME P450 85 (DWARF PROTETN)
Seq ID: 159
Accession: O43791
Swissprot_id: SPOP_HUMAN
Gi_number: 8134708
Description: Speckle-type POZ protein
Seq ID: 161
Accession: P57708
Swissprot_id: ISPF_PSEAE
Gi_number: 12643672
Description: 2C-METHYL-D-ERYTHRITOL 2,4-CYCLODIPHOSPHATE SYNTHASE

(MECPS)

Seq ID: 165
Accession: Q04677
Swissprot_id: THIB_CANTR
Gi_number: 418002
Description: ACETYL-COA ACETYLTRANSFERASE IB (PEROXISOMAL

ACETOACETYL-COA THIOLASE) (THIOLASE IB)

Seq ID: 169
Accession: P53799
Swissprot_id: FDFT_ARATH
Gi_number: 1706772
Description: Farnesyl-diphosphate farnesyltransferase (Squalene

synthetase) (SQS) (SS) (FPP:FPP farnesyltransferase)

Seq ID: 173
Accession: P22503
Swissprot_id: GUN_PHAVU
Gi_number: 1346225
Description: ENDOGLUCANASE PRECURSOR (ENDO-1,4-BETA-GLUCANASE)

(ABSCISSLON CELLULASE)

Seq ID: 179
Accession: Q62598
Swissprot_id: DSPP_RAT
Gi_number: 17865451
Description: Dentin sialophosphoprotein precursor [Contains: Dentin

phosphoprotein (Dentin phosphophoryn) (DPP); Dentin
sialoprotein (DSP)]

Seq ID: 181
Accession: P10978
Swissprot_id: POLX_TOBAC
Gi_number: 130582
Description: Retrovirus-related Pol polyprotein from transposon TNT

1-94 [Contains: Protease ; Reverse transcriptase;
Endonuclease]

Seq ID: 183
Accession: P14328
Swissprot_id: SP96_DICDI
Gi_number: 134780
Description: SPORE COAT PROTEIN SP96
Seq ID: 185
Accession: P04323
Swissprot_id: POL3_DROME
Gi_number: 130405
Description: Retrovirus-related Pol polyprotein from transposon 17.6

[Contains: Protease ; Reverse transcriptase ;
Endonuclease]

Seq ID: 189
Accession: Q9LX33
Swissprot_id: PFTA_ARATH
Gi_number: 12643807
Description: Protein farnesyltransferase alpha subunit (CAAX

farnesyltransferase alpha subunit) (RAS proteins
prenyltransferase alpha) (FTase-alpha)

Seq ID: 191
Accession: Q9WTV7
Swissprot_id: RNFB_MOUSE
Gi_number: 13124535
Description: RING FINGER PROTEIN 12 (LIM DOMAIN INTERACTING RING FINGER

PROTEIN) (RING FINGER LIM DOMAIN-BINDING PROTEIN) (R-LIM)

Seq ID: 193
Accession: Q05001
Swissprot_id: NCPR_CATRO
Gi_number: 730125
Description: NADPH-cytochrome P450 reductase (CPR) (P450R)
Seq ID: 197
Accession: P52835
Swissprot_id: F3ST_FLABI
Gi_number: 1706738
Description: FLAVONOL 3-SULFOTRANSFERASE (F3-ST)
Seq ID: 205
Accession: P11675
Swissprot_id: IE18_PRVIF
Gi_number: 124178
Description: IMMEDIATE-EARLY PROTEIN IE180
Seq ID: 207
Accession: Q912W7
Swissprot_id: MENG_PSEAE
Gi_number: 17369015
Description: S-adenosylmethionine:2-demethylmenaquinone

methyltransferase

Seq ID: 211
Accession: P36940
Swissprot_id: FASL_RAT
Gi_number: 544277
Description: FAS ANTIGEN LIGAND
Seq ID: 213
Accession: Q9Z5D6
Swissprot_id: BCHG_RHOSH
Gi_number: 13878356
Description: Bacteriochlorophyll synthase 33 kDa chain (Geranylgeranyl

bacteriochlorophyll synthase)

Seq ID: 215
Accession: P47735
Swissprot_id: RLK5_ARATH
Gi_number: 1350783
Description: Receptor-like protein kinase 5 precursor
Seq ID: 217
Accession: P11369
Swissprot_id: POL2_MOUSE
Gi_number: 130402
Description: Retrovirus-related POL polyprotein [Contains: Reverse

transcriptase ; Endonuclease]

Seq ID: 219
Accession: Q15800
Swissprot_id: ER25_HUMAN
Gi_number: 2498340
Description: C-4 methyl sterol oxidase
Seq ID: 221
Accession: Q9LTI3
Swissprot_id: MPU1_ARATH
Gi_number: 12644539
Description: Mannose-P-dolichol utilization defect 1 protein homolog
Seq ID: 227
Accession: P33485
Swissprot_id: VNUA_PRVKA
Gi_number: 465445
Description: PROBABLE NUCLEAR ANTIGEN
Seq ID: 229
Accession: P08640
Swissprot_id: AMYH_YEAST
Gi_number: 728850
Description: GLUCOAMYLASE S1/S2 PRECURSOR (GLUCAN

1,4-ALPHA-GLUCOSIDASE) (1,4-ALPHA-D-GLUCAN GLUCOHYDROLASE)

Seq ID: 235
Accession: P16157
Swissprot_id: ANK1_HUMAN
Gi_number: 113884
Description: Ankyrin 1 (Erythrocyte ankyrin) (Ankyrin R)
Seq ID: 239
Accession: Q9ZCP3
Swissprot_id: UBIE_RICPR
Gi_number: 6647890
Description: Probable ubiqumone/menaquinone biosynthesis

methyltransferase UBIE

Seq ID: 241
Accession: Q9S1G2
Swissprot_id: DPO1_RHILE
Gi_number: 12229815
Description: DNA polymerase I (POL I)
Seq ID: 243
Accession: O23051
Swissprot_id: C883_ARATH
Gi_number: 5915848
Description: Cytochrome P450 88A3
Seq ID: 245
Accession: Q06548
Swissprot_id: APKA_ARATH
Gi_number: 1168470
Description: Protein kinase APK1A
Seq ID: 247
Accession: P46604
Swissprot_id: HT22_ARATH
Gi_number: 1170409
Description: Homeobox-leucine zipper protein HAT22 (HD-ZIP protein 22)
Seq ID: 249
Accession: O49853
Swissprot_id: DCS4_GOSAR
Gi_number: 3287826
Description: (+)-delta-cadinene synthase isozyme C2 (D-cadinene

synthase)

Seq ID: 251
Accession: O14321
Swissprot_id: ERG6_SCHPO
Gi_number: 6166151
Description: PROBABLE DELTA(24)-STEROL C-METHYLTRANSFERASE
Seq ID: 253
Accession: P17816
Swissprot_id: GRP_HORVU
Gi_number: 121644
Description: GLYCINE-RICH CELL WALL STRUCTURAL PROTEIN PRECURSOR
Seq ID: 255
Accession: Q9P2Y4
Swissprot_id: Z219_HUMAN
Gi_number: 12230861
Description: Zinc finger protein 219
Seq ID: 257
Accession: P53683
Swissprot_id: CDP2_ORYSA
Gi_number: 1705734
Description: Calcium-dependent protein kinase, isoform 2 (CDPK 2)
Seq ID: 259
Accession: O60610
Swissprot_id: DIA1_HUMAN
Gi_number: 6225268
Description: DIAPHANOUS PROTEIN HOMOLOG 1 (DIAPHANOUS-RELATED FORMIN 1)

(DRF1)

Seq ID: 261
Accession: P93163
Swissprot_id: GBA2_SOYBN
Gi_number: 3913724
Description: Guanine nucleotide-binding protein alpha-2 subunit

(GP-alpha-2)

Seq ID: 263
Accession: P82094
Swissprot_id: TMF1_HUMAN
Gi_number: 8134736
Description: TATA element modulatory factor (TMF)
Seq ID: 265
Accession: P49353
Swissprot_id: FPPS_MAIZE
Gi_number: 1346033
Description: Famesyl pyrophosphate synthetase (FPP synthetase) (FPS)

(Farnesyl diphosphate synthetase) [Includes:
Dimethylallyltransferase ; Geranyltranstransferase]

Seq ID: 267
Accession: Q9XFS9
Swissprot_id: DXR_ARATH
Gi_number: 12644032
Description: 1-deoxy-D-xylulose 5-phosphate reductoisomerase,

chloroplast precursor (DXP reductoisomerase)
(1-deoxyxylulose-5-phosphate reductoisomerase)

Seq ID: 271
Accession: P28968
Swissprot_id: VGLX_HSVEB
Gi_number: 138350
Description: GLYCOPROTEIN X PRECURSOR
Seq ID: 273
Accession: P35579
Swissprot_id: MYH9_HUMAN
Gi_number: 6166599
Description: Myosin heavy chain, nonmuscle type A (Cellular myosin

heavy chain, type A) (Nonmuscle myosin heavy chain-A)
(NMMHC-A)

Seq ID: 275
Accession: P03211
Swissprot_id: EBN1_EBV
Gi_number: 119110
Description: EBNA-1 NUCLEAR PROTEIN
Seq ID: 277
Accession: P14284
Swissprot_id: DPOZ_YEAST
Gi_number: 118901
Description: DNA polymerase zeta catalytic subunit
Seq ID: 279
Accession: Q02817
Swissprot_id: MUC2_HUMAN
Gi_number: 2506877
Description: MUCIN 2 PRECURSOR (INTESTINAL MUCIN 2)
Seq ID: 283
Accession: Q9UQ13
Swissprot_id: SHO2_HUMAN
Gi_number: 14423936
Description: LEUCINE-RICH REPEAT PROTEIN SHOC-2 (RAS-BINDING PROTEIN

SUR-8)

Seq ID: 285
Accession: Q912W7
Swissprot_id: MENG_PSEAE
Gi_number: 17369015
Description: S-adenosylmethionine:2-demethylmenaquinone

methyltransferase

Seq ID: 287
Accession: P50860
Swissprot_id: ERG3_CANGA
Gi_number: 1706691
Description: C-5 sterol desaturase (Sterol-C5-desaturase)
Seq ID: 289
Accession: P16970
Swissprot_id: ABD3_RAT
Gi_number: 130359
Description: ATP-binding cassette, sub-family D, member 3 (70 kDa

peroxisomal membrane protein) (PMP70)

Seq ID: 291
Accession: P07237
Swissprot_id: PDI_HUMAN
Gi_number: 2507460
Description: PROTEIN DISULFIDE ISOMERASE PRECURSOR (PDI) (PROLYL

4-HYDROXYLASE BETA SUBUNIT) (CELLULAR THYROID HORMONE
BINDING PROTEIN) (P55)

Seq ID: 293
Accession: Q62967
Swissprot_id: ER19_RAT
Gi_number: 2498339
Description: Diphosphomevalonate decarboxylase (Mevalonate

pyrophosphate decarboxylase) (Mevalonate
(diphospho)decarboxylase)

Seq ID: 297
Accession: P10967
Swissprot_id: ACC3_LYCES
Gi_number: 119640
Description: 1-AMINOCYCLOPROPANE-1-CARBOXYLATE OXIDASE HOMOLOG (PROTEIN

E8)

Seq ID: 299
Accession: P40603
Swissprot_id: APG_BRANA
Gi_number: 728868
Description: ANTER-SPECIFIC PROLINE-RICH PROTEIN APG (PROTEIN CEX)
Seq ID: 301
Accession: P08640
Swissprot_id: AMYH_YEAST
Gi_number: 728850
Description: GLUCOAMYLASE S1/S2 PRECURSOR (GLUCAN

1,4-ALPHA-GLUCOSIDASE) (1,4-ALPHA-D-GLUCAN GLUCOHYDROLASE)

Seq ID: 303
Accession: Q28092
Swissprot_id: CYL2_BOVIN
Gi_number: 2498277
Description: CYLICIN II (MULTIPLE-BAND POLYPEPTIDE II)
Seq ID: 307
Accession: P46573
Swissprot_id: APKB_ARATH
Gi_number: 12644274
Description: PROTEIN KINASE APK1B
Seq ID: 309
Accession: Q05654
Swissprot_id: RDPO_SCHPO
Gi_number: 1710054
Description: RETROTRANSPOSABLE ELEMENT TF2 155 KDA PROTEIN
Seq ID: 311
Accession: Q05609
Swissprot_id: CTR1_ARATH
Gi_number: 1169128
Description: Serine/threonine-protein kinase CTR1
Seq ID: 313
Accession: P47989
Swissprot_id: XDH_HUMAN
Gi_number: 2506326
Description: XANTHINE DEHYDROGENASE/OXIDASE [INCLUDES: XANTHINE

DEHYDROGENASE (XD); XANTHINE OXIDASE (XO) (XANTHINE
OXIDOREDUCTASE)]

Seq ID: 315
Accession: P54873
Swissprot_id: HMCS_ARATH
Gi_number: 1708236
Description: HYDROXYMETHYLGLUTARYL-COA SYNTHASE (HMG-COA SYNTHASE)

(3-HYDROXY-3-METHYLGLUTARYL COENZYME A SYNTHASE)

Seq ID: 317
Accession: Q9SSU8
Swissprot_id: PSY_DAUCA
Gi_number: 8928282
Description: Phytoene synthase, chloroplast precursor
Seq ID: 319
Accession: Q9U7E0
Swissprot_id: ATRX_CAEEL
Gi_number: 17367114
Description: Transcriptional regulator ATRX homolog (X-linked nuclear

protein-1)

Seq ID: 321
Accession: P51617
Swissprot_id: IRA1_HUMAN
Gi_number: 8928535
Description: Interleukin-1 receptor-associated kinase 1 (IRAK-1)
Seq ID: 327
Accession: P10978
Swissprot_id: POLX_TOBAC
Gi_number: 130582
Description: Retrovirus-related Pol polyprotein from transposon TNT

1-94 [Contains: Protease ; Reverse transcriptase ;
Endonuclease]

Seq ID: 329
Accession: P52837
Swissprot_id: F4ST_FLACH
Gi_number: 1706740
Description: FLAVONOL 4′-SULFOTRANSFERASE (F4-ST)
Seq ID: 333
Accession: Q39085
Swissprot_id: DIM_ARATH
Gi_number: 17380361
Description: Cell elongation protein DIMINUTO (Cell elongation protein

Dwarf1)

Seq ID: 335
Accession: O81220
Swissprot_id: DCUP_MAIZE
Gi_number: 6014938
Description: Uroporphyrinogen decarboxylase, chloroplast precursor

(UPD)

Seq ID: 341
Accession: P50172
Swissprot_id: DHI1_MOUSE
Gi_number: 1706408
Description: Corticosteroid 11-beta-dehydrogenase, isozyme 1 (11-DH)

(11-beta-hydroxysteroid dehydrogenase 1) (11-beta-HSD1)
(11beta-HSD1A)

Seq ID: 343
Accession: P53611
Swissprot_id: PGTB_HUMAN
Gi_number: 2506788
Description: GERANYLGERANYL TRANSFERASE TYPE II BETA SUBUNIT (RAB
GERANYLGERANYLTRANSFERASE BETA SUBUNIT) (RAB
GERANYL-GERANYLTRANSFERASE BETA SUBUNIT) (RAB GG
TRANSFERASE BETA) (RAB GGTASE BETA)
Seq ID: 345
Accession: Q02629
Swissprot_id: N100_YEAST
Gi_number: 400320
Description: NUCLEOPORIN NUP100/NSP100 (NUCLEAR PORE PROTEIN

NUP100/NSP100)

Seq ID: 347
Accession: P43216
Swissprot_id: MPH1_HOLLA
Gi_number: 1171005
Description: MAJOR POLLEN ALLERGEN HOL L 1 PRECURSOR (HOL L I) (HOL L

1.0101 AND 1.0102)

Seq ID: 349
Accession: P46573
Swissprot_id: APKB_ARATH
Gi_number: 12644274
Description: PROTEIN KINASE APK1B
Seq ID: 353
Accession: P48019
Swissprot_id: HMD1_ORYSA
Gi_number: 1346301
Description: 3-hydroxy-3-methylglutaryl-coenzyme A reductase (HMG-CoA

reductase)

Seq ID: 357
Accession: Q39760
Swissprot_id: DCS2_GOSAR
Gi_number: 3287834
Description: (+)-delta-cadinene synthase isozyme XC14 (D-cadinene

synthase)

Seq ID: 359
Accession: O64411
Swissprot_id: PAO_MAIZE
Gi_number: 6225822
Description: Polyamine oxidase precursor
Seq ID: 361
Accession: P38605
Swissprot_id: CAS1_ARATH
Gi_number: 584882
Description: CYCLOARTENOL SYNTHASE (2,3-EPOXYSQUALENE--CYCLOARTENOL

CYCLASE)

Seq ID: 363
Accession: P13728
Swissprot_id: SGS3_DROYA
Gi_number: 134469
Description: Salivary glue protein SGS-3 precursor
Seq ID: 365
Accession: Q62967
Swissprot_id: ER19_RAT
Gi_number: 2498339
Description: Diphosphomevalonate decarboxylase (Mevalonate

pyrophosphate decarboxylase) (Mevalonate
(diphospho)decarboxylase)

Seq ID: 375
Accession: Q9NZ52
Swissprot_id: GGA3_HUMAN
Gi_number: 14548064
Description: ADP-RIBOSYLATION FACTOR BINDING PROTEIN GGA3

(GOLGI-LOCALIZED, GAMMA EAR-CONTAINING, ARF-BINDING
PROTEIN 3)

Seq ID: 379
Accession: P52835
Swissprot_id: F3ST_FLABI
Gi_number: 1706738
Description: FLAVONOL 3-SULFOTRANSFERASE (F3-ST)
Seq ID: 381
Accession: P91428
Swissprot_id: COQ4_CAEEL
Gi_number: 3121872
Description: UBIQUINONE BIOSYNTHESIS PROTEIN COQ4 HOMOLOG
Seq ID: 383
Accession: Q9ZTP4
Swissprot_id: ZDS_MAIZE
Gi_number: 17367864
Description: Zeta-carotene desaturase, chloroplast precursor (Carotene

7,8-desaturase)

Seq ID: 391
Accession: P54980
Swissprot_id: CRTI_RHOSH
Gi_number: 1706146
Description: Phytoene dehydrogenase (Phytoene desaturase)
Seq ID: 392
Accession: P54980
Swissprot_id: CRTI_RHOSH
Gi_number: 1706146
Description: Phytoene dehydrogenase (Phytoene desaturase)
Seq ID: 393
Accession: Q912W7
Swissprot_id: MENG_PSEAE
Gi_number: 17369015
Description: 5-adenosylmethionine:2-demethylmenaquinone

methyltransferase

Seq ID: 397
Accession: Q01332
Swissprot_id: CRTZ_ERWHE
Gi_number: 231913
Description: BETA-CAROTENE HYDROXYLASE
Seq ID: 398
Accession: Q01332
Swissprot_id: CRTZ_ERWHE
Gi_number: 231913
Description: BETA-CAROTENE HYDROXYLASE
Seq ID: 399
Accession: Q9ZWQ9
Swissprot_id: FLS_CITUN
Gi_number: 14916566
Description: Flavonol synthase (FLS) (CitFLS)
Seq ID: 400
Accession: Q9ZWQ9
Swissprot_id: FLS_CITUN
Gi_number: 14916566
Description: Flavonol synthase (FLS) (CitFLS)
Seq ID: 401
Accession: Q42525
Swissprot_id: HXK1_ARATH
Gi_number: 12644433
Description: HEXOKINASE 1

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. An isolated polynucleotide comprising nucleotide sequence encoding a polypeptide involved in or associated with the biosynthesis of isoprenoids in a plant, wherein said nucleotide sequence comprises
a) a nucleotide sequence selected from the group consisting of SEQ ID NO 1, 3, 5, 7, 9, 11, 13, 15, 17, 19, 21, 23, 25, 27, 28, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, and 413;
b) a nucleotide sequence substantially similar to a nucleotide sequence of a);
c) a nucleotide sequence of at least 15 nucleotides in length that hybridizes with a nucleotide sequence of a) or the complement thereof under stringent conditions.
d) a fragment of a nucleotide sequence of a) wherein said fragment encodes a polypeptide involved in or associated with the biosynthesis of isoprenoids in a plant;
e) a nucleotide sequence complementary to a nucleotide sequence of a), b) or c); or
f) a nucleotide sequence comprising a reverse complement to a nucleotide sequence of a), b) or c).

2. An isolated polynucleotide comprising a nucleotide sequence that is at least 90% identical to the nucleotide sequence of claim 1.

3. An isolated polynucleotide comprising nucleotide sequence encoding a polypeptide involved in or associated with the biosynthesis of isoprenoids in a plant, wherein said polypeptide comprises an amino acid sequence selected from the group consisting of SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 314, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412 and 414.

4. An isolated polypeptide involved in or associated with the biosynthesis of isoprenoids in a plant, comprising an amino acid sequence selected from the group consisting of SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 314, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412 and 414.

5. An isolated polypeptide comprising an amino acid sequence substantially similar to the amino acid sequence of the polypeptide of claim 4.

6. An isolated polynucleotide comprising a nucleotide sequence or fragment thereof encoding a polypeptide involved in or associated with the biosynthesis of isopentenyl diphosphate (IPP) and dimethylallyl alcohol (DMAPP) in a plant said nucleotide sequence selected from the group consisting of SEQ ID NO 99, 113, 153, 161, 267 and 413.

7. An isolated polynucleotide comprising a nucleotide sequence substantially similar to the nucleotide sequence of the polynucleotide of claim 6.

8. An expression cassette comprising the polynucleotide of claim 6.

9. A host cell comprising the expression cassette of claim 8.

10. A transgenic plant comprising the expression cassette of claim 8.

11. An isolated polynucleotide comprising a nucleotide sequence encoding a polypeptide involved in or associated with the biosynthesis of isopentenyl diphosphate (IPP) and dimethylallyl alcohol (DMAPP) in a plant, said polypeptide comprising an amino acid sequence selected from the group consisting of SEQ ID NO 100, 114, 154, 162, 268 and 414.

12. An expression cassette comprising the polynucleotide of claim 11.

13. A host cell comprising the expression cassette of claim 12.

14. A transgenic plant comprising the expression cassette of claim 12.

15. An isolated polynucleotide comprising a nucleotide sequence or fragment thereof encoding a polypeptide with enzymatic activity involved in or associated with the biosynthesis of short-chain plastid prenyltransferases, said nucleotide sequence selected from the group consisting of SEQ ID NO 11, 47, 83, 125, 135, 229, 235, 243, 265, 299, 345, 371, 409 and 411.

16. An isolated polynucleotide comprising a nucleotide sequence substantially similar to the nucleotide sequence of the polynucleotide of claim 15.

17. An expression cassette comprising the polynucleotide of claim 15.

18. A host cell comprising the expression cassette of claim 17.

19. A transgenic plant comprising the expression cassette of claim 17.

20. An isolated polynucleotide comprising a nucleotide sequence encoding a polypeptide with enzymatic activity involved in or associated with the biosynthesis of short-chain plastid prenyltransferases said polypeptide comprising an amino acid sequence selected from the group consisting of SEQ ID NO 12, 48, 84, 126, 136, 230, 236, 244, 266, 300, 346, 372, 410 and 412.

21. An expression cassette comprising the polynucleotide of claim 20.

22. A host cell comprising the expression cassette of claim 21.

23. A transgenic plant comprising the expression cassette of claim 21.

24. An isolated polynucleotide comprising a nucleotide sequence or fragment thereof encoding a polypeptide the activity of which is involved in or associated with the biosynthesis of gibberellins in plants, said nucleotide sequence selected from the group consisting of SEQ ID NO 25, 67, 75, 79, 97, 107, 141, 155, 173, 193, 203, 205, 223, 225, 253, 259, 261, 291, 297, 349, 359, 375, 377, and 399.

25. An isolated polynucleotide comprising a nucleotide sequence substantially similar to the nucleotide sequence of the polynucleotide of claim 24.

26. An expression cassette comprising the polynucleotide of claim 24.

27. A host cell comprising the expression cassette of claim 26.

28. A transgenic plant comprising the expression cassette of claim 26.

29. An isolated polynucleotide comprising a nucleotide sequence encoding a polypeptide the activity of which is involved in or associated with the biosynthesis of gibberellins in plants, said polypeptide comprising an amino acid sequence selected from the group consisting of SEQ ID NO 26, 68, 76, 80, 98, 108, 142, 156, 174, 194, 204, 206, 224, 226, 254, 260, 262, 292, 298, 350, 360, 376, 378, and 400.

30. An expression cassette comprising the polynucleotide of claim 29.

31. A host cell comprising the expression cassette of claim 30.

32. A transgenic plant comprising the expression cassette of claim 30.

33. An isolated polynucleotide comprising a nucleotide sequence or fragment thereof encoding a polypeptide which exhibits enzymatic activity involved in or associated with the biosynthesis of carotenoids and/or abscisic acids in plants, said nucleotide sequence selected from the group consisting of SEQ ID NO 27, 89, 103, 121, 205, 237, 245, 271, 275, 301, 313, 317, 383, 391, 395, 397 and 407.

34. An isolated polynucleotide comprising a nucleotide sequence substantially similar to the nucleotide sequence of the polynucleotide of claim 33.

35. An expression cassette comprising the polynucleotide of claim 33.

36. A host cell comprising the expression cassette of claim 35.

37. A transgenic plant comprising the expression cassette of claim 35.

38. An isolated polynucleotide comprising a nucleotide sequence encoding a polypeptide which exhibits enzymatic activity involved in or associated with the biosynthesis of carotenoids and/or abscisic acids in plants, said polypeptide comprising an amino acid sequence selected from the group consisting of SEQ ID NO 28, 90, 104, 122, 206, 238, 246, 272, 276, 302, 314, 318, 384, 392, 396, 398 and 408.

39. An expression cassette comprising the polynucleotide of claim 38.

40. A host cell comprising the expression cassette of claim 39.

41. A transgenic plant comprising the expression cassette of claim 39.

42. An isolated polynucleotide comprising a nucleotide sequence or fragment thereof encoding a polypeptide which has enzymatic activity involved in or associated with the biosynthesis of tocopherols in plants, said nucleotide sequence selected from the group consisting of SEQ ID NO 89 and 275.

43. An isolated polynucleotide comprising a nucleotide sequence substantially similar to the nucleotide sequence of the polynucleotide of claim 42.

44. An expression cassette comprising the polynucleotide of claim 42.

45. A host cell comprising the expression cassette of claim 44.

46. A transgenic plant comprising the expression cassette of claim 44.

47. A isolated polynucleotide comprising a nucleotide sequence encoding a polypeptide which has enzymatic activity involved in or associated with the biosynthesis of tocopherols in plants, said polypeptide comprising an amino acid sequence selected from the group consisting of SEQ ID NO 90 and 276.

48. An expression cassette comprising the polynucleotide of claim 47.

49. A host cell comprising the expression cassette of claim 48.

50. A transgenic plant comprising the expression cassette of claim 48.

51. An isolated polynucleotide comprising a nucleotide sequence or fragment thereof encoding a polypeptide with enzymatic activity involved in or associated with plastoquinone and/or phylloquinone biosynthesis in a plant, said nucleotide sequence selected from the group consisting of SEQ ID NO 17, 29, 85, 87, 89, 207, 213, 285, 393, 401.

52. An isolated polynucleotide comprising a nucleotide sequence substantially similar to the nucleotide sequence of the polynucleotide of claim 51.

53. An expression cassette comprising the nucleotide sequence of claim 51.

54. A host cell comprising the expression cassette of claim 53.

55. A transgenic plant comprising the expression cassette of claim 53.

56. An isolated polynucleotide comprising a nucleotide sequence encoding a polypeptide with enzymatic activity involved in or associated with plastoquinone and/or phylloquinone biosynthesis in a plant, said polypeptide comprising an amino acid sequence selected from the group consisting of SEQ ID NO 18, 30, 86, 88, 90, 208, 214, 286, 394, 402.

57. An expression cassette comprising the polynucleotide of claim 56.

58. A host cell comprising the expression cassette of claim 57.

59. A transgenic plant comprising the expression cassette of claim 57.

60. An isolated polynucleotide comprising a nucleotide sequence or fragment thereof encoding a polypeptide with enzymatic activity involved in or associated with the mevalonate pathway in plants, said nucleotide sequence selected from the group consisting of SEQ ID NO 111, 133, 165, 293, 315, 353, 365.

61. An isolated polynucleotide comprising a nucleotide sequence substantially similar to the nucleotide sequence of the polynucleotide of claim 60.

62. An expression cassette comprising the polynucleotide of claim 60.

63. A host cell comprising the expression cassette of claim 62.

64. A transgenic plant comprising the expression cassette of claim 62.

65. An isolated polynucleotide comprising a nucleotide sequence encoding a polypeptide with enzymatic activity involved in or associated with the mevalonate pathway in plants, said polypeptide comprising an amino acid sequence selected from the group consisting of SEQ ID NO 112, 134, 166, 294, 316, 354, 366.

66. An expression cassette comprising the polynucleotide of claim 65.

67. A host cell comprising the expression cassette of claim 66.

68. A transgenic plant comprising the expression cassette of claim 66.

69. An isolated polypeptide comprising a nucleotide sequence or a fragment thereof encoding a polypeptide with enzymatic activity involved in or associated with phytosterol and brassinosteroid metabolism in a plant, said nucleotide sequence selected from the group consisting of SEQ ID NO 3, 5, 13, 15, 21, 33, 35, 37, 45, 49, 59, 61, 65, 91, 95, 119, 123, 131, 147, 163, 169, 173, 183, 187, 195, 197, 219, 251, 269, 287, 303, 309, 311, 323, 329, 331, 333, 337, 341, 361, 367, 379, 389, and 405.

70. An isolated polypeptide comprising a nucleotide sequence substantially similar to the nucleotide sequence of the polynucleotide of claim 69.

71. An expression cassette comprising the polynucleotide of claim 69.

72. A host cell comprising the expression cassette of claim 71.

73. A transgenic plant comprising the expression cassette of claim 71.

74. An isolated polypeptide comprising a nucleotide sequence encoding a polypeptide with enzymatic activity involved in or associated with phytosterol and brassinosteroid metabolism in a plant, said polypeptide comprising an amino acid sequence selected from the group consisting of SEQ ID NO 4, 6, 14, 16, 22, 34, 36, 38, 46, 50, 60, 62, 66, 92, 96, 120, 124, 132, 148, 164, 170, 174, 184, 188, 196, 198, 220, 252, 270, 288, 304, 310, 312, 324, 330, 332, 334, 338, 342, 362, 368, 380, 390, and 406.

75. A expression cassette comprising the polynucleotide of claim 74.

76. A host cell comprising the expression cassette of claim 75.

77. A transgenic plant comprising the expression cassette of claim 75.

78. An isolated polypeptide comprising a nucleotide sequence or fragment thereof encoding a polypeptide with activity involved in or associated with biosynthesis of ubiquinone in a plant said nucleotide sequence selected from the group consisting of SEQ ID NO 7, 239 and 381.

79. An isolated polypeptide comprising a nucleotide sequence substantially similar to the nucleotide sequence of the polynucleotide of claim 78.

80. An expression cassette comprising the polynucleotide of claim 78.

81. A host cell comprising the expression cassette of claim 80.

82. A transgenic plant comprising the expression cassette of claim 81.

83. An isolated polypeptide comprising a nucleotide sequence encoding a polypeptide with activity involved in or associated with biosynthesis of ubiquinone in a plant said polypeptide comprising an amino acid sequence selected from the group consisting of SEQ ID NO 8, 240 and 382.

84. An expression cassette comprising the polynucleotide of claim 83.

85. A host cell comprising the expression cassette of claim 84.

86. A transgenic plant comprising the expression cassette of claim 84.

87. An isolated polynucleotide comprising a nucleotide sequence or fragment thereof encoding a polypeptide with activity involved in or associated with the biosynthesis of monterpenes and sesquiterpenes in a plant, said polynucleotide comprising a nucleotide sequence selected from the group consisting of SEQ ID NO 1, 9, 19, 39, 41, 43, 51, 55, 57, 69, 71, 77, 81, 99, 101, 109, 115, 129, 137, 145, 151, 157, 159, 167, 171, 179, 181, 185, 201, 209, 211, 217, 227, 231, 233, 241, 247, 249, 257, 263, 273, 279, 281, 283, 305, 319, 321, 327, 347, 351, 357, 369, 375, 385, 387 and 403.

88. An isolated polynucleotide comprising a nucleotide sequence substantially similar to the nucleotide sequence of the polynucleotide of claim 87.

89. An expression cassette comprising the polynucleotide of claim 87.

90. A host cell comprising the expression cassette of claim 89.

91. A transgenic plant comprising the expression cassette of claim 89.

92. An isolated polynucleotide comprising a nucleotide sequence encoding a polypeptide with activity involved in or associated with the biosynthesis of monterpenes and sesquiterpenes in a plant, said polypeptide comprising an amino acid sequence selected from the group consisting of SEQ ID NO 2, 10, 20, 40, 42, 44, 52, 56, 58, 70, 72, 78, 82, 100, 102, 110, 116, 130, 138, 146, 152, 158, 160, 168, 172, 180, 182, 186, 202, 210, 212, 218, 228, 232, 234, 242, 248, 250, 258, 264, 274, 280, 282, 284, 306, 320, 322, 328, 348, 352, 358, 370, 376, 386, 388 and 404.

93. An expression cassette comprising the polynucleotide of claim 92.

94. A host cell comprising the expression cassette of claim 93.

95. A transgenic plant comprising the expression cassette of claim 93.

96. An isolated polynucleotide comprising a nucleotide sequence or fragment thereof encoding a polypeptide that is involved in or associated with protein prenylation, said nucleotide sequence selected from the group consisting of SEQ ID NO 23, 63, 189, 255, 289, 343, and 373.

97. An isolated polynucleotide comprising a nucleotide sequence substantially similar to the nucleotide sequence of the polynucleotide of claim 96.

98. An expression cassette comprising the polynucleotide of claim 96.

99. A host cell comprising the expression cassette of claim 98.

100. A transgenic plant comprising the expression cassette of claim 98.

101. An isolated polynucleotide comprising a nucleotide sequence encoding a polypeptide that is involved in or associated with protein prenylation, said polypeptide comprising an amino acid sequence selected from the group consisting of SEQ ID NO 24, 64, 190, 256, 290, 344, and 374.

102. An expression cassette comprising the polynucleotide of claim 101.

103. A host cell comprising the expression cassette of claim 102.

104. A transgenic plant comprising the expression cassette of claim 102.

105. An isolated polynucleotide comprising a nucleotide sequence or fragment thereof encoding a polypeptide involved in or associated with the biosynthesis of chlorophyll or heme, said nucleotide sequence selected from the group consisting of SEQ ID NO 31, 47, 53, 105, 117, 127, 139, 143, 149, 175, 177, 191, 199, 221, 277, 295, 307, 325, 335, 339, 355, and 363.

106. An isolated polynucleotide comprising a nucleotide sequence substantially similar to the nucleotide sequence of the polynucleotide of claim 105.

107. An expression cassette comprising the polynucleotide of claim 105.

108. A host cell comprising the expression cassette of claim 107.

109. A transgenic plant comprising the expression cassette of claim 107.

110. An isolated polynucleotide comprising a nucleotide sequence encoding a polypeptide involved in or associated with the biosynthesis of chlorophyll or heme, said polypeptide comprising an amino acid sequence selected from the group consisting of SEQ ID NO 32, 48, 54, 106, 118, 128, 140, 144, 150, 176, 178, 192, 200, 222, 278, 296, 308, 326, 336, 340, 356, and 364.

111. An expression cassette comprising the polynucleotide of claim 110.

112. A host cell comprising the expression cassette of claim 111.

113. A transgenic plant comprising the expression cassette of claim 111.
